(19) United States
(12) Patent Application Publication
(10) Pub. No.: US 2026/0139273 A1
(43) Pub. Date: May 21, 2026

(54) Synergistic Nucleic Acid Based Therapeutics and Methods of Use for Treating Genetic Disorders

(71) Applicant: UNIVERSITY OF MIAMI, Miami, FL (US)

(72) Inventor: Claes Wahlestedt, Miami, FL (US)

(21) Appl. No.: 19/103,051

(22) PCT Filed: Aug. 14, 2023
(86) PCT No.: PCT/US23/72179

§ 371 Date: Feb. 11, 2025

Related U.S. Application Data
(60) Provisional application No. 63/371,351, filed on Aug. 12, 2022.

(51) Int. Cl. C12N 15/864; A61K 31/7105; A61P 25/28; C12N 15/113; C12N 15/867

(52) U.S. Cl. CPC C12N 15/8645; A61K 31/7105; A61P 25/28; C12N 15/1136; C12N 15/867; C12N 2310/11; C12N 2740/15043; C12N 2750/14143; C12N 2800/107

(57) ABSTRACT

The present disclosure relates, in general, to methods for the modulation of a target gene expression by the combined use of nucleic acid based therapeutics targeting complementary gene regulation mechanisms (upNA), leading to desired effects in excess of sum of the effects of each treatment alone, as well as the use of the combinations for the treatment of genetic (e.g., neurological) diseases and disorders associated with aberrant expression of the target gene(s).

### CROSS REFERENCE TO RELATED APPLICATIONS

The present application claims the priority benefit of U.S. Provisional Patent Application No. 63/371,351, filed Aug. 12, 2022, herein incorporated by reference in its entirety.

### INCORPORATION BY REFERENCE OF SEQUENCE LISTING

The Sequence Listing, which is a part of the present disclosure, is submitted concurrently with the specification as an XML file. The name of the XML file containing the Sequence Listing is “58047_Seqlisting.XML”, which was created on Aug. 14, 2023 and is 813,371 bytes in size. The subject matter of the Sequence Listing is incorporated by reference herein in its entirety.

### FIELD OF THE DISCLOSURE

The present invention relates to means for synergistically modulating expression of target genes via multiple regulatory mechanisms using combinations of nucleic acid based drugs to achieve amplified effect and higher potency than the sum of effects of each drug alone, and, in particular, means for synergistically modulating the expression of target genes whose aberrant expression is associated with genetic diseases and disorders, including neurological disorders.

### BACKGROUND

Conventional drug strategy relies on the ability of small molecule drugs to target active sites of proteins so as to inhibit or alter their function. However, only ˜1.5% of the human genome encodes proteins (Ezkurdia et al., 2014) and only 10-14% of proteins have suitable binding sites that are “druggable” by small molecules (Hopkins and Groom, 2002). Thus the “druggable” targets for small molecule therapies are limited. This limitation was addressed in part by revolution in recombinant protein technology. However, recombinant proteins have limitations as drugs, particularly due to size and stability issues (Antosova et al., 2009; Lam et al., 2015). Furthermore, they must be properly folded and often require post-translational modifications (Li et al., 2015) which complicates their manufacturing process. In recent years, nucleic acid-based drugs (NBTs) have emerged as an alternative to the conventional protein-based therapies for undruggable targets.

The primary focus of NBTs includes gene silencing or activation and splice modulation that provides an extended range of potential targets beyond what is targeted by conventional pharmacological strategies. Majority of the NBT modalities follow the universal Watson-Crick base pairing rule of complementarity, thus providing the direct interrogation of different putative target sequences. With the understanding of new biological processes and development of novel technologies, diverse NBTs platforms have been developed to modulate potential therapeutic targets.

For example: RNA aptamers can directly bind to extracellular, cell surface, or intracellular proteins (Gragoudas et al., 2004; Gryziewicz, 2005) that are traditionally targeted by small-molecule and protein drugs. ASOs, RNAi (siRNAs, and miRNA mimics) may be delivered into cells to target intracellular mRNAs or functional non-coding (ncRNAs) through complementary base pairings, leading to gene silencing or activation and control of gene expression for the treatment of diseases. An mRNA molecule can be introduced into cells and then translated into target proteins for protein replacement therapy or vaccination. A non-coding RNA (ncRNA) molecule can be similarly introduced into cells to control gene expression. In addition, the genetic sequences dictating disease initiation and progression may be directly changed by using guide RNAs (gRNAs) along with other necessary components to directly edit the target gene sequences for the treatment of particular diseases.

### SUMMARY

The present disclosure provides combinations of nucleic acid based molecules (ultra-potent nucleic acids, or upNA) that act on multiple regulatory mechanisms that control the expression of one or several target genes, thereby synergistically modulating the expression of the target gene(s) to achieve amplified effect and a higher potency than the sum of effects of each molecule alone. Methods are provided wherein synergistic up-regulation or inhibition of target genes using the combination of nucleic acid based molecules described herein, are effective in the treatment of diseases and disorders, such as genetic diseases or disorders, such as neurological disorders, associated with aberrant expression of said target genes. Compositions of the nucleic acid based molecule combinations and methods of treatment are encompassed within the disclosure.

In one aspect, the disclosure provides a method for modulating the expression level of a target gene in a cell. In various embodiments, the disclosure provides a method for modulating the expression level of a target gene in a cell comprising: contacting the cell with a combination of two or more nucleic acid based molecules (upNA), including at least a first nucleic acid based molecule and a second nucleic acid based molecule, wherein the nucleic acid based molecules each has one of the following effects;
a) modulating the action of a natural antisense transcript (NAT) that controls the expression of the target protein;
b) modulating the splicing of a mutated exon or intron or a nonsense mediated RNA decay-inducing exon in mRNA encoding a target protein encoded by the target gene or a target regulatory non-coding RNA (ncRNA);
c) inhibiting the effect of a translation suppression element in the 5′ untranslated region of a transcript of the target gene, wherein the translation suppression element is an upstream open reading frame (uORF);
d) knocking down an mRNA encoding a pathogenic isoform of the target protein or of a target regulatory ncRNA involved in a pathogenic process;
e) knocking down all isoforms of the mRNA of the target protein or target ncRNA;
f) modulating effects of promoter RNAs affecting in cis or in trans the expression from the genomic locus encoding the target protein or target ncRNA;
g) modulating effects of enhancer RNAs affecting in cis or in trans the expression from the genomic locus encoding the target protein or target ncRNA, or
h) replicating the effects of the natural SINEUP transcript through inclusion of its essential parts.

In various embodiments, contacting the cell with a combination of two or more nucleic acid based molecules (e.g., upNA) leads to effects in excess of the sum of the effects of each treatment alone.

Also contemplated is a method for modulating the expression level of a target gene, wherein the target gene can be modulated through multiple regulatory mechanisms in a cell, comprising: contacting the cell with a nucleic acid based molecule, including at least a first functional domain and a second functional domain, wherein each one of the functional domains has a different effect. In various embodiments, contacting the cell with two functional domains leads to desired effects in excess of sum of the effects of each domain alone.

The disclosure also provides a method for modulating the expression level of two or more target genes in a cell comprising: contacting the cell with two or more nucleic acid based molecules, including at least a first nucleic acid based molecule and a second nucleic acid based molecule, wherein each nucleic acid based molecule regulates the expression of a different target gene. In various embodiments, contacting the cell with two nucleic acid based molecules leads to desired effects in excess of sum of the effects of each alone.

In various embodiments, the disclosure contemplates a method for modulating the expression level of two or more target genes in a cell comprising: contacting the cell with a nucleic acid based molecule, including at least a first functional domain and a second functional domain, wherein each one of the functional domains regulates the expression of a different gene. In various embodiments, contacting the cell with two functional domains leads to desired effects in excess of sum of the effects of each domain alone.

Further provided is a method of treating a disease or disorder in a subject in need thereof by modulating the expression level of a target gene in a cell of the subject comprising contacting the cell with one or more nucleic acid based molecules, including at least a first nucleic acid based molecule and a second nucleic acid based molecule, wherein the gene associated with a disease or condition can be modulated through multiple regulatory mechanisms. In various embodiments, contacting the cell with two nucleic acid based molecule leads to desired effects in excess of sum of the effects of each molecule alone

In various embodiments, the disclosure provides a method of treating a disease or disorder in a subject in need thereof by modulating the expression level of a target gene in a cell of the subject comprising contacting the cell with one or more nucleic acid based molecules, including at least a first functional domain and a second functional domain, wherein each functional domain has a different effect, wherein the gene associated with a disease or condition can be modulated through multiple regulatory mechanisms. In various embodiments, contacting the cell with two functional domains leads to desired effects in excess of sum of the effects of each domain alone

In various embodiments, provided herein is a method of treating a disease or disorder in a subject in need thereof by modulating the expression level of two or more target genes in a cell of the subject comprising contacting the cell with one or more nucleic acid based molecules, including at least a first nucleic acid based molecule and a second nucleic acid based molecule that modulate two or more different target genes associated with a disease or condition, wherein a disease or condition can be modulated through simultaneous regulation of multiple target genes. In various embodiments, contacting the cell with two functional nucleic acid based molecules leads to desired effects in excess of sum of the effects of each molecule alone

The disclosure also contemplates a method of treating a disease or disorder in a subject in need thereof by modulating the expression level of two or more target genes in a cell of the subject comprising contacting the cell with one or more nucleic acid based molecules, including at least a first functional domain and a second functional domain that modulate two or more different target genes associated with a disease or condition, wherein a disease(s) or condition(s) can be modulated through simultaneous regulation of multiple target genes In various embodiments, contacting the cell with two functional domains leads to desired effects in excess of sum of the effects of each domain alone.

In various embodiments, the method comprises one or more nucleic acid based molecules (e.g., upNAs), wherein at least one of the domains of the nucleic acid based molecules has one of the following effects;
a) modulating the action of a natural antisense transcript (NAT) that controls the expression of the target protein;
b) modulating the splicing of a mutated exon or intron or a nonsense mediated RNA decay-inducing exon in mRNA encoding a target protein or target mRNA encoded by the target gene;
c) inhibiting the effect of a translation suppression element in the 5′ untranslated region of a transcript of the target gene, wherein the translation suppression element is an upstream open reading frame (uORF);
d) knocking down an mRNA encoding a pathogenic isoform of the target protein or of a target regulatory ncRNA involved in a pathogenic process;
e) knocking down all isoforms of the mRNA of the target protein or target ncRNA;
f) modulating effects of promoter RNAs affecting in cis or in trans the expression from the genomic locus encoding the target protein or target ncRNA;
g) modulating effects of enhancer RNAs affecting in cis or in trans the expression from the genomic locus encoding the target protein or target ncRNA, or
h) replicating the effects of the natural SINEUP transcript through inclusion of its essential parts.

In various embodiments, the nucleic acid based molecule with multiple functional domains (e.g., upNA) is selected from a multi-domain nucleic acid based molecule, plasmid vector, lentiviral vector or rAAV-based vector wherein the rAAV is an AAV1, AAV2, AAV5, AAV6, AAV7, AAV8, AAV9 or AAV-recombinant human 10 (AAVrh.10).

In various embodiments, the nucleic acid based molecules are selected from the group consisting of an antisense oligonucleotide (ASO), a guide RNA (gRNA), an siRNA, an shRNA, a small nuclear RNA (snRNA), a mini SINEUP, and an miRNA. In various embodiments, at least one of the nucleic acid based molecules is an antisense oligonucleotide (ASO).

In various embodiments, the ASO (AntagoNAT) binds to a natural antisense transcript (NAT), thereby increasing the expression of the sense transcript (mRNA) that encodes a target protein encoded by the target gene.

In various embodiments, the ASO is 20 to 30 nucleotides long.

In various embodiments, the ASO is at least 90% complementary to the target site. In various embodiments, the ASO is 100% complementary to the target site.

In various embodiments, the ASO is single-stranded.

In various embodiments, the method further comprises a second nucleic acid based molecule.

In various embodiments, the treatment with a combination of nucleic acid molecules results in synergistic effect, exceeding the sum of the effects of the two separate treatments by at least 30%, e.g., at least 30%, at least 35%, at least 40%, at least 45%, at least 50%, at least 55%, at least 60%, at least 65%, at least 70%, at least 75%, at least 80%, at least 85%, at least 90%, or 100%

In various embodiments, the molecules comprising upNA are present in concentrations reflecting their individual potencies.

In various embodiments, the target gene is human C90RF72.

In various embodiments, the ASO binds a target site on a human C90RF72 natural antisense transcript (NAT).

In various embodiments, the natural antisense transcript for C90RF72 has the nucleic acid sequence as set forth in SEQ ID NOs: 522-525 and 536-539.

In various embodiments, the ASO comprises a sequence has at least about 80%, 85%, 90%, 95%, 97%, or 100% complementarity to all or a portion of any one of SEQ ID NOs: 522-525 and 536-539. In various embodiments, the ASO comprises the sequences set forth as SEQ ID NOs: 540-551. In various embodiments, the ASO binds a target site on a human C90RF72 natural antisense transcript, wherein said ASO increases the expression of human C90RF72 (SEQ ID NOs: 533-535).

In various embodiments, the NAT-targeting oligonucleotide is an ASO and binds a target site on a human SCN1A natural antisense transcript, wherein said ASO increases the expression of human SCN1A (SEQ ID NO: 575), and wherein the natural antisense transcript has the nucleic acid sequence as set forth in any one of SEQ ID NOs: 576-582. In various embodiments, the ASO comprises a sequence that is at least about 80%, 85%, 90%, 95%, 97%, or 100% complementary to all or a portion of any one of SEQ ID NOs: 576-582.

In various embodiments, the NAT-targeting oligonucleotide is an ASO and binds a target site on a human SLC2A1 (solute carrier family 2 member 1) natural antisense transcript, wherein said ASO increases the expression of human SLC2A1 (SEQ ID NO: 287), and wherein the natural antisense transcript has the nucleic acid sequence as set forth in any one of SEQ ID NOs: 146-148. In various embodiments, the ASO comprises a sequence that is at least about 80%, 85%, 90%, 95%, 97%, or 100% complementary to all or a portion of any one of SEQ ID NOs: 146-148.

In various embodiments, the NAT-targeting oligonucleotide is an ASO and binds a target site on a human optic atrophy type 1 (OPA1) natural antisense transcript, wherein said ASO increases the expression of human OPA1 (SEQ ID NO: 116), and wherein the natural antisense transcript has the nucleic acid sequence as set forth in any one of SEQ ID NO: 117. In various embodiments, the ASO comprises a sequence that is at least about 80%, 85%, 90%, 95%, 97%, or 100% complementary to all or a portion of any one of SEQ ID NO: 117.

In various embodiments, the NAT-targeting oligonucleotide is an ASO and binds a target site on a human phosphatidylinositol binding clathrin-assembly protein (PICALM) natural antisense transcript, wherein said ASO increases the expression of human PICALM (SEQ ID NO: 345), and wherein the natural antisense transcript has the nucleic acid sequence as set forth in any one of SEQ ID NO: 346-350. In various embodiments, the ASO comprises a sequence that is at least about 80%, 85%, 90%, 95%, 97%, or 100% complementary to all or a portion of any one of SEQ ID NO: 346-350.

In various embodiments, the NAT-targeting oligonucleotide is an ASO and binds a target site on a human Low density lipoprotein receptor-related protein 1 (LRP1) natural antisense transcript, wherein said ASO increases the expression of human LRP1, and wherein the natural antisense transcript has the nucleic acid sequence as set forth in any one of SEQ ID NO: 467-473. In various embodiments, the ASO comprises a sequence that is at least about 80%, 85%, 90%, 95%, 97%, or 100% complementary to all or a portion of any one of SEQ ID NO: 467-473.

In various embodiments, the NAT-targeting oligonucleotide is an ASO and binds a target site on a human frataxin (FXN) natural antisense transcript, wherein said ASO increases the expression of human FXN, and wherein the natural antisense transcript has the nucleic acid sequence as set forth in any one of SEQ ID NO: 526-531. In various embodiments, the ASO comprises a sequence that is at least about 80%, 85%, 90%, 95%, 97%, or 100% complementary to all or a portion of any one of SEQ ID NO: 526-531.

In various embodiments, the first or second nucleic acid based molecule is an siRNA.

In various embodiments, the siRNA is specific for RAPTOR and is set out in any one of SEQ ID NOs: 33-36.

In various embodiments, the siRNA is specific for RICTOR and is set out in any one of SEQ ID NOs: 56-115.

In various embodiments, the siRNA is specific for SCN1A and is set out in any one of SEQ ID NOs: 1-32.

In various embodiments, the siRNA is specific for SLC2A1 and is set out in any one of SEQ ID NOs: 149-344.

In various embodiments, the siRNA is specific for OPA1 and is set out in any one of SEQ ID NOs: 118-145.

In various embodiments, the siRNA is specific for PICALM and is set out in any one of SEQ ID NOs: 351-466.

In various embodiments, the siRNA is specific for LRP1 and is set out in any one of SEQ ID NOs: 474-517.

In various embodiments, the ASOs or siRNA can comprise one or more modified nucleotides, modified bonds and the like.

In various embodiments, the target gene is selected from the group consisting of genes as set out in FIG. 8.

In various embodiments, the ASO increases the expression of human C90RF72 (SEQ ID NOs: 533-535), RAPTOR, RICTOR, SCN1A, SLC2A1, LRP1, OPA1, or PICALM.

In various embodiments, the ASO increases the expression of human C90RF72 by at least 30%, at least 50%, at least 55%, at least 60%, at least 65%, at least 70%, at least 75%, at least 80%, at least 85%, at least 90%, or 100%.

In various embodiments, the ASO increases the expression of human C90RF72, RAPTOR, RICTOR, SCN1A, SLC2A1, LRP1, OPA1 or PICALM by at least 30%, at least 50%, at least 55%, at least 60%, at least 65%, at least 70%, at least 75%, at least 80%, at least 85%, at least 90%, or 100%.

In various embodiments, the ASO alters (e.g., decreases) the activity of a natural antisense transcript of human C90RF72 by at least 30%, at least 50%, at least 55%, at least 60%, at least 65%, at least 70%, at least 75%, at least 80%, at least 85%, at least 90%, or 100%.

In various embodiments, the ASO alters (e.g., decreases) the activity of a natural antisense transcript of human C90RF72, RAPTOR, RICTOR, SCN1A, SLC2A1, LRP1, OPA1 or PICALM by at least 30%, at least 50%, at least 55%, at least 60%, at least 65%, at least 70%, at least 75%, at least 80%, at least 85%, at least 90%, or 100%.

In various embodiments, one or more nucleic acid based molecules is a gapmer ASO. In various embodiments, one or more nucleic acid based molecules is a gapmer ASO comprising a 5′ “wing” comprising of 1-6 locked nucleic acid (LNA) nucleotides, a core “gap” comprising between 5-10 contiguous DNA nucleotides and a 3′ “wing” comprising of 1-6 locked nucleic acid (LNA) nucleotides.

In various embodiments, the “gap” sequence is capable of recruiting RNAseH after forming a DNA/RNA duplex with a complementary RNA molecule.

In various embodiments, the “gap” sequence is complementary to a region within a C90RF72 transcript. In various embodiments, the “gap” sequence is complementary to a region within a C90RF72, RAPTOR, RICTOR, SCN1A, SLC2A1, LRP1, OPA1 or PICALM transcript.

In various embodiments, the “gap” sequence is at least 50%, at least 55%, at least 60%, at least 65%, at least 70%, at least 75%, at least 80%, at least 85%, at least 90%, at least 95%, or 100% complementary to an equal length portion of a region within a C90RF72 transcript. In various embodiments, the “gap” sequence is at least 50%, at least 55%, at least 60%, at least 65%, at least 70%, at least 75%, at least 80%, at least 85%, at least 90%, at least 95%, or 100% complementary to an equal length portion of a region within a C90RF72, RAPTOR, RICTOR, SCN1A, SLC2A1, LRP1, OPA1 or PICALM transcript.

In various embodiments, binding of the gapmer ASO to a region within a C90RF72 transcript preferentially inhibits the expression of C90RF72 isoforms V1 and V3.

In various embodiments, the V1 and V3 isoforms of C90RF72 contain a hexanucleotide repeat expansion.

In various embodiments, the expression of mutant C90RF72 isoforms V1 and V3 is decreased by at least 30%, at least 50%, at least 55%, at least 60%, at least 65%, at least 70%, at least 75%, at least 80%, at least 85%, at least 90%, at least 95%, or 100%.

In various embodiments, the gapmer ASO comprises a sequence that is at least about 80%, 85%, 90%, 95%, 97%, or 100% complementary to all or a portion of any one of C90RF72 NATs having the nucleic acid sequences as set forth in SEQ ID NOs: 522-525 and 536-539.

In various embodiments, the gapmer ASO increases the levels of isoform V2 of C90RF72 by at least 50%, at least 55%, at least 60%, at least 65%, at least 70%, at least 75%, at least 80%, at least 85%, at least 90%, or 100%.

In various embodiments, the at least one nucleic acid based molecule is a synthetic U7 snRNA.

Endogenous U7 snRNAs participate in histone pre-mRNA processing due to the presence of two domains: 1) a domain that interacts with histone pre-mRNA through complementary base-pairing, and 2) a domain that interacts with small nuclear ribonucleoprotein (snRNP) complex and positions it at histone pre-mRNA molecule. In various embodiments, the synthetic U7 snRNA is constructed by replacing domain 1 of U7 snRNA with a sequence from the vicinity of the intron 1 splice sites of C90RF72 pre-mRNA.

In various embodiments, the synthetic U7 snRNAs facilitate intron 1 excision. In various embodiments, excision of intron 1 reduces the expression of the mRNA transcripts harboring the repeat expansion mutation and increases the expression of productive C90RF72 transcript that can be translated into functional protein.

In various embodiments, the synthetic U7 snRNA decreases the expression of the mutant C90RF72 isoforms by at least 50%, at least 55%, at least 60%, at least 65%, at least 70%, at least 75%, at least 80%, at least 85%, at least 90%, or 100% and increases expression of productive C90RF72 transcript by at least 50%, at least 55%, at least 60%, at least 65%, at least 70%, at least 75%, at least 80%, at least 85%, at least 90%, or 100%.

In various embodiments, the synthetic U7snRNA constructs can be encoded by a viral vector, which will enable the delivery of this splicing modulator into a range of cells and tissues.

In various embodiments, the ASOs (components of upNAs) binds a target site on a human RICTOR natural antisense transcript (RICTOR NAT), wherein said ASOs increase the expression of human RICTOR (SEQ ID NO: 552), and wherein the natural antisense transcripts have the nucleic acid sequences as set forth in SEQ ID NOs: 37, 38-40, 41-55, 553-573.

In various embodiments, the RICTOR upNAs comprise sequences that have at least about 80%, 85%, 90%, 95%, 97%, or 100% complementarity to all or a portion of any one of SEQ ID NOs: 37, 38-40, 41-55, 553-573.

In various embodiments, the ASO is 20 to 30 nucleotides long.

In various embodiments, the ASO is at least 90% complementary to the target site. In various embodiments, the ASO is 100% complementary to the target site.

In various embodiments, the ASO is single-stranded.

In various embodiments, the method further comprises a second nucleic acid based molecule. In various embodiments, the second nucleic acid based molecule is an ASO or an siRNA, wherein such nucleic acid based molecules knock down the mRNA encoding RAPTOR protein having the sequence set forth in SEQ ID NO: 574. In various embodiments, the oligonucleotides comprise a sequence that is at least about 80%, 85%, 90%, 95%, 97%, or 100% complementary to all or a portion of SEQ ID NO:574.

In various embodiments, the knockdown of RAPTOR simultaneously induces upregulation of RICTOR independently of upNA-mediated modulation of RICTOR NAT activity.

In various embodiments, the nucleic acid based molecules are 20 to 30 nucleotides long.

In various embodiments, the expression of said RICTOR gene is increased by at least 30% and expression of the RAPTOR gene is decreased at least 30%.

In one aspect, the disclosure provides a composition comprising two or more nucleic acid based molecules as described herein.

In various embodiments, the compositions comprise one or more ASO and or first or second nucleic acid based molecules as disclosed herein useful in the methods. It is further contemplated that the methods and compositions can be carried out with a mix of ASO or nucleic based molecules, such as siRNA that target the same or related molecular pathways associated with a genetic disease or disorder.

In various embodiments, the composition comprises NAT-targeting ASO that binds a target site on a human C90RF72 natural antisense transcript, wherein said oligonucleotide increases the expression of human C90RF72 (SEQ ID NOs: 533-535), and wherein the natural antisense transcript has the nucleic acid sequence as set forth in any one of SEQ ID NOs: 522-525 and 536-539. In various embodiments, the ASO comprises a sequence that is at least about 80%, 85%, 90%, 95%, 97%, or 100% complementarity to all or a portion of any one of SEQ ID NOs: 522-525 and 536-539.

In various embodiments, the composition comprises a NAT-targeting ASO that binds a target site on a human RICTOR natural antisense transcript, wherein the natural antisense transcript has the nucleic acid sequence as set forth in any one of SEQ ID NOs: 37, 38-40, 41-55, and 553-573. In various embodiments, wherein the ASO comprises a sequence that is at least about 80%, 85%, 90%, 95%, 97%, or 100% complementary to all or a portion of any one of SEQ ID NOs: 522-525 and 536-539.

In various embodiments, the composition comprises a NAT-targeting ASO that binds a target site on a human SCN1A natural antisense transcript, wherein the natural antisense transcript has the nucleic acid sequence as set forth in any one of SEQ ID NOs: 576-582. In various embodiments, the ASO comprises a sequence that is at least about 80%, 85%, 90%, 95%, 97%, or 100% complementary to all or a portion of any one of SEQ ID NOs: 576-582.

In various embodiments, the composition comprises a NAT-targeting ASO that binds a target site on a human SLC2A1 (solute carrier family 2 member 1) natural antisense transcript, wherein the natural antisense transcript has the nucleic acid sequence as set forth in any one of SEQ ID NOs: 146-148. In various embodiments, the ASO comprises a sequence that is at least about 80%, 85%, 90%, 95%, 97%, or 100% complementary to all or a portion of any one of SEQ ID NOs: 146-148.

In various embodiments, the composition comprises a NAT-targeting ASO that binds a target site on a human optic atrophy type 1 (OPA1) natural antisense transcript, wherein said ASO increases the expression of human OPA1 (SEQ ID NO: 116), and wherein the natural antisense transcript has the nucleic acid sequence as set forth in any one of SEQ ID NO: 117. In various embodiments, the ASO comprises a sequence that is at least about 80%, 85%, 90%, 95%, 97%, or 100% complementary to all or a portion of any one of SEQ ID NO: 117.

In various embodiments, the composition comprises a NAT-targeting ASO that binds a target site on a human phosphatidylinositol binding clathrin-assembly protein (PICALM) natural antisense transcript, wherein said ASO increases the expression of human PICALM (SEQ ID NO: 345), and wherein the natural antisense transcript has the nucleic acid sequence as set forth in any one of SEQ ID NO: 346-350. In various embodiments, the ASO comprises a sequence that is at least about 80%, 85%, 90%, 95%, 97%, or 100% complementary to all or a portion of any one of SEQ ID NO: 346-350.

In various embodiments, the composition comprises a NAT-targeting ASO that binds a target site on a human Low density lipoprotein receptor-related protein 1 (LRP1) natural antisense transcript, wherein said ASO increases the expression of human LRP1, and wherein the natural antisense transcript has the nucleic acid sequence as set forth in any one of SEQ ID NO: 467-473. In various embodiments, the ASO comprises a sequence that is at least about 80%, 85%, 90%, 95%, 97%, or 100% complementary to all or a portion of any one of SEQ ID NO: 467-473.

In various embodiments, the composition comprises a NAT-targeting ASO that binds a target site on a human frataxin (FXN) natural antisense transcript, wherein said ASO increases the expression of human FXN, and wherein the natural antisense transcript has the nucleic acid sequence as set forth in any one of SEQ ID NO: 526-531. In various embodiments, the ASO comprises a sequence that is at least about 80%, 85%, 90%, 95%, 97%, or 100% complementary to all or a portion of any one of SEQ ID NO: 526-531.

In various embodiments, the composition comprises an siRNA.

In various embodiments, the composition comprises siRNA specific for C90RF72 set out in any one of SEQ ID NOs: 33-36.

In various embodiments, the composition comprises siRNA specific for RAPTOR set out in any one of SEQ ID NOs: 33-36.

In various embodiments, the composition comprises siRNA specific for RICTOR set out in any one of SEQ ID NOs: 56-115.

In various embodiments, the composition comprises siRNA specific for SCN1A set out in any one of SEQ ID NOs: 1-32.

In various embodiments, the composition comprises siRNA specific for SLC2A1 set out in any one of SEQ ID NOs: 149-344.

In various embodiments, the composition comprises siRNA specific for OPA1 set out in any one of SEQ ID NOs: 118-145.

In various embodiments, the composition comprises siRNA specific for PICALM set out in any one of SEQ ID NOs: 351-466.

In various embodiments, the composition comprises siRNA specific for LRP1 set out in any one of SEQ ID NOs: 474-517. In various embodiments, the two or more nucleic acid based molecules are formulated in a liposome, a nanoparticle, a lipid nanoparticle, an exosome, or a microvesicle in concentrations that are effective for the activity of corresponding molecules.

In one aspect, the disclosure further provides a vector comprising two or more nucleic acid based molecules as described herein. In various embodiments, the nucleic acid based molecules are expressed by a plasmid vector or a viral vector. In various embodiments, the viral vector is a recombinant adeno-associated virus (rAAV) or a lentivirus. In various embodiments, the two or more nucleic acid based molecules are formulated in a liposome, a nanoparticle, a lipid nanoparticle, an exosome, or a microvesicle.

In various embodiments, the two or more nucleic acid based molecules are expressed from a recombinant adeno-associated virus vector (rAAV). In various embodiments, the vector is a rAAV vector selected from the group consisting of AAV1, AAV2, AAV5, AAV6, AAV7, AAV8, AAV9 or AAV-recombinant human 10 (AAVrh.10). In various embodiments, the vector is AAV9.

In various embodiments, the two or more nucleic acid based molecules are expressed by the same vector or by different vectors.

In various embodiments, at least one nucleic acid based molecule alters the action of a natural antisense transcript (NAT). In various embodiments, the altering can be inhibiting the action of a NAT or enhancing the action of a NAT.

In various embodiments, the method of treatment comprises contacting a cell in a subject in need thereof with the nucleic acid based molecules, vector or compositions described herein. In various embodiments, the method leads to desired effects in excess of sum of the effects of each treatment alone.

In various embodiments, the disease or disorder is a genetic disease or disorder associated with aberrant gene expression. In various embodiments, the disease or disorder is a CNS disease or disorder associated with aberrant gene expression.

In various embodiments, the CNS disease or disorder associated with aberrant expression of the target gene(s) is selected from the group consisting of: Alzheimer's disease (AD), Friedrich's ataxia (FA), Huntington's disease (HD), sodium channel a2 subunit (SCN2A) encephalopathy, sodium channel a8 subunit (SCN8A) encephalopathy, SCN1A-Associated Dravet Syndrome, C9orf72-mediated amyotrophic lateral sclerosis (ALS) and frontotemporal dementia (FTD), sodium channel a5 subunit (SCN5A).

In various embodiments, the genetic disease or disorder associated with aberrant expression of the target gene(s) is selected from the group consisting of: Alzheimer's disease (AD), Friedrich's ataxia (FA), Huntington's disease (HD), sodium channel a2 subunit (SCN2A) encephalopathy, sodium channel a8 subunit (SCN8A) encephalopathy, SCN1A-Associated Dravet Syndrome, C9orf72-mediated amyotrophic lateral sclerosis (ALS) and frontotemporal dementia (FTD), sodium channel a5 subunit (SCN5A) arrhythmia or tauopathies, such as Pick disease, progressive supranuclear palsy, corticobasal degeneration, argyrophilic grain disease, globular glial tauopathies, neurofibrillary tangle dementia, chronic traumatic encephalopathy or aging-related tau astrogliopathy.

In various embodiments, the disease or disorder is C9orf72-mediated amyotrophic lateral sclerosis (ALS) and frontotemporal dementia (FTD).

In various embodiments, the two or more nucleic acid based molecules, compositions or vectors are administered to the subject intrathecally, intranasally, intranasally via nasal depot, intracerebroventricularly, intraperitoneally, intramuscularly, subcutaneously, orally, synovially, intravitreally, subretinally, or intravenously.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1A is a bar graph showing total SCN1A transcripts in SK-N-AS human neuroblastoma cell line treated with different concentrations of AntagoNATs targeting SCN1ANAT (1002) and splice modulating ASOs targeting the SCN1A pre-mRNA (CTI-1008) for 48 hours. Transcript levels were measured by qPCR, normalized to GAPDH, and compared to cells treated with inactive control oligonucleotide. FIG. 1B is a bar graph showing fold change of productive SCN1A transcripts expressed in the SK-N-AS human neuroblastoma cell line treated with different concentrations of ASOs targeting SCN1ANAT (UPNA-3, 1002) and splice modulating ASOs targeting the SCN1A pre-mRNA (UPNA-4, CTI-1008) for 48 hours, alone and in combinations. FIG. 1C is a bar graph showing a subset of the data from FIG. 1A. FIG. 1D is a bar graph showing fold change of productive SCN1A transcripts in SK-N-AS human neuroblastoma cell line treated with 10 nM of ASO targeting SCN1ANAT (UPNA-3) or 10 nM of dsRNAs targeting SCN1ANAT (UPNA-5 and UPNA-9) for 48 hours. Transcript levels were measured by qPCR, normalized to RPL37A, and compared to cells treated with 10 nM inactive control oligonucleotide (UPNA-2).

FIG. 2A is a bar graph showing fold change of RAPTOR mRNA in HEK293, SK-N-AS, SH-SY5Y cells, human primary astrocytes and N2A cells treated with 10 nM of dsRNA targeting RAPTOR mRNA (UPNA-20 or UPNA-21) for 48 hours. Transcript levels were measured by qPCR, normalized to RPL37A, and compared to cells treated with 10 nM inactive control oligonucleotide (UPNA-2). FIG. 2B is an image of a Western blot displaying levels of RAPTOR, RICTOR, phospho-AKT (Ser473), AKT, and GAPDH (control) protein from SK-N-AS human neuroblastoma cell line treated with 10-20 nM inactive control oligo (UPNA-2), 10 nM RAPTOR mRNA-targeting dsRNA (UPNA-20), 10 nM Rictor NAT-targeting dsRNA (UPNA-23), or 10 nM UPNA-20 and 10 nM UPNA-23 in combination for 72 hours. FIG. 2C is a bar graph quantifying fold change of RAPTOR protein from FIG. 2B (Western blot) in SK-N-AS human neuroblastoma cell line treated with 10 nM RAPTOR mRNA-targeting dsRNA (UPNA-20) compared to 10 nM inactive control dsRNA (UPNA-2) for 72 hours.

FIG. 3A is a bar graph showing fold change of RICTOR mRNA in HEK293T human embryonic kidney cell line treated with 10 nM RICTOR NAT-targeting dsRNAs (UPNA-22 through UPNA-31) for 48 hours. Transcript levels were measured by qPCR, normalized to RPL37A, and compared to cells treated with 10 nM inactive control dsRNA (UPNA-2). FIG. 3B is a bar graph showing fold change of RICTOR mRNA in SK-N-AS human neuroblastoma cell line treated with 10 nM RICTOR NAT-targeting dsRNAs (UPNA-23 and UPNA-29) for 48 hours. Transcript levels were measured by qPCR, normalized to RPL37A, and compared to cells treated with 10 nM inactive control dsRNA (UPNA-2). FIG. 3C is a bar graph quantifying fold change of RICTOR protein from FIG. 2B (western blot) in SK-N-AS human neuroblastoma cell line treated with 10 nM RICTOR NAT-targeting dsRNA (UPNA-23) compared to 10 nM inactive control dsRNA (UPNA-2) for 72 hours. FIG. 3D is a bar graph showing fold change of RICTOR mRNA in SK-N-AS human neuroblastoma cell line treated with 10 nM RICTOR NAT-targeting dsRNAs (UPNA-23 and UPNA-32 through UPNA-41) for 48 hours. Transcript levels were measured by qPCR, normalized to RPL37A, and compared to cells treated with 10 nM inactive control dsRNA (UPNA-2). FIG. 3E is a bar graph showing fold change of RICTOR mRNA in SK-N-AS human neuroblastoma cell line treated with 10 nM RICTOR NAT-targeting dsRNAs (UPNA-29 and UPNA-42 through UPNA-51) for 48 hours. Transcript levels were measured by qPCR, normalized to RPL37A, and compared to cells treated with 10 nM inactive control dsRNA (UPNA-2).

FIG. 4A is a bar graph showing fold change of RAPTOR and RICTOR mRNA in SK-N-AS human neuroblastoma cell line treated with 10 nM RAPTOR mRNA-targeting dsRNA (UPNA-20) and 10 nM RICTOR NAT-targeting dsRNA (UPNA-23) in combination for 48 hours. Transcript levels were measured by qPCR, normalized to RPL37A, and compared to cells treated with 20 nM inactive control dsRNA (UPNA-2). FIG. 4B isa bar graph quantifying fold change of RAPTOR and RICTOR protein from FIG. 2B (western blot) in SK-N-AS human neuroblastoma cell line treated with 10 nM RAPTOR mRNA-targeting dsRNA (UPNA-20) and 10 nM RICTOR NAT-targeting dsRNA (UPNA-23) in combination compared to 20 nM inactive control dsRNA (UPNA-2) for 72 hours. FIG. 4C is a bar graph quantifying fold change of AKT protein phosphorylation in SK-N-AS human neuroblastoma cell line treated with 10 nM RICTOR NAT-targeting dsRNA (UPNA-23) or 10 nM RAPTOR mRNA-targeting dsRNA (UPNA-20) and 10 nM RICTOR NAT-targeting dsRNA (UPNA-23) in combination compared to 10 nM inactive control dsRNA (UPNA-2) for 72 hours.

FIG. 5A is an image of a Western blot displaying levels of LC3B (1 and 2) and GAPDH (control) protein from SK-N-AS human neuroblastoma cell line treated with 10 nM inactive control dsRNA (UPNA-2) or 10 nM RAPTOR mRNA-targeting dsRNA (UPNA-20) for 72 hours. FIG. 5B is a bar graph quantifying the ratio of LC3B2 to LC3B1 protein from FIG. 16 (western blot) in SK-N-AS human neuroblastoma cell line treated with 10 nM RAPTOR mRNA-targeting dsRNA (UPNA-20) compared to 10 nM inactive control dsRNA (UPNA-2) for 72 hours. FIG. 5C is a bar graph showing levels of human total Tau protein from an ELISA in SK-N-AS human neuroblastoma cell line treated with 10-20 nM inactive control oligo (UPNA-2), 10 nM RAPTOR mRNA-targeting dsRNA (UPNA-20), 10 nM Rictor NAT-targeting dsRNA (UPNA-23), or 10 nM UPNA-20 and 10 nM UPNA-23 in combination for 72 hours. FIG. 5D is a bar graph showing levels of human phospho-Tau181 protein from an ELISA in SK-N-AS human neuroblastoma cell line treated with 10-20 nM inactive control oligo (UPNA-2), 10 nM RAPTOR mRNA-targeting dsRNA (UPNA-20), 10 nM Rictor NAT-targeting dsRNA (UPNA-23), or 10 nM UPNA-20 and 10 nM UPNA-23 in combination for 72 hours. FIG. 5E is a bar graph showing levels of human phospho-tau231 protein from an ELISA in SK-N-AS human neuroblastoma cell line treated with 10-20 nM inactive control oligo (UPNA-2), 10 nM RAPTOR mRNA-targeting dsRNA (UPNA-20), 10 nM Rictor NAT-targeting dsRNA (UPNA-23), or 10 nM UPNA-20 and 10 nM UPNA-23 in combination for 72 hours. FIG. 5F is a bar graph showing levels of human phospho-tau396 protein from an ELISA in SK-N-AS human neuroblastoma cell line treated with 10-20 nM inactive control oligo (UPNA-2), 10 nM RAPTOR mRNA-targeting dsRNA (UPNA-20), 10 nM Rictor NAT-targeting dsRNA (UPNA-23), or 10 nM UPNA-20 and 10 nM UPNA-23 in combination for 72 hours.

FIG. 6 is a schematic of the human C9orf72 locus adapted from Rizzu et al., (Acta Neuropathol Commun. 2016; 4(1):37) depicting the three coding transcripts for the C9orf72 gene located on the reverse strand of chromosome 9: NM_145005 (transcript 1), NM_018325 (transcript 2) and NM_001256054 (transcript 3). The hexanucleotide repeat expansion (HRE) is located either in intron 1 considering transcripts 1 and 3, or at the promoter region considering transcript 2. The region captured in the figure encompasses the 41 kb identified by the hg19 coordinates chr9:27539713-27580694.

FIG. 7 is a schematic depicting human C9ORF 72 isoforms. Shaded box indicates position of expanded repeats, the star indicates splice site 1b (SS1b). Isoform V2 encodes C9orf72-S(short), a 222-amino-acid protein of 24 kDa, unaffected by intron 1 repeat expansion, while V3 and V1, which incorporate distinct non-coding first exons (1a or 1b, respectively), encode C9orf72-L (long), a 481-amino-acid protein of 54 kDa.

FIG. 8 is a table listing genes with one or more regulatory mechanism.

FIG. 9 is a table of nucleotide sequences of genes and nucleic acid based molecules.

### DETAILED DESCRIPTION

Due to the diversity of their targets, NBTs lend themselves to multiplexing strategies so as to engage multiple complementary gene regulatory mechanisms, with low possibilities of toxic drug-drug interactions. The present disclosure describes at least 4,000 disease-associated loci with multiple regulatory mechanisms, including natural antisense transcripts (NATs), upstream open reading frames (uORFs) and nonsense-mediated decay exons and methods to target them synergistically using a combinatorial NBT approach. Furthermore, the number of such loci is likely to increase in the future as more and more diseases are associated with defined genetic mutations.

The present disclosure provides oligonucleotide sequences, methods, and compositions for targeting at least two or more gene expression regulatory mechanisms to synergistically modulate the expression level of a target gene. The present disclosure is based on the finding that the combined use of oligonucleotides or other nucleic acid based molecules that target different regulatory mechanisms of the same target gene, for example, NAT-mediated regulation and splicing, results in modulation of the expression of the target gene in excess of the sum of effects observed when each nucleic acid based molecule is used separately.

The present disclosure provides nucleic acid based molecule compounds, compositions and methods for the treatment, prevention, or amelioration of diseases, disorders, and conditions associated with genes whose expression is controlled by multiple regulatory mechanisms. The regulatory mechanisms include for example, natural antisense transcripts (NATs), NMD exons and upstream open reading frame (uORF). The associated diseases and disorders include, for example, genetic diseases and disorders, including CNS disorders such as Dravet syndrome (DS), familial frontotemporal dementia (FTD) and amyotrophic lateral sclerosis (ALS).

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

The term “Nucleic acid-based therapeutics (NBTs)” as used herein refers to nucleic acids and their analogues that are used to treat a disease or a disorder.

The term “target gene” or “target RNA transcript” as used herein refer to a gene or transcript (e.g., a pre-mRNA) whose expression is to be substantially modulated.

As used herein, “modulation” means either an increase (stimulation) or a decrease (inhibition) in the expression of a gene.

The term “nucleic acid based molecules” as used herein refers to an oligomer or polymer of either ribonucleic acid (RNA) or deoxyribonucleic acid (DNA), as well as non-naturally occurring oligonucleotides. Non-naturally occurring nucleic acid based molecules are oligomers or polymers which contain nucleobase sequences which do not occur in nature, or species which contain functional equivalents of naturally occurring nucleobases, sugars, or inter-sugar linkages, like aptamers, spiegelmers, peptide nucleid acids (PNA), threose nucleic acids (TNA), locked nucleic acids (LNA), or glycerol nucleic acids (GNA). This term includes oligomers that contain the naturally occurring nucleic acid nucleobases adenine (A), guanine (G), thymine (T), cytosine (C) and uracil (U), as well as oligomers that contain base analogs or modified nucleobases. Therefore the person skilled in the art understands that the term “oligonucleotide” comprises but is not limited to RNA, DNA and mixed oligonucleotides, antisense oligonucleotides, short interfering RNA (siRNA), microRNAs (miRNAs), guideRNAs (gRNAs), aptamers and also spiegelmers. The exact size of the nucleic acid based molecules will depend on many factors, which in turn depend on the ultimate function or use of the oligonucleotide. In some embodiments, the oligonucleotide is, e.g., 8-50 nucleotides, 15-45 nucleotides, 20-30 nucleotides or 17-35 nucleotides in length.

Nucleic acid based molecules can derive from a variety of natural sources such as viral, bacterial and eukaryotic DNAs and RNAs. Other oligonucleotides can be derived from synthetic sources, and include any of the multiple oligonucleotides that are being manufactured for use as research reagents, diagnostic agents or potential and definite therapeutic agents. The term includes oligomers comprising a single strand nucleic acid or a double strand nucleic acid. The two strands of a double strand nucleic acid are defined as “sense strand” and “antisense strand”. A “nucleic acid based molecule” may hybridize to other polynucleotides, therefore serving as a probe for polynucleotide detection, or a primer for polynucleotide chain extension.

The term “primer” as used herein refers to a type of oligonucleotide having or containing the length limits of a “nucleic acid based molecules” as defined above, and having or containing a sequence complementary to a target polynucleotide, which hybridizes to the target polynucleotide through base pairing so to initiate an elongation (extension) reaction to add nucleotides into the oligonucleotide primer. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present disclosure are generally between about 10 and 100 nucleotides in length, preferably between about 17 and 50 nucleotides in length, and most preferably between about 17 and 45 nucleotides in length. An “amplification primer” is a primer for amplification of a target sequence by primer extension. As no special sequences or structures are required to drive the amplification reaction, amplification primers for PCR may consist only of target binding sequences.

The term “probe” as used herein refers to a type of oligonucleotide having or containing a sequence which is complementary to another polynucleotide, e.g., a target polynucleotide or another oligonucleotide and is used to detect the presence of such other polynucleotide. The probe of the present invention is ideally less than or equal to 150 nucleotides in length, typically less than or equal to 100 nucleotides, for example less than or equal to 80, 70, 60 or 50 nucleotides in length.

The term “hybridization” as used herein refers to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

The term “antisense” as used herein refers generally to any agent, e.g., single-stranded oligonucleotides, that are sufficiently complementary to a target sequence to associate with the target sequence in a sequence-specific manner (e.g., hybridize to the target sequence).

The term “complementarity” as used herein refers to a structural relationship between two nucleotides that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. “Fully complementary” or “100% complementarity” refers to the situation in which each nucleotide monomer of a first oligonucleotide strand or of a segment of a first oligonucleotide strand can form a base pair with each nucleotide monomer of a second oligonucleotide strand or of a segment of a second oligonucleotide strand. “Less than 100% complementarity” refers to the situation in which some, but not all, nucleotide monomers of two oligonucleotide strands (or two segments of two oligonucleotide strands) can form base pairs with each other. “Substantial complementarity” refers to two oligonucleotide strands (or segments of two oligonucleotide strands) exhibiting 90% or greater complementarity to each other. “Sufficiently complementary” refers to complementarity between a target mRNA and a nucleic acid molecule, such that there is a change in the amount of protein encoded by a target mRNA. “Complementary strand” refers to a strand of a double-stranded nucleic acid molecule that is partially, substantially or fully complementary to the other strand.

The term “natural antisense transcripts (NATs)” as used herein, refers to endogenous RNAs transcribed from the opposite strand of the same genomic locus as other transcripts (sense transcripts), with or without sequence complementarity to such other transcripts.

The term “antagoNATs” refers to nucleic acid molecules complementary to natural antisense transcripts (NATs).

The term “nonsense-mediated mRNA decay (NMD)” as used herein refers to a post-transcriptional surveillance mechanism that degrades transcripts with nonsense mutations in their open reading frame (ORF).

The terms “non-sense mediated RNA decay-inducing exon (NMD exon),” is used herein to refer to coding exons that contain a premature termination codon. When incorporated into a transcript, this type of exon targets the transcript for NMD, decreasing the amount of protein produced.

The term “upstream open reading frames (uORFs)” refers to a sequence beginning with an initiation codon, located within the 5′ untranslated region (5′UTR) of a transcript, in frame with a termination codon positioned upstream or downstream (overlapped uORF) of the main ORF initiation codon. uORFs constitute a class of cis-acting elements that regulate translation initiation.

The term “splice modulatory element” as used herein refers to a nucleic acid region in a target RNA transcript, which either enhances or silences the splicing of introns in the pre-mRNA, or in general regulates the constitutive or alternative splicing of the pre-mRNA. Examples of splice modulatory elements include, but are not limited to, non-productive splice sites, exonic splicing enhancers, exonic splicing silencers, intronic splicing enhancers, and intronic splicing silencers.

The term “non-productive splice site” or “cryptic splice site” is splice site in a pre-mRNA that is used by the cellular splicing machinery that leads to the inappropriate inclusion and/or exclusion of introns and/or exons, thereby producing a non-functional transcript. The non-functional transcript can be rapidly degraded in the cell via one or more mechanisms, such as nonsense-mediated decay (NMD). The non-functional transcript may be translated into a non-functional or deleterious protein.

The term “SINEUP” refers to sequences in lncRNAs that increase the translation of partially overlapping mRNAs. SINEUP activity depends on two distinct RNA elements, the binding domain (BD) at the 5′ end, is a sequence that overlaps, in antisense orientation, to the sense protein-coding mRNA and determines target specificity, and an effector domain (ED), an inverted SINE (short interspersed nuclear elements) B2 sequence embedded in the non-overlapping part of the transcript that enhances mRNA translation. SINEUPs' modular architecture allows redirection of translation enhancement activity to any target mRNA by swapping its BD with the appropriate antisense sequence. SINEUPs are naturally occurring and by genetic engineering, synthetic SINEUPs can potentially target any mRNA of interest increasing translation and therefore the endogenous level of the encoded target protein.

The term “natural SINEUP transcript” means a transcript of a naturally occurring SINEUP.

The term “mini SINEUP” means RNAs exclusively composed of BD and ED sequences.

The term “functional domain” as used herein refers to a part of an mRNA or protein that is essential for carrying out a particular biological function, e.g. genomic DNA binding, catalytic activity, etc., of such mRNA or protein. Each RNA or protein molecule can contain multiple functional domains, each essential for a specific function. In this case the domains can be referred to as “first functional domain”, “second functional domain”, etc.

The term “about” or “approximately” means within 20%, within 10%, within 5%, or within 1% or less of a given value or range.

As used herein, “administer” or “administration” refers to the act of injecting or otherwise physically delivering a substance as it exists outside the body (e.g., an antisense compound provided herein) into a patient. The nucleic acid based molecules described herein may be administered to the central nervous system of a patient. The central nervous system includes the brain and spinal cord. Administration methods to the central nervous system include, but are not limited to, intrathecal, intraventricular or intrastriatal infusion or delivery and/or any other method of physical delivery described herein or known in the art. Intraventricular infusion can comprise administration using an Ommaya reservoir.

When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and can be continued chronically to defer or reduce the appearance or magnitude of disease-associated symptoms, e.g., damage to the involved tissues and airways.

As used herein, the term “composition” is intended to encompass a product containing the specified ingredients (e.g., an oligonucleotide compound provided herein) in the specified amounts, as well as any product which results, directly or indirectly, from combination of the specified ingredients in the specified amounts.

“Effective amount” means the amount of active pharmaceutical agent (e.g., a nucleic acid based molecule of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

The terms “subject” and “patient” are used interchangeably herein. For instance, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sports animals, and pets. In one embodiment, the subject is a mammal, such as a human, having C9orf72-mediated amyotrophic lateral sclerosis (ALS). In another embodiment, the subject is a mammal, such as a human, that is at risk for developing C9orf72-mediated amyotrophic lateral sclerosis (ALS).

The term “neurodegenerative disease or disorder” as used herein refers to a group of diseases or disorders that affect the structure or function of the brain and/or spinal cord. Neurodegenerative diseases occur as a result of neurodegenerative processes, e.g. the progressive loss of structure or function of neurons, including but not limited to the death of neurons. Neurodegenerative diseases include, but are not limited to, Amyotrophic lateral sclerosis (ALS), Frontotemporal dementia (FTD), Friedrich's Ataxia, Alzheimer's disease, Huntington's disease, and Dravet Syndrome.

### Target Genes

In various embodiments, the methods of the present invention exploit the presence of at least two distinct expression regulatory mechanisms associated with the target gene or several target genes involved the same disease or condition. In various aspects, these expression regulatory mechanisms are mediated by a NAT, a promoter RNA, an enhancer RNA, a promoter sequence, a splicing complex, an NMD exon in mRNA, a uORF, a translation suppression element in the 5′ UTR, a splice modulatory element, or RNA interference (RNAi) targeting the 3′ UTR or ORF or other sequence of the mRNA encoding the target protein or target ncRNA.

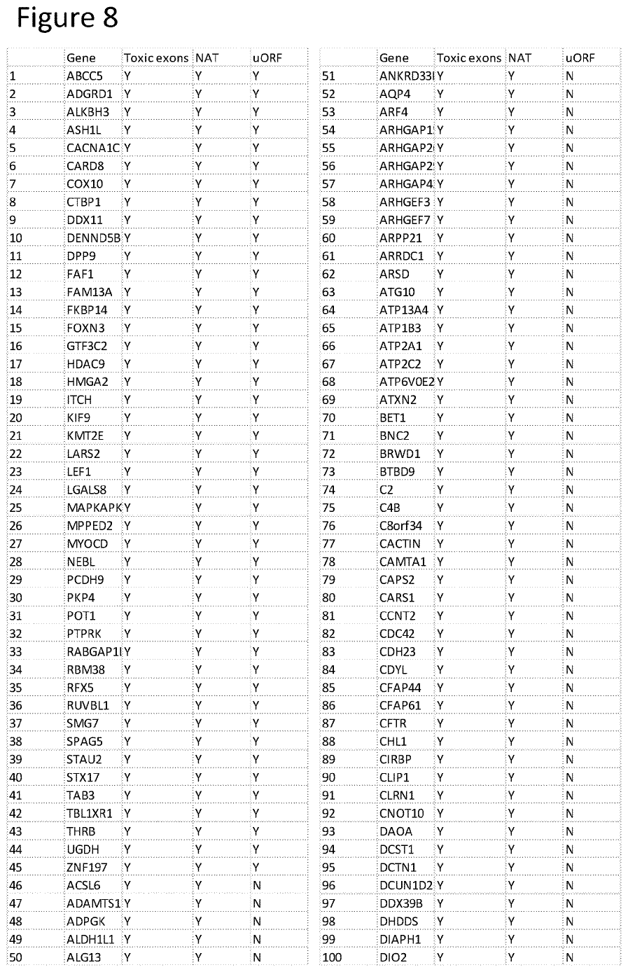

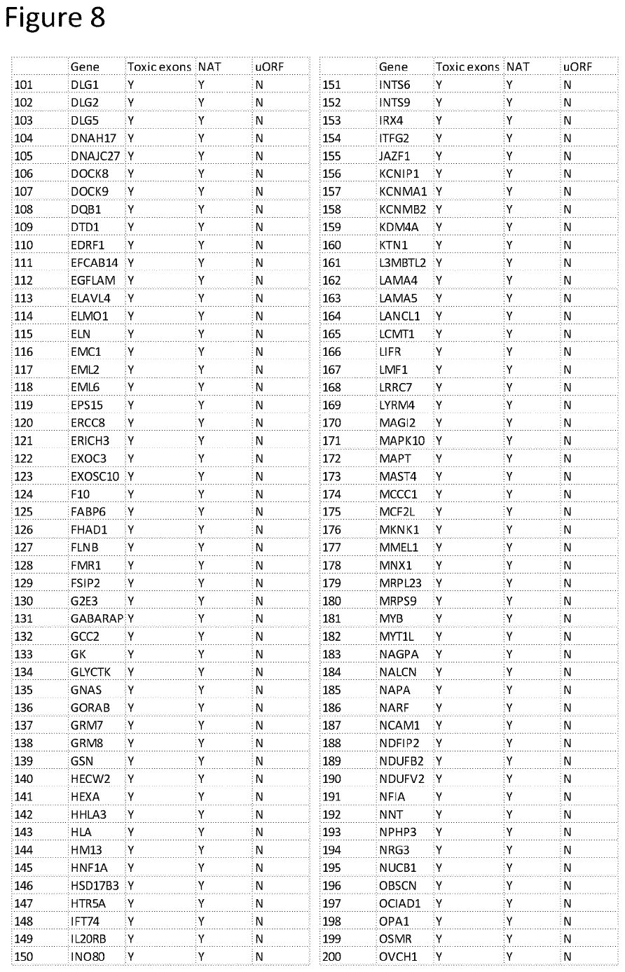

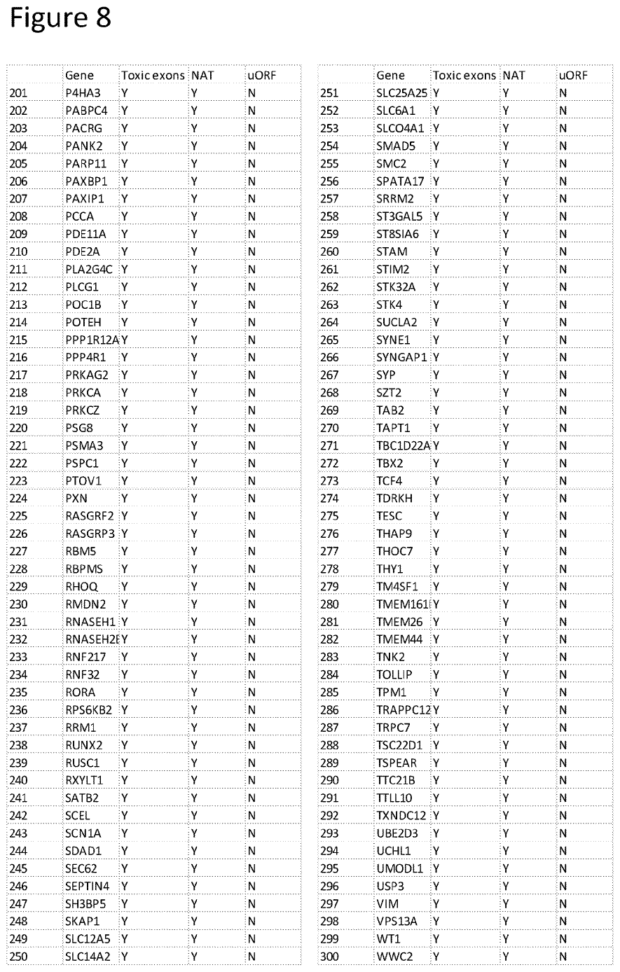

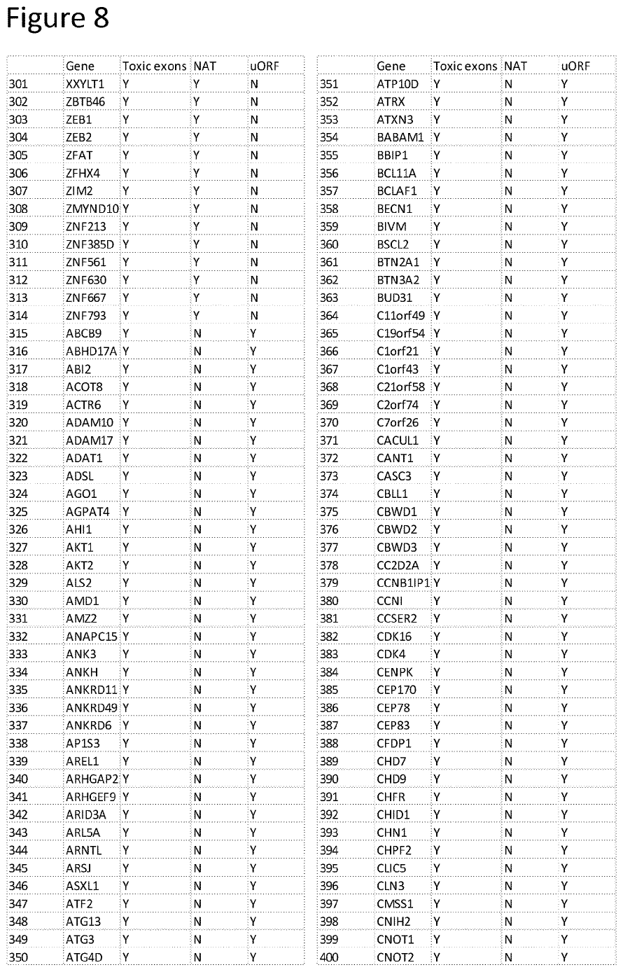

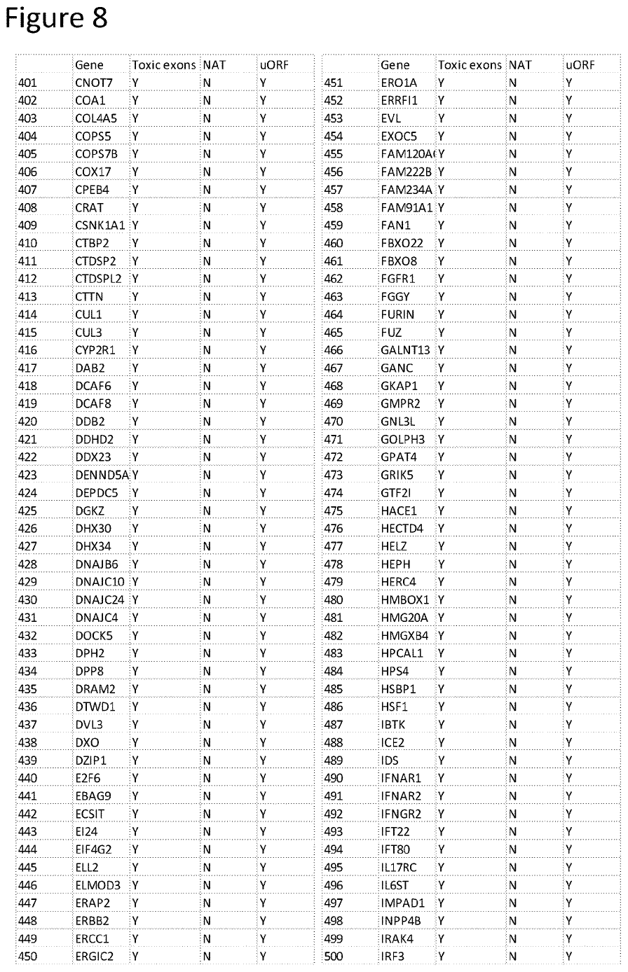

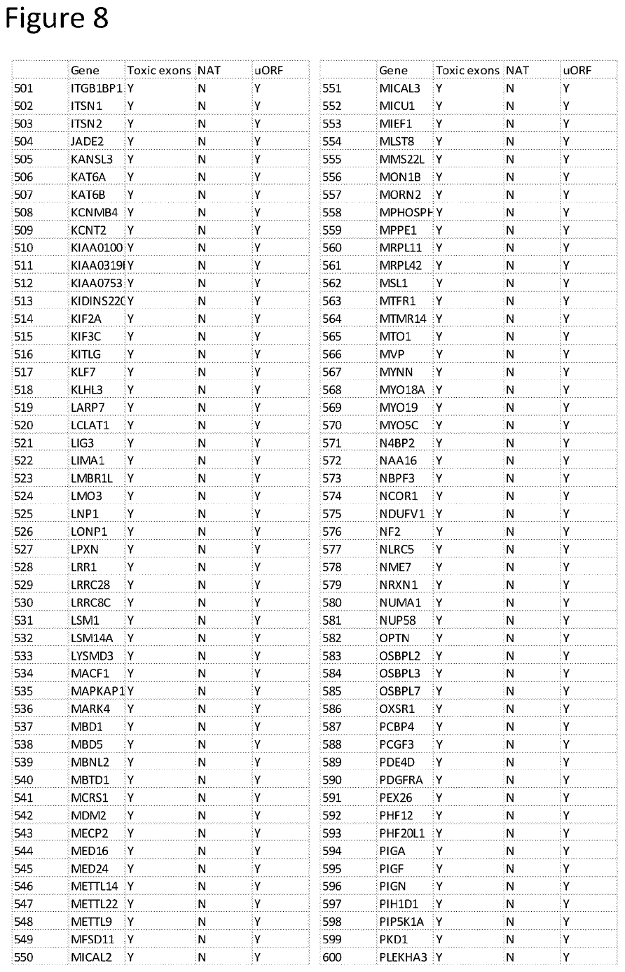

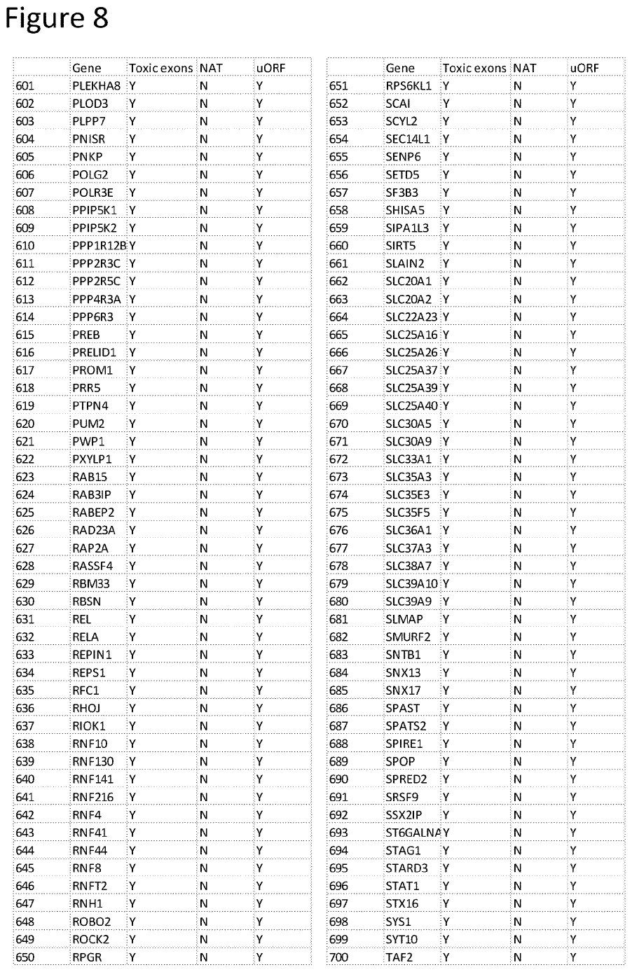

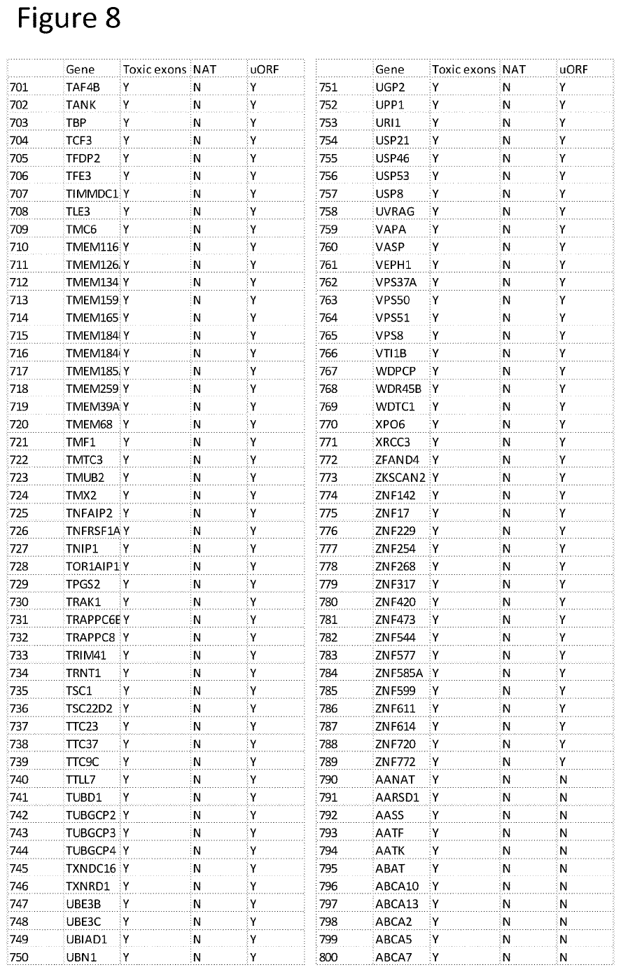

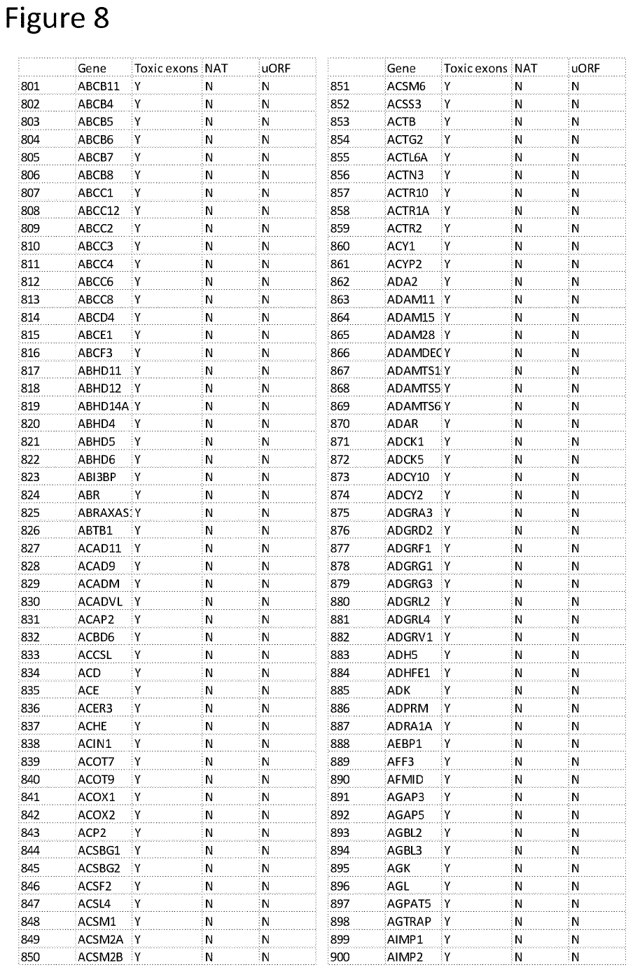

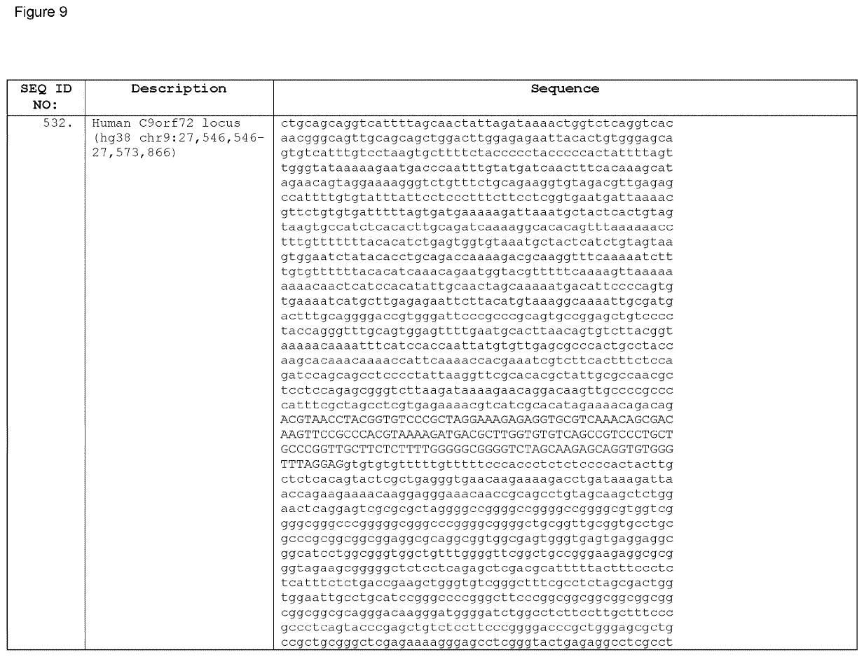

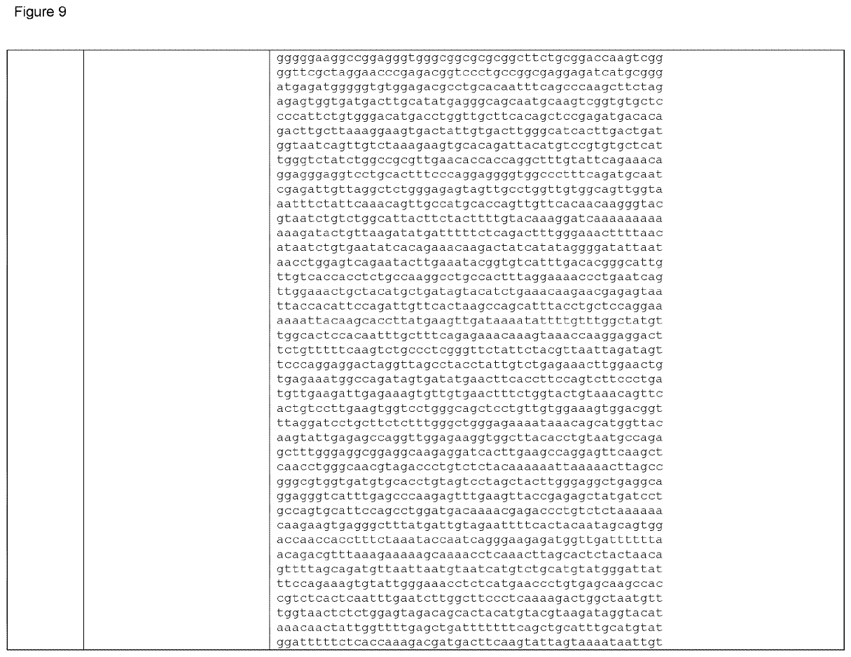

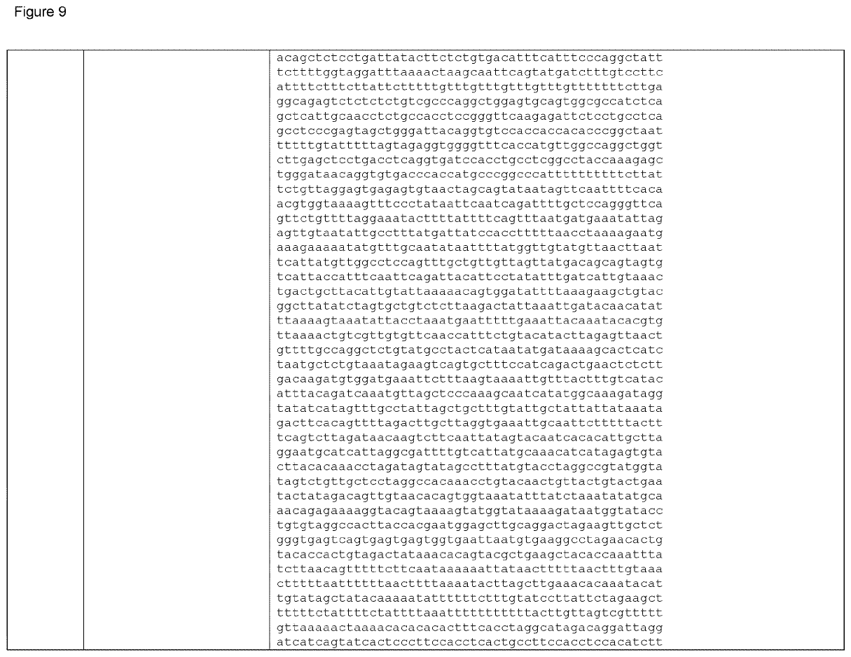

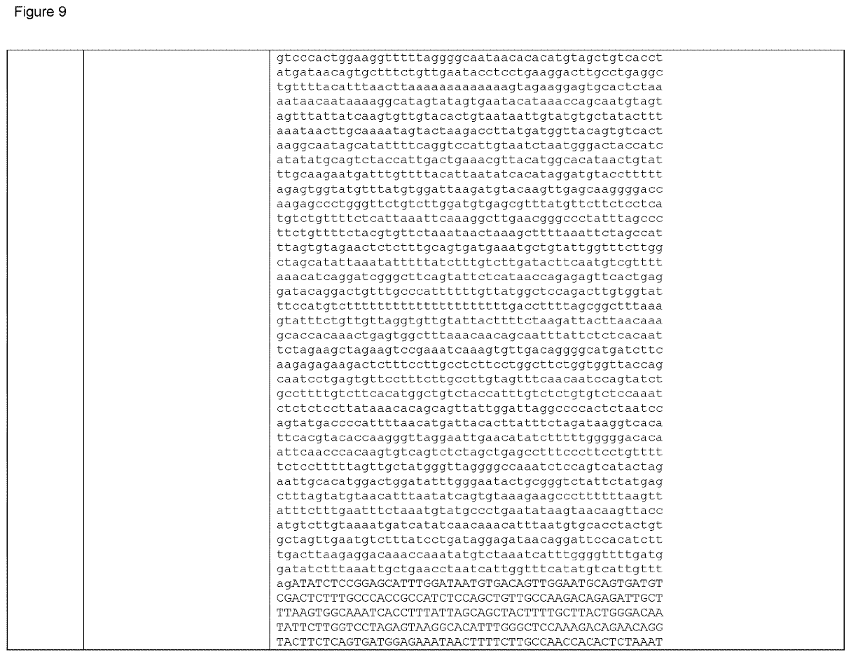

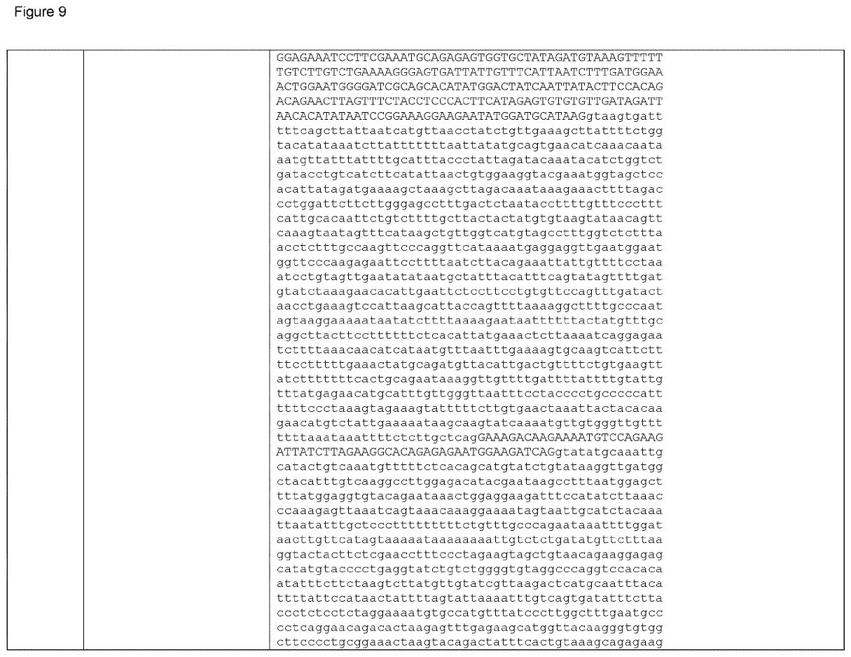

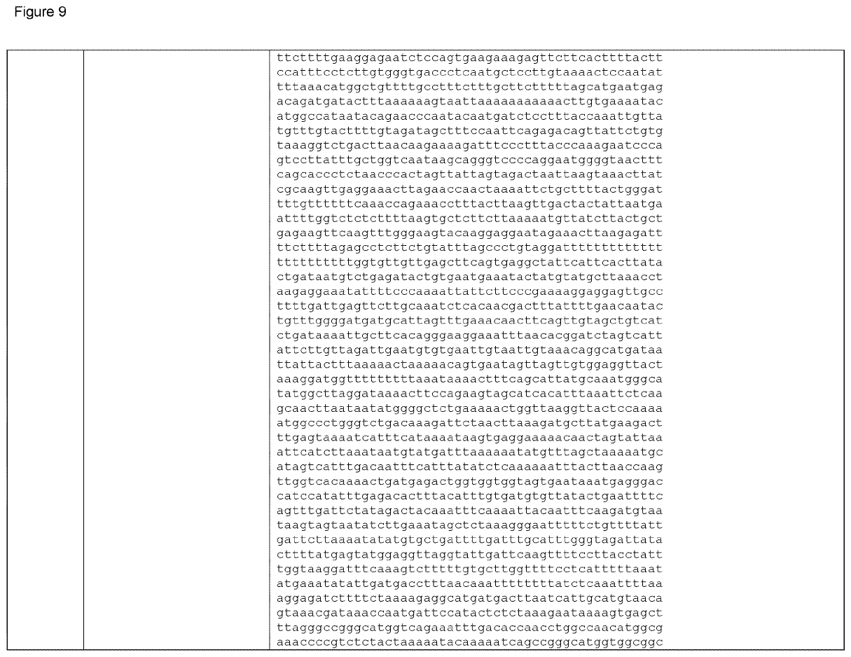

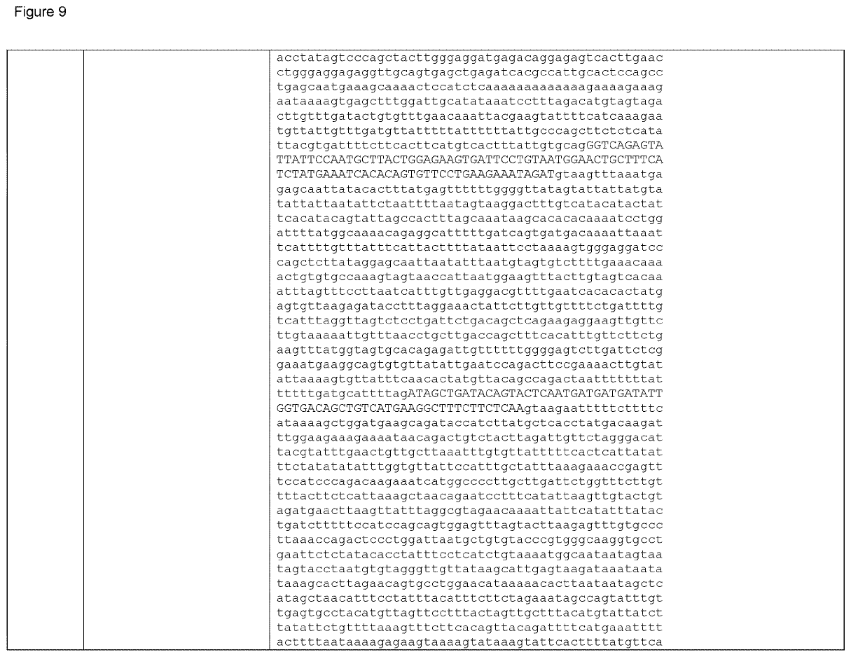

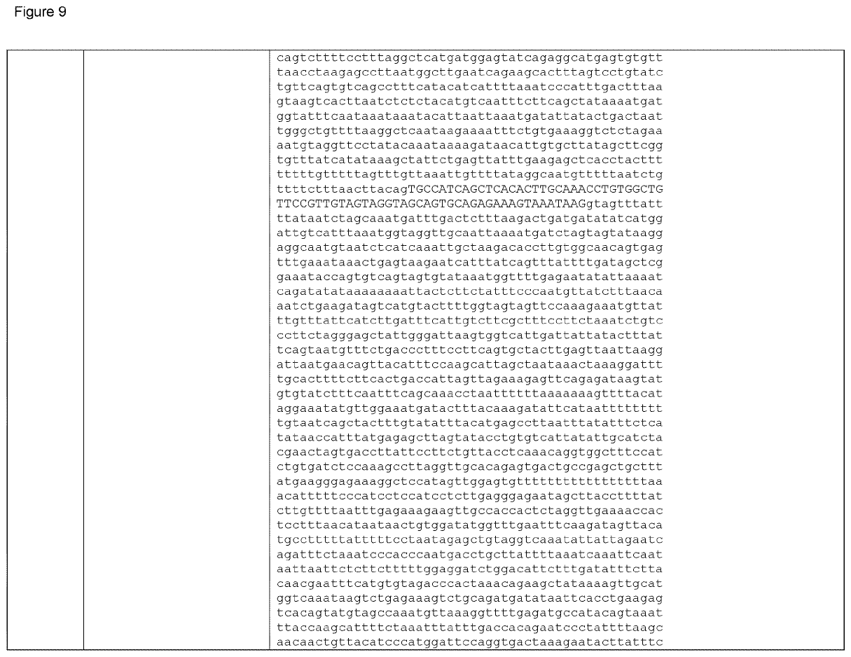

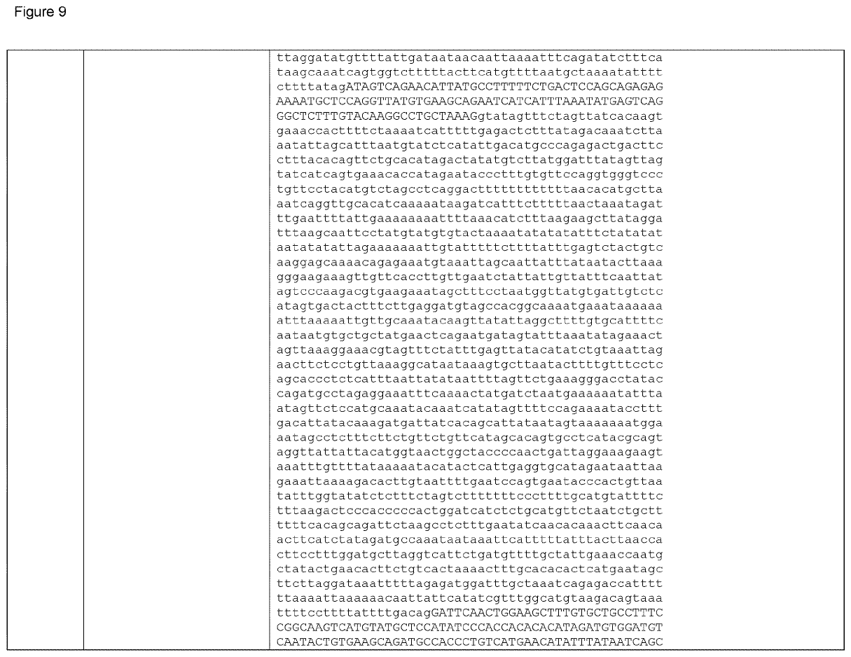

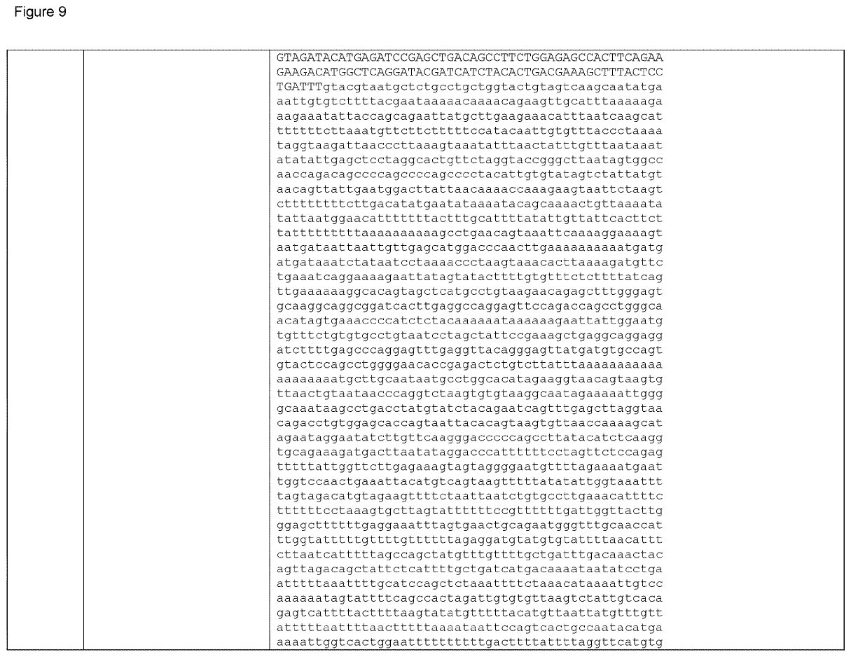

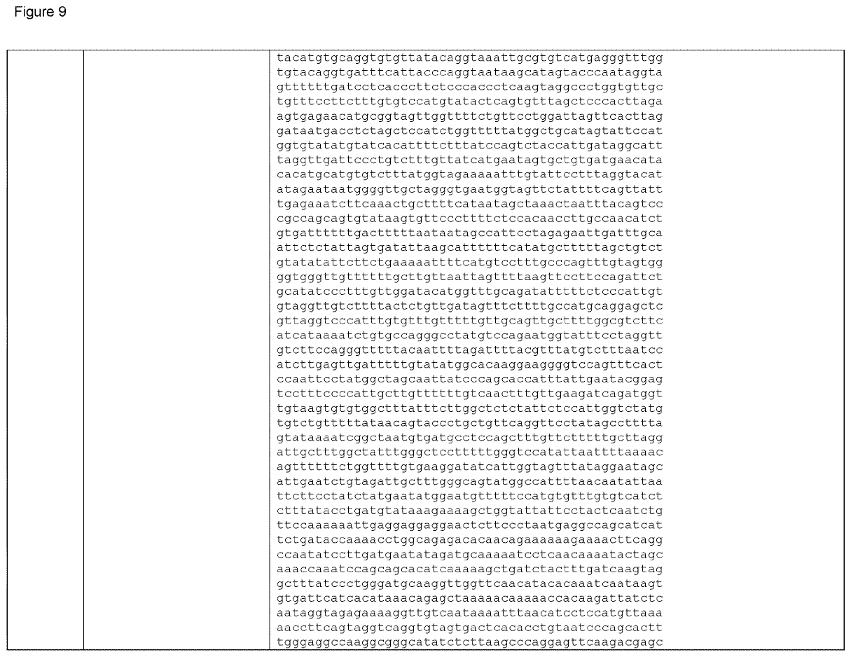

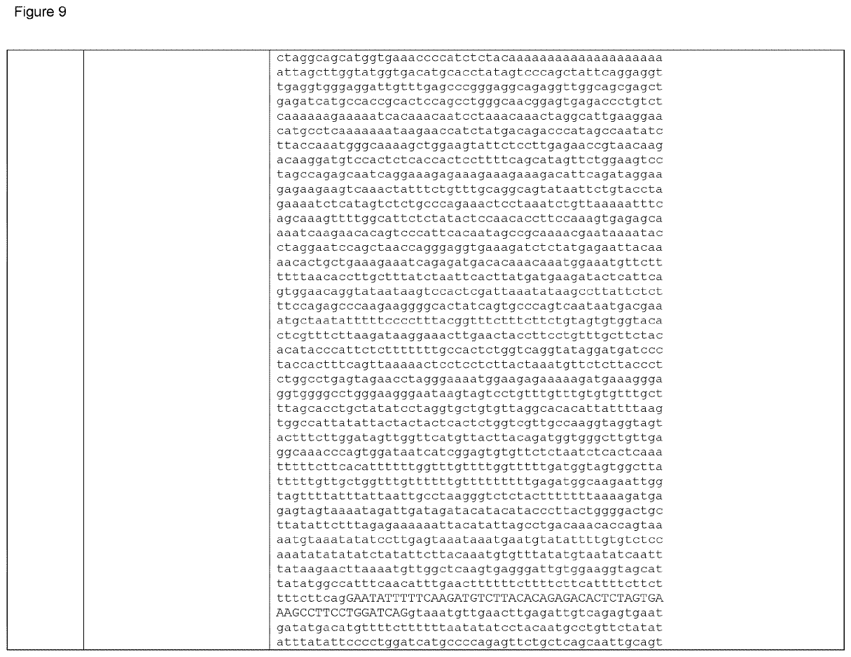

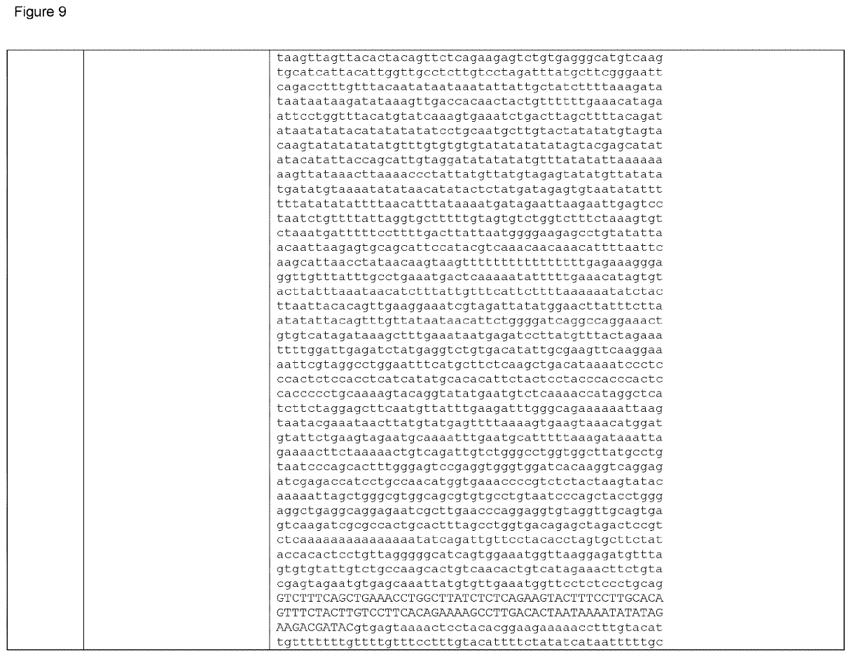

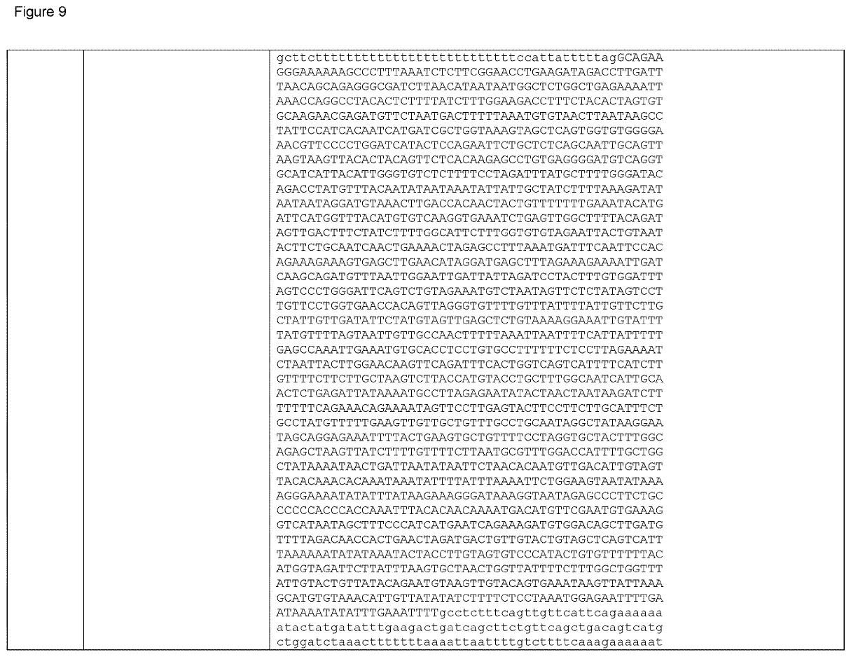

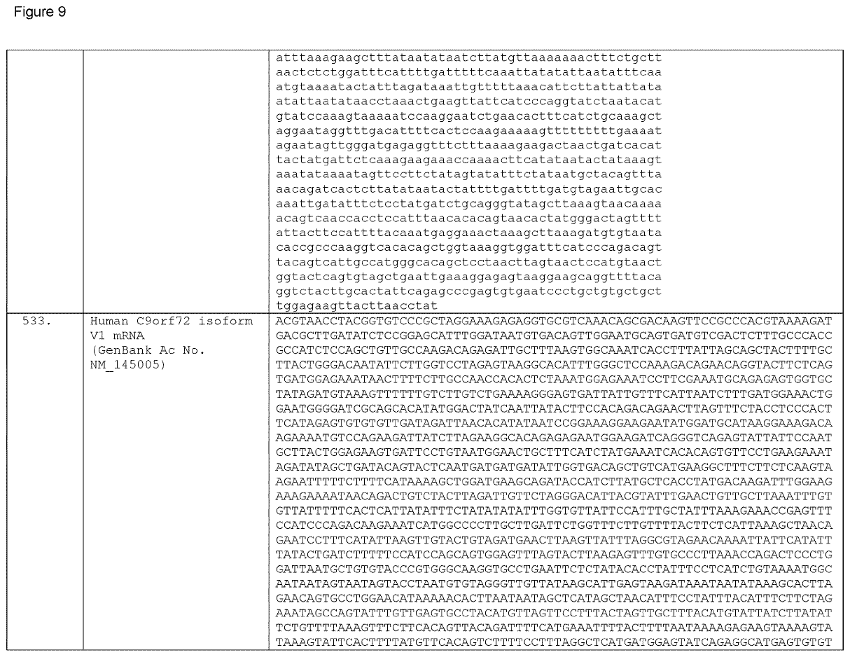

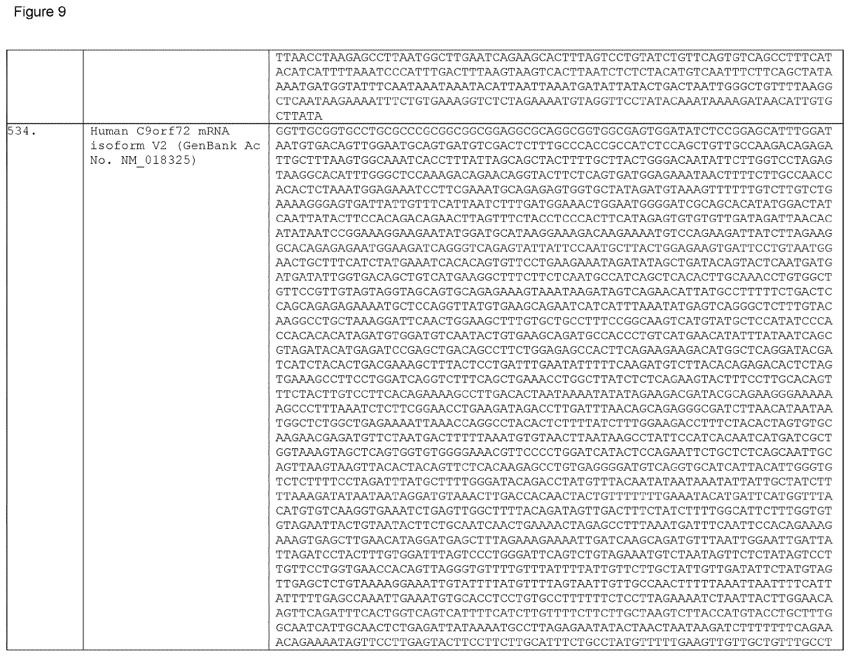

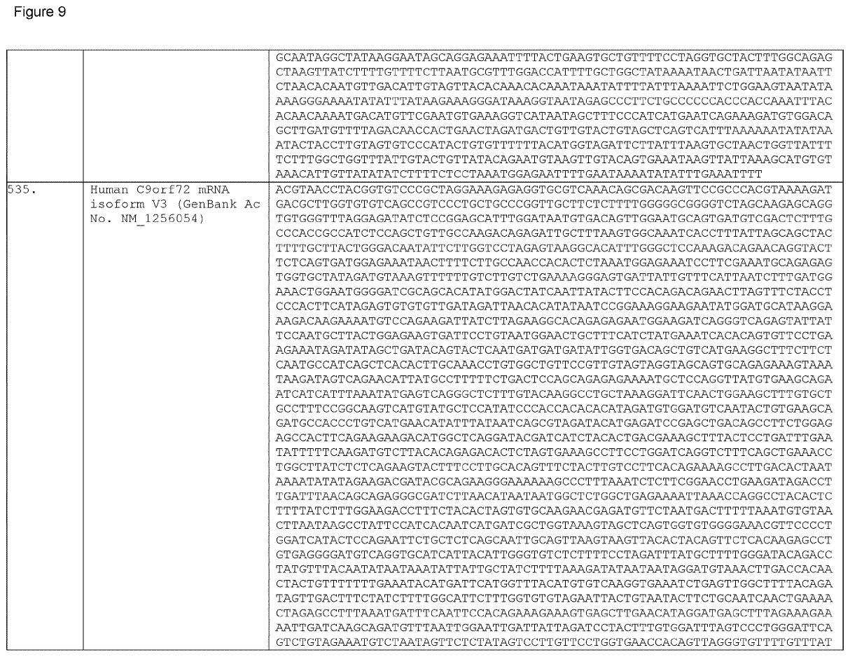

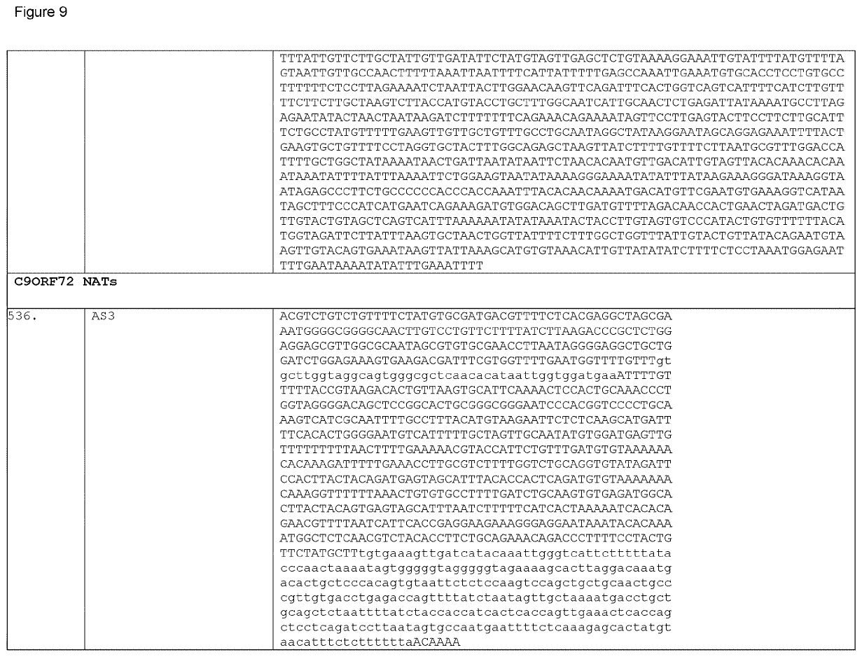

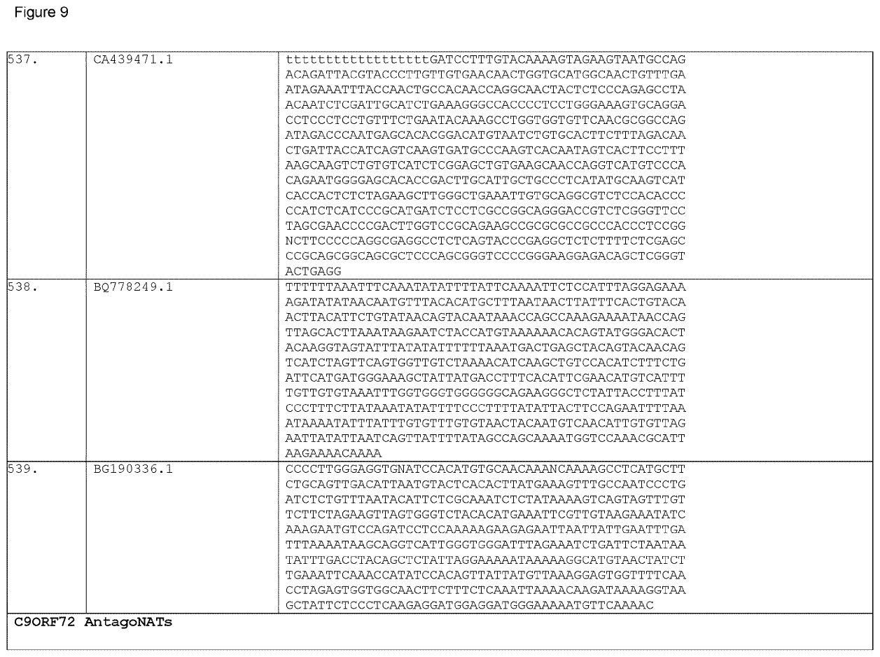

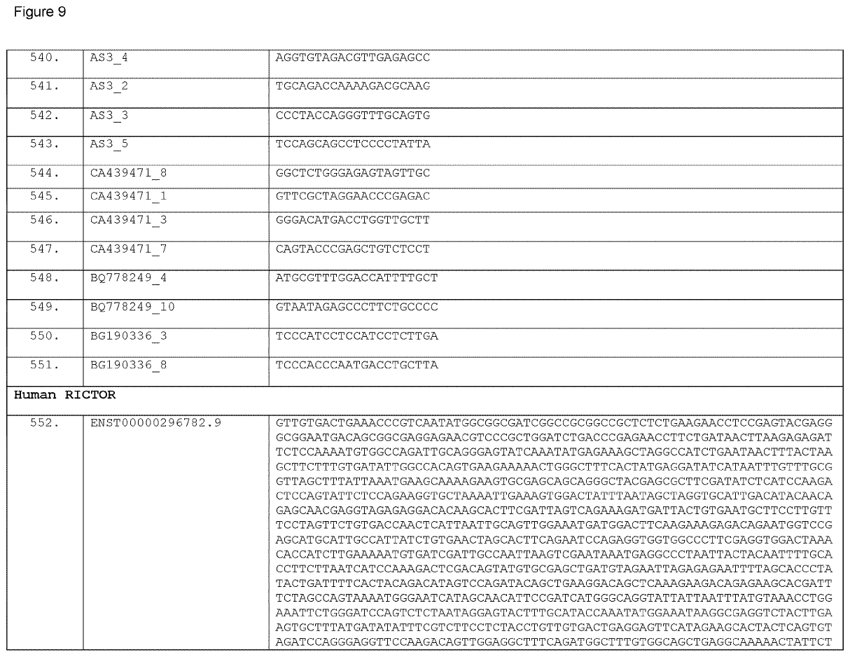

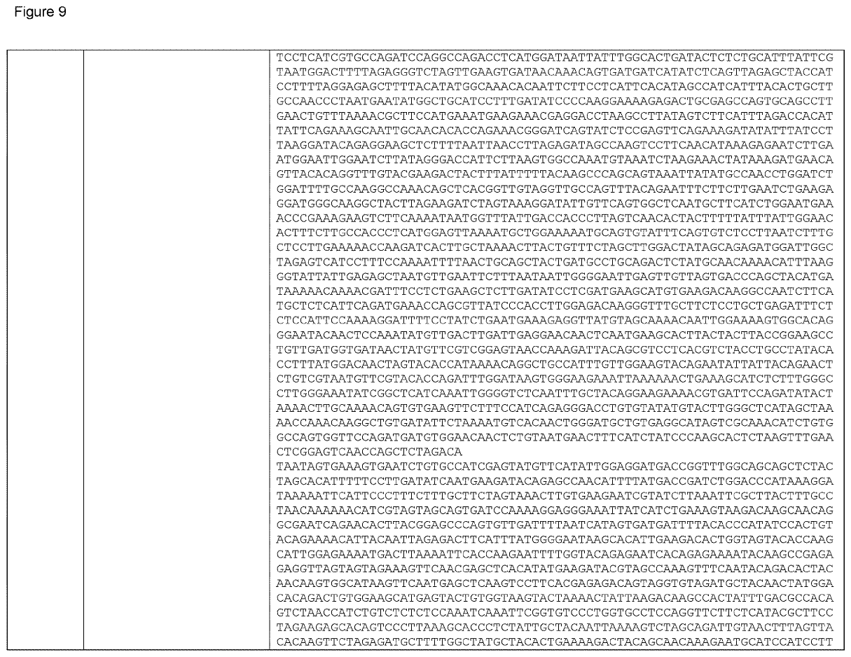

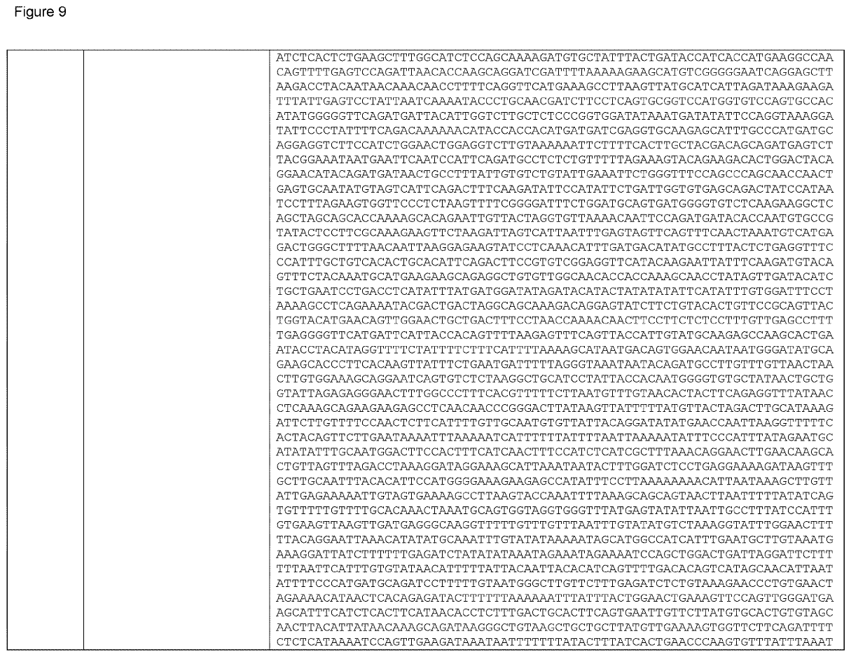

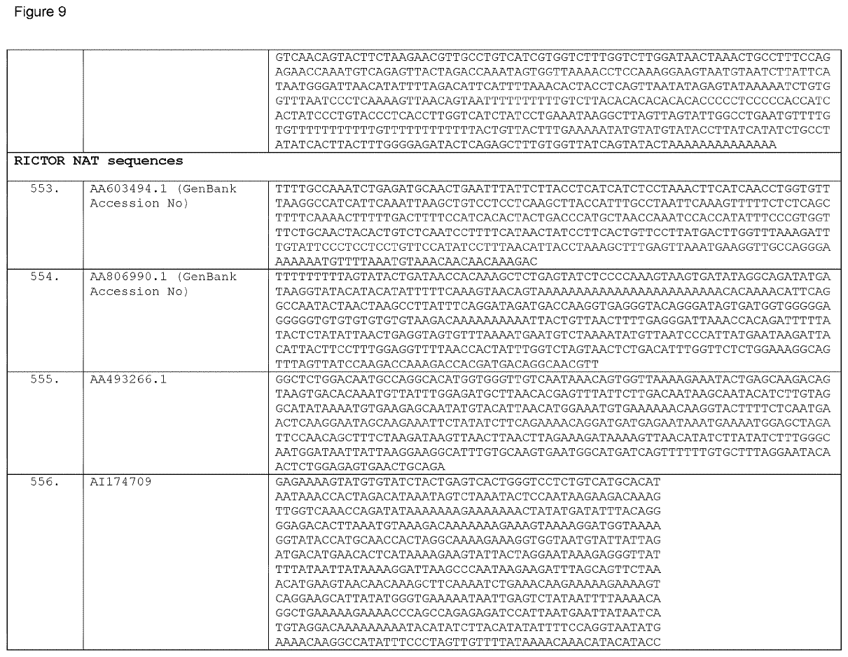

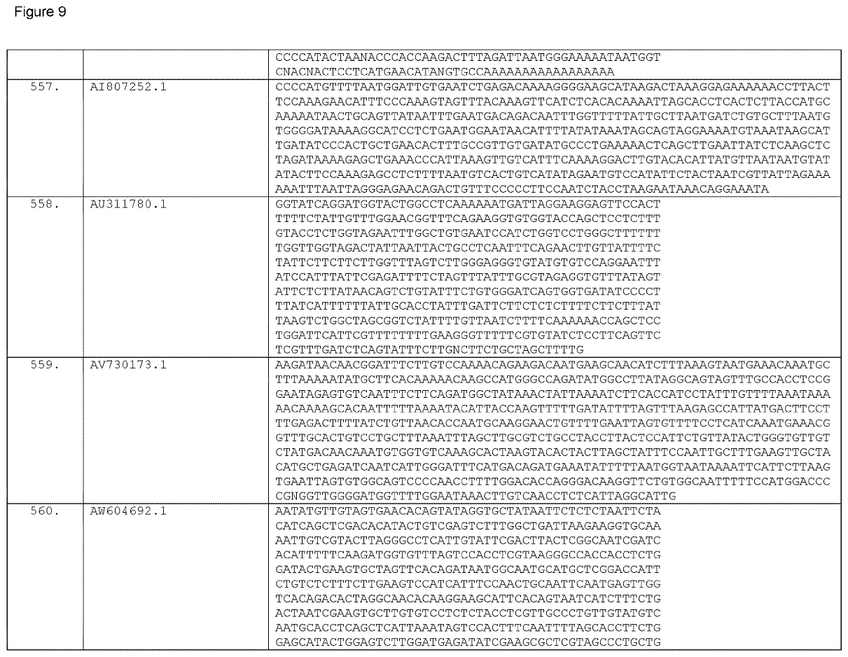

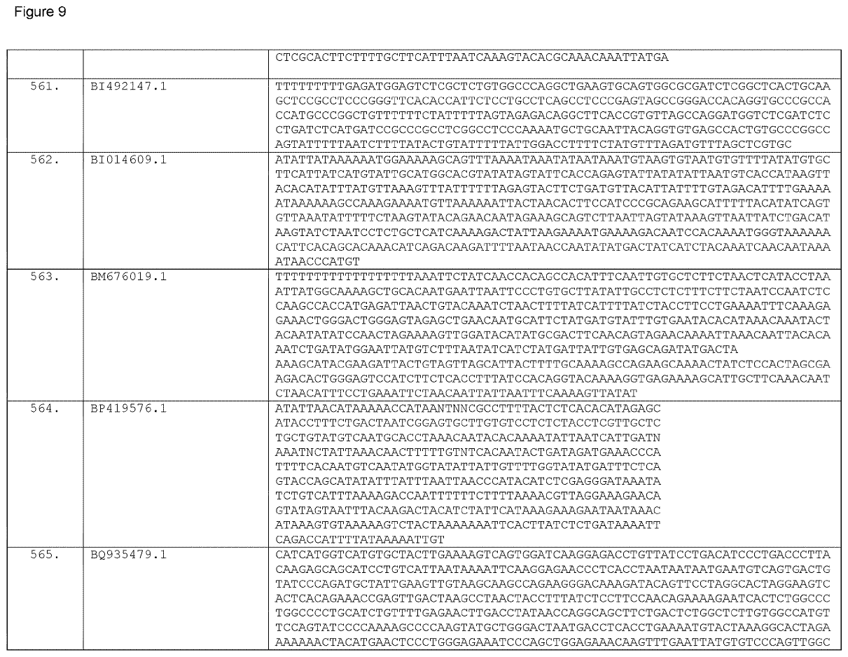

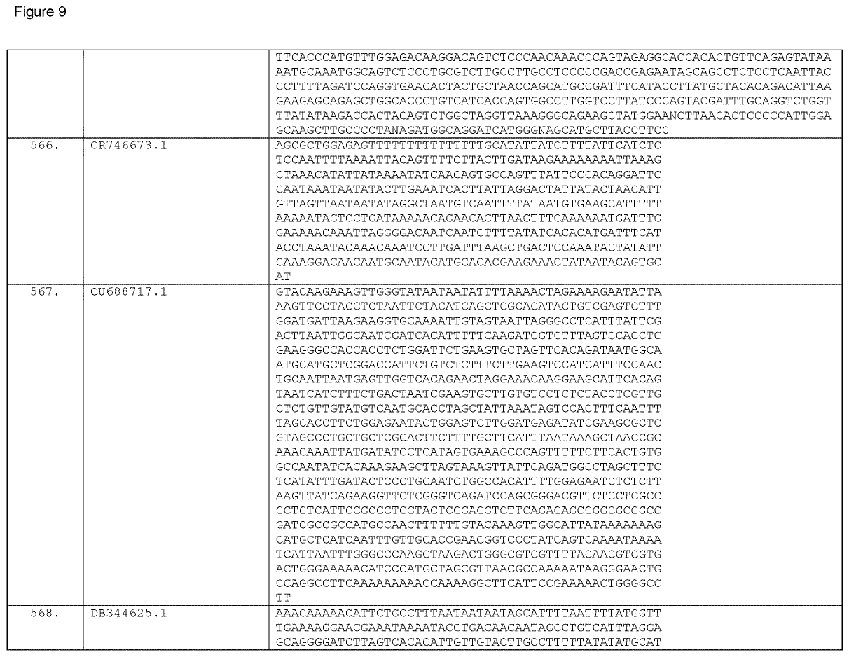

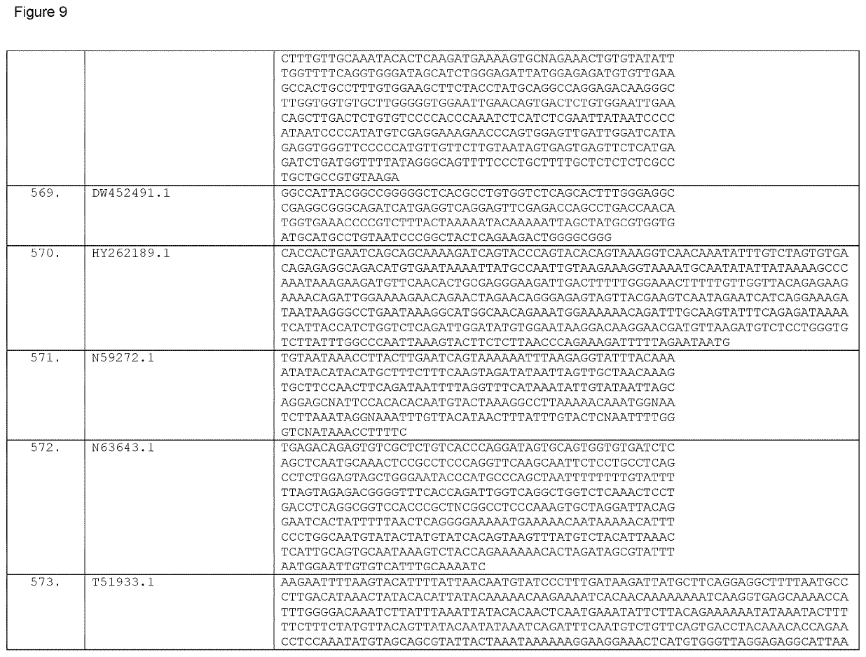

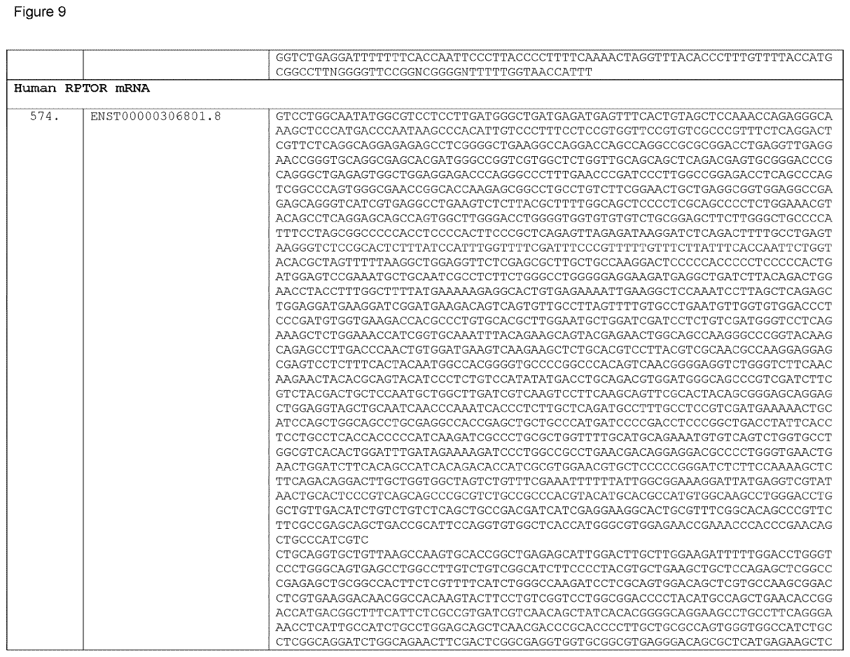

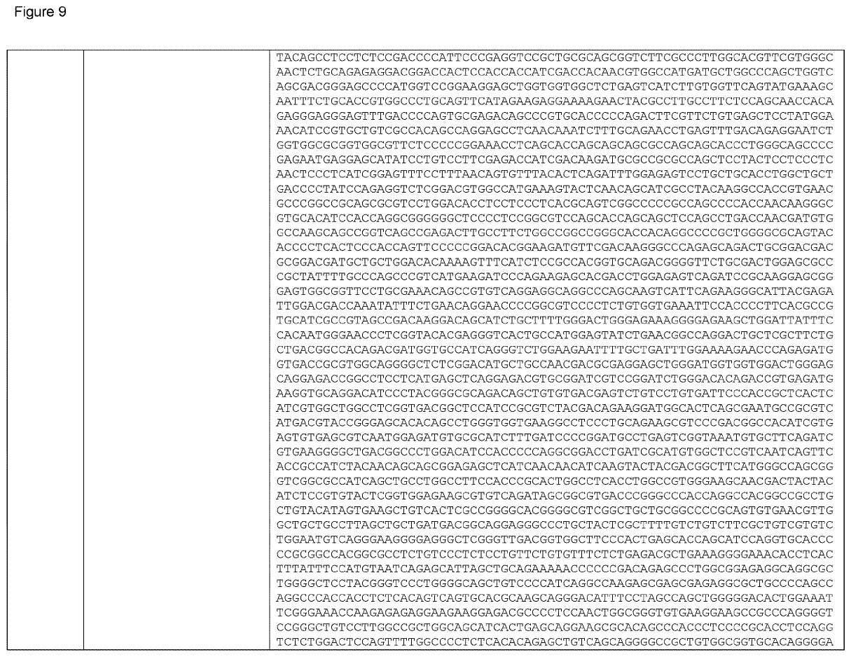

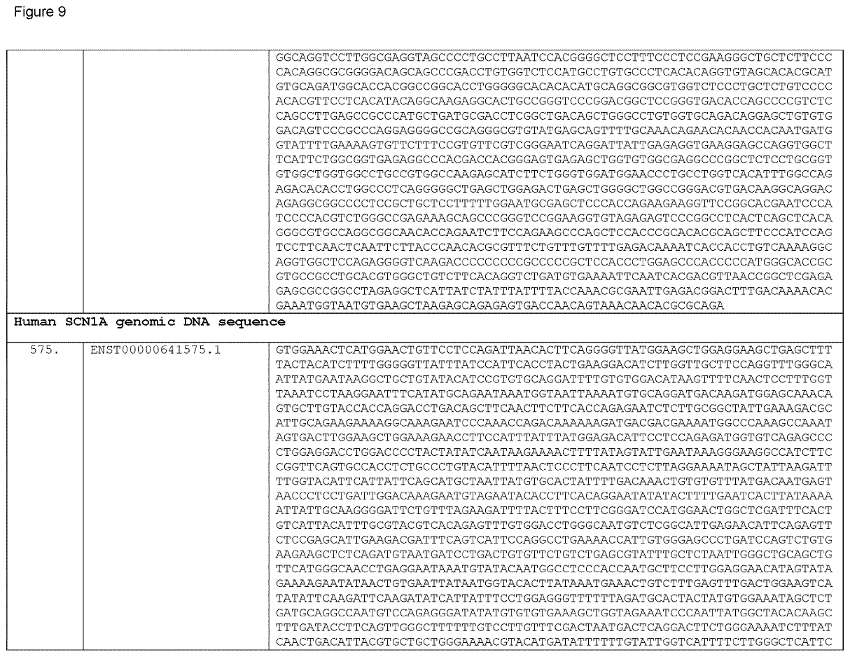

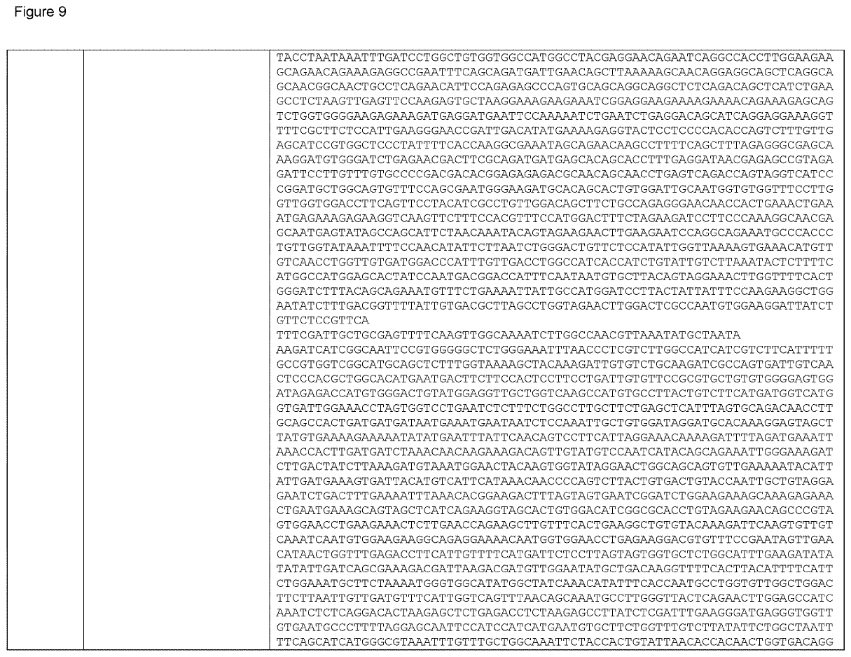

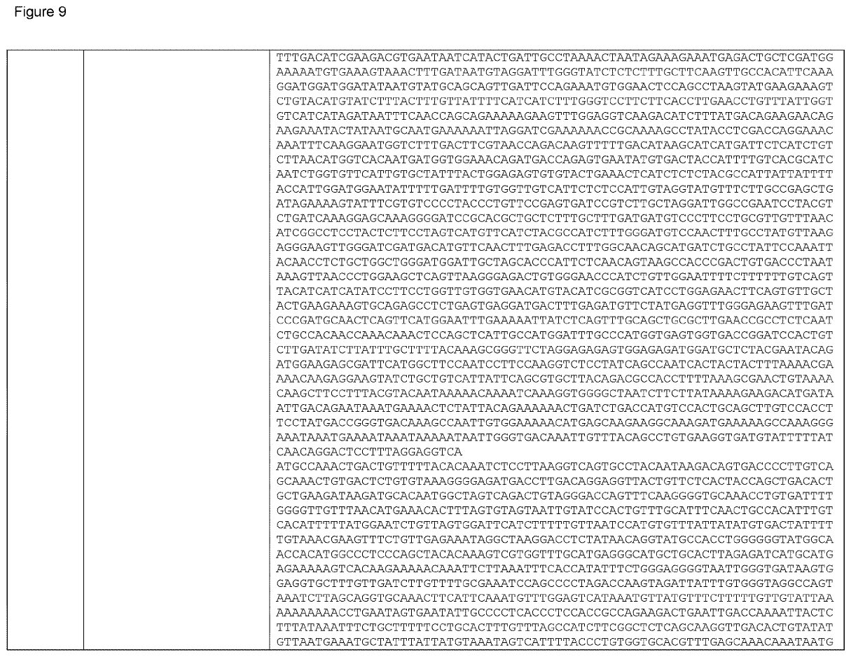

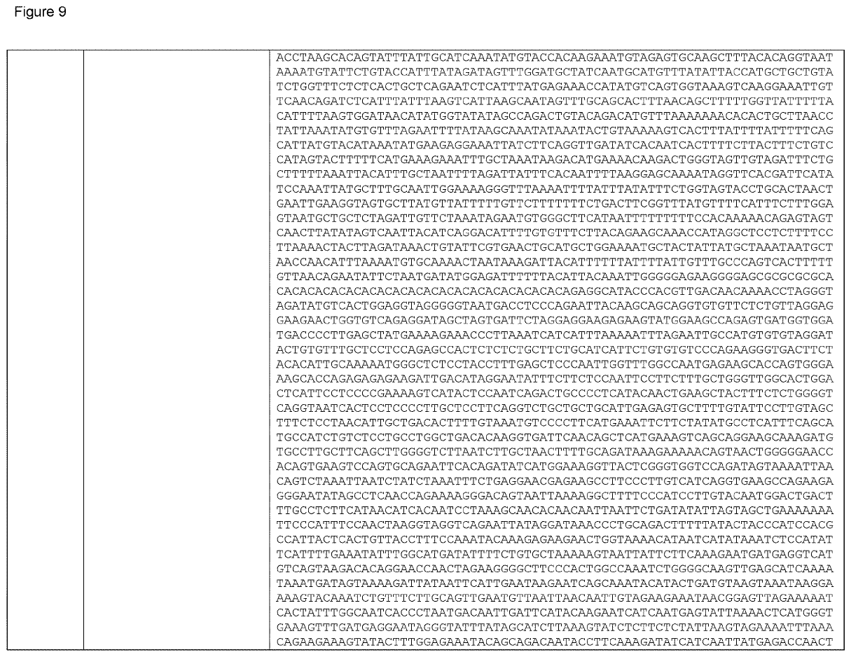

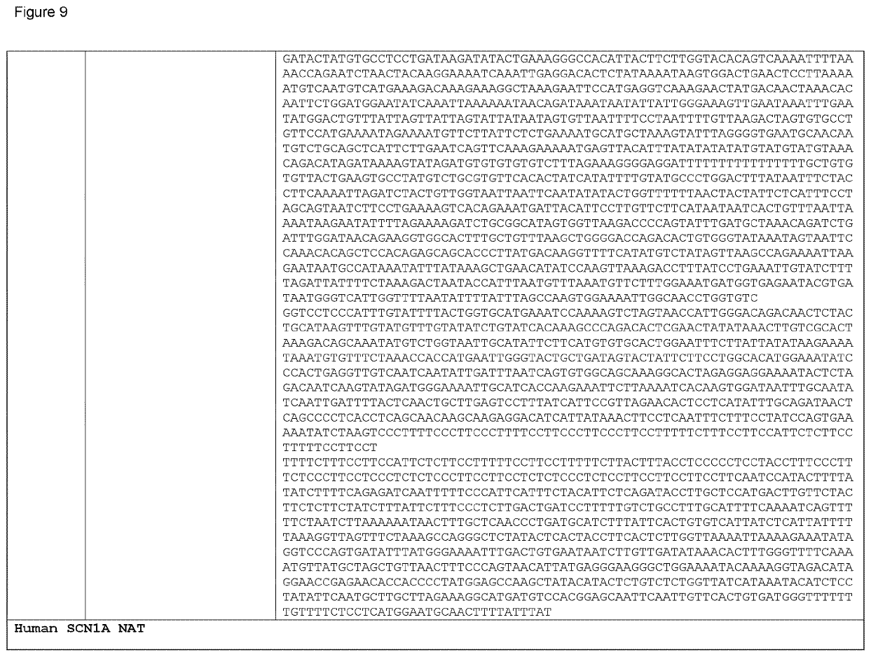

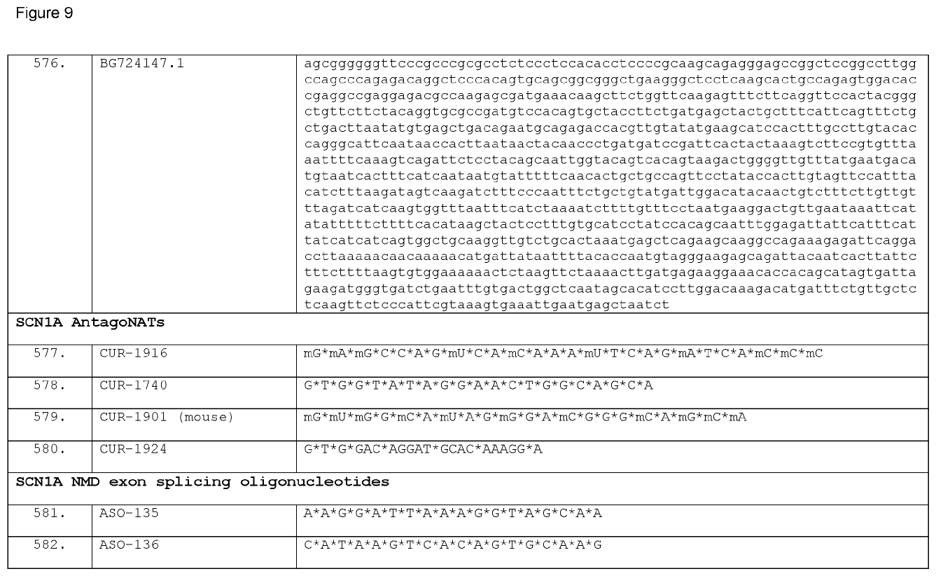

In various embodiments, expression of the target gene is modulated by 1, 2 or 3 gene regulatory mechanisms as recited in FIG. 8. In further embodiments, expression of the target gene is modulated by 3 gene regulatory mechanisms as recited in gene numbers 1-45 of FIG. 9. In other embodiments, the target gene comprises 2 gene regulatory mechanisms as recited in gene numbers 46-789 of FIG. 9.

In various embodiments, the target genes are associated with neurological disorders or diseases. Neurological diseases include, but are not limited to, Amyotrophic lateral sclerosis (ALS), Frontotemporal dementia (FTD), Friedrich's Ataxia, Alzheimer's disease, Huntington's disease, and Dravet Syndrome.

In various embodiments, the target genes are associated with genetic disorders or diseases, including neurological disorders. Genetic diseases include, but are not limited to, Friedrich's ataxia, mTORopathies, such as tuberous sclerosis complex (TSC) and Alzheimer's disease (AD), Huntington's disease, sodium channel a2 subunit (SCN2A) encephalopathy, sodium channel a8 subunit (SCN8A) encephalopathy, SCN1A-associated Dravet syndrome, C9orf72-mediated amyotrophic lateral sclerosis (ALS) and frontotemporal dementia (FTD), sodium channel a5 subunit (SCN5A) arrhythmia, tauopathies, such as Pick disease, progressive supranuclear palsy, corticobasal degeneration, argyrophilic grain disease, globular glial tauopathies, neurofibrillary tangle dementia, chronic traumatic encephalopathy or aging-related tau astrogliopathy.

### Nucleic Acid Based Molecules

In some embodiments, two or more nucleic acid based molecules are provided herein for modulating the expression level of a target gene in a cell. In some embodiments, at least two or more nucleic acid based molecules are used and each has one of the following effects:
a) modulating the action of a natural antisense transcript (NAT) that controls the expression of the target protein;
b) modulating the splicing of a mutated exon or intron or a nonsense mediated RNA decay-inducing exon in mRNA encoding a target protein or target ncRNA encoded by the target gene;
c) inhibiting the effect of a translation suppression element in the 5′ untranslated region of a transcript of the target gene, wherein the translation suppression element is an upstream open reading frame (uORF);
d) knocking down an mRNA encoding a pathogenic isoform of the target protein or of a target regulatory ncRNA involved in a pathogenic process;
e) knocking down all isoforms of the mRNA of the target protein or of target ncRNA;
f) modulating effects of promoter RNAs affecting in cis or in trans the expression from the genomic locus encoding the target protein or of target ncRNA;
g) modulating effects of enhancer RNAs affecting in cis or in trans the expression from the genomic locus encoding the target protein or of target ncRNA, or
h) replicating the effects of the natural SINEUP transcript through inclusion of its essential parts.

In some embodiments, the nucleic acid based molecules are selected from the group consisting of an antisense oligonucleotide (ASO), a guide RNA (gRNA), an siRNA, an shRNA, a small nuclear RNA (snRNA), a mini SINEUP, and an miRNA.

In various embodiments, one nucleic acid based molecule is an antisense oligonucleotide (ASO).

In various embodiments, the ASO binds to a natural antisense transcript (NAT), thereby modulating the expression of the sense transcript that encodes a target protein encoded by the target gene.

In various embodiments, two, three, four or more nucleic acid based molecules are provided.

In various embodiment, the nucleic acid based molecules can comprise one or more modified nucleotides, modified bonds and the like. Examples of modified bonds or internucleotide linkages comprise phosphorothioate, phosphorodithioate or the like. The preparation of modified nucleotides and oligonucleotides is known in the art and need not be described here.

An ASO is specifically hybridizable when its binding to a target gene interferes with the normal function of the target gene to cause a modulation of function, and there is a sufficient degree of complementarily to avoid non-specific binding of the ASO to non-target sequences under conditions in which specific binding is desired, i.e., under physiological conditions in the case of in vivo assays or therapeutic treatment, and in the case of in vitro assays, under conditions in which the assays are performed.

In various embodiments, the ASO is complementary to an equal length portion of a transcript of a target gene.

In various embodiments, the ASO, is at least 50%, at least 55%, at least 60%, at least 65%, at least 70%, at least 75%, at least 80%, at least 85%, at least 90%, at least 95%, or 100% complementary to an equal length portion of a transcript of a target gene.

In various embodiments, the ASO binds to a natural antisense sequence of C90RF72 (SEQ ID NOs: 522-525 and 536-539).

In various embodiments, binding of the ASO to a target site on the natural antisense transcript of C90RF72 decreases the expression or function of the natural antisense transcript.

In various embodiments, binding of the ASO to a target site on the natural antisense transcript of C90RF72 increases the expression or function of C90RF72 (SEQ ID NOs: 533-535), as compared to a control.

In various embodiments, the ASO is a single stranded DNA molecule.

In various embodiments, the ASO is 10 to 50 nucleotides in length. In various embodiments, the ASO is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or ranges between any of these lengths. In some embodiments, the ASO is 20 to 30 nucleotides in length.

In various embodiments, the ASO comprises a sequence that has at least about 80%, 85%, 90%, 95%, 97%, or 100% complementarity to all or a portion of any one of SEQ ID NOs: 522-525 and 536-539. In various embodiments, the ASO comprises the sequences set forth as SEQ ID NOs: 540-551. These ASOs can comprise one or more modified nucleotides, shorter or longer fragments, modified bonds and the like.

In various embodiments, a nucleic acid based molecule is a gapmer ASO.

Introduction of a gapmer ASO into a cell leads to the formation of a partial DNA/RNA hetero-duplex, a structure that is recognized by RNase H. Recruitment of RNase H leads to the degradation of the mRNA of the target gene thereby decreasing the expression of the target gene.

In various embodiments, the gapmer ASO comprises three regions; a 5′ and 3′ “wing” regions flanking a core, “gap” region.

In various embodiments, the S′ wing comprises between 1-6 locked nucleic acid (LNA) nucleotides. In various embodiments, the core “gap” region comprises between 5-10 contiguous DNA nucleotides. In an embodiment, the 3 “wing” comprises between 1-6 locked nucleic acid (LNA) nucleotides.

In various embodiments, the gapmer ASO comprises a sequence that is at least about 80%, 85%, 90%, 95%, 97%, or 100% complementary to all or a portion of any one of SEQ ID NOs: 522-525 and 536-539.

In various embodiments, the “gap” DNA recruits RNAseH when formed in a duplex with a complementary RNA molecule. In an embodiment, the “gap” DNA is complementary to a region within an isoform C90RF72 transcript.

In various embodiments, the “gap” of the gapmer ASO is at least 50%, at least 55%, at least 60%, at least 65%, at least 70%, at least 75%, at least 80%, at least 85%, at least 90%, at least 95%, or 100% complementary to an equal length portion of an intron region within a C90RF72 transcript.

In various embodiments, contacting a cell with the gapmer ASO preferentially inhibits the expression of C9orf72 isoforms V1 and V3 that are mRNA transcripts containing a hexanucleotide repeat expansion.

In various embodiments, the expression of mutant C90RF72 isoforms V1 and V3 decreases by at least 50%, at least 55%, at least 60%, at least 65%, at least 70%, at least 75%, at least 80%, at least 85%, at least 90%, at least 95%, or 100%.

In various embodiments, the expression of C9orf72 isoform V2 increases by at least 50%, at least 55%, at least 60%, at least 65%, at least 70%, at least 75%, at least 80%, at least 85%, at least 90%, at least 95%, or 100%.

In various embodiments, contacting a cell with the mix of ASOs described herein synergistically increases the expression and function of C9orf72 isoform V2 and decreases the expression and function of the mutant C90RF72 isoforms V1 and V3 by 30% or more.

In various embodiments, the NAT-targeting oligonucleotide is an ASO and binds a target site on a human SCN1A natural antisense transcript, wherein said ASO increases the expression of human SCN1A (SEQ ID NO: 575), and wherein the natural antisense transcript has the nucleic acid sequence as set forth in any one of SEQ ID NOs: 576-582. In various embodiments, the ASO comprises a sequence that is at least about 80%, 85%, 90%, 95%, 97%, or 100% complementary to all or a portion of any one of SEQ ID NOs: 576-582.

In various embodiments, the NAT-targeting oligonucleotide is an ASO and binds a target site on a human SLC2A1 (solute carrier family 2 member 1) natural antisense transcript, wherein said ASO increases the expression of human SLC2A1 (SEQ ID NO: 287), and wherein the natural antisense transcript has the nucleic acid sequence as set forth in any one of SEQ ID NOs: 146-148. In various embodiments, the ASO comprises a sequence that is at least about 80%, 85%, 90%, 95%, 97%, or 100% complementary to all or a portion of any one of SEQ ID NOs: 146-148.

In various embodiments, the NAT-targeting oligonucleotide is an ASO and binds a target site on a human optic atrophy type 1 (OPA1) natural antisense transcript, wherein said ASO increases the expression of human OPA1 (SEQ ID NO: 116), and wherein the natural antisense transcript has the nucleic acid sequence as set forth in any one of SEQ ID NO: 117. In various embodiments, the ASO comprises a sequence that is at least about 80%, 85%, 90%, 95%, 97%, or 100% complementary to all or a portion of any one of SEQ ID NO: 117.

In various embodiments, the NAT-targeting oligonucleotide is an ASO and binds a target site on a human phosphatidylinositol binding clathrin-assembly protein (PICALM) natural antisense transcript, wherein said ASO increases the expression of human PICALM (SEQ ID NO: 345), and wherein the natural antisense transcript has the nucleic acid sequence as set forth in any one of SEQ ID NO: 346-350. In various embodiments, the ASO comprises a sequence that is at least about 80%, 85%, 90%, 95%, 97%, or 100% complementary to all or a portion of any one of SEQ ID NO: 346-350.

In various embodiments, the NAT-targeting oligonucleotide is an ASO and binds a target site on a human Low density lipoprotein receptor-related protein 1 (LRP1) natural antisense transcript, wherein said ASO increases the expression of human LRP1, and wherein the natural antisense transcript has the nucleic acid sequence as set forth in any one of SEQ ID NO: 467-473. In various embodiments, the ASO comprises a sequence that is at least about 80%, 85%, 90%, 95%, 97%, or 100% complementary to all or a portion of any one of SEQ ID NO: 467-473.

In various embodiments, the NAT-targeting oligonucleotide is an ASO and binds a target site on a human frataxin (FXN) natural antisense transcript, wherein said ASO increases the expression of human FXN, and wherein the natural antisense transcript has the nucleic acid sequence as set forth in any one of SEQ ID NO: 526-531. In various embodiments, the ASO comprises a sequence that is at least about 80%, 85%, 90%, 95%, 97%, or 100% complementary to all or a portion of any one of SEQ ID NO: 526-531.

In various embodiments, the first or second nucleic acid based molecule is an siRNA.

In various embodiments, the siRNA is specific for RAPTOR and is set out in any one of SEQ ID NOs: 33-36.

In various embodiments, the siRNA is specific for RICTOR and is set out in any one of SEQ ID NOs: 56-115.

In various embodiments, the siRNA is specific for SCN1A and is set out in any one of SEQ ID NOs: 1-32.

In various embodiments, the siRNA is specific for SLC2A1 and is set out in any one of SEQ ID NOs: 149-344.

In various embodiments, the siRNA is specific for OPA1 and is set out in any one of SEQ ID NOs: 118-145.

In various embodiments, the siRNA is specific for PICALM and is set out in any one of SEQ ID NOs: 351-466.

In various embodiments, the siRNA is specific for LRP1 and is set out in any one of SEQ ID NOs: 474-517.

In various embodiments, the ASO increases or decreases the expression of human C90RF72, RAPTOR, RICTOR, SCN1A, SLC2A1, LRP1, OPA1 or PICALM by at least 30%, at least 50%, at least 55%, at least 60%, at least 65%, at least 70%, at least 75%, at least 80%, at least 85%, at least 90%, or 100%.

Up-regulation or inhibition of gene expression may be quantified by measuring either the endogenous target RNA or the protein produced by translation of the target RNA. Techniques for quantifying RNA and proteins are known to one of ordinary skill in the art.

### Compositions and Formulations

Nucleic acid based molecule sequences can be introduced into cells using known techniques. Transfection, electroporation, fusion, liposomes, colloidal polymeric particles and viral and non-viral vectors as well as other means known in the art may be used to deliver the nucleic acid based molecules to the cell. The method of delivery selected will depend at least on the cells to be treated and the location of the cells and will be known to those skilled in the art. Localization can be achieved by liposomes, having specific markers on the surface for directing the liposome, by having injection directly into the tissue containing the target cells, by having depot associated in spatial proximity with the target cells, specific receptor mediated uptake, viral vectors, or the like.

Provided herein are compositions comprising two or more nucleic acid based molecules as described herein.

In various embodiments, the compositions comprise one or more ASO and or first or second nucleic acid based molecules as disclosed herein useful in the methods. It is further contemplated that the methods and compositions can be carried out with a mix of ASO or nucleic based molecules, such as siRNA that target the same or related molecular pathways associated with a genetic disease or disorder.

In various embodiments, the nucleic acid based molecules of the present disclosure are formulated in a nanoparticle, a liposome, a microparticle, a microsphere, a nanosphere, a unilamellar vesicle, a multilamellar vesicle, a virus-like particle (VLP).

In various embodiments, the nucleic acid based molecules are formulated in a nanoparticle (e.g., lipid nanoparticle). In various embodiments, two or more nucleic acid based molecules are formulated in a nanoparticle. In various embodiments, the nanoparticle is a lipid nanoparticle. Nanoparticles can be produced using methods known in the art.

In various embodiments, the nucleic acid based molecules are present in concentrations reflecting their individual potency.

In various embodiments, the nucleic acid based molecules are delivered with a suitable non-viral vector, for example, as an expression plasmid which, when transcribed in the cell, produces the nucleic acid based molecules.

Additionally, viral vectors comprising an expression control sequence operatively linked to the nucleic acid based molecule sequences of the disclosure may also be used to deliver the oligonucleotides described herein.

Vectors are known or can be constructed by those skilled in the art and should contain all expression elements necessary to achieve the desired transcription of the nucleic acid based molecules. Phagemids are a specific example of such beneficial vectors because they can be used either as plasmids or as bacteriophage vectors. Examples of other vectors include viruses such as bacteriophages, baculoviruses and retroviruses, DNA viruses and other recombination vectors. The vectors can also contain elements for use in either prokaryotic or eukaryotic host systems. The vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al., BioTechniques 4:504-512 (1986) and include, for example, stable or transient transfection, lipofection, electroporation and infection with recombinant viral vectors.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

In various embodiments two or more nucleic acid based molecules of the present disclosure are expressed from a recombinant adeno-associated virus vector (rAAV).

In various embodiments, the two or more nucleic acid based molecules are expressed from AAV serotypes with blood brain barrier (BBB)-penetrating properties. Suitable AAV serotypes for CNS delivery are available and include for example AAV1, AAV2, AAV5, AAV6, AAV7, AAV8, AAV9, and AAVrh10.

In various embodiments, the nucleic acid based molecules are delivered using AAV9. In various embodiments, the two or more nucleic acid based molecules are delivered using the same vector or different vectors.

In various embodiments, the disclosure provides compositions comprising two or more nucleic acid based molecules, as described herein, and a pharmaceutically acceptable carrier.

In various embodiments, the composition comprising two or more nucleic acid based molecules, as described herein, is formulated in a liposome, a nanoparticle, a lipid nanoparticle, an exosome, or a microvesicle.

In various embodiments, the composition comprises two or more nucleic acid based molecules, as described herein, formulated as a lipid nanoparticle.

In various embodiments, the composition further comprises a pharmaceutically acceptable excipient.

In various embodiments, the composition or vector described herein is useful in modulating the expression level of a target gene in a cell, and or in a method of treating disease.

### Administration

The nucleic acid based molecules, vectors and compositions described herein can be administered to a subject in need thereof by any appropriate route which results in an effective treatment in the subject. Administration may be topical (including ophthalmic and to mucous membranes including vaginal and rectal delivery), pulmonary, e.g., by inhalation or insufflation of powders or aerosols, including by nebulizer, intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

In various embodiments, the nucleic acid based molecules, vectors and compositions of the present disclosure are administered to the subject intrathecally, intranasally, intranasally via nasal depot, intracerebroventricularly, intraperitoneally, intramuscularly, subcutaneously, orally, synovially, intravitreally, subretinally, or intravenously.

In various embodiments, the nucleic acid based molecules, vectors and compositions described herein may be conveniently prepared in unit dosage form, according to standard procedures of pharmaceutical formulation. The quantity of active compound per unit dose may be varied according to the nature of the active compound and the intended dosage regime and can be different for each compound in the mix. A unit dosage form will typically be adapted to one or more specific routes of administration of the pharmaceutical composition.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vivo and in vitro animal models. In general, dosage is from 0.001 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses.

### Methods of Treatment

In various embodiments the nucleic acid based molecules, vectors and compositions herein can be used in a method of treating a disease or disorder in a subject in need thereof by modulating the expression level of a target gene in a cell of the subject, when administered in a therapeutically effective amount.

Contemplated is a method for modulating the expression level of a target gene, wherein the target gene can be modulated through multiple regulatory mechanisms in a cell, comprising: contacting the cell with one or more nucleic acid based molecules, including at least a first functional domain and a second functional domain, wherein each one of the nucleic acid based molecules or functional domains has a different effect.

In some embodiments, the disclosure provides a method for modulating the expression level of two or more target genes in a cell comprising: contacting the cell with two or more nucleic acid based molecules, including at least a first nucleic acid based molecule and a second nucleic acid based molecule, wherein each nucleic acid based molecule regulates the expression of a different target genee.

Also contemplated is a method for modulating the expression level of 2 or more target genes in a cell comprising: contacting the cell with a nucleic acid based molecule, including at least a first functional domain and a second functional domain, wherein each one of the functional domains regulates the expression of a different gene.

In some embodiments, the disclosure provides a method of treating a disease or disorder in a subject in need thereof by modulating the expression level of a target gene in a cell of the subject comprising contacting the cell with two or more nucleic acid based molecules, including at least a first nucleic acid based molecule and a second nucleic acid based molecule, wherein the gene(s) associated with a disease or condition can be modulated through multiple regulatory mechanisms.

Also provided is a method of treating a disease or disorder in a subject in need thereof by modulating the expression level of a target gene in a cell of the subject comprising contacting the cell with one or more nucleic acid based molecules, including at least a first functional domain and a second functional domain, wherein each functional domain has a different effect, wherein the gene(s) associated with a disease or condition can be modulated through multiple regulatory mechanisms.

Further contemplated is a method of treating a disease or disorder in a subject in need thereof by modulating the expression level of two or more target genes in a cell of the subject comprising contacting the cell with two or more nucleic acid based molecules, including at least a first nucleic acid based molecule and a second nucleic acid based molecule that modulate two or more different target genes associated with a disease or condition, wherein a disease or condition can be modulated through simultaneous regulation of multiple target genes.

Additionally, the disclosure provides a method of treating a disease or disorder in a subject in need thereof by modulating the expression level of two or more target genes in a cell of the subject comprising contacting the cell with one or more nucleic acid based molecules, including at least a first functional domain and a second functional domain that modulate two or more different target genes associated with a disease or condition, wherein a disease(s) or condition(s) can be modulated through simultaneous regulation of multiple target genes.

In various embodiments, the method comprises contacting the cell with two or more nucleic acid based molecules. In various embodiments, the two or more nucleic acid based molecules comprise at least a two, three, four, five or more nucleic acid based molecules.

It is provided that in various embodiments, the methods herein lead to desired effects in excess of sum of the effects of each treatment alone, the use of the nucleic acid molecules alone or use of each functional domain alone.

In various embodiments, the disease or condition can be modulated through multiple regulatory mechanisms.

In various embodiments, the disease or disorder is a genetic disease or disorder.

In various embodiments, the disease or disorder is a CNS disease or disorder.

In various embodiments, the CNS disease or disorder is selected from the group consisting of: Alzheimer's disease (AD), Friedrich's ataxia (FA), Huntington's disease (HD), sodium channel a2 subunit (SCN2A) encephalopathy, sodium channel a8 subunit (SCN8A) encephalopathy, SCN1A-Associated Dravet Syndrome, C9orf72-mediated amyotrophic lateral sclerosis (ALS) and frontotemporal dementia (FTD), sodium channel a5 subunit (SCN5A) arrhythmia.

In various embodiments, the CNS disease or disorder is a tauopathy, such as Pick disease, progressive supranuclear palsy, corticobasal degeneration, argyrophilic grain disease, globular glial tauopathies, neurofibrillary tangle dementia, chronic traumatic encephalopathy or aging-related tau astrogliopathy.

In various embodiments, the CNS disease or disorder is C9orf72-mediated amyotrophic lateral sclerosis (ALS) and frontotemporal dementia (FTD).

In various embodiments, the subject is a human.

The following examples are intended to illustrate but not limit the invention. While they are typical of those that might be used, other procedures known to those skilled in the art may alternatively be used.

### EXAMPLES

### Materials and Methods

### Cell Culture

SK-N-AS cells were cultured in DMEM (Gibco, USA) with 10% FBS (Gibco, USA), 0.1 mM NEA (Gibco, USA), and 1× Pen/Strep (Gibco, USA) at 37° C. and 5% CO₂. Cells were split upon reaching 80-90% confluency. HEK293T cells were cultured in DMEM (Gibco, USA) with 10% FBS (Gibco, USA), 1× GlutaMAX (Gibco, USA), and 1× Pen/Strep (Gibco, USA) at 37° C. and 5% C02. SH-SY5Y cells were cultured in a 1:1 mixture of EMEM (ATCC, USA) and F12 (Gibco, USA) with 10% FBS (Gibco, USA) and 1× Pen/Strep (Gibco, USA) at 37° C. and 5% C02. Primary Human Astrocytes (ScienCell) were cultured with complete human astrocyte medium (ScienCell) at 37° C. and 5% C02. N2A cells were cultured in EMEM (ATCC, USA) with 10% FBS (Gibco, USA) and 1× Pen/Strep (Gibco, USA) at 37° C. and 5% C02.

Transfection with ASOs or siRNAs

2.5×10⁵ cells in 1 mL culture media were plated per well in 6 well plates and incubated at 37° C. and 5% CO2 for 24 hours prior to transfection. On the day of transfection, oligonucleotides were diluted in ULTRAPURE™ Distilled Water (Invitrogen, USA) to desired concentrations.

For transfecting ASOs, or single-stranded DNA oligonucleotides, 2 μL of each dilution were incubated with a mix of 400 μL of Opti-MEM media (Gibco, USA) and 4 μL of Lipofectamine 2000 (Invitrogen, USA) at room temperature for 20 min. These mixtures were added to appropriate wells harboring the cells in the 6 well plates. A similar mixture with 2 μL of water instead of oligonucleotide solution was used to mock-transfect the control group. The cells were further cultured under normal growth conditions for another 48 hours for downstream RNA analysis, or 72 hours for downstream protein analysis. Media was replaced with fresh growth media 24 hours post-transfection. Following the incubation, the media was discarded, the cells were washed with PBS and processed for RNA or protein extraction.

For transfecting siRNA, or double-stranded RNA oligonucleotides, 1.5 μL of diluted oligonucleotide were incubated with 300 μL of Opti-MEM media (Gibco, USA) and 9 μL Lipofectamine RNAiMAX (Invitrogen, USA) for five minutes at room temperature. For each mixture, 250 μL were added to appropriate wells harboring the cells in the 6 well plates. A similar mixture with 1.5 μL of water instead of oligonucleotide solution was used to mock-transfect the control group. The cells were further cultured under normal growth conditions for another 48 hours for downstream RNA analysis, or 72 hours for downstream protein analysis. Following the incubation, the media was discarded, the cells were washed with PBS and processed for RNA or protein extraction.

### RNA Extraction, Reverse Transcription and Quantitative Real Time PCR

Cultured cells were lysed with 300 μl of TRIzol™ Reagent (ThermoFisher, USA) per well. RNA was extracted from the samples using Direct-zol RNA Miniprep kit (Zymo Research, USA) following manufacturer's protocol. The quality and concentration of extracted RNA was determined using Nanodrop 2000 (ThermoFisher, USA) as described by the manufacturer prior to reverse transcription. cDNA was synthesized with qScript™ cDNA synthesis kit (Quantabio, USA) using 1000 ng of total RNA based on manufacturer's protocol. 2 μl of RT reaction containing cDNA were mixed with TaqMan® Fast Advanced MasterMix and TaqMan® Gene Expression Assays labeled with FAM (Hs00374696_ml specific for human or Mm00450580_ml assay specific for mouse Scn1a RNA; Custom designed Hu-SCN1A with forward primer 5′-TGGGTTACTCAGAACTTGGA-3′ SEQ ID NO: 583), reverse primer 5′-GCATTCACAACCACCCTC-3′ (SEQ ID NO: 584), and probe 5′-/56-FAM/CAAATCTCT/ZEN/CAGGACACTAAGAGCTCTGAGAC/3IABkFQ/-3 (SEQ ID NO: 585) for human or Mm00450583_mH for mouse productive SCN1A (ThermoFisher, USA)) in Quantstudio 6 Flex (Applied Biosystems) [J Hsiao et. al., 2016; Lim K et al., 2020].

For detection of C9orf72 isoforms the following Taqman assays can be used: Hs00376619_m1 (FAM) (Catalog #4351368, ThermoFisher) for transcripts V1-V3; assay for intron 1: Intron1 forward primer GGTCAGAGAAATGAGAGGGAAAG (SEQ ID NO: 586); Intron1 reverse primer CGAGTGGGTGAGTGAGGA (SEQ ID NO: 587); Intron 1 probe (FAM) AAATGCGTCGAGCTCTGAGGAGAG (SEQ ID NO: 588). Levels of V1 transcript can be estimated based on the difference between the results from the 2 assays. Taqman assays were also used for the detection of Raptor (Hs00375332_ml for human, Mm01242613_m1 for mouse) and Rictor (Hs00380903_ml for human, Mm01307318_m1 for mouse). Taqman assays can be used for detection of OPA1 (Hs01047013_m1), SLC2A1 (Hs00892681_ml), PICALM (Hs00200318_ml), and LRP1 (Hs00233856_ml).

### Western Blots

Transfected cells were rinsed with PBS and lysed with MPER buffer (Thermo Scientific, USA). Proteins from MPER extracts were size fractionated in precast 4-12% SDS-PAGE (Criterion gels, Bio-Rad) and transferred onto PVDF membranes. Primary antibodies from Cell Signaling were used to detect Raptor (2280, 1:1000), Rictor (2114, 1:1000), LC3B (3868, 1:1000), AKT (9272, 1:1000), and phospho-AKT (4060, 1:2000), as well as GAPDH as an endogenous control (BioLegend, 607902). To detect C9orf72, mouse monoclonal anti-C9orf72 antibody GT779 (1:2000, GeneTex Inc, Cat. no. GTX632041) and 1:1000 dilution of the secondary DyLight® 594 goat anti-rabbit antibody (Thermo Fisher, Cat. no. 35560) were used according to manufacturer's protocol. Blots were visualized and quantified using the Odyssey imaging system (LI-COR Biosciences Image studio V5.2). Alternatively, western blot analysis was carried out using ImageJ.

### ELISA

Transfected cells were rinsed with PBS and lysed with MPER buffer (Thermo Scientific, USA). Proteins from MPER extracts were loaded into 96-well ELISA microplates for total Tau (KHB0041, Thermo Scientific, USA), phospho-Tau181 (KH00631, Thermo Scientific, USA), phospho-Tau231 (KHB8051, Thermo Scientific, USA), and phospho-Tau396 (KHB7031, Thermo Scientific, USA) and processed according to manufacturer's protocol.

iPSC-Derived Neurons

iPSCs were maintained as colonies on Corning Matrigel matrix (Millipore Sigma) in mTeSR1 medium (STEMCELL Technologies). Neural progenitors were produced using STEMdiff Neural System (STEMCELL Technologies). iPSCs were suspended in an AggreWell800 plate and allowed to grow as embryoid bodies in STEMdiff Neural Induction Medium for 5 days, with daily 75% medium changes.

Embryoid bodies were harvested with a 37 μm cell strainer and plated onto Matrigel-coated plates in STEMdiff Neural Induction Medium, that was changed daily for 7 days, with 85-95% of embryoid bodies exhibiting neural rosettes 2-days post-plating. Rosettes were manually selected and transferred to plates coated with poly-L-ornithine and laminin in STEMdiff Neural Induction Medium (STEMCELL Technologies). The medium was changed daily until cells reached 90% confluence (7 days) and considered to be neural progenitor cells (NPCs). NPCs were disassociated with TrypLE (ThermoFisher) and passaged at a ratio of 1:2 or 1:3 on poly-L-ornithine/laminin plates in a neural maintenance medium (NMM; 70% DMEM, 30% Ham's F12, 1X B27 supplement) supplemented with growth factors (20 ng/mL FGF2, 20 ng/mL EGF, 5 μg/mL heparin).

For maturation into neurons, NPCs were maintained and expanded for <5 passages, and at >90% confluence were passaged 1:4 onto poly-L-orinithine/laminin-coated plates in NMM supplemented with growth factors. The next day, Day 0 of differentiation, the medium was changed to fresh NMM without growth factors. Differentiating neurons will be maintained in NMM for ≥4 weeks, with twice weekly 50% medium changes. Cortical neurons were replated with TrypLE at a density of 125,000 cells/cm² as needed.

Primary neurons were transfected with ASO gymnotically at the indicated dose 5 days after culture and collected 15 days after treatment.

### Example 1

This example lists disease-relevant genomic loci with multiple nucleic acid associated regulatory mechanisms (FIG. 8). Identification of disease associated genes whose loci contain NMD exons, NATs, upstream open reading frames and/or other regulatory elements was carried out by mining public variant databases as described by e.g., Yan et al., Proc Natl Acad Sci. 112(11): 3445-50 (2015).

### Example 2

This example describes synergistic upregulation of SCN1A expression in vitro using a combination of ASOs (termed ultra-potent nucleic acid or upNA) with NAT-targeting and NMD exon splicing activity (SEQ ID NOs: 577-582). SCN1A siRNA are set out in SEQ ID NO: 1-32.

The SCN1A gene encodes an alpha subunit of the voltage-gated sodium channel Nay1.1. Loss of function mutations in one copy of this gene (haploinsufficiency) is known to cause Dravet syndrome (DS) or genetic epilepsy with febrile seizures plus (GEFS+), severe childhood encephalopathies. In most studied DS cases, no mutant protein is produced and the characteristics of the Nay1.1-mediated sodium current are not significantly altered. However, the amplitude of the sodium current and SCN1A mRNA and protein levels are diminished. Therefore, upregulating the expression of the normal SCN1A allele represents a therapeutic strategy for DS. Augmentation of SCN1A expression improved seizure phenotype in a mouse model of Dravet syndrome [Hsiao et al. 2016].

It was shown that the SCN1A loci in human and mouse genomes express NATs [Hsiao et al. 2016]. While NATs have diverse regulatory functions, many of them act to repress expression of their target coding gene. Accordingly, targeting NATs with antisense oligonucleotides can result in de-repression of the sense gene and increased protein expression [Katayama et al. 2005; Modarresi et al. 2012]. Indeed, treatment with antisense oligonucleotides targeted against SCN1A NAT induced specific upregulation of SCN1A both in vitro and in vivo and significantly reduced the occurrence of seizures in a mouse model of DS [Hsiao et al. 2016].

Additionally, SCN1A expression is regulated by the presence of NMD exons in its mRNAs. The majority of mRNA molecules containing these exons are destroyed through nonsense mediated decay (NMD) pathway and are not available for translation [Lim et al. 2020]. Splice-switching ASOs that can prevent the inclusion of toxic exons in SCN1A were shown to increase the levels of protein-producing (productive) mRNA transcripts and corresponding protein levels [Lim et al. 2020].

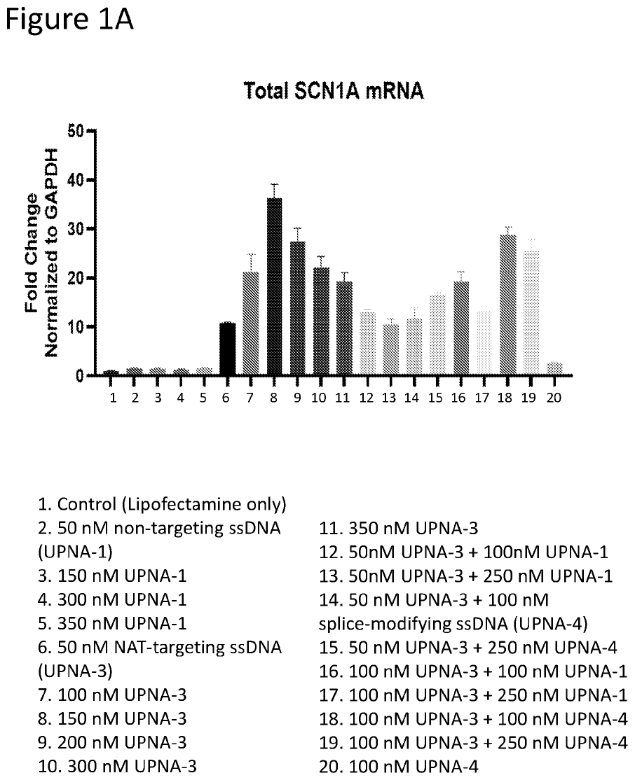

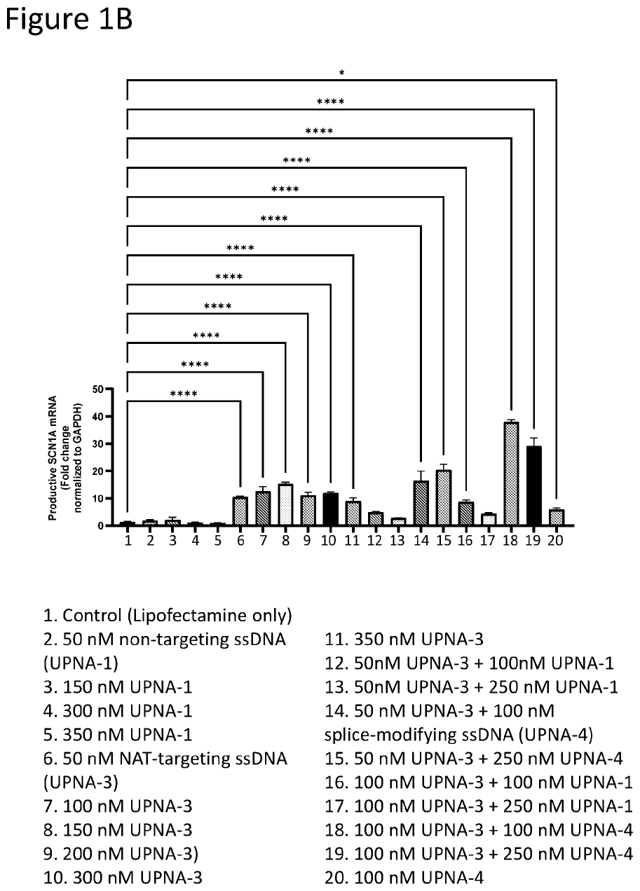

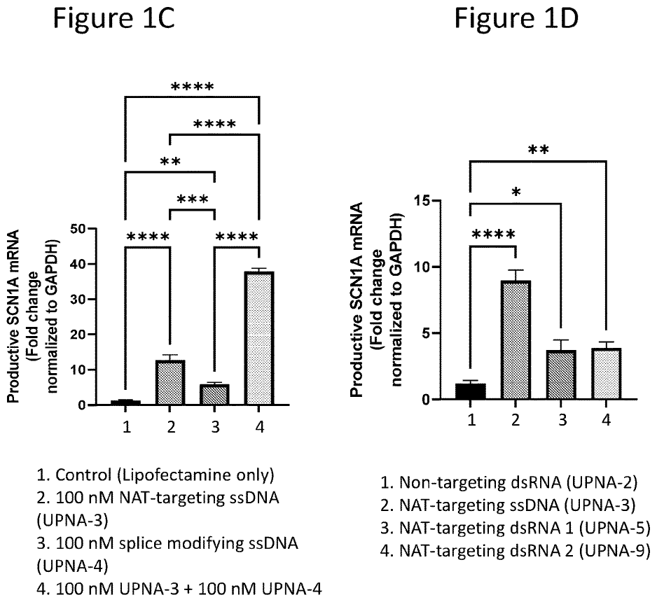

As the NAT-targeted and splice-switching approaches target different mechanisms, it is hypothesized herein that combining NBTs targeting these processes may result in a synergistic effect. Synergy is in fact observed when SK-N-AS cells are transfected in vitro with a mix of ASOs with NAT-blocking and NMD exon splicing actions, leading to amplification of expression in excess of the sum of effects achieved by treatment with each ASO alone (FIGS. 1A-1C).

The synergistic effect on SCN1A protein expression in immortalized cell lines and patient fibroblasts is confirmed using Western blots. Combinations of NAT-targeting and NMD exon-targeting oligonucleotides that produce the highest upregulation of SCN1A protein are further tested to develop efficient treatment for Dravet syndrome and other diseases that can benefit from SCN1A upregulation, such as Alzheimer's disease (Martinez-Losa et al. 2018).

Different classes of ultra-potent nucleic acids (upNAs) for example ssDNA and dsRNA can also be used to synergistically target the same transcript as they exert their function by utilizing different enzymes. For example, single-stranded DNAs (ASOs) cleave RNA targets by forming a DNA:RNA duplex which is recognized and cleaved by the enzyme RNase H. Double-stranded RNA (siRNA) cleaves RNA targets by engaging AGO2/RISC pathway. Both RNase H and AGO2/RISC each have a finite efficiency and limit for activity. Thus, combining NBTs with different mechanisms of action can help maximize the effect by engaging two independent processes, which allows for overall lower doses of upNA to be used.

Indeed SK-N-AS cells treated with either ASOs or siRNAs targeting SCN1A NAT for 48 hours showed a significant upregulation of productive SCN1A mRNA expression (FIG. 1D). SK-N-AS cells treated with combinations of ASOs and siRNAs targeting the SCN1A NAT will show a higher degree of productive SCN1A mRNA upregulation relative to cells treated with either ASOs or siRNAs alone at the same total oligonucleotide concentration. Furthermore, treating SK-N-AS cells with both ASOs and siRNAs simultaneously effectively reduces the load on either RNaseH or RISC, allowing for proper DNA stability and endogenous miRNA function.

The synergistic upregulation of SCN1A observed in SK-N-AS cells treated with a combination of ASOs which have NAT-targeting and NMD exon splicing activity at an equivalent, or lower, total oligonucleotide concentration compared to cells treated with either ASO alone (FIG. 1C) indicate that upNAs can be utilized to reduce drug doses without reducing efficacy. This ultimately minimizes the risk of toxicity in the treatment of multiple genetic disorders. For further therapeutic application, ultra-potent nucleic acids (upNAs) described in Example 2 will be further screened in available disease models, such as mice carrying known Dravet-causing mutations, and non-human primates to assess their beneficial effects on SCN1A levels and disease symptoms. upNAs will then be injected intrathecally or intracerebroventricularly and animal tissues collected at defined periods of time to evaluate Scn1a levels. Additionally, animals will be monitored for improvements in disease phenotype. upNAs that show significant efficacy in these studies will be further screened in in vivo toxicology experiments and progressed to clinical trials

Furthermore, to improve the delivery of the synergistic ASO combination for the treatment of Dravet syndrome the following approach is proposed. In the clinic the naked oligonucleotides targeting both mechanisms will have to be regularly re-administered IT, which could expose patients to significant burden and a possibility of IT injection-associated adverse events. To circumvent these problems, it is contemplated to vectorize NAT-targeting and poison exon-targeting NBTs using a CNS-penetrant strain of adeno-associated virus, AAV9.

AAV9-based NBT expressing constructs are advantageous because of the availability of central nervous system (CNS) penetrant AAV9 capsids that permit systemic (e.g., intravenous or subcutaneous) administration for CNS targeting NBTs. Vectors also permit the use of cell-type specific promoter (e.g., GABAergic neuron targeted promoter/enhancer (mDLX) which ensures tissue specific expression of the payload. Furthermore, vectors offer the possibility of co-expressing multiple NBTs targeting different biological mechanisms in one construct. Finally, unlike naked NBTs, vectorized NBTs require a single or infrequent dosing.

Plasmid multiplex constructs are designed and tested in cell culture. Variants that induce the highest expression of SCN1A RNA and protein are selected for packaging in to AAV9 vector. Vectorized constructs are injected IV in non-human primates. Animal tissues are collected after defined time periods and assessed for SCN1A expression using real time PCR. Constructs that induce therapeutically-relevant levels of SCN1A protein upregulation are further studied in clinical trials in Dravet syndrome.

Alternatively, naked oligonucleotides or multiplex constructs expressing NBTs targeted at both NATs and NMD exon mechanisms are delivered intranasally, encapsulated or not in LNP particles that increase stability and CNS penetrance. The intranasal route to the brain utilizes a highly permeable patch of nasal cavity termed olfactory epithelium. Intranasal administration at the olfactory epithelium area could be achieved using a commercially available nebulizer, either propellant activated (e.g., Kurve Technologies' Vianase (Craft et al., 2017), Impel Neuropharma's Precision Olfactory Device [Shrewsbury et al., 2019], Alchemy Pharmatech's Naltos Device or breath activated (e.g., Optinose [Quintana et al., 2017]). Alternatively, minimally invasive intranasal depot (MIND) technique [Padmakumar et al. 2020] is used to deliver a more precise dose of NBTs.

### Example 3

This example describes synergistic gene targeting strategies in ALS associated with C9orf72 repeat expansions.

Hexanucleotide GGGGCC repeat expansions in the first intron of the chromosome 9 open reading frame 72 (C9orf72, SEQ ID NO: 518-521) gene are the most common genetic cause of ALS, constituting approximately 35-50% of genetic cases. The expanded GGGGCC repeats are bidirectionally transcribed into repetitive RNA, which forms sense and antisense RNA foci. These repetitive RNAs can be translated in every reading frame to form five different dipeptide repeat proteins (DPRs)—poly-GA, poly-GP poly-GR, poly-PA and poly-PR—via a non-canonical mechanism known as repeat-associated non-ATG (RAN) translation. Three non-exclusive mechanisms have been proposed as the basis of the pathogenic effects of the repeat expansions: 1) diminished expression of C9orf72 protein due to steric inhibition of transcription or splicing by the repeats, 2) toxic gain of function from sense and antisense C9orf72 repeat-containing RNA or, 3) toxicity of DPRs. Notably, transcription of one of the three alternatively spliced isoforms of C90RF72, V2, is not affected by repeat expansion.

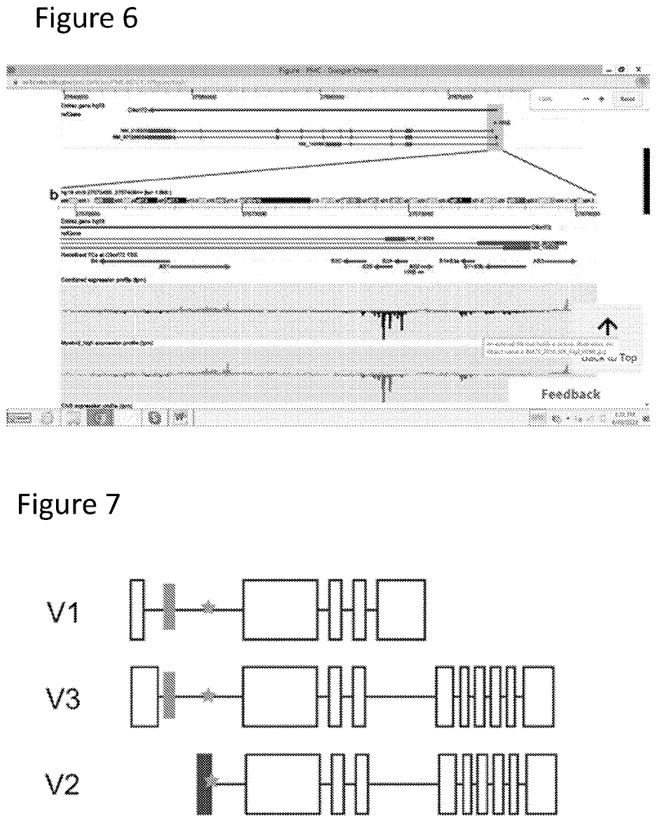

A therapeutic strategy for ALS targets upregulating the expression of the non-pathogenic isoform V2 that encodes C9orf72-S(short) (FIG. 6 and FIG. 7) while downregulating the mutated isoforms V1 and V3. This can be achieved by combining 3 simultaneous treatments: 1) isoform V2 upregulation using NAT-targeting ASOs; 2) knockdown of isoforms V1 and V3 using allele-specific siRNAs; 3) facilitation of splicing-out of intron 1 using synthetic U7 constructs.

Novel potential NAT sequences were identified (SEQ ID NOs: 522-525) and tested for their role in regulating expression of the C90RF72 transcripts. For this purpose, specific ASOs (SEQ ID NOs: 540-551) were designed to target each of the potential C90RF72 NATs. These ASOs were transfected into HEK293 cells and cells were harvested for RNA extraction as described in Example 1. The RNA is reverse transcribed and used in the real time quantitative PCR reaction to specifically assay the levels of C90RF72 short isoform.

Western blots are used to determine the levels of C90RF72 protein. Additionally, analysis of the epigenetic status of the promoter is performed to detect H3K9me3 and DNA methylation levels that determine protein expression. The selected ASOs that induce the highest expression of the C90RF72 short isoform are further tested in cultured neurons. These cells are derived from iPSCs as described in Methods.

Oligonucleotides that induce transcript knockdown of the mutant V1 and V3 sense transcripts, thus eliminating toxic dipeptide repeat proteins, are designed to span the exon 1-2 junction. These oligonucleotides are tested in cell culture as described for in Methods. Transcript V1, V2 and V3 levels are monitored using RT PCR to ensure reduction of variants V1 and V3 and preservation of V2 expression. Western blots are used to determine the levels of C90RF72 protein.

NBTs that enhance splicing out of the intron containing the expanded repeats can increase C90RF72 protein production, shown to be beneficial in ALS, synergistically with NAT-targeting ASOs and oligonucleotides reducing V1 and V3 expression. Such enhanced splicing can be achieved using synthetic constructs based on U7 small nuclear RNA (U7 snRNA). Endogenous U7 snRNAs participate in histone pre-mRNA processing and comprise 2 domains: 1) a domain that interacts with histone pre-mRNA through complementary base-pairing, and 2) a domain that interacts with small nuclear ribonucleoprotein (snRNP) complex and positions it at a pre-mRNA molecule. Domain 1 of U7 snRNA can be replaced with a sequence from the vicinity of the intron 1 splice sites of C90RF72 pre-mRNA, which will facilitate intron 1 excision. Constructs containing these 2 domains can be encoded by a viral vector such as AAV9, which enables the delivery of this splicing modulator into a range of cells and tissues, including the CNS.

A multiplexing strategy involves ASOs that target C90RF72 NAT(s) to increase production of a shorter isoform, or of all isoforms, and/or oligonucleotides that knockdown the expression of the mutant V1 and V3 sense transcripts, thus eliminating toxic dipeptide repeat RNAs and proteins. NBTs that that facilitate splicing out of the expanded intron and therefore increase the availability of productive C90RF72 mRNA could also be included. These NBTs can be administered as a mix of oligonucleotides or expressed from a single construct as described herein.

### Example 4

This example describes a synergistic combination of NAT-targeting and splicing modulation approaches for upregulating frataxin (FXN) expression in Friedreich's ataxia (FA).

Friedreich's Ataxia (FA) is an incurable inherited neurologic disorder caused by an expanded trinucleotide (GAA) repeat within intron 1 of the frataxin (FXN) gene. In a normal human genome there are from 7 to 22 copies of a GAA repeat in intron 1 of the FXN gene. However, most patients with FA (98%) have a repeat expansion of between 200 and 900 copies. Patients are homozygous or compound heterozygous for expansion. GAA-TTC repeats of more than 59 copies form ‘sticky DNA’ and inhibit FXN transcription in vivo and in vitro leading to FXN protein insufficiency. FXN locus also contains a NAT [U.S. Pat. No. 9,593,330] and an NMD exon [Lim et al. 2020]. While this expansion does not change the coding region of F×N and does not result in expression of a mutant protein, it reduces expression of FXN protein. Agents that increase expression of FXN protein to restore it to normal levels have the potential to alleviate the disease.

Treatment of patient fibroblasts with ASOs against FXN NAT resulted in significant upregulation of FXN mRNA [U.S. Pat. No. 9,593,330]. At the same time, it has been shown that targeting 3′ and 5′ untranslated regions (UTRs) of FXN mRNA with ASOs stabilizes it and increases protein production [Li et al. 2021, Shen et al. 2020]. Encoding such ASOs in the same AAV construct as NAT-targeting ASOs or administering them together in a lipid nanoparticle in concentrations that reflect their individual potency results in synergistic effects on FXN protein upregulation. Exemplary FXN NAT are set out in SEQ ID NOs: 526-531.

Another NBT type shown to upregulate FXN protein is SINEUPs [Bon et al. 2019]. Endogenous SINEUPs are encoded head-to-head antisense to the 5′ end of the target sense mRNA and can enhance its translation without upregulating mRNA levels. SINEUPs are distinguished by the presence of specific binding and effector domains. Binding domain is formed by an antisense region overlapping the start codon of the target mRNA and confers specificity to a particular protein coding transcript. Effector domain at the 3′ end of a SINEUP comprises embedded transposable element sequences, such as inverted short interspersed nuclear element B2 (invSINEB2), Alu or MIR, that are capable of upregulating translation by binding activating protein complexes. Synthetic miniSINEUP molecules, which combine essential elements of binding domains complementary to a specific gene and invSINEB2 effector domains have been developed for multiple genes [Arnoldi et al. 2022]. FXN-targeted miniSINEUP can be expressed by the same vector as AntagoNATs and 3′ and 5′ UTR-targeting ASOs or administered in the same lipid nanoparticle in concentrations optimal for each NBT.

It is contemplated that NBTs that enhance splicing out of the intron containing the expanded repeats will increase FXN protein production, shown to be beneficial in FA, synergistically with NAT-targeting oligonucleotides and miniSINEUPs. Such enhanced splicing can be achieved using synthetic constructs based on U7 small nuclear RNA (U7 snRNA) as described above. Domain 1 of U7 snRNA is replaced with a sequence from the vicinity of the intron 1 splice sites of FXN pre-mRNA, which facilitates exon 1 excision.

Combinatorial constructs encompassing several or all of the protein upregulation methods described above can be encoded by a viral vector such as AAV9, which will enable the delivery of this splicing modulator into a range of cells and tissues including CNS, after systemic delivery. Such constructs will be further tested in vitro and in vivo as described herein and advanced to clinical trials and clinical use.

### Example 5

This example describes combining NBTs to alter the ratio of protein complexes with common binding partners to enhance a biological effect in the treatment of mTORopathies by simultaneously targeting RAPTOR and RICTOR.

mTORopathies are defined as diseases related to abnormal function of mechanistic/mammalian target of rapamycin complex (mTORC). mTORC relays an array of intra- and extracellular stimuli to control multiple cellular anabolic and catabolic processes thus affecting cell growth and survival. Representative examples of mTORC-related diseases are tuberous sclerosis complex (TSC) and Alzheimer's disease (AD). TSC is a rare genetic disease that causes benign tumors to grow in the brain and in other vital organs such as the kidneys, heart, eyes, lungs, and skin, thus impairing their function. AD is characterized by progressive mental deterioration that can occur in middle or old age, due to generalized neurodegeneration [Rapaka et al. 2022].

The kinase, mechanistic target of rapamycin (mTOR), is present in two distinct complexes—mTORC1 and mTORC2. RAPTOR is an essential factor specific to mTORC1 while RICTOR is an essential factor specific to mTORC2. As the cellular pool of mTOR kinase binds either RAPTOR to form mTORC1 or RICTOR to form mTORC2, knocking down RAPTOR decreases mTORC1 while increasing mTORC2 by increasing the total amount of mTOR available to bind RICTOR. Furthermore, upregulating RICTOR further increases mTORC2 while further diminishing the total amount of mTOR available to bind RAPTOR, thereby shifting the ratio of mTORC1 to mTORC2 to a greater extent than either individual treatment.

In both TSC and AD, simultaneous upregulation of RICTOR and downregulation of RAPTOR, the two components of mTORC, would lead to significant therapeutic benefits. Such effect can be achieved by application of upNAs combining oligonucleotides targeting RICTOR NAT and RAPTOR mRNA. Such upNAs can be designed as a combination of oligonucleotides, as divalent siRNA constructs, that exert both activities, or as vectorized constructs that can induce down-regulation of RAPTOR and up-regulation of RICTOR.

For example, RAPTOR (SEQ ID NO: 574) downregulation was achieved using an siRNA targeting RAPTOR mRNA. siRNAs targeting RAPTOR mRNA (SEQ ID NOs: 33-36) were designed and transfected at a concentration of 10 nM in SK-N-AS cell culture as described in the methods. After 24 h total RNA was extracted and the expression of RAPTOR mRNA quantified using real time PCR as described in methods.

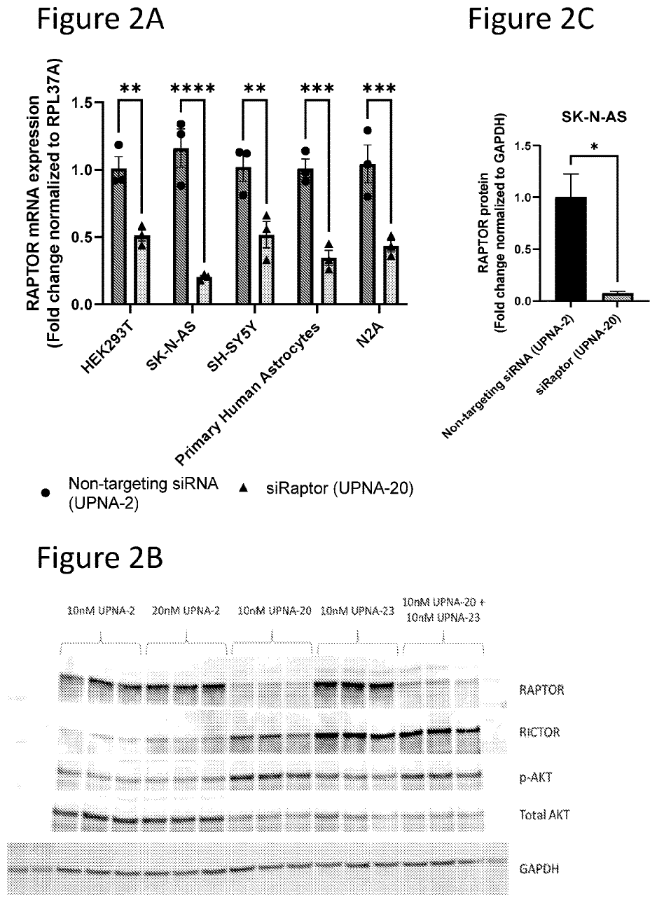

In SK-N-AS cells transfected with RAPTOR siRNA, RAPTOR mRNA levels are reduced by 80% after 48 hours of treatment and RAPTOR protein levels are reduced by 93% after 72 hours of treatment (FIGS. 2A-C). The siRNA that induced the highest downregulation of RAPTOR in vitro will be tested in a mix or in a combined viral vector construct with RICTOR-targeting NBTs.

To upregulate RICTOR (SEQ ID NO: 552), a promoter bidirectional transcript (SEQ ID NO: 37, ENST00000692264.1) has been identified herein, as well as others in the promoter region—(SEQ ID NOs: 38-40, DB344625, AA905279, DW452491) and several potential NATs in the human RICTOR locus ((SEQ ID NOs: 41-55, AA493266, B1492147, DB344625, B1014609, BQ935479, A1807252, AA806990, HY262189, AA603494, T51933, AV730173, CR746673, N63643, N59272, BM676019). Promoter transcript sequences contain Alu, LINE and LTR elements and may function as SINEUPs, lncRNA molecules that regulate their partner gene translation. siRNAs targeting each identified transcript were designed (SEQ ID NOs: 56-115). HEK293T cells were cultured, oligonucleotides were transfected at a concentration of 10 nM, and downstream mRNA and protein expression were analyzed using RT-PCR and ELISA as described in Methods.

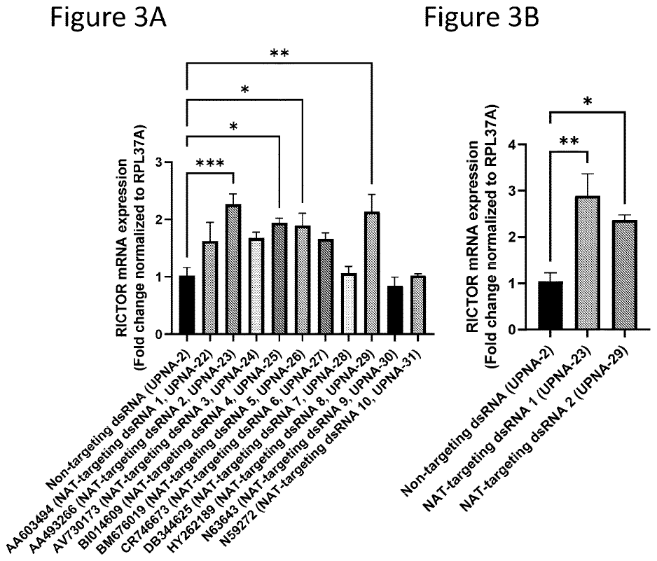

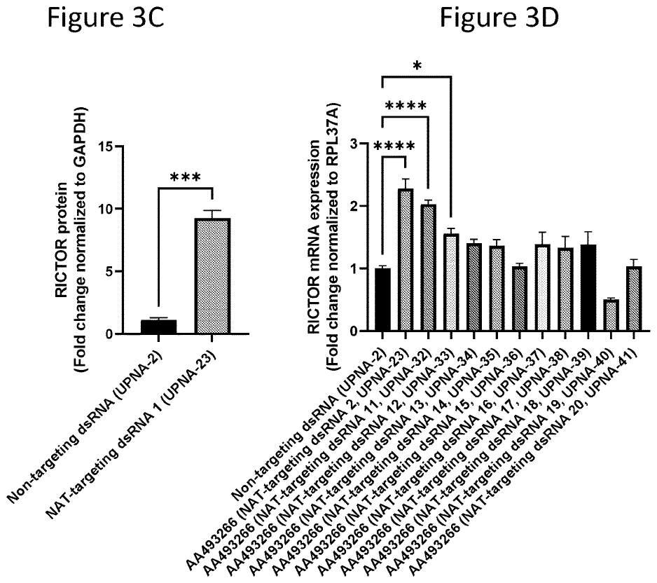

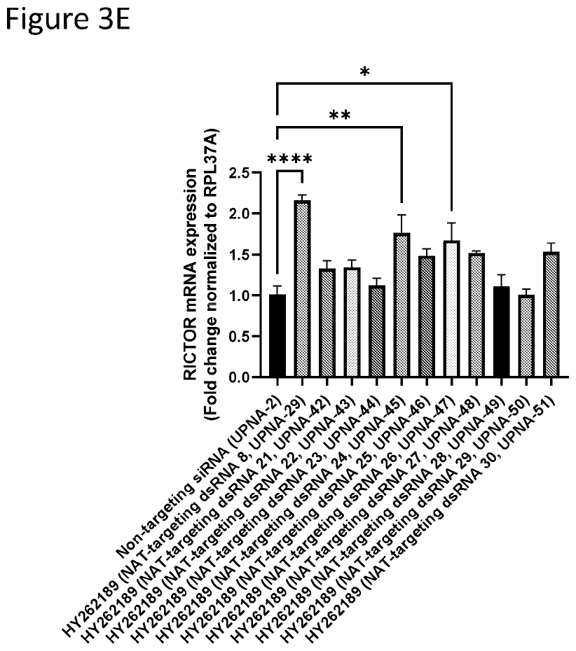

The siRNAs targeting AA493266 (SEQ ID NO: 41) and HY262189 (SEQ ID NO:48) significantly upregulated RICTOR mRNA ˜3-fold after 48 hours and increased RICTOR protein ˜9-fold after 72 hours (FIGS. 3A-E). ASOs or siRNAs that induced the highest upregulation of RICTOR in vitro can then be tested together with oligonucleotides downregulating RAPTOR or both NBTs could be co-expressed from a single (e.g., combined) viral vector construct.

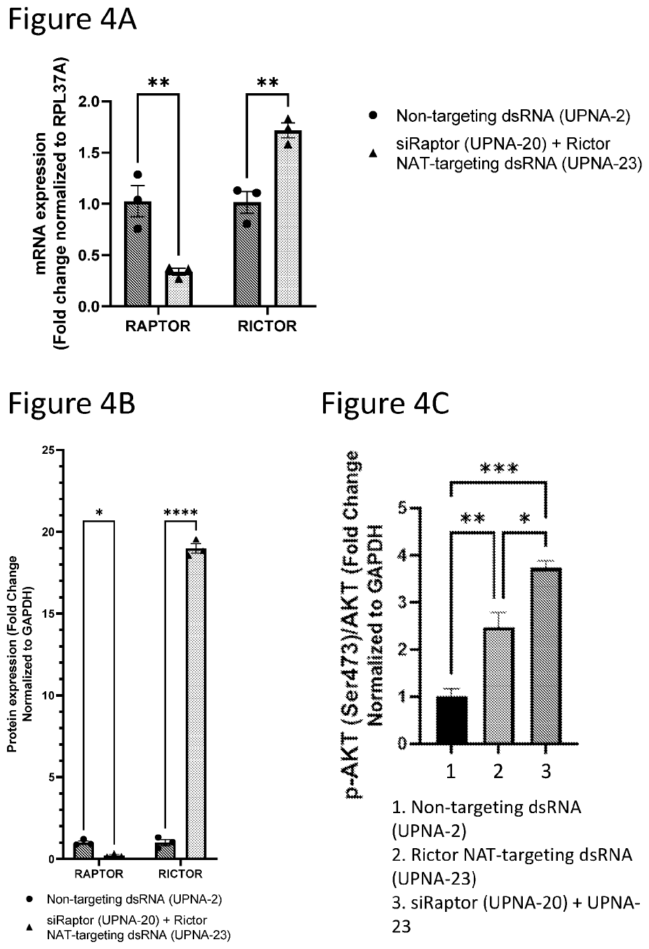

In SK-N-AS cells cotransfected with RICTOR NAT-targeting siRNA and RAPTOR mRNA-targeting siRNA for 48 hours, RAPTOR mRNA is reduced by 66% and RICTOR mRNA is upregulated 1.7-fold (FIG. 4A). After 72 hours of cotransfection in SK-N-AS cells, RAPTOR protein was knocked down 79% and RICTOR protein was upregulated 19-fold (FIG. 4B). This degree of RICTOR protein upregulation is greater than that seen with the same concentration of RICTOR NAT-targeting siRNA alone, suggesting an enhanced upregulation of the protein by reducing the quantity of a competing binding partner (RAPTOR) for mTOR with a RAPTOR mRNA-targeting siRNA.

Furthermore, upregulation of AKT phosphorylation (downstream target of mTORC2) is higher after treatment with the combination of RICTOR NAT-targeting siRNA and RAPTOR mRNA-targeting siRNA relative to RICTOR NAT-targeting siRNA alone (FIG. 4C).

An AAV-based viral vector construct is designed to express oligonucleotides that induce the highest upregulation of RICTOR and downregulation of RAPTOR mRNA or protein. This construct is transduced into cell culture and expression of RICTOR and RAPTOR determined using real time PCR and ELISA or western blotting as described in Methods. The expression of the constructs is tested in vivo in non-human primate model and in mouse models of AD (double PS2/APP mutants) and TSC (TSC1 and TSC2 knockout mice) and effects on treatment of disease are measured, e.g., desired upregulation of RICTOR and downregulation of RAPTOR.

### Example 6

This example describes synergistic upregulation of OPA1 expression using an upNA comprising a combination of NBTs with NAT-targeting and NMD exon splicing activity.

The OPA1 (optic atrophy type 1) gene encodes a dynamin-like GTPase of the inner mitochondrial membrane which regulates the equilibrium of mitochondrial fusion and fission. Mutations in OPA1 commonly result in haploinsufficiency, reducing the overall OPA1 protein levels, which causes autosomal dominant optical atrophy (ADOA). Approximately 1 in 30,000 people are affected by ADOA, which results in progressive and irreversible vision loss within the first decade of life (Jüschke et al. 2021).

Human OPA1 transcripts have been shown to contain an in-frame NMD-inducing stop codon arising from an alternatively spliced exon, resulting in non-productive mRNA, which is degraded rather than translated. ASOs which prevent the inclusion of the NMD-inducing exon in mature mRNA reduce non-productive OPA1 mRNA and increase expression of productive OPA1 mRNA and protein (Venkatesh et al. 2020).

Using the UCSC genome browser, OPA1 NAT sequences overlapping the coding region of OPA1 were identified in the human genomic locus (OPA1-AS1 or ENST00000433105.5; SEQ ID NO: 116-117). To upregulate OPA1, siRNAs targeting OPA1-AS1 were generated using IDT siRNA design tool (SEQ ID NO: 118-145) and are tested in vitro. For this purpose, cells are transfected using Lipofectamine RNAiMAX as described in Methods for 48 hours prior to collecting and purifying total cellular RNA. The RNA is used to quantify OPA1 mRNA with real time PCR. Additionally, cells are transfected with OPA1-AS1-targeting siRNAs for 72 hours prior to extracting cellular protein to quantify OPA1 protein levels with either western blot or ELISA. Here, combinations of oligonucleotides targeting the various regulatory mechanisms (upNAs) show enhanced efficacy relative to sum of effects of individual oligonucleotides alone. For in vivo treatment, combinations of NBTs in concentrations determined by their individual potencies (upNAs) are incorporated in appropriate carrier, such as a lipid nanoparticle or a liposome, or cloned in an appropriate plasmid or viral vector.

To develop treatments for ADOA, and other diseases caused by OPA1 deficiency, such as Behr syndrome, dominant optic atrophy plus syndrome and mitochondrial DNA depletion syndrome-14 (cardioencephalomyopathic type, MTDPS14), upNAs showing the highest upregulation capacity in vitro are delivered intravitreally to animal disease models and non-human primates, upNAs with the highest activity in animal models are further tested for toxicity in rodents and non-human primates. Well-tolerated upNAs with highest potency on OPA1 protein upregulation and vision improvement are studied in clinical trials and approved for marketing by regulatory agencies.

### Example 7

This example describes synergistic upregulation of SLC2A1 expression using a combination of NBTs with promoter-targeting and NAT-targeting activity.

The SLC2A1 (solute carrier family 2 member 1 or GLUT1) gene encodes the glucose transporter which plays an especially important role in shuttling glucose across the blood brain barrier. GLUT1 is highly expressed by brain endothelial cells and vasculature-associated astrocytes in the brain. Heterozygous mutations in SLC2A1 cause dystonia-9 (DYT9), a disorder estimated to affect 1 in 24,000 newborns in Scotland, characterized by childhood-onset seizures, delayed neurological function, microcephaly, and complex movement disorders. GLUT1 insufficiency can also cause GLUT1 deficiency syndromes 1 and 2.

Using UCSC genome browser NATs overlapping either an exon of SLC2A1 (ENST00000691915.1, SEQ ID NO: 146) or the promoter region of SLC2A1 (ENST00000653200.1, SEQ ID NO: 147) were identified, as well as a sense transcript overlapping the SLC2A1 promoter region (ENST00000640492.1, SEQ ID NO: 148) and designed siRNAs targeting each transcript (SEQ ID NO: 88-185) that can be used in upNA to upregulate SLC2A1.

dsRNA sequences were generated using IDT siRNA design tool (SEQ ID NO: 149-344). It has been shown that targeting promoter regions of genes with small double-stranded RNA can activate gene expression and ultimately increase protein levels (JCschke et al. 2021).

Here, SK-N-AS cells are transfected with dsRNAs targeting either promoter-overlapping sense and antisense transcripts, or NATs overlapping SLC2A1 exons for 48 hours prior to collecting cellular RNA for real time PCR quantification of SLC2A1 mRNA. Oligonucleotides which induce significant upregulation of SLC2A1 mRNA are then transfected in SK-N-AS cells for 72 hours and cellular protein is collected to quantify levels of GLUT1 protein using either western blot or ELISA to assess degree of protein upregulation. Here, combinations of oligonucleotides targeting the various regulatory mechanisms (upNAs) show enhanced efficacy relative to individual oligonucleotides alone. Therapeutic upNas for the treatment of SLC2A1 deficiency-related diseases are then developed as described in Example 6.

### Example 8

This example describes synergistic upregulation of PICALM expression using a combination of NBTs with promoter-targeting and NAT-targeting activity.

The gene PICALM encodes phosphatidylinositol binding clathrin-assembly protein which plays a critical role in endocytosis and autophagy. PICALM is the 3rd most significant risk gene for Alzheimer's disease (AD) after APOE and BIN1, likely due to its role in modulation of tau pathology. In mice, point mutations of PICALM cause shortened lifespan resulting from various abnormalities, suggesting PICALM may play a role in aging and longevity (Ando et al., 2022).

Using the UCSC genome browser a putative promoter region of 1,000 bases located upstream of the PICALM gene (ENST00000393346.8, SEQ ID NO: 345) as well as several NATs overlapping PICALM sense transcript (SEQ ID NO: 346-350, A1694932.1, BF910880.1, BQ016971.1, AL135735.1, MN309288.1) were identified. These sequences were targeted with custom siRNAs designed using IDT siRNA design tool (SEQ ID NO: 351-466).

SK-N-AS cells are transfected with dsRNAs targeting either the promoter region or NATs of PICALM locus for 48 hours prior to collecting cellular RNA for real time PCR quantification of PICALM mRNA. Oligonucleotides which induce significant upregulation of PICALM mRNA are then transfected in SK-N-AS cells for 72 hours and cellular protein is collected to quantify levels of PICALM protein using either western blot or ELISA to assess degree of protein upregulation. Here, combinations of oligonucleotides targeting the various regulatory mechanisms (upNAs) show enhanced efficacy relative to individual oligonucleotides alone. Therapeutic upNas for the treatment of PICALM deficiency-related diseases are then developed as described in Example 6.

### Example 9

This example describes synergistic upregulation of LRP1 expression using a combination of NBTs with promoter-targeting and NAT-targeting activity.

The LRP1 gene encodes low-density lipoprotein receptor-related protein 1, an endocytic receptor that regulates cellular cholesterol homeostasis. Additionally, LRP1 is a receptor for the Alzheimer's disease (AD) pathology-related proteins tau and APOE. Knocking down LRP1 significantly reduces tau uptake in vitro and has an effect on tau spreading in vivo (Rauch et al. 2020). It is proposed that upregulating LRP1 to increase tau uptake under conditions of enhanced autophagy is a therapeutic strategy to reduce levels of tau in AD brains, which cannot be accomplished by any currently available AD therapeutics.

Upregulation of LRP1 is achieved by targeting NATs in human LRP1 locus (SEQ ID NO: 467-470, LRP1-AS1 (ENST00000555461.1), BX477794, DC310139, DC401271), a microRNA overlapping LRP1 coding region (SEQ ID NO: 472, MIR1228 (ENST00000408438.1)), and the promoter region of 1,000 bases upstream of LRP1 (SEQ ID NO: 473, ENST00000243077.8) and promoter NAT (SEQ ID NO: 471, AA704922) with custom designed oligonucleotides (SEQ ID NO: 474-517). LRP1 NAT sequences were identified using UCSC genome browser and dsRNA oligonucleotide sequences were generated using IDT siRNA design tool.

SK-N-AS cells are transfected with dsRNAs targeting either the promoter region, NAT, or miRNA regulating LRP1 levels for 48 hours prior to collecting cellular RNA for real time PCR quantification of LRP1 mRNA. Oligonucleotides which induce significant upregulation of LRP1 mRNA are then transfected in SK-N-AS cells for 72 hours and cellular protein is collected to quantify levels of LRP1 protein using either western blot or ELISA as described in Methods to assess degree of protein upregulation. Combinations of oligonucleotides targeting the various regulatory mechanisms (upNAs) tested in a similar manner show enhanced efficacy relative to sum of effects of individual oligonucleotides alone. Therapeutic upNAs for the treatment of LRP1 deficiency-related diseases are then developed as described in Example 6.

### Example 10

This example describes simultaneous treatment with upNAs with different gene targets resulting in a synergistic biological effect beneficial in the treatment of multiple diseases.

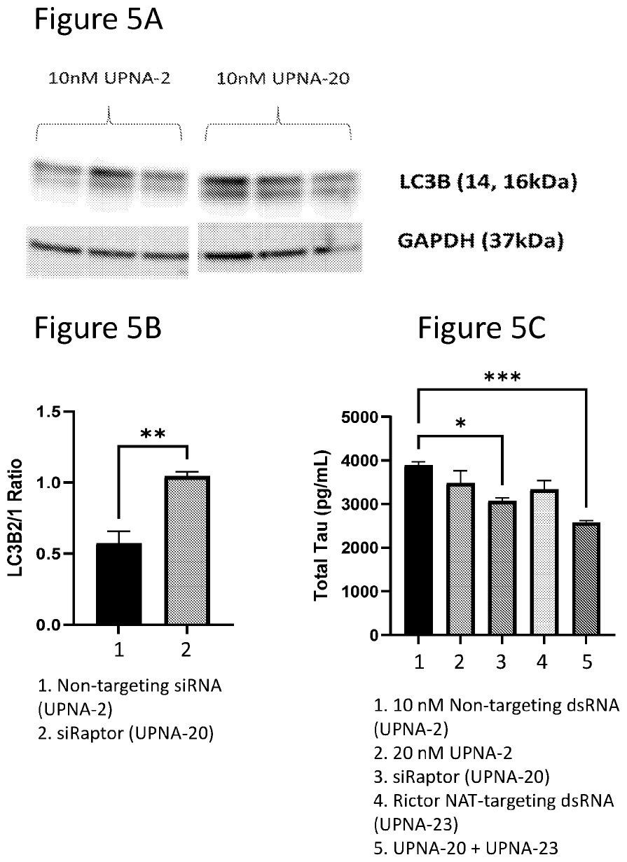

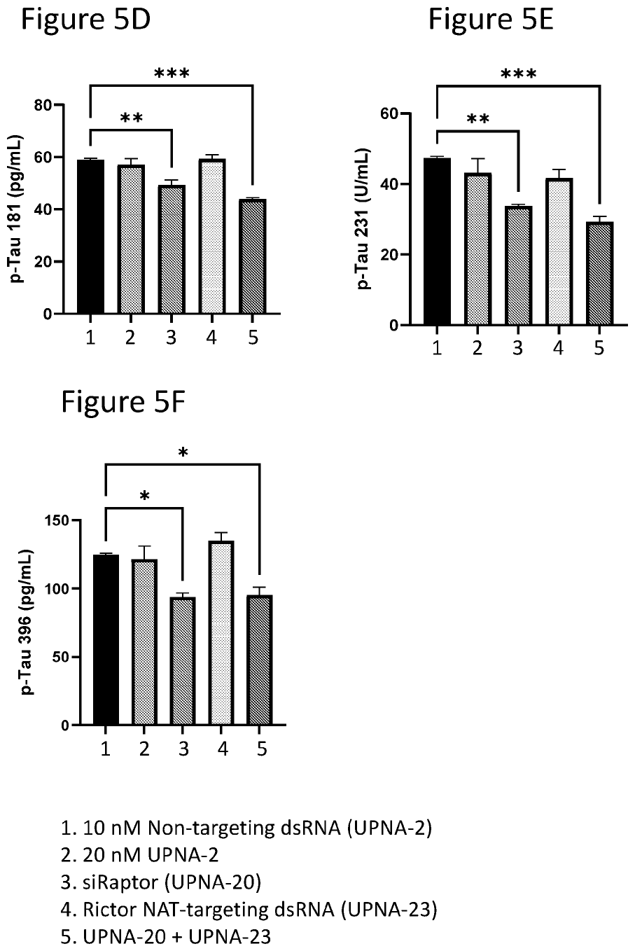

Upregulation of either PICALM or LRP1 enhances cellular endocytosis, specifically of pathological proteins such as tau. The increased uptake may overload the intracellular space with pathological protein and ultimately increase tau spreading between cells. However, under conditions of enhanced autophagy, the pathological proteins endocytosed by the increased PICALM and LRP1 are rapidly degraded, thereby reducing the spreading and total concentrations of pathological protein. Notably, mTORC-targeting upNAs that inhibit mTORC1 via Raptor knockdown, have been shown to increase autophagy, as evidenced by an increased ratio of LC3B2 to LC3B1 and an overall decrease in total and phospho-tau in vitro (FIGS. 5A-F).

Therefore, combining upNAs that increase endocytosis of pathological proteins from the extracellular space, such as PICALM and LRP1 upNAs, with upNAs that upregulate autophagy via inhibition of mTORC1 results in synergistic amplification of tau degradation, beneficial in the treatment of AD and tauopathies, such as Pick disease, progressive supranuclear palsy, corticobasal degeneration, argyrophilic grain disease, globular glial tauopathies, neurofibrillary tangle dementia, chronic traumatic encephalopathy and aging-related tau astrogliopathy. Moreover, this strategy is generalizable to combine upNAs against different targets which are all related with the same biological process.

Numerous modifications and variations to the disclosure, as set forth in the embodiments and illustrative examples described herein, are expected to occur to those skilled in the art. Consequently only such limitations as appear in the appended claims should be placed on the disclosure.

### REFERENCES

1. Ando et al. 2022. Cells. 11(24):3994.
2. Antosova et al. 2009. Trends Biotechnol. 27(11):628-635.
3. Arnoldi et al. (2022) Methods Mol Biol. 2434:63-87.
4. Bon et al. 2019. Nucleic Acids Res. 47(20):10728-10743.
5. Ezkurdia et al. 2014. Hum Mol Genet. 23(22):5866-5878.
6. Gragoudas et al. 2004. N Engl J Med. 351(27):2805-2816.
7. Gryziewicz L. 2005. Adv Drug Deliv Rev. 57(14):2092-2098.
8. Hsiao et al. 2016. EBioMedicine. 9:257-277.
9. Hume et al. 2005. Science. 309(5740):1564-6.
10. Juschke et al. 2021. Mol Ther Nucleic Acids. 26:1186-1197.
11. Katayama et al. 2017. Nat Biotechnol. 35(3):249-263.
12. Li et al. 2015. Curr Opin Virol. 11:130-136.
13. Li et al. 2021. Nucleic Acids Res. 49(20):11560-11574.
14. Lim et al. 2020. Nat Commun. 11(1):3501.
15. Martinez-Losa et al. 2018. Neuron. 98(1):75-89.e5.
16. Mendell et al. 2004. Nat Genet. 36(10):1073-1078.
17. Modarresi et al. 2012. Nat Biotechnol. 30(5):453-459.
18. Padmakumar et al. 2021. J Control Release. 331:176-186.
19. Rapaka et al. 2022. Neurochem Int. 155:105311.
20. Rauch et al. 2020. Nature. 580(7803):381-385.
21. Shen et al. 2020. Bioorg Med Chem. 28(11):115472.
22. Venkatesh et al. 2020. Invest. Ophthalmol. Vis. Sci. 61(7):2755.
23. Wahlestedt C. 2013. Nat Rev Drug Discov. 12(6):433-46.
24. Wengertet al. 2022. Brain Res. 1775:147743.
25. Yan et al. 2015. Proc Natl Acad Sci USA. 112(11):3445-3450.

1. A method for modulating the expression level of a target gene, wherein the target gene can be modulated through multiple regulatory mechanisms in a cell, comprising: contacting the cell with two or more nucleic acid based molecules, including at least a first nucleic acid based molecule and a second nucleic acid based molecule, wherein each nucleic acid based molecule has a different effect, wherein the effect is one of the following:
a) modulating the action of a natural antisense transcript (NAT) that controls the expression of the target protein;
b) modulating the splicing of a mutated exon or intron or a nonsense mediated RNA decay-inducing exon in mRNA encoding a target protein encoded by the target gene or a mutated exon in non-coding RNA (ncRNA);
c) inhibiting the effect of a translation suppression element in the 5′ untranslated region of a transcript of the target gene, wherein the translation suppression element is an upstream open reading frame (uORF);
d) knocking down an mRNA encoding a pathogenic isoform of the target protein or of a target regulatory ncRNA involved in a pathogenic process;
e) knocking down all isoforms of the mRNA of the target protein or of a target regulatory ncRNA;
f) modulating effects of promoter RNAs affecting in cis or in trans the expression from the genomic locus encoding the target protein or target ncRNA;
g) modulating effects of enhancer RNAs affecting in cis or in trans the expression from the genomic locus encoding the target protein or target ncRNA, or
h) replicating the effects of the natural SINEUP transcript through inclusion of its essential parts.

2. (canceled)

3. A method for modulating the expression level of 2 or more target genes in a cell comprising: contacting the cell with two or more nucleic acid based molecules, including at least a first nucleic acid based molecule and a second nucleic acid based molecule, wherein each nucleic acid based molecule regulates the expression of a different target gene.

4-6. (canceled)

7. A method of treating a disease or disorder in a subject in need thereof by modulating the expression level of a target gene in a cell of the subject comprising contacting the cell with two or more nucleic acid based molecules, including at least a first nucleic acid based molecule and a second nucleic acid based molecule, wherein the gene associated with a disease(s) or condition(s) can be modulated through multiple regulatory mechanisms.

8. (canceled)

9. A method of treating a disease or disorder in a subject in need thereof by modulating the expression level of two or more target genes in a cell of the subject comprising contacting the cell with two or more nucleic acid based molecules, including at least a first nucleic acid based molecule and a second nucleic acid based molecule that modulate 2 or more different target genes associated with a disease or condition, wherein a disease) or condition can be modulated through simultaneous regulation of multiple target genes.

10-11. (canceled)

12. The method of claim 1, further comprising a third oligonucleotide, fourth nucleic acid based molecule or more, wherein the nucleic acid based molecule has one of the following effects:
a) modulating the action of a natural antisense transcript (NAT) that controls the expression of the target protein;
b) modulating the splicing of a mutated exon or intron or a nonsense mediated RNA decay-inducing exon in mRNA encoding a target protein encoded by the target gene or of a ncRNA;
c) inhibiting the effect of a translation suppression element in the 5′ untranslated region of a transcript of the target gene, wherein the translation suppression element is an upstream open reading frame (uORF);
d) knocking down an mRNA encoding a pathogenic isoform of the target protein or of a target regulatory ncRNA involved in a pathogenic process;
e) knocking down all isoforms of the mRNA of the target protein or target ncRNA;
f) modulating effects of promoter RNAs affecting in cis or in trans the expression from the genomic locus encoding the target protein or target ncRNA;
g) modulating effects of enhancer RNAs affecting in cis or in trans the expression from the genomic locus encoding the target protein or target ncRNA, or
h) replicating the effects of the natural SINEUP transcript through inclusion of its essential parts.

13. The method of claim 1, wherein the target gene is selected from the group consisting of genes as set out in FIG. 8.

14. (canceled)

15. The method of claim 7, wherein the genetic disease or disorder is selected from the group consisting of Friedrich's ataxia, mTORopathies, such as tuberous sclerosis complex (TSC) and Alzheimer's disease (AD), Huntington's disease, sodium channel a2 subunit (SCN2A) encephalopathy, sodium channel a8 subunit (SCN8A) encephalopathy, SCN1A-associated Dravet syndrome, C9orf72-mediated amyotrophic lateral sclerosis (ALS) and frontotemporal dementia (FTD), sodium channel a5 subunit (SCN5A) arrhythmia, tauopathies, Pick disease, progressive supranuclear palsy, corticobasal degeneration, argyrophilic grain disease, globular glial tauopathies, neurofibrillary tangle dementia, chronic traumatic encephalopathy or aging-related tau astrogliopathy.

16. The method of claim 1, wherein the first and second nucleic acid based molecules are selected from the group consisting of an antisense oligonucleotide (ASO), a guide RNA (gRNA), an siRNA, an shRNA, a small nuclear RNA (snRNA), a mini SINEUP, and a miRNA.

17. (canceled)

18. The method of claim 1, wherein the first nucleic acid based molecule is an antisense oliqonucleotide (ASO), and wherein the ASO binds to a natural antisense transcript (NAT).

19. The method of claim 18, wherein the NAT-targeting oligonucleotide binds a target site on a human natural antisense transcript, and wherein the ASO comprises a sequence that is at least about 80%, 85%, 90%, 95%, 97%, or 100% complementarity to all or a portion of:
i) a C90RF72 natural antisense transcript as set out in any one of SEQ ID NOs: 522-525 and 536-539;
ii) a rapamycin-insensitive companion of mammalian target of rapamycin (RICTOR) natural antisense transcript set out in any one of SEQ ID NOs: 37, 38-40, 41-55, and/or 553-573;
iii) a sodium channel protein type 1 subunit alpha (SCN1A) natural antisense transcript set out in any one of SEQ ID NOs: 576-582;
iv) a solute carrier family 2 member 1 (SLC2A1) natural antisense transcript set out in any one of SEQ ID NOs: 146-148;
v) an optic atrophy type 1 (OPA1) natural antisense transcript set out in SEQ ID NO: 117;
vi) a human phosphatidylinositol binding clathrin-assembly protein (PICALM) natural antisense transcript set out in any one of SEQ ID NO: 346-350;
vii) a low density lipoprotein receptor-related protein 1 (LRP1) natural antisense transcript set out in any one of SEQ ID NO: 467-473; or
viii) a frataxin (FXN) natural antisense transcript set out in any one of SEQ ID NO: 526-531.

20-23. (canceled)

24. The method of claim 18, wherein the ASO is single-stranded.

25. (canceled)

26. The method of claim 19, wherein the ASO comprises a sequence that is at least about 80%, 85%, 90%, 95%, 97%, or 100% complementarity to all or a portion of i) a C90RF72 natural antisense transcript as set out in any one of SEQ ID NOs: 522-525 and 536-539, and wherein the second nucleic acid based molecule is a splice-modulating ASO, and
wherein the splice-modulating ASO:
(i) targets the exon 1-2 splicing of C90RF72;
(ii) knocks down mutant C90RF72 isoforms V1 (SEQ ID NO: 533) and V3 (SEQ ID NO: 535);
(iii) spans the exon 1-2 junction of C90RF72; and/or
(iv) comprises a sequence that is at least about 80%, 85%, 90%, 95%, 97%, or 100% complementary to all or a portion of any one of SEQ ID NOs: 540-551.

27-38. (canceled)

39. The method of claim 19, wherein the ASO comprises a sequence that is at least about 80%, 85%, 90%, 95%, 97%, or 100% complementarity to all or a portion of ii) a rapamycin-insensitive companion of mammalian target of rapamycin (RICTOR) natural antisense transcript set out in any one of SEQ ID NOs: 37, 38-40, 41-55, and/or 553-573; and wherein the second nucleic acid based molecule is an ASO or an siRNA, wherein such nucleic acid based molecules knock down the mRNA encoding RAPTOR protein having the sequence set forth in SEQ ID NO: 574, or wherein the oligonucleotides comprise a sequence that is at least about 80%, 85%, 90%, 95%, 97%, or 100% complementary to all or a portion of SEQ ID NO: 574.

40-54. (canceled)

55. The method of claim 1, wherein the first nucleic acid based molecule is an siRNA.

56. The method of claim 55,
wherein the siRNA is specific for RAPTOR and is set out in any one of SEQ ID NOs: 33-36,
wherein the siRNA is specific for RICTOR and is set out in any one of SEQ ID NOs: 56-115,
wherein the siRNA is specific for SCN1A and is set out in any one of SEQ ID NOs: 1-32,
wherein the siRNA is specific for SLC2A1 and is set out in any one of SEQ ID NOs: 149-344,
wherein the siRNA is specific for OPA1 and is set out in any one of SEQ ID NOs: 118-145,
wherein the siRNA is specific for PICALM and is set out in any one of SEQ ID NOs: 351-466, or
wherein the siRNA is specific for LRP1 and is set out in any one of SEQ ID NOs: 474-517.

57-62. (canceled)

63. The method of claim 1, wherein the nucleic acid based molecules are expressed by plasmid vector or a viral vector.

64. The method of claim 1, wherein the two or more nucleic acid based molecules are expressed from a recombinant adeno-associated virus (rAAV) or a lentivirus.

65-66. (canceled)

67. The method of claim 64, wherein the two or more nucleic acid based molecules are expressed on the same vector or on different vectors.

68. The method of claim 7, wherein the two or more nucleic acid based molecules are administered to the subject intrathecally, intranasally, intranasally via nasal depot, intracerebroventricularly, intraperitoneally, intramuscularly, subcutaneously, orally, synovially, intravitreally, subretinally, or intravenously.

69. A composition comprising two or more nucleic acid based molecules, including at least a first nucleic acid based molecule and a second nucleic acid based molecule, wherein each nucleic acid based molecule has a different effect, wherein the effect is one of the following:
a) modulating the action of a natural antisense transcript (NAT) that controls the expression of the target protein;
b) modulating the splicing of a mutated exon or intron or a nonsense mediated RNA decay-inducing exon in mRNA encoding a target protein or target ncRNA encoded by the target gene;
c) inhibiting the effect of a translation suppression element in the 5′ untranslated region of a transcript of the target gene, wherein the translation suppression element is an upstream open reading frame (uORF);
d) knocking down an mRNA encoding a pathogenic isoform of the target protein or of a target regulatory ncRNA involved in a pathogenic process;
e) knocking down all isoforms of the mRNA of the target protein or target ncRNA;
f) modulating effects of promoter RNAs affecting in cis or in trans the expression from the genomic locus encoding the target protein or target ncRNA;
g) modulating effects of enhancer RNAs affecting in cis or in trans the expression from the genomic locus encoding the target protein or target ncRNA, or
h) replicating the effects of the natural SINEUP transcript through inclusion of its essential parts.

70-95. (canceled)


SEQUENCE LISTING (588 sequences)

SEQ ID NO: 1 - DNA, 21 bp, synthetic construct
  source 1..21: mol_type = other DNA; note = SCN1ANAT-targeting oligonucleotide (UPNA-3)
gtggtataggaactggcagca

SEQ ID NO: 2 - DNA, 18 bp, synthetic construct
  source 1..18: mol_type = other DNA; note = SCN1ANAT-targeting oligonucleotide (UPNA-4)
aaggattaaaggtagcaa

SEQ ID NO: 3 - RNA, 25 nt, synthetic construct
  source 1..25: mol_type = other RNA; note = SCN1ANAT-targeting oligonucleotide (UPNA-5)
agcagattacaatcacttattcttt

SEQ ID NO: 4 - RNA, 27 nt, synthetic construct
  source 1..27: mol_type = other RNA; note = UPNA5 complementary strand 5 prime to 3 prime
aaagaataagtgattgtaatctgctct

SEQ ID NO: 5 - RNA, 25 nt, synthetic construct
  source 1..25: mol_type = other RNA; note = SCN1ANAT-targeting oligonucleotide (UPNA-6)
aaagtgaaattgaatgagctaatct

SEQ ID NO: 6 - RNA, 27 nt, synthetic construct
  source 1..27: mol_type = other RNA; note = UPNA-6 complementary strand 5 prime to 3 prime
agattagctcattcaatttcactttac

SEQ ID NO: 7 - RNA, 25 nt, synthetic construct
  source 1..25: mol_type = other RNA; note = SCN1ANAT-targeting oligonucleotide (UPNA-7)
aaaacttgatgagaaggaaacacca

SEQ ID NO: 8 - RNA, 27 nt, synthetic construct
  source 1..27: mol_type = other RNA; note = UPNA-7 complementary strand 5 prime to 3 prime
tggtgtttccttctcatcaagttttag

SEQ ID NO: 9 - RNA, 25 nt, synthetic construct
  source 1..25: mol_type = other RNA; note = SCN1ANAT-targeting oligonucleotide (UPNA-8)
gttctaaaacttgatgagaaggaaa

SEQ ID NO: 10 - RNA, 27 nt, synthetic construct
  source 1..27: mol_type = other RNA; note = UPNA-8 complementary strand 5 prime to 3 prime
tttccttctcatcaagttttagaactt

SEQ ID NO: 11 - RNA, 25 nt, synthetic construct
  source 1..25: mol_type = other RNA; note = SCN1ANAT-targeting oligonucleotide (UPNA-9)
gaaggactgttgaataaattcatca

SEQ ID NO: 12 - RNA, 27 nt, synthetic construct
  source 1..27: mol_type = other RNA; note = UPNA-9 complementary strand 5 prime to 3 prime
tgatgaatttattcaacagtccttcat

SEQ ID NO: 13 - RNA, 25 nt, synthetic construct
  source 1..25: mol_type = other RNA; note = SCN1ANAT-targeting oligonucleotide (UPNA-10)
gaataatcttgttgatataaacact

SEQ ID NO: 14 - RNA, 27 nt, synthetic construct
  source 1..27: mol_type = other RNA; note = UPNA-10 complementary strand 5 prime to 3 prime
agtgtttatatcaacaagattattcac

SEQ ID NO: 15 - RNA, 25 nt, synthetic construct
  source 1..25: mol_type = other RNA; note = SCN1ANAT-targeting oligonucleotide (UPNA-11)
catcaccaagaaattcttaaaatca

SEQ ID NO: 16 - RNA, 27 nt, synthetic construct
  source 1..27: mol_type = other RNA; note = UPNA-11 complementary strand 5 prime to 3 prime
tgattttaagaatttcttggtgatgca

SEQ ID NO: 17 - RNA, 25 nt, synthetic construct
  source 1..25: mol_type = other RNA; note = SCN1ANAT-targeting oligonucleotide (UPNA-12)
gaggttgtcaatcaatattgattta

SEQ ID NO: 18 - RNA, 27 nt, synthetic construct
  source 1..27: mol_type = other RNA; note = UPNA-12 complementary strand 5 prime to 3 prime
taaatcaatattgattgacaacctcag

SEQ ID NO: 19 - RNA, 25 nt, synthetic construct
  source 1..25: mol_type = other RNA; note = SCN1ANAT-targeting oligonucleotide (UPNA-13)
catttctacattctcagataccttg

SEQ ID NO: 20 - RNA, 27 nt, synthetic construct
  source 1..27: mol_type = other RNA; note = UPNA-13 complementary strand 5 prime to 3 prime
caaggtatctgagaatgtagaaatgaa

SEQ ID NO: 21 - RNA, 25 nt, synthetic construct
  source 1..25: mol_type = other RNA; note = SCN1ANAT-targeting oligonucleotide (UPNA-14)
gggtactgctgatagtactattctt

SEQ ID NO: 22 - RNA, 27 nt, synthetic construct
  source 1..27: mol_type = other RNA; note = UPNA-14 complementary strand 5 prime to 3 prime
aagaatagtactatcagcagtacccaa

SEQ ID NO: 23 - RNA, 25 nt, synthetic construct
  source 1..25: mol_type = other RNA; note = SCN1ANAT-targeting oligonucleotide (UPNA-15)
gtacttgcatattcagctatcacct

SEQ ID NO: 24 - RNA, 27 nt, synthetic construct
  source 1..27: mol_type = other RNA; note = UPNA-15 complementary strand 5 prime to 3 prime
aggtgatagctgaatatgcaagtacaa

SEQ ID NO: 25 - RNA, 25 nt, synthetic construct
  source 1..25: mol_type = other RNA; note = SCN1ANAT-targeting oligonucleotide (UPNA-16)
gctatcaccttcaacttaatatgtc

SEQ ID NO: 26 - RNA, 27 nt, synthetic construct
  source 1..27: mol_type = other RNA; note = UPNA-16 complementary strand 5 prime to 3 prime
gacatattaagttgaaggtgatagctg

SEQ ID NO: 27 - RNA, 25 nt, synthetic construct
  source 1..25: mol_type = other RNA; note = SCN1ANAT-targeting oligonucleotide (UPNA-17)
ttggaaaccattgagatacaagtct

SEQ ID NO: 28 - RNA, 27 nt, synthetic construct
  source 1..27: mol_type = other RNA; note = UPNA-17 complementary strand 5 prime to 3 prime
agacttgtatctcaatggtttccaaca

SEQ ID NO: 29 - RNA, 25 nt, synthetic construct
  source 1..25: mol_type = other RNA; note = SCN1ANAT-targeting oligonucleotide (UPNA-18)
cattgagatacaagtcttatttttc

SEQ ID NO: 30 - RNA, 27 nt, synthetic construct
  source 1..27: mol_type = other RNA; note = UPNA-18 complementary strand 5 prime to 3 prime
gaaaaataagacttgtatctcaatggt

SEQ ID NO: 31 - RNA, 25 nt, synthetic construct
  source 1..25: mol_type = other RNA; note = SCN1ANAT-targeting oligonucleotide (UPNA-19)
gcagatttttagaattcagtgatga

SEQ ID NO: 32 - RNA, 27 nt, synthetic construct
  source 1..27: mol_type = other RNA; note = UPNA-19 complementary strand 5 prime to 3 prime
tcatcactgaattctaaaaatctgcca

SEQ ID NO: 33 - RNA, 25 nt, synthetic construct
  source 1..25: mol_type = other RNA; note = RAPTOR siRNA (UPNA-20)
ggcgtcacactggatttgatagaaa

SEQ ID NO: 34 - RNA, 25 nt, synthetic construct
  source 1..25: mol_type = other RNA; note = UPNA-20 complementary strand 5 prime to 3 prime
tttctatcaaatccagtgtgacgcc

SEQ ID NO: 35 - RNA, 25 nt, synthetic construct
  source 1..25: mol_type = other RNA; note = RAPTOR siRNA (UPNA-21)
ggaccttacagattggaacttgcct

SEQ ID NO: 36 - RNA, 25 nt, synthetic construct
  source 1..25: mol_type = other RNA; note = UPNA-21 complementary strand 5 prime to 3 prime
aggcaagttccaatctgtaaggtcc

SEQ ID NO: 37 - DNA, 1075 bp, Homo sapiens
  source 1..1075: mol_type = other DNA; note = RICTOR NAT (ENST00000692264.2 (ENSG00000289196))
ggcggcgggtggtgcaggctcggccccagcgctgagggagcactgagccgacccaaggtctgcaaggagttcgcggcacccgcccgcccacgccggacccggagggcacgtccgccgggaggggcagttcccacgaggaaagtcccattgatgcgagttggcagtgtagaggatgctcccctccggccgccgcggaagccgtcgtccaaatgcaggcgagcggggaggaggaggccgggatgcggccggcgggccacctgctctcgccggcctgccagtctcctgcttgctctgaagccctgccatagacggccccgctcccccgtgtatccactgaaacatttttttataaaaacgctgcgcacagttttagactcctttcctggaatcgctttgctccaactaagttaacacgtaacaaagtttgttttccagcctcgcccttgatcttcagaatgcccctctcggctaggagggcttctccaagtggaagtggttaggcagttttgtttttggccgccaggcgtaggacgcgagaagggggtgcagcggccggggaccagggcggcggtccccgcggtgggatcgcgccagcgccgggaggccggggagagggtgttctctgccgggagccgaggcgaggctgctgggtactgtgtccggcttttatggctctgcagctcctaggatcgtggctcatgcaggtgttgtggattaatctattttatgcataagcctattaacattagggattttaattctagaaactgcttagttaatacgtatgtgtacacacacacacacacacacacacacacacacacacacacacacacacacacacacacacacacacacctggccactgacccagcagctttctcttgaactacatttactttaaagagaggagcgatttgtcctctccagttcctcagctcatctttctattccatgagactgaaagttaattaaaagtggagatctagccttaaccagttctgttttcccagctcccagttaatgcaatatctggcacaaataagtattcaataaatatctgttggatgagtaa

SEQ ID NO: 38 - DNA, 565 bp, Homo sapiens
  source 1..565: mol_type = other DNA; note = RICTOR NAT (DB344625.1)
aaacaaaaacattctgcctttaataataatagcattttaattttatggtttgaaaaggaacgaaataaaatacctgacaacaatagcctgtcatttaggagcaggggatcttagtcacacattgttgtacttgcctttttatatatgcatctttgttgcaaatacactcaagatgaaaagtgcnagaaactgtgtatatttggttttcaggtgggatagcatctgggagattatggagagatgtgttgaagccactgcctttgtggaagcttctacctatgcaggccaggagacaagggcttggtggtgtgcttgggggtggaattgaacagtgactctgtggaattgaacagcttgactctgtgtccccacccaaatctcatctcgaattataatccccataatccccatatgtcgaggaaagaacccagtggagttgattggatcatagaggtgggttcccccatgttgttcttgtaatagtgagtgagttctcatgagatctgatggttttatagggcagttttccctgcttttgctctctctcgcctgctgccgtgtaaga

SEQ ID NO: 39 - DNA, 467 bp, Homo sapiens
  source 1..467: mol_type = other DNA; note = RICTOR NAT (AA905279.1)
ttttacagtcatactcatggctatgatttattacagtgaaaggattcaaagcaaaacaagcaaaaggaaagggcacaatgagtgaagtccagaggaaaccaggtacaaacttccaagaattcttctcctgtagagtcgcacacatacttaattcctccagcaacagtttatgacagcatgtttgaaatgttgtttcctaagtctgcgtgttaaagacttagtacccaaggtttttattgagggctggtcacataagtaccctctgcccagcatgagccaacattccagacttccagggtaaagcagatgttcagcgtaaatcatattgtttgtacaaacagtctaggcacagcaagccactcttatcagttctgggaatggtggaaatcctctcaaaatccaagtccccagacaccaacccaaagccaacttttcaagcaggattttttaaggcagcagtttcaggt

SEQ ID NO: 40 - DNA, 192 bp, Homo sapiens
  source 1..192: mol_type = other DNA; note = RICTOR NAT (DW452491.1)
ggccattacggccgggggctcacgcctgtggtctcagcactttgggaggccgaggcgggcagatcatgaggtcaggagttcgagaccagcctgaccaacatggtgaaaccccgtctttactaaaaatacaaaaattagctatgcgtggtgatgcatgcctgtaatcccggctactcagaagactggggcggg

SEQ ID NO: 41 - DNA, 454 bp, Homo sapiens
  source 1..454: mol_type = other DNA; note = RICTOR NAT (AA493266.1)
ggctctggacaatgccaggcacatggtgggttgtcaataaacagtggttaaaagaaatactgagcaagacagtaagtgacacaaatgttatttggagatgcttaacacgagtttattcttgacaataagcaatacatcttgtaggcatataaaatgtgaagagcaatatgtacattaacatggaaatgtgaaaaaacaaggtacttttctcaatgaactcaaggaatagcaagaaattctatatcttcagaaaacaggatgatgagaataaatgaaaatggagctagattccaacagctttctaagataagttaacttaacttagaaagataaaagttaacatatcttatatctttgggcaatggataattattaaggaaggcatttgtgcaagtgaatggcatgatcagttttttgtgctttaggaatacaactctggagagtgaactgcaga

SEQ ID NO: 42 - DNA, 355 bp, Homo sapiens
  source 1..355: mol_type = other DNA; note = RICTOR NAT (BI492147.1)
tttttttttgagatggagtctcgctctgtggcccaggctgaagtgcagtggcgcgatctcggctcactgcaagctccgcctcccgggttcacaccattctcctgcctcagcctcccgagtagccgggaccacaggtgcccgccaccatgcccggctgttttttctatttttagtagagacaggcttcaccgtgttagccaggatggtctcgatctcctgatctcatgatccgcccgcctcggcctcccaaaatgctgcaattacaggtgtgagccactgtgcccggccagtatttttaatcttttatactgtatttttattggaccttttctatgtttagatgtttagctcgtgc

SEQ ID NO: 43 - DNA, 565 bp, Homo sapiens; identical to SEQ ID NO: 38
  source 1..565: mol_type = other DNA; note = RICTOR NAT (DB344625.1)

SEQ ID NO: 44 - DNA, 515 bp, Homo sapiens
  source 1..515: mol_type = other DNA; note = RICTOR NAT (BI014609.1)
atattataaaaaatggaaaaagcagtttaaaataaatataataaatgtaagtgtaatgtgttttatatgtgcttcattatcatgtattgcatggcacgtatatagtattcaccagagtattatatattaatgtcaccataagttacacatatttatgttaaagtttattttttagagtacttctgatgttacattattttgtagacattttgaaaaataaaaaagccaaagaaaatgttaaaaaattactaacacttccatcccgcagaagcatttttacatatcagtgttaaatatttttctaagtatacagaacaatagaaagcagtcttaattagtataaagttaattatctgacataagtatctaatcctctgctcatcaaaagactattaagaaaatgaaaagacaatccacaaaatgggtaaaaaacattcacagcacaaacatcagacaagattttaataaccaatatatgactatcatctacaaatcaacaataaaataacccatgt

SEQ ID NO: 45 - DNA, 915 bp, Homo sapiens
  source 1..915: mol_type = other DNA; note = RICTOR NAT (BQ935479.1)
catcatggtcatgtgctacttgaaaagtcagtggatcaaggagacctgttatcctgacatccctgacccttacaagagcagcatcctgtcattaataaaattcaaggagaaccctcacctaataataatgaatgtcagtgactgtatcccagatgctattgaagttgtaagcaagccagaagggacaaagatacagttcctaggcactaggaagtcactcacagaaaccgagttgactaagcctaactacctttatctccttccaacagaaaagaatcactctggccctggcccctgcatctgttttgagaacttgacctataaccaggcagcttctgactctggctcttgtggccatgttccagtatccccaaaagccccaagtatgctgggactaatgacctcacctgaaaatgtactaaaggcactagaaaaaaactacatgaactccctgggagaaatcccagctggagaaacaagtttgaattatgtgtcccagttggcttcacccatgtttggagacaaggacagtctcccaacaaacccagtagaggcaccacactgttcagagtataaaatgcaaatggcagtctccctgcgtcttgccttgcctcccccgaccgagaatagcagcctctcctcaattacccttttagatccaggtgaacactactgctaaccagcatgccgatttcataccttatgctacacagacattaagaagagcagagctggcaccctgtcatcaccagtggccttggtccttatcccagtacgatttgcaggtctggtttatataagaccactacagtctggctaggttaaagggcagaagctatggaancttaacactcccccattggagcaagcttgcccctanagatggcaggatcatgggnagcatgcttaccttcc

SEQ ID NO: 46 - DNA, 568 bp, Homo sapiens
  source 1..568: mol_type = other DNA; note = RICTOR NAT (AI807252.1)
ccccatgttttaatggattgtgaatctgagacaaaaggggaagcataagactaaaggagaaaaaaccttacttccaaagaacatttcccaaagtagtttacaaagttcatctcacacaaaattagcacctcactcttaccatgcaaaaataactgcagttataatttgaatgacagacaatttggtttttattgcttaatgatctgtgctttaatgtggggataaaaggcatcctctgaatggaataacattttatataaatagcagtaggaaaatgtaaataagcattgatatcccactgctgaacactttgccgttgtgatatgccctgaaaaactcagcttgaattatctcaagctctagataaaagagctgaaacccattaaagttgtcatttcaaaaggacttgtacacattatgttaataatgtatatacttccaaagagcctcttttaatgtcactgtcatatagaatgtccatattctactaatcgttattagaaaaaatttaattagggagaacagactgtttcccccttccaatctacctaagaataaacaggaaata

SEQ ID NO: 47 - DNA, 474 bp, Homo sapiens
  source 1..474: mol_type = other DNA; note = RICTOR NAT (AA806990.1)
tttttttttagtatactgataaccacaaagctctgagtatctccccaaagtaagtgatataggcagatatgataaggtatacatacatatttttcaaagtaacagtaaaaaaaaaaaaaaaaaaaaaaaacacaaaacattcaggccaatactaactaagccttatttcaggatagatgaccaaggtgagggtacagggatagtgatggtgggggagggggtgtgtgtgtgtgtaagacaaaaaaaaaattactgttaacttttgagggattaaaccacagatttttatactctatattaactgaggtagtgtttaaaatgaatgtctaaaatatgttaatcccattatgaataagattacattacttcctttggaggttttaaccactatttggtctagtaactctgacatttggttctctggaaaggcagtttagttatccaagaccaaagaccacgatgacaggcaacgtt

SEQ ID NO: 48 - DNA, 489 bp, Homo sapiens
  source 1..489: mol_type = other DNA; note = RICTOR NAT (HY262189.1)
caccactgaatcagcagcaaaagatcagtacccagtacacagtaaaggtcaacaaatatttgtctagtgtgacagagaggcagacatgtgaataaaattatgccaattgtaagaaaggtaaaatgcaatatattataaaagcccaaataaagaagatgttcaacactgcgagggaagattgactttttgggaaactttttgttggttacagagaagaaaacagattggaaaagaacagaactagaacagggagagtagttacgaagtcaatagaatcatcaggaaagataataagggcctgaataaaggcatggcaacagaaatggaaaaaacagatttgcaagtatttcagagataaaatcattaccatctggtctcagattggatatgtggaataaggacaaggaacgatgttaagatgtctcctgggtgtcttatttggcccaattaaagtacttctcttaacccagaaagatttttagaataatg

SEQ ID NO: 49 - DNA, 394 bp, Homo sapiens
  source 1..394: mol_type = other DNA; note = RICTOR NAT (AA603494.1)
ttttgccaaatctgagatgcaactgaatttattcttacctcatcatctcctaaacttcatcaacctggtgtttaaggccatcattcaaattaagctgtcctcctcaagcttaccatttgcctaattcaaagtttttctctcagcttttcaaaactttttgacttttccatcacactactgacccatgctaaccaaatccaccatatttcccgtggtttctgcaactacactgtctcaatccttttcataactatccttcactgttccttatgacttggtttaaagatttgtattccctcctcctgttccatatcctttaacattacctaaagctttgagttaaatgaaggttgccagggaaaaaaatgttttaaatgtaaacaacaacaaagac

SEQ ID NO: 50 - DNA, 473 bp, Homo sapiens
  source 1..473: mol_type = other DNA; note = RICTOR NAT (T51933.1)
aagaattttaagtacattttattaacaatgtatccctttgataagattatgcttcaggaggcttttaatgcccttgacataaactatacacattatacaaaaacaagaaaatcacaacaaaaaaaatcaaggtgagcaaaaccatttggggacaaatcttatttaaattatacacaactcaatgaaatattcttacagaaaaaatataaatactttttctttctatgttacagttatacaatataaatcagatttcaatgtctgttcagtgacctacaaacaccagaacctccaaatatgtagcagcgtattactaaataaaaaaggaaggaaactcatgtgggttaggagaggcattaaggtctgaggatttttttcaccaattcccttaccccttttcaaaactaggtttacaccctttgttttaccatgcggccttnggggttccggncggggntttttggtaaccattt

SEQ ID NO: 51 - DNA, 700 bp, Homo sapiens
  source 1..700: mol_type = other DNA; note = RICTOR NAT (AV730173.1 (AV730173))
aagataacaacggatttcttgtccaaaacagaagacaatgaagcaacatctttaaagtaatgaaacaaatgctttaaaaatatgcttcacaaaaacaagccatgggccagatatggccttataggcagtagtttgccacctccggaatagagtgtcaatttcttcagatggctataaactattaaaatcttcaccatcctatttgttttaaataaaaacaaaagcacaatttttaaaatacattaccaagtttttgatattttagtttaagagccattatgacttcctttgagacttttatctgttaacaccaatgcaaggaactgttttgaattagtgttttcctcatcaaatgaaacggtttgcactgtcctgctttaaatttagcttgcgtctgcctaccttactccattctgttatactgggtgttgtctatgacaacaaatgtggtgtcaaagcactaagtacactacttagctatttccaattgctttgaagttgctacatgctgagatcaatcattgggatttcatgacagatgaaatatttttaatggtaataaaattcattcttaagtgaattagtgtggcagtccccaaccttttggacaccagggacaaggttctgtggcaatttttccatggaccccgnggttggggatggttttggaataaacttgtcaacctctcattaggcattg

SEQ ID NO: 52 - DNA, 452 bp, Homo sapiens
  source 1..452: mol_type = other DNA; note = RICTOR NAT (CR746673.1)
agcgctggagagtttttttttttttttgcatattatcttttattcatctctccaattttaaaattacagttttcttacttgataagaaaaaaaattaaagctaaacatattataaaatatcaacagtgccagtttattcccacaggattccaataaataatatacttgaaatcacttattaggactattatactaacattgttagttaataatataggctaatgtcaattttataatgtgaagcatttttaaaaatagtcctgataaaaacagaacacttaagtttcaaaaaatgatttggaaaaacaaattaggggacaatcaatcttttatatcacacatgatttcatacctaaatacaaacaaatccttgatttaagctgactccaaatactatattcaaaggacaacaatgcaatacatgcacacgaagaaactataatacagtgcat

SEQ ID NO: 53 - DNA, 426 bp, Homo sapiens
  source 1..426: mol_type = other DNA; note = RICTOR NAT (N63643.1)
tgagacagagtgtcgctctgtcacccaggatagtgcagtggtgtgatctcagctcaatgcaaactccgcctcccaggttcaagcaattctcctgcctcagcctctggagtagctgggaatacccatgcccagctaattttttttgtatttttagtagagacggggtttcaccagattggtcaggctggtctcaaactcctgacctcaggcggtccacccgctncggcctcccaaagtgctaggattacaggaatcactatttttaactcaggggaaaaatgaaaaacaataaaaacatttccctggcaatgtatactatgtatcacagtaagtttatgtctacattaaactcattgcagtgcaataaagtctaccagaaaaaacactagatagcgtatttaatggaattgtgtcatttgcaaaatc

SEQ ID NO: 54 - DNA, 266 bp, Homo sapiens
  source 1..266: mol_type = other DNA; note = RICTOR NAT (N59272.1)
tgtaataaaccttacttgaatcagtaaaaaatttaagaggtatttacaaaatatacatacatgctttctttcaagtagatataattagttgctaacaaagtgcttccaacttcagataattttaggtttcataaatattgtataattagcaggagcnattccacacacaatgtactaaaggccttaaaaacaaatggnaatcttaaataggnaaatttgttacataactttatttgtactcnaattttgggtcnataaaccttttc

SEQ ID NO: 55 - DNA, 611 bp, Homo sapiens
  source 1..611: mol_type = other DNA; note = RICTOR NAT (BM676019.1)
ttttttttttttttttttaaattctatcaaccacagccacatttcaattgtgctcttctaactcatacctaaattatggcaaaagctgcacaatgaattaattccctgtgcttatattgcctctctttcttctaatccaatctccaagccaccatgagattaactgtacaaatctaacttttatcattttatctaccttcctgaaaatttcaaagagaaactgggactgggagtagagctgaacaatgcattctatgatgtatttgtgaatacacataaacaaatactacaatatatccaactagaaaagttggatacatatgcgacttcaacagtagaacaaaattaaacaattacacaaatctgatatggaattatgtctttaatatcatctatgattattgtgagcagatatgactaaaagcatacgaagattactgtagttagcattacttttgcaaaagccagaagcaaaactatctccactagcgaagacactgggagtccatcttctcacctttatccacaggtacaaaaggtgagaaaagcattgcttcaaacaatctaacatttcctgaaattctaacaattattaatttcaaaagttatat

SEQ ID NO: 56 - RNA, 25 nt, synthetic construct
  source 1..25: mol_type = other RNA; note = RICTOR si-RNA (UPNA-22)
caagcttaccatttgcctaattcaa

SEQ ID NO: 57 - RNA, 27 nt, synthetic construct
  source 1..27: mol_type = other RNA; note = UPNA-22 complementary strand 5 prime to 3 prime
ttgaattaggcaaatggtaagcttgag

SEQ ID NO: 58 - RNA, 25 nt, synthetic construct
  source 1..25: mol_type = other RNA; note = RICTOR si-RNA (UPNA-23)
ggagctagattccaacagctttcta

SEQ ID NO: 59 - RNA, 27 nt, synthetic construct
  source 1..27: mol_type = other RNA; note = UPNA-23 complementary strand 5 prime to 3 prime
tagaaagctgttggaatctagctccat

SEQ ID NO: 60 - RNA, 25 nt, synthetic construct
  source 1..25: mol_type = other RNA; note = RICTOR si-RNA (UPNA-24)
caatttcttcagatggctataaact

SEQ ID NO: 61 - RNA, 27 nt, synthetic construct
  source 1..27: mol_type = other RNA; note = UPNA-24 complementary strand 5 prime to 3 prime
agtttatagccatctgaagaaattgac

SEQ ID NO: 62 - RNA, 25 nt, synthetic construct
  source 1..25: mol_type = other RNA; note = RICTOR si-RNA (UPNA-25)
gtgcttcattatcatgtattgcatg

SEQ ID NO: 63 - RNA, 27 nt, synthetic construct
  source 1..27: mol_type = other RNA; note = UPNA-25 complementary strand 5 prime to 3 prime
catgcaatacatgataatgaagcacat

SEQ ID NO: 64 - RNA, 25 nt, synthetic construct
  source 1..25: mol_type = other RNA; note = RICTOR si-RNA (UPNA-26)
gtgagaaaagcattgcttcaaacaa

SEQ ID NO: 65 - RNA, 27 nt, synthetic construct
  source 1..27: mol_type = other RNA; note = UPNA-26 complementary strand 5 prime to 3 prime
ttgtttgaagcaatgcttttctcacct

SEQ ID NO: 66 - RNA, 25 nt, synthetic construct
  source 1..25: mol_type = other RNA; note = RICTOR si-RNA (UPNA-27)
atatcaacagtgccagtttattccc

SEQ ID NO: 67 - RNA, 27 nt, synthetic construct
  source 1..27: mol_type = other RNA; note = UPNA-27 complementary strand 5 prime to 3 prime
gggaataaactggcactgttgatattt

SEQ ID NO: 68 - RNA, 25 nt, synthetic construct
  source 1..25: mol_type = other RNA; note = RICTOR si-RNA (UPNA-28)
cattgttgtacttgcctttttatat

SEQ ID NO: 69 - RNA, 27 nt, synthetic construct
  source 1..27: mol_type = other RNA; note = UPNA-28 complementary strand 5 prime to 3 prime
atataaaaaggcaagtacaacaatgtg

SEQ ID NO: 70 - RNA, 25 nt, synthetic construct
  source 1..25: mol_type = other RNA; note = RICTOR si-RNA (UPNA-29)
gcccaaataaagaagatgttcaaca

SEQ ID NO: 71 - RNA, 27 nt, synthetic construct
  source 1..27: mol_type = other RNA; note = UPNA-29 complementary strand 5 prime to 3 prime
tgttgaacatcttctttatttgggctt

SEQ ID NO: 72 - RNA, 25 nt, synthetic construct
  source 1..25: mol_type = other RNA; note = RICTOR si-RNA (UPNA-30)
gtaagtttatgtctacattaaactc

SEQ ID NO: 73 - RNA, 27 nt, synthetic construct
  source 1..27: mol_type = other RNA; note = UPNA-30 complementary strand 5 prime to 3 prime
gagtttaatgtagacataaacttactg

SEQ ID NO: 74 - RNA, 25 nt, synthetic construct
  source 1..25: mol_type = other RNA; note = RICTOR si-RNA (UPNA-31)
gtgcttccaacttcagataatttta

SEQ ID NO: 75 - RNA, 27 nt, synthetic construct
  source 1..27: mol_type = other RNA; note = UPNA-31 complementary strand 5 prime to 3 prime
taaaattatctgaagttggaagcactt

SEQ ID NO: 76 - RNA, 25 nt, synthetic construct
  source 1..25: mol_type = other RNA; note = RICTOR si-RNA (UPNA-32)
gagctagattccaacagctttctaa

SEQ ID NO: 77 - RNA, 27 nt, synthetic construct
  source 1..27: mol_type = other RNA; note = UPNA-32 complementary strand 5 prime to 3 prime
ttagaaagctgttggaatctagctcca

SEQ ID NO: 78 - RNA, 25 nt, synthetic construct
  source 1..25: mol_type = other RNA; note = RICTOR si-RNA (UPNA-33)
gctagattccaacagctttctaaga

SEQ ID NO: 79 - RNA, 27 nt, synthetic construct
  source 1..27: mol_type = other RNA; note = UPNA-33 complementary strand 5 prime to 3 prime
tcttagaaagctgttggaatctagctc

SEQ ID NO: 80 - RNA, 25 nt, synthetic construct
  source 1..25: mol_type = other RNA; note = RICTOR si-RNA (UPNA-34)
tagattccaacagctttctaagata

SEQ ID NO: 81 - RNA, 27 nt, synthetic construct
  source 1..27: mol_type = other RNA; note = UPNA-34 complementary strand 5 prime to 3 prime
tatcttagaaagctgttggaatctagc

SEQ ID NO: 82 - RNA, 25 nt, synthetic construct
  source 1..25: mol_type = other RNA; note = RICTOR si-RNA (UPNA-35)
caagaaattctatatcttcagaaaa

SEQ ID NO: 83 - RNA, 27 nt, synthetic construct
  source 1..27: mol_type = other RNA; note = UPNA-35 complementary strand 5 prime to 3 prime
ttttctgaagatatagaatttcttgct

SEQ ID NO: 84 - RNA, 25 nt, synthetic construct
  source 1..25: mol_type = other RNA; note = RICTOR si-RNA (UPNA-36)
ccaacagctttctaagataagttaa

SEQ ID NO: 85 - RNA, 27 nt, synthetic construct
  source 1..27: mol_type = other RNA; note = UPNA-36 complementary strand 5 prime to 3 prime
ttaacttatcttagaaagctgttggaa

SEQ ID NO: 86 - RNA, 25 nt, synthetic construct
  source 1..25: mol_type = other RNA; note = RICTOR si-RNA (UPNA-37)
ctagattccaacagctttctaagat

SEQ ID NO: 87 - RNA, 27 nt, synthetic construct
  source 1..27: mol_type = other RNA; note = UPNA-37 complementary strand 5 prime to 3 prime
atcttagaaagctgttggaatctagct

SEQ ID NO: 88 - RNA, 25 nt, synthetic construct
  source 1..25: mol_type = other RNA; note = RICTOR si-RNA (UPNA-38)
agctagattccaacagctttctaag

SEQ ID NO: 89 - RNA, 27 nt, synthetic construct
  source 1..27: mol_type = other RNA; note = UPNA-38 complementary strand 5 prime to 3 prime
cttagaaagctgttggaatctagctcc

SEQ ID NO: 90 - RNA, 25 nt, synthetic construct
  source 1..25: mol_type = other RNA; note = RICTOR si-RNA (UPNA-39)
gaaaatggagctagattccaacagc

SEQ ID NO: 91 - RNA, 27 nt, synthetic construct
  source 1..27: mol_type = other RNA; note = UPNA-39 complementary strand 5 prime to 3 prime
gctgttggaatctagctccattttcat

SEQ ID NO: 92 - RNA, 25 nt, synthetic construct
  source 1..25: mol_type = other RNA; note = RICTOR si-RNA (UPNA-40)
atgtgaaaaaacaaggtacttttct

SEQ ID NO: 93 - RNA, 27 nt, synthetic construct
  source 1..27: mol_type = other RNA; note = UPNA-40 complementary strand 5 prime to 3 prime
agaaaagtaccttgttttttcacattt

SEQ ID NO: 94 - RNA, 25 nt, synthetic construct
  source 1..25: mol_type = other RNA; note = RICTOR si-RNA (UPNA-41)
tgaaaaaacaaggtacttttctcaa

SEQ ID NO: 95 - RNA, 27 nt, synthetic construct
  source 1..27: mol_type = other RNA; note = UPNA-41 complementary strand 5 prime to 3 prime
ttgagaaaagtaccttgttttttcaca

SEQ ID NO: 96 - RNA, 25 nt, synthetic construct
  source 1..25: mol_type = other RNA; note = RICTOR si-RNA (UPNA-42)
gtaaaggtcaacaaatatttgtcta

SEQ ID NO: 97 - RNA, 27 nt, synthetic construct
  source 1..27: mol_type = other RNA; note = UPNA-42 complementary strand 5 prime to 3 prime
tagacaaatatttgttgacctttactg

SEQ ID NO: 98 - RNA, 25 nt, synthetic construct
  source 1..25: mol_type = other RNA; note = RICTOR si-RNA (UPNA-43)
agtaaaggtcaacaaatatttgtct

SEQ ID NO: 99 - RNA, 27 nt, synthetic construct
  source 1..27: mol_type = other RNA; note = UPNA-43 complementary strand 5 prime to 3 prime
agacaaatatttgttgacctttactgt

SEQ ID NO: 100 - RNA, 25 nt, synthetic construct
  source 1..25: mol_type = other RNA; note = RICTOR si-RNA (UPNA-44)
cagtaaaggtcaacaaatatttgtc

SEQ ID NO: 101 - RNA, 27 nt, synthetic construct
  source 1..27: mol_type = other RNA; note = UPNA-44 complementary strand 5 prime to 3 prime
gacaaatatttgttgacctttactgtg

SEQ ID NO: 102 - RNA, 25 nt, synthetic construct
  source 1..25: mol_type = other RNA; note = RICTOR si-RNA (UPNA-45)
gaatcagcagcaaaagatcagtacc

SEQ ID NO: 103 - RNA, 27 nt, synthetic construct
  source 1..27: mol_type = other RNA; note = UPNA-45 complementary strand 5 prime to 3 prime
ggtactgatcttttgctgctgattcag

SEQ ID NO: 104 - RNA, 25 nt, synthetic construct
  source 1..25: mol_type = other RNA; note = RICTOR si-RNA (UPNA-46)
caaataaagaagatgttcaacactg

SEQ ID NO: 105 - RNA, 27 nt, synthetic construct
  source 1..27: mol_type = other RNA; note = UPNA-46 complementary strand 5 prime to 3 prime
cagtgttgaacatcttctttatttggg

SEQ ID NO: 106 - RNA, 25 nt, synthetic construct
  source 1..25: mol_type = other RNA; note = RICTOR si-RNA (UPNA-47)
ccaaataaagaagatgttcaacact

SEQ ID NO: 107 - RNA, 27 nt, synthetic construct
  source 1..27: mol_type = other RNA; note = UPNA-47 complementary strand 5 prime to 3 prime
agtgttgaacatcttctttatttgggc

SEQ ID NO: 108 - RNA, 25 nt, synthetic construct
  source 1..25: mol_type = other RNA; note = RICTOR si-RNA (UPNA-48)
ggtcaacaaatatttgtctagtgtg

SEQ ID NO: 109 - RNA, 27 nt, synthetic construct
  source 1..27: mol_type = other RNA; note = UPNA-48 complementary strand 5 prime to 3 prime
cacactagacaaatatttgttgacctt

SEQ ID NO: 110 - RNA, 25 nt, synthetic construct
  source 1..25: mol_type = other RNA; note = RICTOR si-RNA (UPNA-49)
gaggcagacatgtgaataaaattat

SEQ ID NO: 111 - RNA, 27 nt, synthetic construct
  source 1..27: mol_type = other RNA; note = UPNA-49 complementary strand 5 prime to 3 prime
ataattttattcacatgtctgcctctc

SEQ ID NO: 112 - RNA, 25 nt, synthetic construct
  source 1..25: mol_type = other RNA; note = RICTOR si-RNA (UPNA-50)
ccactgaatcagcagcaaaagatca

SEQ ID NO: 113 - RNA, 27 nt, synthetic construct
  source 1..27: mol_type = other RNA; note = UPNA-50 complementary strand 5 prime to 3 prime
tgatcttttgctgctgattcagtggtg

SEQ ID NO: 114 - RNA, 25 nt, synthetic construct
  source 1..25: mol_type = other RNA; note = RICTOR si-RNA (UPNA-51)
aaataaagaagatgttcaacactgc

SEQ ID NO: 115 - RNA, 27 nt, synthetic construct
  source 1..27: mol_type = other RNA; note = UPNA-51 complementary strand 5 prime to 3 prime
gcagtgttgaacatcttctttatttgg

SEQ ID NO: 116 - DNA, 8724 bp, Homo sapiens
  source 1..8724: mol_type = other DNA; note = OPA1 (ENST00000433105.5; range = chr3:193618609-193627332)
ctatgtttttgctcaccgaagctaaatatacatatgaattgattccacagccatggaattgagaaaaaccaggaagtgatgctgaaaacataaatgaaactatggtgagtactctgaagtactaaccaccaccaaatgagataaggttagccagcagaattactgcactaaatcataatctataatattggttgatttaaatagtagaatgtacaagaattacatgactgaatcttttttcctgactagcatttgtatataacttcaaaacttttctgagctaataagatgtaacataattagctgatttcaagatctcagagtataattttaaattcattacactgatttcactttgacccaaaagtctcttccaaacatgaactttgggatctccgttgtgggattacctaaccattaaaatcatatttttacggacctgctgattgcaccaacagccacatattggtatacacttaagcttttattagaacttgttctttagcaaccagaattatatcaaatttccagtgttcaaaccctccgtggtccaaggtgaaaatttatttgtgctgactttgattaatgtgaaaaatgtacgcaatgagtttgtcaaagtgtataagaactacatgcttttgcccaaaagtcaaaataattttcttctacaagaatatgaatgttccctgtccatatactttagctaaaacaaataaataaatgttattctttagtgtaaactggtcatctacagcaatgcttttcattttagaaataaaaacttggggagcaagtaggtaggtaagatgcctacaaacttagggtgactcaataactggaccacataaacagaaattcctaattttgttctaagtataaatacagggaccctacagacaacctaattttcagaaaccaaaattcctctgcttgaatttgtttcttttggctttacctatactacccactccagaaaagttacatatttatatgatgcatcttttatattttcactttgtcaaattaatttcttgtatttggagtccatgaacagattgaggtgacatgatcttggcagaaatggctgattagtgtgaatgtatcggtagcccccttaaaccatccatcaccttgcgcttctgttgggcatagctcgtgctatattggccagcggctctgcgcgcttcctcttcatgctcttgaatttgttgttgtaagagaatgagctcaccaagcagaccctgccacgagttcacaatgaggagaagaggaaattggggaggagaataatgttaaaccaagggtgcaacaaaggaacagatgtaagctagggagggatggttaacttaaaacagaaatggaaactcatttaaagccgtcatacccttgggctctgtaccgcatttccattttcctcctcagtaaatcagatgtctcagaaaaataaaacagcatagtttaaaaagtgaaaaggaaagtgcaataataagtgctaaataaaatgatccagatatataaaaaatatggagaaaaaatactgatttttttttttttttaacagtgttgatcttactggtctaaattttctaattctcaaccatgattctctcaggactgattgaagatcataatagcttacttacttattaaattattattcttaagttagtattttttcttgatatttcatttattcaagtaaaggtccctaggaacaggtttccatcatcaagttaataaaggttgtacattaagtcatatattctatgtgcctttgcatacatttgattaaaatttggggttctttatttaaattaaaagataatgtttcaggttgttttgatttcatatataatatcaaataactagtctgaccgaattttcactttttatgagctgttttgacaacattaaatagaaacagcctctgagaatacagccaagcatatacaaattactaaacaaaagtgctataaaaaaacacaattaagatatggagacaatatagttattatgaaattctgaaatgaaaacaaaacacagtttggacctatgcacaaaaaatcaccttttctatttctaaaattcattccacttcataattataaaatagtatattgatacaatttttaatacaggatattcacatgtggcatgatgtcatttcctgcatagctttattaactgatggcctcttagataatcgacctcaaaaaacgtggaagaaagaccaccaagatttgacaatgttacaagcctaatgttttcttaattagaaatcctaaaaaaagtaaagttctaatgaaggaggtaaccatttgtttaagaacagcgttctgggaaaacaatggactcaatttttaccaagttataaaaataaaaagaggaaaatgggtatttattaagatattctaaattaggagttttaaatagtttcagagttaaaaaatgactgcccaaaatattttattttatataattagaaagtgtcttaaagtctggatataacttggagaatttttattctgttagttttgatacattaggctgtgttcattgccagtattagacaataaatgaaagtggttgatttattataaatcaaaggttaaaactagttgatttcttccacatgacaaatgaaattcaatatttgtttccagatacaacagtataaaatagttataaaaagacatgtctttaaataaatcagttttatgatcattaacaaacctatcaaagaaagacaatgctaactactatcctcatcattctcataatttttacctttaaaaaaaattattgctttatcaagcagaaagaagagtaacctgatttattttctttcatttgatcatttatcacaatcctttattcaaattatccaagtgttcaaatttctcaaacaattccctttaaagaaggcttcctatgttggtttatacagtagtgtacctgctgtatttacataccactagtattctaatttataaaatcccaaagccgtgagaaaatattccacatttctttcttaagactagtcaagagcaatagtgagaagggggcaaagagtagaacaaagagttcaatctgtaactgactgtgattaatcaattgagataactcattccctttggatcagccatattccacatttctcactcgcgtctataaggcacacaatgcttttttttacagcttaaaaattcctctcgaattcactgcttatgtatatacacaactgcatggtaaatactccaatcatctattagatttcaagaaaacactctagggacatgtctctttccttccatgcctgttactaaagcactattatgtaaagatagttgaaatgaactgaaattgacctcctgaaaaagtaggaagaaaacaagaattagacatagaaaagctatttacatgatactacacttataccaaggttacctgtctttgattttgtgcaccagtcttaccctaaataccaagtgatggccacacagcctctaagctaaggccatgtggaacaggaagaggaagaaatctatgcacctactgtgcaagatacactcctcagatgtacaaaccacatttttcaataaatattttcccaagcttagacatttttctttcaagaaatggtacaaaaataatttttaaaacaatgtatataatatataaaatacagtagtatttataaataacaataaataatcattaacaatcacagatgatatagtctcttaatatctcctgactcctggctattgtcagtttgacactgcagatctcaatctctcatctctctttttttaaatctccatctctgaatcagcttccttatctcagtctcatttcacgtacaaagtcctgtttttttttaattataacatcagatcaagaaaattagtaataactccagaaaccaaaatatcaatgaaaatactgtgctaacctaaaagttaatgagtttggtagagaagagtttaagtaatcttgaaatattatactatggtctgaatttttgtctcccacaattcatatgttgaaacctagcctcacaatgtgatagtattgagaggtggggcctttgggaggtgatgaggtcatgaggtttctggagttattggccttacaaaaaaaggccagagctgcctggccccttttgctatgtaaggatatagggagaaggcatcatctatgaagccaagggtcctcaaaagacatcgaatctgccagtgtcttgaacttggatttccagttactacattatttgttgcataagaaatcccagaaaagcaagtcctacttaagcaactctgcttaattccaattcagaatatcaagtacatgaactacttttacagccttgttctcctcaatggttgccaaatatgaaggccagggagaatttgcaggtaatgagagcaaaaaattgaggggaccaaaattttgttggcatcaaccagatttatttactttaatcagcctctactctaccgagttaaatcaatcaactgattttactaagacacaagactatcccaggtgctctgagaaaaagatttagtaacagaaatggccgattagatagtaagaatgtaagccaaagtaaatagtgtttcatatggcaagtgttttaaacactcagaagaatataattaatggagctacaaaggttatggaaggcttcatgaagtgacaattgagctatgctataaagatgagtgaacactaaacacgcgggaaaaaagaaaagagatacttcagctggggaggtacaacataaactataaaattaagaatcagactgctgggtggctcacgcctgtaatcccaacactttgggaggcagaggtggacggaccatgaggtcaagagatcaagaccatcctggccaacctggtgaaaccctgtctctactaaaaatacaaaaattagctaggcgtggtggcaggagcctgtaatcccagctacttgggaggctgaggcaggagaatcgcttgaacccgggaggcagaggttgcagtgggccaagatcgtaccactgtactccagccgggtgacagagtgagactccgtctcaaaaaaaaaaaaaaaaaaaaagaatgagagtaatatgttgcaagaaagaacattaatgtattataatttaataattatacttaaaacaatactactacttttcatgatttgggccatgaaaaagtccttcaaatattttctataaattataaatgtgcttgttgccttctgaatcgccatatttcttacttctgtgagatgcttatgaccaatcagaaacatgatagaacaaaagcacagaaaataaaatgtctcattttgcaaatcaatttcactggaacacaataatcttacagaatctaaaaaagggaatgaactaagtggtcttgtcaccggtatatatgatgactgatatacagcaatctgctgactcccaaattaaattagtccaattctctaaagtaggcagcacttcgattcatcttcaggtagttctgaaggtctccgagtctcagtaatattcagttcacatccagggctatgtcttttttcctccagtggacatacgttttcaaatttttgattaacaagaagcatttattaagaggtggttttaatgtatgtcaatcatacctcaagatagtagtgtaaggttaaaaatggttggttttactatttttttcaatgtttcattaatcaaatcatatcagaacttttgatcaacatgacataaataattcagtagtgtccacaaactgaatggatataaatccaggccaaaccaaagaaacttgattaattcgttagcctgtgcagcattatgtggaaatctatgctttctgtagactttttgaagaccggtattataactttccaggacacctaaaactccagtagtaaccccaggctgggaatcacaggttatcggttcatcatgtttattctgtctgactgaagtcaaactatctgtgttcccctcagctccccgtcactttcacttcagttgattctgaatggtcctaactgttctgctagttgccagcacttctccagatgtgttttcctattagaaaggacaaatcttttctttagttctagttgtagaaatgtttttcaattattattacttttttaaaaagctgattcacatgcacttttacatttctagtagaagtctagccagaaactttgttacaaacctgacaaccattaaactaatacttgactgcttcaaattctaaatactgtttaaatctggggctttcagccctcagactgaaccagttaaaaaagtattcagctaaagcctggccaccttaccacctctgattattgcccacattttcaaacttgatgtcagagtgatttcaaaaagggtactgctaagctaaggtctctctttgcatgcttacattcaaccatcaatttgtacacaagtctcaaaatcactcagtttagctgagtgactcatcattctcaagcatcacaagtaacagccaagctttgcaaacacaatgctgcatattattaataacagaatttgaaaccatcagttggggcttgagacacatttttggatagtaaatttttatagtagcatttgctataatttgacttaaccagtaatgaaagtagcttaattacaactctagattcagtatgtgtaacaaatagttaaaaaaaaaattggtaagtctttaatggggaaaataagcaccataaattgagaaaaagttaaaaggaaatagtttacaaggacatgtattgtcaaacaagcaaatcaaaacaagcaaagagtaaattagaacataatgtaataaatttaattccatgtctactgtataaaataaaatctgtaaatataattaaatttccaaaaataattatctggcttcttttctattgcagagcttctgttcaattactgattattcatctatgaccttagccgggttttaagggaattatctgagtcaggccctgagacaggtttttcattcaccacagtgatcctaggccataagagagaaagcagacacacagacttctcctaaccaatctggcaaacagaaatcttgtcactcaaagaaccacactaccacacctgtaacaaaggctccttctccccattagattagctcttctctccacagtaaatttaattgccttaagggctaaggccttcactcaaaataagccacaactattaggctaatttctacttatttttttttcccccagtagcatgaacaatcaagagtgtcctttggttaaaataatgtcaatgcaaaagagggtcaaaaaagatcaaatcaaaagacaaatacacgtacatacattttgacatacatttttctccaaaagataaaattaaaaggcaaaagaaaaaaatcttttgaaatataattaaaatttggaatataaaaagttcaaagttgaagaacaaggaaagagatgaaaacctattcatagaaattatttcttctttctgcttccaaaataaacattatttctttgatgtattaaaactataataacctccagtataaaaattacatgttccatattacatgaatgttaaaacattcatacaatattttttctataaatctattatctaggtattatttagaaaaaaactttcaaggaatcagttattttaccaattccaaaagaatgaaaaaagtgaagactcagaaaacgtcaggtaaatatatgagtattataaatacaaacaaagtatgttattctacctcagcaattctcctataactttcatatggattttgttaatgaatatgtgttgaaaataacataccataaataaccccatacataactttgttaactatcaatatcagggaattgatttatttcctaataacccaattcctattccctttctagtttccccaaagtgaaatctagtccctatacgacgttccatgagcaaaagggttctgttgataaataatttgggaaaacactgtatgctttgactccttggaaattcacaatgcgcatgagtatattacagtatgagaggtcctagggaaagaaaagaagtccgcttgccttgtctaaacgagcctttctccccccacacacttaacaacacaactctttcttggggacaaccctcgaaactaatgcttcactggacatatgtgaaagaaatgggcttctcaattatatttccagaactgccacgtaataccttgtactttgcactaagattagtaacgatatctctacttggatgtttttgtatttgcttctcagatgttatcattttaaaatcttccaaagaagtaaaaactttaaaaaatctttcaagactacctacatgaacaattctcttttacacttacctttctaaaatgcttgtcactttcagatccacgatctgttgctctaaacgccgtttcttccggagaaccttaaagataaatatgcaaccctttacattctccagtgatgtcaacagcctaagctgaatttcaaattacatcgagtcaatgaaaaggtgaatggatttttagagcaactcagattcaaagaatcaaatcgcctttattcttattttaaggaacatgactgataacctgtgcagaaaataaaaaaaaaactaactatagcaatcgcagcatatgtttaacctaacaattaaaaatatataactgaaaattacaataaaaa

SEQ ID NO: 117 - DNA, 492 bp, Homo sapiens
  source 1..492: mol_type = other DNA; note = OPA1 antisense RNA 1 (ENST00000433105.5)
ctatgtttttgctcaccgaagctaaatatacatatgaattgattccacagccatggaattgagaaaaaccaggaagtgatgctgaaaacataaatgaaactatggtattatttagaaaaaaactttcaaggaatcagttattttaccaattccaaaagaatgaaaaaagtgaagactcagaaaacgtcagatccacgatctgttgctctaaacgccgtttcttccggagaaccttaaagataaatatgcaaccctttacattctccagtgatgtcaacagcctaagctgaatttcaaattacatcgagtcaatgaaaaggtgaatggatttttagagcaactcagattcaaagaatcaaatcgcctttattcttattttaaggaacatgactgataacctgtgcagaaaataaaaaaaaaactaactatagcaatcgcagcatatgtttaacctaacaattaaaaatatataactgaaaattacaataaaaa

SEQ ID NO: 118 - RNA, 25 nt, synthetic construct
  source 1..25: mol_type = other RNA; note = OPA1 NAT siRNA (UPNA 52)
aagctgaatttcaaattacatcgag

SEQ ID NO: 119 - RNA, 27 nt, synthetic construct
  source 1..27: mol_type = other RNA; note = UPNA-52 complementary strand 5 prime to 3 prime
ctcgatgtaatttgaaattcagcttag

SEQ ID NO: 120 - RNA, 25 nt, synthetic construct
  source 1..25: mol_type = other RNA; note = OPA1 NAT siRNA (UPNA 53)
aacaattctcttttacacttacctt

SEQ ID NO: 121 - RNA, 27 nt, synthetic construct
  source 1..27: mol_type = other RNA; note = UPNA-53 complementary strand 5 prime to 3 prime
aaggtaagtgtaaaagagaattgttca

SEQ ID NO: 122 - RNA, 25 nt, synthetic construct
  source 1..25: mol_type = other RNA; note = OPA1 NAT siRNA (UPNA 54)
ccggagaaccttaaagataaatatg

SEQ ID NO: 123 - RNA, 27 nt, synthetic construct
  source 1..27: mol_type = other RNA; note = UPNA-54 complementary strand 5 prime to 3 prime
catatttatctttaaggttctccggaa

SEQ ID NO: 124 - RNA, 25 nt, synthetic construct
  source 1..25: mol_type = other RNA; note = OPA1 NAT siRNA (UPNA 55)
gtaacgatatctctacttggatgtt

SEQ ID NO: 125 - RNA, 27 nt, synthetic construct
  source 1..27: mol_type = other RNA; note = UPNA-55 complementary strand 5 prime to 3 prime
aacatccaagtagagatatcgttacta

SEQ ID NO: 126 - RNA, 25 nt, synthetic construct
  source 1..25: mol_type = other RNA; note = OPA1 NAT siRNA (UPNA 56)
caatcagaaacatgatagaacaaaa

SEQ ID NO: 127 - RNA, 27 nt, synthetic construct
  source 1..27: mol_type = other RNA; note = UPNA-56 complementary strand 5 prime to 3 prime
ttttgttctatcatgtttctgattggt

SEQ ID NO: 128 - RNA, 25 nt, synthetic construct
  source 1..25: mol_type = other RNA; note = OPA1 NAT siRNA (UPNA 57)
ctgaatcagcttccttatctcagtc

SEQ ID NO: 129 - RNA, 27 nt, synthetic construct
  source 1..27: mol_type = other RNA; note = UPNA-57 complementary strand 5 prime to 3 prime
gactgagataaggaagctgattcagag

SEQ ID NO: 130 - RNA, 25 nt, synthetic construct
  source 1..25: mol_type = other RNA; note = OPA1 NAT siRNA (UPNA 58)
gtgagatgcttatgaccaatcagaa

SEQ ID NO: 131 - RNA, 27 nt, synthetic construct
  source 1..27: mol_type = other RNA; note = UPNA-58 complementary strand 5 prime to 3 prime
ttctgattggtcataagcatctcacag

SEQ ID NO: 132 - RNA, 25 nt, synthetic construct
  source 1..25: mol_type = other RNA; note = OPA1 NAT siRNA (UPNA 59)
gtagtgtccacaaactgaatggata

SEQ ID NO: 133 - RNA, 27 nt, synthetic construct
  source 1..27: mol_type = other RNA; note = UPNA-59 complementary strand 5 prime to 3 prime
tatccattcagtttgtggacactactg

SEQ ID NO: 134 - RNA, 25 nt, synthetic construct
  source 1..25: mol_type = other RNA; note = OPA1 NAT siRNA (UPNA 60)
ctgtgagatgcttatgaccaatcag

SEQ ID NO: 135 - RNA, 27 nt, synthetic construct
  source 1..27: mol_type = other RNA; note = UPNA-60 complementary strand 5 prime to 3 prime
ctgattggtcataagcatctcacagaa

SEQ ID NO: 136 - RNA, 25 nt, synthetic construct
  source 1..25: mol_type = other RNA; note = OPA1 NAT siRNA (UPNA 61)
catgcttacattcaaccatcaattt

SEQ ID NO: 137 - RNA, 27 nt, synthetic construct
  source 1..27: mol_type = other RNA; note = UPNA-61 complementary strand 5 prime to 3 prime
aaattgatggttgaatgtaagcatgca

SEQ ID NO: 138 - RNA, 25 nt, synthetic construct
  source 1..25: mol_type = other RNA; note = OPA1 NAT siRNA (UPNA 62)
gaccaatcagaaacatgatagaaca

SEQ ID NO: 139 - RNA, 27 nt, synthetic construct
  source 1..27: mol_type = other RNA; note = UPNA-62 complementary strand 5 prime to 3 prime
tgttctatcatgtttctgattggtcat

SEQ ID NO: 140 - RNA, 25 nt, synthetic construct
  source 1..25: mol_type = other RNA; note = OPA1 NAT siRNA (UPNA 63)
agaattacatgactgaatctttttt

SEQ ID NO: 141 - RNA, 27 nt, synthetic construct
  source 1..27: mol_type = other RNA; note = UPNA-63 complementary strand 5 prime to 3 prime
aaaaaagattcagtcatgtaattcttg

SEQ ID NO: 142 - RNA, 25 nt, synthetic construct
  source 1..25: mol_type = other RNA; note = OPA1 NAT siRNA (UPNA 64)
gctatttacatgatactacacttat

SEQ ID NO: 143 - RNA, 27 nt, synthetic construct
  source 1..27: mol_type = other RNA; note = UPNA-64 complementary strand 5 prime to 3 prime
ataagtgtagtatcatgtaaatagctt

SEQ ID NO: 144 - RNA, 25 nt, synthetic construct
  source 1..25: mol_type = other RNA; note = OPA1 NAT siRNA (UPNA 65)
ggacatgtattgtcaaacaagcaaa

SEQ ID NO: 145 - RNA, 27 nt, synthetic construct
  source 1..27: mol_type = other RNA; note = UPNA-65 complementary strand 5 prime to 3 prime
tttgcttgtttgacaatacatgtcctt

SEQ ID NO: 146 - DNA, 1390 bp, Homo sapiens
  source 1..1390: mol_type = other DNA; note = Natural Antisense Transcript (NAT) to SLC2A1 (ENST00000691915.1 (ENSG00000288955))
aggggaccgctgtgaggtctggagaggatgcgccatcgaccccggcagtaggcggccgaaccttggccccaaatcttctaccccggggtggattgcggcccctagaggaaaggcgtcaggggaccgaccggcagctgtgccgtcgcggcactctggaccacgctgagggcgggccggcttccctgcgcctgcgctgacattttgcagtttgggaaatggcggtgctcggggacgtcgatactgacccagggcgggaggcagctgcgagtggattgtcgggccttttcagaccggggtctgaacctctgggccggggcggggctcgggtgctgcctggctctgccaacgttctgggccctcgagtgtgaatgagctccgcccgccatccgccttgtagtcctcgcgtccggccaccggcgcggggctcccacccatccccaccccaccccaccccaccccaccccgtctgtttcctttctggtccagtggggacagccctgagtgagcagcgtcgtaggagtaagatacctgggcccccggcctctccttcttgccctgcccccgcaccccctcccgcacggggacccggataactcctttcaacagaagtcttttgggagcacacgataaggcttcctttcatctggcctcagcccttcaccagttaaccgccctgtccccaggctcaggcagggcatattcttcgcaacctgctcttctttgttgagatgcagatggatgtcctcatagattctttatacctaatggtaatacatataggtatttccatttgctaagtaccagttttcctttctcacacccttcttacaacccctcttcatgtcagggagaaaaaagcctgcctgtagttacaaaagcaaacttctcccagattttgtcagctgagcctctagagacaaatatctttggtgttttatttcctccagtattttctgagtgcctgctgtgcatcaagcactgtactgaatagtgctgaggacccacaggaaccaaatcggcatcttctcatgaaattcagactagtggcagaaaactggacttgtgatgaaggagcagatgaagtgctctggaaattcaggggtgaaggaggaggatgagctgtctttagagatgaagcagtagtatacagaggaaccagagggggcaattccaaatgaaatgaacacagggcagcttgacagctcattgggcccatacaaagaacgtggttcattttgtgtgtgtggggaggggtgcaggaggtgggtggagttaatggagtagtggttgtatggtaccattgttaaaagcaggtgttttagcttattcactgtgcgacttcaggcacataacctctttgagcctgtccaataaaggtacagtatttact

SEQ ID NO: 147 - DNA, 1758 bp, Homo sapiens
  source 1..1758: mol_type = other DNA; note = SLC2A1 divergent transcript (ENST00000653200.1 (SLC2A1-DT))
ctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctcgtagggttgtagaaagaccaagtgagttaatatttgtaaagtgtttagaacagcgtctactgcatggaaagtagtatttatttgttagagagactccaggaggtcttgcccagggattgacttctcccttccctgtctccaccccccacattcccgtttgtgagccaagccttgcttttgcaccggccgggctctcttgcctggaaatgcatacccatctcaaacctgggctttaaaatcacctcctctgggacgccttcctctacttctcacagttcaggcctgttctgccccacacagcagaaaatgcccacgatgcaataccagagtctgcatgggaccatttcaacaacccttagaagctgactcagctgtctcctggccctgacttttctaagatgagaggagctggtaggaacaatttgggaaccctcaaaggctgagagtatcccacccacttcacagatgaagcaaactgaggcccagagaagggaaattacttgcccaagatcacccagcaagtaagaaacagagctggagatgagctcaggccagcacggaaccacgcaaactccttctgtaggctttgtaaaggattgatgagaaaagcagatggttaagagctctacaacttctgtgagctcatgcctaaggaatctacagtgtttaaagctccccagggccaggcgtggtggttcacacctgtaatcccagcactttgggaggctgaggcaggcggatcacctggggtcaggagttcaagaccagcctggctgacatggtgaaaccccatctctactaataatacaaaaaaaaaagctgggcatggtggcatgcacctgtagtcccagctactagggaggctgaggcacaagaatggcttgaacctgggaggcgaaggttgcagtgaaccaagatcgcgccactgtactccagcctgggcaacagagcaagacagagactccatctcaaaataaataaatagaaggctctccaggacattttgatgcccactgggatatgggaactcctgccatggggaggagggcgtctttaagaaggaataggccctattgagatgccagctgtagacctctaggagttgaccatcgagcctgagaaggtaaggagagggtgtaggagacccctgactctgacctccaccctctcattcctctccagggcagtcccgcactctgatggccacatctggtgccaggtagtttctcgtaaagatctacactcacattacaacctagacatcccactagtaggtatttacccaagacacctgaaagcatgtgtgtacccaaagactcgtaaatgaatgcaactctatatgtaatagccaaaattagaaacaatccaaatgttcatcagcaggtgagtggtaaaacaagttgtgatatgttcatacaatgttgctactcagtaataaaaagaaatgagctacagatacagataaggaaacttggaggtgatatgttcataatcttgtttgggtcaaaggtttcagggatgtatacatgtgtcaaaac

SEQ ID NO: 148 - DNA, 759 bp, Homo sapiens
  source 1..759: mol_type = other DNA; note = SLC2A1 divergent transcript (ENST00000640492.1 (ENSG00000283973))
gtcttcaggctcagagaagagaaggcattgtttgcccatgaatgtttgcaagtaaatagcagagctgggactcaaagagtccggatctggggctccatcatgaatctctaagagtggcccagagattctcacgttccctagcttggggcctccatgaggctaacaatggggtcacatgtctctgtccctgggaaagtggccagcatggggctggcccagagaaggacaaggtggctgtttggtgagtgtgacccatgggctgagtaaggcagacccgcaactgggaaaaggcatagactggatgtgctaagaaacaaggagctgtggagagactttgaggagggaagtagcaaaacccaaaagtcattcacccacgtgcctcagctgcagaacctgtcctcaaggtgctcacaggctgagggaaggcagacagaggacaagtggcaacctacaaaacaccaaatgggctccatactcaaccaaggtgcccctcagccagaactggacacattgccagagaactccaggggtcatctggtcggctcagcctcccggtttccgacagacagtcaccatagactcacctgaactgtgagaagtagaggaaggcgtcccagaggaggtggtactgttactatccctagtgcaccgaagtcacccagcggccgagtgagaactccagtccagctttccacccgctactccgcgcatcccagcttgccttacagccgggtaccggctccaccattttgctagaga

SEQ ID NO: 149 - RNA, 25 nt, synthetic construct
  source 1..25: mol_type = other RNA; note = SLC2A1 NAT siRNA (UPNA-66)
atgttgctactcagtaataaaaaga

SEQ ID NO: 150 - RNA, 27 nt, synthetic construct
  source 1..27: mol_type = other RNA; note = UPNA-66 complementary strand 5 prime to 3 prime
tctttttattactgagtagcaacattg

SEQ ID NO: 151 - RNA, 25 nt, synthetic construct
  source 1..25: mol_type = other RNA; note = SLC2A1 NAT siRNA (UPNA-67)
tgttgctactcagtaataaaaagaa

SEQ ID NO: 152 - RNA, 27 nt, synthetic construct
  source 1..27: mol_type = other RNA; note = UPNA-67 complementary strand 5 prime to 3 prime
ttctttttattactgagtagcaacatt

SEQ ID NO: 153 - RNA, 25 nt, synthetic construct
  source 1..25: mol_type = other RNA; note = SLC2A1 NAT siRNA (UPNA-68)
ggtaaaacaagttgtgatatgttca

SEQ ID NO: 154 - RNA, 27 nt, synthetic construct
  source 1..27: mol_type = other RNA; note = UPNA-68 complementary strand 5 prime to 3 prime
tgaacatatcacaacttgttttaccac

SEQ ID NO: 155 - RNA, 25 nt, synthetic construct
  source 1..25: mol_type = other RNA; note = SLC2A1 NAT siRNA (UPNA-69)
gtaaaacaagttgtgatatgttcat

SEQ ID NO: 156 - RNA, 27 nt, synthetic construct
  source 1..27: mol_type = other RNA; note = UPNA-69 complementary strand 5 prime to 3 prime
atgaacatatcacaacttgttttacca

SEQ ID NO: 157 - RNA, 25 nt, synthetic construct
  source 1..25: mol_type = other RNA; note = SLC2A1 NAT siRNA (UPNA-70)
ggaggtgatatgttcataatcttgt

SEQ ID NO: 158 - RNA, 27 nt, synthetic construct
  source 1..27: mol_type = other RNA; note = UPNA-70 complementary strand 5 prime to 3 prime
acaagattatgaacatatcacctccaa

SEQ ID NO: 159 - RNA, 25 nt, synthetic construct
  source 1..25: mol_type = other RNA; note = SLC2A1 NAT siRNA (UPNA-71)
gtctactgcatggaaagtagtattt

SEQ ID NO: 160 - RNA, 27 nt, synthetic construct
  source 1..27: mol_type = other RNA; note = UPNA-71 complementary strand 5 prime to 3 prime
aaatactactttccatgcagtagacgc

SEQ ID NO: 161 - RNA, 25 nt, synthetic construct
  source 1..25: mol_type = other RNA; note = SLC2A1 NAT siRNA (UPNA-72)
ctaaggaatctacagtgtttaaagc

SEQ ID NO: 162 - RNA, 27 nt, synthetic construct
  source 1..27: mol_type = other RNA; note = UPNA-72 complementary strand 5 prime to 3 prime
gctttaaacactgtagattccttaggc

SEQ ID NO: 163 - RNA, 25 nt, synthetic construct
  source 1..25: mol_type = other RNA; note = SLC2A1 NAT siRNA (UPNA-73)
gtatttacccaagacacctgaaagc

SEQ ID NO: 164 - RNA, 27 nt, synthetic construct
  source 1..27: mol_type = other RNA; note = UPNA-73 complementary strand 5 prime to 3 prime
gctttcaggtgtcttgggtaaatacct

SEQ ID NO: 165 - RNA, 25 nt, synthetic construct
  source 1..25: mol_type = other RNA; note = SLC2A1 NAT siRNA (UPNA-74)
agatctacactcacattacaaccta

SEQ ID NO: 166 - RNA, 27 nt, synthetic construct
  source 1..27: mol_type = other RNA; note = UPNA-74 complementary strand 5 prime to 3 prime
taggttgtaatgtgagtgtagatcttt

SEQ ID NO: 167 - RNA, 25 nt, synthetic construct
  source 1..25: mol_type = other RNA; note = SLC2A1 NAT siRNA (UPNA-75)
gtggtaaaacaagttgtgatatgtt

SEQ ID NO: 168 - RNA, 27 nt, synthetic construct
  source 1..27: mol_type = other RNA; note = UPNA-75 complementary strand 5 prime to 3 prime
aacatatcacaacttgttttaccactc

SEQ ID NO: 169 - RNA, 25 nt, synthetic construct
  source 1..25: mol_type = other RNA; note = SLC2A1 NAT siRNA (UPNA-76)
ggtgatatgttcataatcttgtttg

SEQ ID NO: 170 - RNA, 27 nt, synthetic construct
  source 1..27: mol_type = other RNA; note = UPNA-76 complementary strand 5 prime to 3 prime
caaacaagattatgaacatatcacctc

SEQ ID NO: 171 - RNA, 25 nt, synthetic construct
  source 1..25: mol_type = other RNA; note = SLC2A1 NAT siRNA (UPNA-77)
cagcgtctactgcatggaaagtagt

SEQ ID NO: 172 - RNA, 27 nt, synthetic construct
  source 1..27: mol_type = other RNA; note = UPNA-77 complementary strand 5 prime to 3 prime
actactttccatgcagtagacgctgtt

SEQ ID NO: 173 - RNA, 25 nt, synthetic construct
  source 1..25: mol_type = other RNA; note = SLC2A1 NAT siRNA (UPNA-78)
cgtctactgcatggaaagtagtatt

SEQ ID NO: 174 - RNA, 27 nt, synthetic construct
  source 1..27: mol_type = other RNA; note = UPNA-78 complementary strand 5 prime to 3 prime
aatactactttccatgcagtagacgct

SEQ ID NO: 175 - RNA, 25 nt, synthetic construct
  source 1..25: mol_type = other RNA; note = SLC2A1 NAT siRNA (UPNA-79)
atgttgctactcagtaataaaaaga

SEQ ID NO: 176 - RNA, 27 nt, synthetic construct
  source 1..27: mol_type = other RNA; note = UPNA-79 complementary strand 5 prime to 3 prime
tctttttattactgagtagcaacattg

SEQ ID NO: 177 - RNA, 25 nt, synthetic construct
  source 1..25: mol_type = other RNA; note = SLC2A1 NAT siRNA (UPNA-80)
tgttgctactcagtaataaaaagaa

SEQ ID NO: 178 - RNA, 27 nt, synthetic construct
  source 1..27: mol_type = other RNA; note = UPNA-80 complementary strand 5 prime to 3 prime
ttctttttattactgagtagcaacatt

SEQ ID NO: 179 - RNA, 25 nt, synthetic construct
  source 1..25: mol_type = other RNA; note = SLC2A1 NAT siRNA (UPNA-81)
ggtaaaacaagttgtgatatgttca

SEQ ID NO: 180 - RNA, 27 nt, synthetic construct
  source 1..27: mol_type = other RNA; note = UPNA-81 complementary strand 5 prime to 3 prime
tgaacatatcacaacttgttttaccac

SEQ ID NO: 181 - RNA, 25 nt, synthetic construct
  source 1..25: mol_type = other RNA; note = SLC2A1 NAT siRNA (UPNA-82)
gtaaaacaagttgtgatatgttcat

SEQ ID NO: 182 - RNA, 27 nt, synthetic construct
  source 1..27: mol_type = other RNA; note = UPNA-82 complementary strand 5 prime to 3 prime
atgaacatatcacaacttgttttacca

SEQ ID NO: 183 - RNA, 25 nt, synthetic construct
  source 1..25: mol_type = other RNA; note = SLC2A1 NAT siRNA (UPNA-83)
ggaggtgatatgttcataatcttgt

SEQ ID NO: 184 - RNA, 27 nt, synthetic construct
  source 1..27: mol_type = other RNA; note = UPNA-83 complementary strand 5 prime to 3 prime
acaagattatgaacatatcacctccaa

SEQ ID NO: 185 - RNA, 25 nt, synthetic construct
  source 1..25: mol_type = other RNA; note = SLC2A1 NAT siRNA (UPNA-84)
gtctactgcatggaaagtagtattt

SEQ ID NO: 186 - RNA, 27 nt, synthetic construct
  source 1..27: mol_type = other RNA; note = UPNA-84 complementary strand 5 prime to 3 prime
aaatactactttccatgcagtagacgc

SEQ ID NO: 187 - RNA, 25 nt, synthetic construct
  source 1..25: mol_type = other RNA; note = SLC2A1 NAT siRNA (UPNA-85)
ctaaggaatctacagtgtttaaagc

SEQ ID NO: 188 - RNA, 27 nt, synthetic construct
  source 1..27: mol_type = other RNA; note = UPNA-85 complementary strand 5 prime to 3 prime
gctttaaacactgtagattccttaggc

SEQ ID NO: 189 - RNA, 25 nt, synthetic construct
  source 1..25: mol_type = other RNA; note = SLC2A1 NAT siRNA (UPNA-86)
gtatttacccaagacacctgaaagc

SEQ ID NO: 190 - RNA, 27 nt, synthetic construct
  source 1..27: mol_type = other RNA; note = UPNA-86 complementary strand 5 prime to 3 prime
gctttcaggtgtcttgggtaaatacct

SEQ ID NO: 191 - RNA, 25 nt, synthetic construct
  source 1..25: mol_type = other RNA; note = SLC2A1 NAT siRNA (UPNA-87)
agatctacactcacattacaaccta

SEQ ID NO: 192 - RNA, 27 nt, synthetic construct
  source 1..27: mol_type = other RNA; note = UPNA-87 complementary strand 5 prime to 3 prime
taggttgtaatgtgagtgtagatcttt

SEQ ID NO: 193 - RNA, 25 nt, synthetic construct
  source 1..25: mol_type = other RNA; note = SLC2A1 NAT siRNA (UPNA-88)
gtggtaaaacaagttgtgatatgtt

SEQ ID NO: 194 - RNA, 27 nt, synthetic construct
  source 1..27: mol_type = other RNA; note = UPNA-88 complementary strand 5 prime to 3 prime
aacatatcacaacttgttttaccactc

SEQ ID NO: 195 - RNA, 25 nt, synthetic construct
  source 1..25: mol_type = other RNA; note = SLC2A1 NAT siRNA (UPNA-89)
ggtgatatgttcataatcttgtttg

SEQ ID NO: 196 - RNA, 27 nt, synthetic construct
  source 1..27: mol_type = other RNA; note = UPNA-89 complementary strand 5 prime to 3 prime
caaacaagattatgaacatatcacctc

SEQ ID NO: 197 - RNA, 25 nt, synthetic construct
  source 1..25: mol_type = other RNA; note = SLC2A1 NAT siRNA (UPNA-90)
cagcgtctactgcatggaaagtagt

SEQ ID NO: 198 - RNA, 27 nt, synthetic construct
  source 1..27: mol_type = other RNA; note = UPNA-90 complementary strand 5 prime to 3 prime
actactttccatgcagtagacgctgtt

SEQ ID NO: 199 - RNA, 25 nt, synthetic construct
  source 1..25: mol_type = other RNA; note = SLC2A1 NAT siRNA (UPNA-91)
cgtctactgcatggaaagtagtatt

SEQ ID NO: 200 - RNA, 27 nt, synthetic construct
  source 1..27: mol_type = other RNA; note = UPNA-91 complementary strand 5 prime to 3 prime
aatactactttccatgcagtagacgct

SEQ ID NO: 201 - RNA, 25 nt, synthetic construct
  source 1..25: mol_type = other RNA; note = SLC2A1 NAT siRNA (UPNA-92)
agtggcaacctacaaaacaccaaat

SEQ ID NO: 202 - RNA, 27 nt, synthetic construct
  source 1..27: mol_type = other RNA; note = UPNA-92 complementary strand 5 prime to 3 prime
atttggtgttttgtaggttgccacttg

SEQ ID NO: 203 - RNA, 25 nt, synthetic construct
  source 1..25: mol_type = other RNA; note = SLC2A1 NAT siRNA (UPNA-93)
gcatagactggatgtgctaagaaac

SEQ ID NO: 204 - RNA, 27 nt, synthetic construct
  source 1..27: mol_type = other RNA; note = UPNA-93 complementary strand 5 prime to 3 prime
gtttcttagcacatccagtctatgcct

SEQ ID NO: 205 - RNA, 25 nt, synthetic construct
  source 1..25: mol_type = other RNA; note = SLC2A1 NAT siRNA (UPNA-94)
gtggcaacctacaaaacaccaaatg

SEQ ID NO: 206 - RNA, 27 nt, synthetic construct
  source 1..27: mol_type = other RNA; note = UPNA-94 complementary strand 5 prime to 3 prime
catttggtgttttgtaggttgccactt

SEQ ID NO: 207 - RNA, 25 nt, synthetic construct
  source 1..25: mol_type = other RNA; note = SLC2A1 NAT siRNA (UPNA-95)
aggcatagactggatgtgctaagaa

SEQ ID NO: 208 - RNA, 27 nt, synthetic construct
  source 1..27: mol_type = other RNA; note = UPNA-95 complementary strand 5 prime to 3 prime
ttcttagcacatccagtctatgccttt

SEQ ID NO: 209 - RNA, 25 nt, synthetic construct
  source 1..25: mol_type = other RNA; note = SLC2A1 NAT siRNA (UPNA-96)
gatgtgctaagaaacaaggagctgt

SEQ ID NO: 210 - RNA, 27 nt, synthetic construct
  source 1..27: mol_type = other RNA; note = UPNA-96 complementary strand 5 prime to 3 prime
acagctccttgtttcttagcacatcca

SEQ ID NO: 211 - RNA, 25 nt, synthetic construct
  source 1..25: mol_type = other RNA; note = SLC2A1 NAT siRNA (UPNA-97)
ggcaacctacaaaacaccaaatggg

SEQ ID NO: 212 - RNA, 27 nt, synthetic construct
  source 1..27: mol_type = other RNA; note = UPNA-97 complementary strand 5 prime to 3 prime
cccatttggtgttttgtaggttgccac

SEQ ID NO: 213 - RNA, 25 nt, synthetic construct
  source 1..25: mol_type = other RNA; note = SLC2A1 NAT siRNA (UPNA-98)
gtactgttactatccctagtgcacc

SEQ ID NO: 214 - RNA, 27 nt, synthetic construct
  source 1..27: mol_type = other RNA; note = UPNA-98 complementary strand 5 prime to 3 prime
ggtgcactagggatagtaacagtacca

SEQ ID NO: 215 - RNA, 25 nt, synthetic construct
  source 1..25: mol_type = other RNA; note = SLC2A1 NAT siRNA (UPNA-99)
gaggtggtactgttactatccctag

SEQ ID NO: 216 - RNA, 27 nt, synthetic construct
  source 1..27: mol_type = other RNA; note = UPNA-99 complementary strand 5 prime to 3 prime
ctagggatagtaacagtaccacctcct

SEQ ID NO: 217 - RNA, 25 nt, synthetic construct
  source 1..25: mol_type = other RNA; note = SLC2A1 NAT siRNA (UPNA-100)
ggctaacaatggggtcacatgtctc

SEQ ID NO: 218 - RNA, 27 nt, synthetic construct
  source 1..27: mol_type = other RNA; note = UPNA-100 complementary strand 5 prime to 3 prime
gagacatgtgaccccattgttagcctc

SEQ ID NO: 219 - RNA, 25 nt, synthetic construct
  source 1..25: mol_type = other RNA; note = SLC2A1 NAT siRNA (UPNA-101)
gcagtagacgctgttctaaacactt

SEQ ID NO: 220 - RNA, 27 nt, synthetic construct
  source 1..27: mol_type = other RNA; note = UPNA-101 complementary strand 5 prime to 3 prime
aagtgtttagaacagcgtctactgcat

SEQ ID NO: 221 - RNA, 25 nt, synthetic construct
  source 1..25: mol_type = other RNA; note = SLC2A1 NAT siRNA (UPNA-102)
gacgctgttctaaacactttacaaa

SEQ ID NO: 222 - RNA, 27 nt, synthetic construct
  source 1..27: mol_type = other RNA; note = UPNA-102 complementary strand 5 prime to 3 prime
tttgtaaagtgtttagaacagcgtcta

SEQ ID NO: 223 - RNA, 25 nt, synthetic construct
  source 1..25: mol_type = other RNA; note = SLC2A1 NAT siRNA (UPNA-103)
gtagacgctgttctaaacactttac

SEQ ID NO: 224 - RNA, 27 nt, synthetic construct
  source 1..27: mol_type = other RNA; note = UPNA-103 complementary strand 5 prime to 3 prime
gtaaagtgtttagaacagcgtctactg

SEQ ID NO: 225 - RNA, 25 nt, synthetic construct
  source 1..25: mol_type = other RNA; note = SLC2A1 NAT siRNA (UPNA-104)
cagtagacgctgttctaaacacttt

SEQ ID NO: 226 - RNA, 27 nt, synthetic construct
  source 1..27: mol_type = other RNA; note = UPNA-104 complementary strand 5 prime to 3 prime
aaagtgtttagaacagcgtctactgca

SEQ ID NO: 227 - RNA, 25 nt, synthetic construct
  source 1..25: mol_type = other RNA; note = SLC2A1 NAT siRNA (UPNA-105)
agtagacgctgttctaaacacttta

SEQ ID NO: 228 - RNA, 27 nt, synthetic construct
  source 1..27: mol_type = other RNA; note = UPNA-105 complementary strand 5 prime to 3 prime
taaagtgtttagaacagcgtctactgc

SEQ ID NO: 229 - RNA, 25 nt, synthetic construct
  source 1..25: mol_type = other RNA; note = SLC2A1 NAT siRNA (UPNA-106)
catgcagtagacgctgttctaaaca

SEQ ID NO: 230 - RNA, 27 nt, synthetic construct
  source 1..27: mol_type = other RNA; note = UPNA-106 complementary strand 5 prime to 3 prime
tgtttagaacagcgtctactgcatgga

SEQ ID NO: 231 - RNA, 25 nt, synthetic construct
  source 1..25: mol_type = other RNA; note = SLC2A1 NAT siRNA (UPNA-107)
agtggcaacctacaaaacaccaaat

SEQ ID NO: 232 - RNA, 27 nt, synthetic construct
  source 1..27: mol_type = other RNA; note = UPNA-107 complementary strand 5 prime to 3 prime
atttggtgttttgtaggttgccacttg

SEQ ID NO: 233 - RNA, 25 nt, synthetic construct
  source 1..25: mol_type = other RNA; note = SLC2A1 NAT siRNA (UPNA-108)
agacgctgttctaaacactttacaa

SEQ ID NO: 234 - RNA, 27 nt, synthetic construct
  source 1..27: mol_type = other RNA; note = UPNA-108 complementary strand 5 prime to 3 prime
ttgtaaagtgtttagaacagcgtctac

SEQ ID NO: 235 - RNA, 25 nt, synthetic construct
  source 1..25: mol_type = other RNA; note = SLC2A1 NAT siRNA (UPNA-109)
gcatagactggatgtgctaagaaac

SEQ ID NO: 236 - RNA, 27 nt, synthetic construct
  source 1..27: mol_type = other RNA; note = UPNA-109 complementary strand 5 prime to 3 prime
gtttcttagcacatccagtctatgcct

SEQ ID NO: 237 - RNA, 25 nt, synthetic construct
  source 1..25: mol_type = other RNA; note = SLC2A1 NAT siRNA (UPNA-110)
gtggcaacctacaaaacaccaaatg

SEQ ID NO: 238 - RNA, 27 nt, synthetic construct
  source 1..27: mol_type = other RNA; note = UPNA-110 complementary strand 5 prime to 3 prime
catttggtgttttgtaggttgccactt

SEQ ID NO: 239 - RNA, 25 nt, synthetic construct
  source 1..25: mol_type = other RNA; note = SLC2A1 NAT siRNA (UPNA-111)
aggcatagactggatgtgctaagaa

SEQ ID NO: 240 - RNA, 27 nt, synthetic construct
  source 1..27: mol_type = other RNA; note = UPNA-111 complementary strand 5 prime to 3 prime
ttcttagcacatccagtctatgccttt

SEQ ID NO: 241 - RNA, 25 nt, synthetic construct
  source 1..25: mol_type = other RNA; note = SLC2A1 NAT siRNA (UPNA-112)
ctttccatgcagtagacgctgttct

SEQ ID NO: 242 - RNA, 27 nt, synthetic construct
  source 1..27: mol_type = other RNA; note = UPNA-112 complementary strand 5 prime to 3 prime
agaacagcgtctactgcatggaaagta

SEQ ID NO: 243 - RNA, 25 nt, synthetic construct
  source 1..25: mol_type = other RNA; note = SLC2A1 NAT siRNA (UPNA-113)
tagacgctgttctaaacactttaca

SEQ ID NO: 244 - RNA, 27 nt, synthetic construct
  source 1..27: mol_type = other RNA; note = UPNA-113 complementary strand 5 prime to 3 prime
tgtaaagtgtttagaacagcgtctact

SEQ ID NO: 245 - RNA, 25 nt, synthetic construct
  source 1..25: mol_type = other RNA; note = SLC2A1 NAT siRNA (UPNA-114)
aaatactactttccatgcagtagac

SEQ ID NO: 246 - RNA, 27 nt, synthetic construct
  source 1..27: mol_type = other RNA; note = UPNA-114 complementary strand 5 prime to 3 prime
gtctactgcatggaaagtagtatttat

SEQ ID NO: 247 - RNA, 25 nt, synthetic construct
  source 1..25: mol_type = other RNA; note = SLC2A1 NAT siRNA (UPNA-115)
actcacttggtctttctacaaccct

SEQ ID NO: 248 - RNA, 27 nt, synthetic construct
  source 1..27: mol_type = other RNA; note = UPNA-115 complementary strand 5 prime to 3 prime
agggttgtagaaagaccaagtgagtta

SEQ ID NO: 249 - RNA, 25 nt, synthetic construct
  source 1..25: mol_type = other RNA; note = SLC2A1 NAT siRNA (UPNA-116)
gcctgtccaataaaggtacagtatt

SEQ ID NO: 250 - RNA, 27 nt, synthetic construct
  source 1..27: mol_type = other RNA; note = UPNA-116 complementary strand 5 prime to 3 prime
aatactgtacctttattggacaggctc

SEQ ID NO: 251 - RNA, 25 nt, synthetic construct
  source 1..25: mol_type = other RNA; note = SLC2A1 NAT siRNA (UPNA-117)
tgtccaataaaggtacagtatttac

SEQ ID NO: 252 - RNA, 27 nt, synthetic construct
  source 1..27: mol_type = other RNA; note = UPNA-117 complementary strand 5 prime to 3 prime
gtaaatactgtacctttattggacagg

SEQ ID NO: 253 - RNA, 25 nt, synthetic construct
  source 1..25: mol_type = other RNA; note = SLC2A1 NAT siRNA (UPNA-118)
ctgtccaataaaggtacagtattta

SEQ ID NO: 254 - RNA, 27 nt, synthetic construct
  source 1..27: mol_type = other RNA; note = UPNA-118 complementary strand 5 prime to 3 prime
taaatactgtacctttattggacaggc

SEQ ID NO: 255 - RNA, 25 nt, synthetic construct
  source 1..25: mol_type = other RNA; note = SLC2A1 NAT siRNA (UPNA-119)
gtaccagttttcctttctcacaccc

SEQ ID NO: 256 - RNA, 27 nt, synthetic construct
  source 1..27: mol_type = other RNA; note = UPNA-119 complementary strand 5 prime to 3 prime
gggtgtgagaaaggaaaactggtactt

SEQ ID NO: 257 - RNA, 25 nt, synthetic construct
  source 1..25: mol_type = other RNA; note = SLC2A1 NAT siRNA (UPNA-120)
cttctcatgaaattcagactagtgg

SEQ ID NO: 258 - RNA, 27 nt, synthetic construct
  source 1..27: mol_type = other RNA; note = UPNA-120 complementary strand 5 prime to 3 prime
ccactagtctgaatttcatgagaagat

SEQ ID NO: 259 - RNA, 25 nt, synthetic construct
  source 1..25: mol_type = other RNA; note = SLC2A1 NAT siRNA (UPNA-121)
aaccaaatcggcatcttctcatgaa

SEQ ID NO: 260 - RNA, 27 nt, synthetic construct
  source 1..27: mol_type = other RNA; note = UPNA-121 complementary strand 5 prime to 3 prime
ttcatgagaagatgccgatttggttcc

SEQ ID NO: 261 - RNA, 25 nt, synthetic construct
  source 1..25: mol_type = other RNA; note = SLC2A1 NAT siRNA (UPNA-122)
aatcggcatcttctcatgaaattca

SEQ ID NO: 262 - RNA, 27 nt, synthetic construct
  source 1..27: mol_type = other RNA; note = UPNA-122 complementary strand 5 prime to 3 prime
tgaatttcatgagaagatgccgatttg

SEQ ID NO: 263 - RNA, 25 nt, synthetic construct
  source 1..25: mol_type = other RNA; note = SLC2A1 NAT siRNA (UPNA-123)
agagatgaagcagtagtatacagag

SEQ ID NO: 264 - RNA, 27 nt, synthetic construct
  source 1..27: mol_type = other RNA; note = UPNA-123 complementary strand 5 prime to 3 prime
ctctgtatactactgcttcatctctaa

SEQ ID NO: 265 - RNA, 25 nt, synthetic construct
  source 1..25: mol_type = other RNA; note = SLC2A1 NAT siRNA (UPNA-124)
ggaatcttaaatcagctatttctga

SEQ ID NO: 266 - RNA, 27 nt, synthetic construct
  source 1..27: mol_type = other RNA; note = UPNA-124 complementary strand 5 prime to 3 prime
tcagaaatagctgatttaagattcctg

SEQ ID NO: 267 - RNA, 25 nt, synthetic construct
  source 1..25: mol_type = other RNA; note = SLC2A1 NAT siRNA (UPNA-125)
aggaatcttaaatcagctatttctg

SEQ ID NO: 268 - RNA, 27 nt, synthetic construct
  source 1..27: mol_type = other RNA; note = UPNA-125 complementary strand 5 prime to 3 prime
cagaaatagctgatttaagattcctgc

SEQ ID NO: 269 - RNA, 25 nt, synthetic construct
  source 1..25: mol_type = other RNA; note = SLC2A1 NAT siRNA (UPNA-126)
gcaggaatcttaaatcagctatttc

SEQ ID NO: 270 - RNA, 27 nt, synthetic construct
  source 1..27: mol_type = other RNA; note = UPNA-126 complementary strand 5 prime to 3 prime
gaaatagctgatttaagattcctgcac

SEQ ID NO: 271 - RNA, 25 nt, synthetic construct
  source 1..25: mol_type = other RNA; note = SLC2A1 NAT siRNA (UPNA-127)
gtgcaggaatcttaaatcagctatt

SEQ ID NO: 272 - RNA, 27 nt, synthetic construct
  source 1..27: mol_type = other RNA; note = UPNA-127 complementary strand 5 prime to 3 prime
aatagctgatttaagattcctgcactg

SEQ ID NO: 273 - RNA, 25 nt, synthetic construct
  source 1..25: mol_type = other RNA; note = SLC2A1 NAT siRNA (UPNA-128)
gggggtgattaccagactttctgaa

SEQ ID NO: 274 - RNA, 27 nt, synthetic construct
  source 1..27: mol_type = other RNA; note = UPNA-128 complementary strand 5 prime to 3 prime
ttcagaaagtctggtaatcacccccaa

SEQ ID NO: 275 - RNA, 25 nt, synthetic construct
  source 1..25: mol_type = other RNA; note = SLC2A1 NAT siRNA (UPNA-129)
gccgatatctgcattacaatatata

SEQ ID NO: 276 - RNA, 27 nt, synthetic construct
  source 1..27: mol_type = other RNA; note = UPNA-129 complementary strand 5 prime to 3 prime
tatatattgtaatgcagatatcggctg

SEQ ID NO: 277 - RNA, 25 nt, synthetic construct
  source 1..25: mol_type = other RNA; note = SLC2A1 NAT siRNA (UPNA-130)
ttgcccaagattattcagctaaata

SEQ ID NO: 278 - RNA, 27 nt, synthetic construct
  source 1..27: mol_type = other RNA; note = UPNA-130 complementary strand 5 prime to 3 prime
tatttagctgaataatcttgggcaagt

SEQ ID NO: 279 - RNA, 25 nt, synthetic construct
  source 1..25: mol_type = other RNA; note = SLC2A1 NAT siRNA (UPNA-131)
tgcaggaatcttaaatcagctattt

SEQ ID NO: 280 - RNA, 27 nt, synthetic construct
  source 1..27: mol_type = other RNA; note = UPNA-131 complementary strand 5 prime to 3 prime
aaatagctgatttaagattcctgcact

SEQ ID NO: 281 - RNA, 25 nt, synthetic construct
  source 1..25: mol_type = other RNA; note = SLC2A1 NAT siRNA (UPNA-132)
agctaaatagcagtgcaggaatctt

SEQ ID NO: 282 - RNA, 27 nt, synthetic construct
  source 1..27: mol_type = other RNA; note = UPNA-132 complementary strand 5 prime to 3 prime
aagattcctgcactgctatttagctga

SEQ ID NO: 283 - RNA, 25 nt, synthetic construct
  source 1..25: mol_type = other RNA; note = SLC2A1 NAT siRNA (UPNA-133)
agccgatatctgcattacaatatat

SEQ ID NO: 284 - RNA, 27 nt, synthetic construct
  source 1..27: mol_type = other RNA; note = UPNA-133 complementary strand 5 prime to 3 prime
atatattgtaatgcagatatcggctgc

SEQ ID NO: 285 - RNA, 25 nt, synthetic construct
  source 1..25: mol_type = other RNA; note = SLC2A1 NAT siRNA (UPNA-134)
tgggggtgattaccagactttctga

SEQ ID NO: 286 - RNA, 27 nt, synthetic construct
  source 1..27: mol_type = other RNA; note = UPNA-134 complementary strand 5 prime to 3 prime
tcagaaagtctggtaatcacccccaag

SEQ ID NO: 287 - RNA, 25 nt, synthetic construct
  source 1..25: mol_type = other RNA; note = SLC2A1 NAT siRNA (UPNA-135)
agcagtgcaggaatcttaaatcagc

SEQ ID NO: 288 - RNA, 27 nt, synthetic construct
  source 1..27: mol_type = other RNA; note = UPNA-135 complementary strand 5 prime to 3 prime
gctgatttaagattcctgcactgctat

SEQ ID NO: 289 - RNA, 25 nt, synthetic construct
  source 1..25: mol_type = other RNA; note = SLC2A1 NAT siRNA (UPNA-136)
gcccaagattattcagctaaatagc

SEQ ID NO: 290 - RNA, 27 nt, synthetic construct
  source 1..27: mol_type = other RNA; note = UPNA-136 complementary strand 5 prime to 3 prime
gctatttagctgaataatcttgggcaa

SEQ ID NO: 291 - RNA, 25 nt, synthetic construct
  source 1..25: mol_type = other RNA; note = SLC2A1 NAT siRNA (UPNA-137)
tgcccaagattattcagctaaatag

SEQ ID NO: 292 - RNA, 27 nt, synthetic construct
  source 1..27: mol_type = other RNA; note = UPNA-137 complementary strand 5 prime to 3 prime
ctatttagctgaataatcttgggcaag

SEQ ID NO: 293 - RNA, 25 nt, synthetic construct
  source 1..25: mol_type = other RNA; note = SLC2A1 NAT siRNA (UPNA-138)
ggggtgattaccagactttctgaaa

SEQ ID NO: 294 - RNA, 27 nt, synthetic construct
  source 1..27: mol_type = other RNA; note = UPNA-138 complementary strand 5 prime to 3 prime
tttcagaaagtctggtaatcaccccca

SEQ ID NO: 295 - RNA, 25 nt, synthetic construct
  source 1..25: mol_type = other RNA; note = SLC2A1 NAT siRNA (UPNA-139)
aactggtctcaggtaaagaaagatt

SEQ ID NO: 296 - RNA, 27 nt, synthetic construct
  source 1..27: mol_type = other RNA; note = UPNA-139 complementary strand 5 prime to 3 prime
aatctttctttacctgagaccagttgg

SEQ ID NO: 297 - RNA, 25 nt, synthetic construct
  source 1..25: mol_type = other RNA; note = SLC2A1 NAT siRNA (UPNA-140)
actggtctcaggtaaagaaagatta

SEQ ID NO: 298 - RNA, 27 nt, synthetic construct
  source 1..27: mol_type = other RNA; note = UPNA-140 complementary strand 5 prime to 3 prime
taatctttctttacctgagaccagttg

SEQ ID NO: 299 - RNA, 25 nt, synthetic construct
  source 1..25: mol_type = other RNA; note = SLC2A1 NAT siRNA (UPNA-141)
caactggtctcaggtaaagaaagat

SEQ ID NO: 300 - RNA, 27 nt, synthetic construct
  source 1..27: mol_type = other RNA; note = UPNA-141 complementary strand 5 prime to 3 prime
atctttctttacctgagaccagttggg

SEQ ID NO: 301 - RNA, 25 nt, synthetic construct
  source 1..25: mol_type = other RNA; note = SLC2A1 NAT siRNA (UPNA-142)
cttagtgtctgtatttaatattgac

SEQ ID NO: 302 - RNA, 27 nt, synthetic construct
  source 1..27: mol_type = other RNA; note = UPNA-142 complementary strand 5 prime to 3 prime
gtcaatattaaatacagacactaagtt

SEQ ID NO: 303 - RNA, 25 nt, synthetic construct
  source 1..25: mol_type = other RNA; note = SLC2A1 NAT siRNA (UPNA-143)
atatctgtttaggtaagtaacagga

SEQ ID NO: 304 - RNA, 27 nt, synthetic construct
  source 1..27: mol_type = other RNA; note = UPNA-143 complementary strand 5 prime to 3 prime
tcctgttacttacctaaacagatataa

SEQ ID NO: 305 - RNA, 25 nt, synthetic construct
  source 1..25: mol_type = other RNA; note = SLC2A1 NAT siRNA (UPNA-144)
gcttgtccagatatactataactta

SEQ ID NO: 306 - RNA, 27 nt, synthetic construct
  source 1..27: mol_type = other RNA; note = UPNA-144 complementary strand 5 prime to 3 prime
taagttatagtatatctggacaagcca

SEQ ID NO: 307 - RNA, 25 nt, synthetic construct
  source 1..25: mol_type = other RNA; note = SLC2A1 NAT siRNA (UPNA-145)
aggtggtgtatttacaagttggctt

SEQ ID NO: 308 - RNA, 27 nt, synthetic construct
  source 1..27: mol_type = other RNA; note = UPNA-145 complementary strand 5 prime to 3 prime
aagccaacttgtaaatacaccacctca

SEQ ID NO: 309 - RNA, 25 nt, synthetic construct
  source 1..25: mol_type = other RNA; note = SLC2A1 NAT siRNA (UPNA-146)
gtgtatttacaagttggcttgtcca

SEQ ID NO: 310 - RNA, 27 nt, synthetic construct
  source 1..27: mol_type = other RNA; note = UPNA-146 complementary strand 5 prime to 3 prime
tggacaagccaacttgtaaatacacca

SEQ ID NO: 311 - RNA, 25 nt, synthetic construct
  source 1..25: mol_type = other RNA; note = SLC2A1 NAT siRNA (UPNA-147)
gtggtgtatttacaagttggcttgt

SEQ ID NO: 312 - RNA, 27 nt, synthetic construct
  source 1..27: mol_type = other RNA; note = UPNA-147 complementary strand 5 prime to 3 prime
acaagccaacttgtaaatacaccacct

SEQ ID NO: 313 - RNA, 25 nt, synthetic construct
  source 1..25: mol_type = other RNA; note = SLC2A1 NAT siRNA (UPNA-148)
ggcttgtccagatatactataactt

SEQ ID NO: 314 - RNA, 27 nt, synthetic construct
  source 1..27: mol_type = other RNA; note = UPNA-148 complementary strand 5 prime to 3 prime
aagttatagtatatctggacaagccaa

SEQ ID NO: 315 - RNA, 25 nt, synthetic construct
  source 1..25: mol_type = other RNA; note = SLC2A1 NAT siRNA (UPNA-149)
atatcttgcatctatacacaacagg

SEQ ID NO: 316 - RNA, 27 nt, synthetic construct
  source 1..27: mol_type = other RNA; note = UPNA-149 complementary strand 5 prime to 3 prime
cctgttgtgtatagatgcaagatattt

SEQ ID NO: 317 - RNA, 25 nt, synthetic construct
  source 1..25: mol_type = other RNA; note = SLC2A1 NAT siRNA (UPNA-150)
aggccctcttctcatggtaatagtg

SEQ ID NO: 318 - RNA, 27 nt, synthetic construct
  source 1..27: mol_type = other RNA; note = UPNA-150 complementary strand 5 prime to 3 prime
cactattaccatgagaagagggcctgt

SEQ ID NO: 319 - RNA, 25 nt, synthetic construct
  source 1..25: mol_type = other RNA; note = SLC2A1 NAT siRNA (UPNA-151)
acctgttgcttgtctgaatagattt

SEQ ID NO: 320 - RNA, 27 nt, synthetic construct
  source 1..27: mol_type = other RNA; note = UPNA-151 complementary strand 5 prime to 3 prime
aaatctattcagacaagcaacaggttt

SEQ ID NO: 321 - RNA, 25 nt, synthetic construct
  source 1..25: mol_type = other RNA; note = SLC2A1 NAT siRNA (UPNA-152)
tgtctgaatagatttgagcaacagt

SEQ ID NO: 322 - RNA, 27 nt, synthetic construct
  source 1..27: mol_type = other RNA; note = UPNA-152 complementary strand 5 prime to 3 prime
actgttgctcaaatctattcagacaag

SEQ ID NO: 323 - RNA, 25 nt, synthetic construct
  source 1..25: mol_type = other RNA; note = SLC2A1 NAT siRNA (UPNA-153)
gcaacagtcttgcttttgttaaatc

SEQ ID NO: 324 - RNA, 27 nt, synthetic construct
  source 1..27: mol_type = other RNA; note = UPNA-153 complementary strand 5 prime to 3 prime
gatttaacaaaagcaagactgttgctc

SEQ ID NO: 325 - RNA, 25 nt, synthetic construct
  source 1..25: mol_type = other RNA; note = SLC2A1 NAT siRNA (UPNA-154)
cttgtctgaatagatttgagcaaca

SEQ ID NO: 326 - RNA, 27 nt, synthetic construct
  source 1..27: mol_type = other RNA; note = UPNA-154 complementary strand 5 prime to 3 prime
tgttgctcaaatctattcagacaagca

SEQ ID NO: 327 - RNA, 25 nt, synthetic construct
  source 1..25: mol_type = other RNA; note = SLC2A1 NAT siRNA (UPNA-155)
aactggtctcaggtaaagaaagatt

SEQ ID NO: 328 - RNA, 27 nt, synthetic construct
  source 1..27: mol_type = other RNA; note = UPNA-155 complementary strand 5 prime to 3 prime
aatctttctttacctgagaccagttgg

SEQ ID NO: 329 - RNA, 25 nt, synthetic construct
  source 1..25: mol_type = other RNA; note = SLC2A1 NAT siRNA (UPNA-156)
actggtctcaggtaaagaaagatta

SEQ ID NO: 330 - RNA, 27 nt, synthetic construct
  source 1..27: mol_type = other RNA; note = UPNA-156 complementary strand 5 prime to 3 prime
taatctttctttacctgagaccagttg

SEQ ID NO: 331 - RNA, 25 nt, synthetic construct
  source 1..25: mol_type = other RNA; note = SLC2A1 NAT siRNA (UPNA-157)
caactggtctcaggtaaagaaagat

SEQ ID NO: 332 - RNA, 27 nt, synthetic construct
  source 1..27: mol_type = other RNA; note = UPNA-157 complementary strand 5 prime to 3 prime
atctttctttacctgagaccagttggg

SEQ ID NO: 333 - RNA, 25 nt, synthetic construct
  source 1..25: mol_type = other RNA; note = SLC2A1 NAT siRNA (UPNA-158)
cttagtgtctgtatttaatattgac

SEQ ID NO: 334 - RNA, 27 nt, synthetic construct
  source 1..27: mol_type = other RNA; note = UPNA-158 complementary strand 5 prime to 3 prime
gtcaatattaaatacagacactaagtt

SEQ ID NO: 335 - RNA, 25 nt, synthetic construct
  source 1..25: mol_type = other RNA; note = SLC2A1 NAT siRNA (UPNA-159)
aagtcctgaatatcttctggacaca

SEQ ID NO: 336 - RNA, 27 nt, synthetic construct
  source 1..27: mol_type = other RNA; note = UPNA-159 complementary strand 5 prime to 3 prime
tgtgtccagaagatattcaggacttaa

SEQ ID NO: 337 - RNA, 25 nt, synthetic construct
  source 1..25: mol_type = other RNA; note = SLC2A1 NAT siRNA (UPNA-160)
gcttgtctgaatagatttgagcaac

SEQ ID NO: 338 - RNA, 27 nt, synthetic construct
  source 1..27: mol_type = other RNA; note = UPNA-160 complementary strand 5 prime to 3 prime
gttgctcaaatctattcagacaagcaa

SEQ ID NO: 339 - RNA, 25 nt, synthetic construct
  source 1..25: mol_type = other RNA; note = SLC2A1 NAT siRNA (UPNA-161)
atcaccttctggggggcattgatga

SEQ ID NO: 340 - RNA, 27 nt, synthetic construct
  source 1..27: mol_type = other RNA; note = UPNA-161 complementary strand 5 prime to 3 prime
tcatcaatgccccccagaaggtgatcg

SEQ ID NO: 341 - RNA, 25 nt, synthetic construct
  source 1..25: mol_type = other RNA; note = SLC2A1 NAT siRNA (UPNA-162)
caccttctggggggcattgatgact

SEQ ID NO: 342 - RNA, 27 nt, synthetic construct
  source 1..27: mol_type = other RNA; note = UPNA-162 complementary strand 5 prime to 3 prime
agtcatcaatgccccccagaaggtgat

SEQ ID NO: 343 - RNA, 25 nt, synthetic construct
  source 1..25: mol_type = other RNA; note = SLC2A1 NAT siRNA (UPNA-163)
ctggggggcattgatgactccagtg

SEQ ID NO: 344 - RNA, 27 nt, synthetic construct
  source 1..27: mol_type = other RNA; note = UPNA-163 complementary strand 5 prime to 3 prime
cactggagtcatcaatgccccccagaa

SEQ ID NO: 345 - DNA, 4134 bp, Homo sapiens
  source 1..4134: mol_type = other DNA; note = PICALM (ENST00000393346.8)
gccattttcctgcagctgcctgttcctcttaccctgcccggctccagctgacccgggaaggggtgggctgaactgaggcgggggcaagggagtgcccgacatcttgtccgactccgcgggtgacacgagccggttctctctggactggtggcagcgcgcggccccgaaccgcgccccaggccggcaggcggggaaggagccggtgggggtagggggtgcggtggggggtggggaccctccggctcttgggggtcccagtccccgccggctgctgagcgggtggggtggtggaggagctgcagagatgtccggccagagcctgacggaccgaatcactgccgcccagcacagtgtcaccggctctgccgtatccaagacagtatgcaaggccacgacccacgagatcatggggcccaagaaaaagcacctggactacttaattcagtgcacaaatgagatgaatgtgaacatcccacagttggcagacagtttatttgaaagaactactaatagtagttgggtggtggtcttcaaatctctcattacaactcatcatttgatggtgtatggaaatgagcgttttattcagtatttggcttcaagaaacacgttgtttaacttaagcaattttttggataaaagtggattgcaaggatatgacatgtctacatttattaggcggtatagtagatatttaaatgagaaagcagtttcatacagacaagttgcatttgatttcacaaaagtgaagagaggggctgatggagttatgagaacaatgaacacagaaaaactcctaaaaactgtaccaattattcagaatcagatggatgcacttcttgattttaatgttaatagcaatgaacttacaaatggggtaataaatgctgccttcatgctcctgttcaaagatgccattagactgtttgcagcatacaatgaaggaattattaatttgttggaaaaatattttgatatgaaaaagaaccaatgcaaagaaggtcttgacatctataagaagttcctaactaggatgacaagaatctcagagttcctcaaagttgcagagcaagttggaattgacagaggtgatataccagacctttcacaggcccctagcagtcttcttgatgctttggaacaacatttagcttccttggaaggaaagaaaatcaaagattctacagctgcaagcagggcaactacactttccaatgcagtgtcttccctggcaagcactggtctatctctgaccaaagtggatgaaagggaaaagcaggcagcattagaggaagaacaggcacgtttgaaagctttaaaggaacagcgcctaaaagaacttgcaaagaaacctcatacctctttaacaactgcagcctctcctgtatccacctcagcaggagggataatgactgcaccagccattgacatattttctacccctagttcttctaacagcacatcaaagctgcccaatgatctgcttgatttgcagcagccaacttttcacccatctgtacatcctatgtcaactgcttctcaggtagcaagtacatggggagatcctttctctgctactgtagatgctgttgatgatgccattccaagcttaaatcctttcctcacaaaaagtagtggtgatgttcacctttccatttcttcagatgtatctacttttactactaggacacctactcatgaaatgtttgttggattcactccttctccagttgcacagccacacccttcagctggccttaatgttgactttgaatctgtgtttggaaataaatctacaaatgttattgtagattctgggggctttgatgaactaggtggacttctcaaaccaacagtggcctctcagaaccagaaccttcctgttgccaaactcccacctagcaagttagtatctgatgacttggattcatctttagccaaccttgtgggcaatcttggcatcggaaatggaaccactaagaatgatgtaaattggagtcaaccaggtgaaaagaagttaactgggggatctaactggcaaccaaaggttgcaccaacaaccgcttggaatgctgcaacaatggcaccccctgtaatggcctatcctgctactacaccaacaggcatgataggatatggaattcctccacaaatgggaagtgttcctgtaatgacgcaaccaaccttaatatacagccagcctgtcatgagacctccaaacccctttggccctgtatcaggagcacagatacagtttatgtaacttgatggaagaaaatggaattactccaaaaagacaagtgctcaagcagcaaaatccttacttccagcaaaatccaaactgctgtctcttaaatctcttaaactctcttcttccattagaatgctacaagtaactcagtgaaggcccatgaaggaaattgggactagtttataggagaacgtatcaatacagtttataaagccaagaattgctatgatttaagactaagatctgtctttttggtgactaacccttcaattctttcaactcctgttaatacccataatcagtaacctatcaagaaaagcccttatttggaaagtgtgaaatttgtatttggaaaagctgcctggagagaagaactgtgtcctttactgtatttcaacaggactcttttgggggatcaaaattaaaattcctaattatgcattatctttcttttctccagtcctcacaaatacagaaacaataactgaaattaacttttctttttttaaaaaaaattatattcagtttgcagtagacattccttaagtatttgtatttatttatgattatcaattttacataacattaatattgtatcagacctccttatgaaaatgagtatggatgtgcacagtatgtttgatttttatccacaagaatgaatctgattcagaatgcttttctcagctgacatacagagcactaaatattttaaggcaagtccataggtctgaatctcttaagaattctcggcctctgtgggatttagggaagcattataaatgcattaatccttatagtcaattctgtgcctaggattttgccagggaacagttcactgactaggaaaagcactacattttaaattcagcattagtgcattgggaaggatctttactgctttgtgcttggcatgtcattattttccatttgacattagggcctttccaaaatgaatgtgaggaattgctttcacttcaagactttccttcttttcactaaaactctagaaggtgttacaagggggagggaaggggggcaaagtccttgaacattttctttggctcgtgccatgttatgatcatataccttttaaataaggggaaatagtatctttaaagttaatgtctagccaagagtttagtaaacgaagaattaaactgcactgttgatcggtgctttgtgtaaatacatctttaacatttgggtggagaggggccttaagaaggacagttcattgtaggaaagcaattctgtacatgagtttaagcattcttgttgcattgtctctgcagattctatttttgtttacaatattaaaatgtatgttagcaaaatgggtggattttcaaataaaatgcagcttccacaaaagttttgttatggtattctggtctgagatgcattttcatttttcctttctctttttattatcaatattgtcatttttccctaataaaatatacccaggtgattatatttgttgatctaataacatggaaggtttgttttatatgaattttcaaaaagatgtctctttacactttttgttaccttgtagactcttattgataaatgcaactacttattaaaattgttcactttttgtcttttgatcagatgcctttagtcaggtaagtttaagggaaaatacgcagtttaatgttttggtacatataattatgtctgccaaagaaacctttgattgtatcatattgcctatttagtagtgcatagggttcagagtacatgataaaggatcaaaagctttgcattgataagtgtctcataatatttgctgtgattggagaaaaaatgtagtcgtagccaataaattttatcagcttttaagtttcagtattattaaaccattttcatataaa

SEQ ID NO: 346 - DNA, 480 bp, Homo sapiens
  source 1..480: mol_type = other DNA; note = PICALM NAT (GenBank: AI694932.1)
tttttttttttctcacaacactcatcatcatttgtcataaaatatttgctaattgcctactctcacaagaaaataagctcaatgaaagaagaatctatgttttgtttattgattcttgcacctagaactactggcacattgaaggcactcagtgtattgaatggatgaacgaagaactcaatctgctttctaaaaatcttccttggtcagtcttttgcttagtatctcttttagttcatagctttttgatatctctgcattttctcaatgccatgtgatctgccttctgtccctgacaattcccaggtatatctaggggtaaggttcaagttcctaattgctagaaccaatgggcaggttttggtcgttattttacttgtgttggccattccctacttcttcagttatatccttgacataattatctttcctggttctccgcctccagctttgattgttctttcctggccttcacccctt

SEQ ID NO: 347 - DNA, 309 bp, Homo sapiens
  source 1..309: mol_type = other DNA; note = PICALM NAT (GenBank: BF910880.1)
acacacatgtcctgaaattcgatagtgatgatgcatctactttgcaattataataaaactactgaattatacacttttaaaagacaacattggccgggcgtggtgtctccatacattgtgacctagtcactgctagcacacaggagccacactcactcttaagagagattttttttctctgtggaattttgttgaataagcaaacagagtttggaagcgtgtttgtgagttacgataaatgagaagaaccttgaaattatttttagttcaagaaaaacatcccattaaggcctatttctcatagtcact

SEQ ID NO: 348 - DNA, 701 bp, Homo sapiens
  source 1..701: mol_type = other DNA; note = PICALM NAT (GenBank: BQ016971.1)
ttttttttttttttttttaaaagctgataaaatttattgggctacgactacattttttctccaatcacagcaaatattatgagacacttatcaatgcaaagcttttgatcctttatcatgtactctgaaccctatgcactactaaataggcaatatgatacaatcaaaggtttctttggcagacataattatatgtaccaaaacattaaactgcgtattttcccttaaacttacctgactaaaggcatctgatcaaaagacaaaaagtgaacaattttaataagtagttgcatttatcaataagagtctacaaggtaacaaaaagtgtaaagagacatctttttgaaaattcatataaaacaaaccttccatgttattagatcaacaaatataatcacctgggtatattttattagggaaaaatgacaatattgataataaaaagagaaaggaaaaatgaaaatgcatctcagaccagaataccataacaaaacttttgtggaagctgcattttatttgaaaatccacccattttgctaacatacattttaatattgtaaacaaaaatagaatctgcagagacaatgcaacaagaatgcttaaactcatgtacagaattgctttcctacaatgaactgtccttcttaaggcccctctccacccaaatgttaaagatgtatttacacaaagcaccgatcaac

SEQ ID NO: 349 - DNA, 786 bp, Homo sapiens
  source 1..786: mol_type = other DNA; note = PICALM NAT (GenBank: AL135735.1)
tttttttttttttttttgtggaagctgcattttatttgaaaatccacccattttgctaacatacattttaatattgtaaacaaaaatagaatctgcagagacaatgcaacaagaatgcttaaactcatgtacagaattgctttcctacaatgaactgtccttcttaaggcccctctccacccaaatgttaaagatgtatttacacaaagcaccgatcaacagtgcagtttaattcttcgtttactaaactcttggctagacattaactttaaagatactatttccccttatttaaaaggtatatgatcataacatggcacgagccaaagaaaatgttcaaggactttgccccccttccctcccccttgtaacaccttctagagttttagtgaaaagaaggaaagtcttgaagtgaaagcaattcctcacattcattttggaaaggccctaatgtcaaatggaaaataatgacatgccaagcacaaagcagtaaagatccttcccaatgcactaatgctgaatttaaaatgtagtgcttttcctagtcagtgaactgttccctggcaaaatcctaggcacagaattgactataaggattaatgcatttataatgcttccctaaatcccacagaggccgagaattcttaagagattcagacctatggacttgccttaaaatatttagtgctctgtatgtcagctgagaaaagcattctgaatcagattcattcttgtggataaaaatcaaacatactggcacatccatactcattttcataaggaggt

SEQ ID NO: 350 - DNA, 578 bp, Homo sapiens
  source 1..578: mol_type = other DNA; note = PICALM NAT (GenBank: MN309288.1)
caaaagagtcctgttgaaatacagtaaaggacacagttcttctctccaggcagcttttccaaatacaaatttcacactttccaaataagggcttttcttgataggttactgattatgggtattaacaggagttgaaagaattgaagggttagtcaccaaaaagacagatcttagtcttaaatcatagcaattcttggctttataaactgtattgatacgttctcctataaactagtcccaatttccttcatgggccttcactgagttacttgtagcattctaatggaagaagagagtttaagagatttaagagacagcagtttggattttgctggaagtaaggattttgctgcttgagcacttgtctttttggagtaattccattttcttccatcaagttacataaactgtatctgtgctcctgatacagggccaaaggggtttggaggtctcatgacaggctggctgtatattaaggttggttgcgtcattacaggaacacttcccatttgtggaggaattccatatcctatcatgcctgttggtgtagtagcaggataggccattacagggggtgc

SEQ ID NO: 351 - RNA, 25 nt, synthetic construct
  source 1..25: mol_type = other RNA; note = PICALM NAT siRNA (UPNA-164)
accttgtagactcttattgataaat

SEQ ID NO: 352 - RNA, 27 nt, synthetic construct
  source 1..27: mol_type = other RNA; note = UPNA-164 complementary strand 5 prime to 3 prime
atttatcaataagagtctacaaggtaa

SEQ ID NO: 353 - RNA, 25 nt, synthetic construct
  source 1..25: mol_type = other RNA; note = PICALM NAT siRNA (UPNA-165)
gactcttattgataaatgcaactac

SEQ ID NO: 354 - RNA, 27 nt, synthetic construct
  source 1..27: mol_type = other RNA; note = UPNA-165 complementary strand 5 prime to 3 prime
gtagttgcatttatcaataagagtcta

SEQ ID NO: 355 - RNA, 25 nt, synthetic construct
  source 1..25: mol_type = other RNA; note = PICALM NAT siRNA (UPNA-166)
ccttgtagactcttattgataaatg

SEQ ID NO: 356 - RNA, 27 nt, synthetic construct
  source 1..27: mol_type = other RNA; note = UPNA-166 complementary strand 5 prime to 3 prime
catttatcaataagagtctacaaggta

SEQ ID NO: 357 - RNA, 25 nt, synthetic construct
  source 1..25: mol_type = other RNA; note = PICALM NAT siRNA (UPNA-167)
ggcaaagtccttgaacattttcttt

SEQ ID NO: 358 - RNA, 27 nt, synthetic construct
  source 1..27: mol_type = other RNA; note = UPNA-167 complementary strand 5 prime to 3 prime
aaagaaaatgttcaaggactttgcccc

SEQ ID NO: 359 - RNA, 25 nt, synthetic construct
  source 1..25: mol_type = other RNA; note = PICALM NAT siRNA (UPNA-168)
actcttattgataaatgcaactact

SEQ ID NO: 360 - RNA, 27 nt, synthetic construct
  source 1..27: mol_type = other RNA; note = UPNA-168 complementary strand 5 prime to 3 prime
agtagttgcatttatcaataagagtct

SEQ ID NO: 361 - RNA, 25 nt, synthetic construct
  source 1..25: mol_type = other RNA; note = PICALM NAT siRNA (UPNA-169)
ctaaaactctagaaggtgttacaag

SEQ ID NO: 362 - RNA, 27 nt, synthetic construct
  source 1..27: mol_type = other RNA; note = UPNA-169 complementary strand 5 prime to 3 prime
cttgtaacaccttctagagttttagtg

SEQ ID NO: 363 - RNA, 25 nt, synthetic construct
  source 1..25: mol_type = other RNA; note = PICALM NAT siRNA (UPNA-170)
gtagactcttattgataaatgcaac

SEQ ID NO: 364 - RNA, 27 nt, synthetic construct
  source 1..27: mol_type = other RNA; note = UPNA-170 complementary strand 5 prime to 3 prime
gttgcatttatcaataagagtctacaa

SEQ ID NO: 365 - RNA, 25 nt, synthetic construct
  source 1..25: mol_type = other RNA; note = PICALM NAT siRNA (UPNA-171)
actaaaactctagaaggtgttacaa

SEQ ID NO: 366 - RNA, 27 nt, synthetic construct
  source 1..27: mol_type = other RNA; note = UPNA-171 complementary strand 5 prime to 3 prime
ttgtaacaccttctagagttttagtga

SEQ ID NO: 367 - RNA, 25 nt, synthetic construct
  source 1..25: mol_type = other RNA; note = PICALM NAT siRNA (UPNA-172)
ctcagtgtattgaatggatgaacga

SEQ ID NO: 368 - RNA, 27 nt, synthetic construct
  source 1..27: mol_type = other RNA; note = UPNA-172 complementary strand 5 prime to 3 prime
tcgttcatccattcaatacactgagtg

SEQ ID NO: 369 - RNA, 25 nt, synthetic construct
  source 1..25: mol_type = other RNA; note = PICALM NAT siRNA (UPNA-173)
aactcaatctgctttctaaaaatct

SEQ ID NO: 370 - RNA, 27 nt, synthetic construct
  source 1..27: mol_type = other RNA; note = UPNA-173 complementary strand 5 prime to 3 prime
agatttttagaaagcagattgagttct

SEQ ID NO: 371 - RNA, 25 nt, synthetic construct
  source 1..25: mol_type = other RNA; note = PICALM NAT siRNA (UPNA-174)
gtcagtcttttgcttagtatctctt

SEQ ID NO: 372 - RNA, 27 nt, synthetic construct
  source 1..27: mol_type = other RNA; note = UPNA-174 complementary strand 5 prime to 3 prime
aagagatactaagcaaaagactgacca

SEQ ID NO: 373 - RNA, 25 nt, synthetic construct
  source 1..25: mol_type = other RNA; note = PICALM NAT siRNA (UPNA-175)
caagttcctaattgctagaaccaat

SEQ ID NO: 374 - RNA, 27 nt, synthetic construct
  source 1..27: mol_type = other RNA; note = UPNA-175 complementary strand 5 prime to 3 prime
attggttctagcaattaggaacttgaa

SEQ ID NO: 375 - RNA, 25 nt, synthetic construct
  source 1..25: mol_type = other RNA; note = PICALM NAT siRNA (UPNA-176)
aacgaagaactcaatctgctttcta

SEQ ID NO: 376 - RNA, 27 nt, synthetic construct
  source 1..27: mol_type = other RNA; note = UPNA-176 complementary strand 5 prime to 3 prime
tagaaagcagattgagttcttcgttca

SEQ ID NO: 377 - RNA, 25 nt, synthetic construct
  source 1..25: mol_type = other RNA; note = PICALM NAT siRNA (UPNA-177)
cttggtcagtcttttgcttagtatc

SEQ ID NO: 378 - RNA, 27 nt, synthetic construct
  source 1..27: mol_type = other RNA; note = UPNA-177 complementary strand 5 prime to 3 prime
gatactaagcaaaagactgaccaagga

SEQ ID NO: 379 - RNA, 25 nt, synthetic construct
  source 1..25: mol_type = other RNA; note = PICALM NAT siRNA (UPNA-178)
tcaagttcctaattgctagaaccaa

SEQ ID NO: 380 - RNA, 27 nt, synthetic construct
  source 1..27: mol_type = other RNA; note = UPNA-178 complementary strand 5 prime to 3 prime
ttggttctagcaattaggaacttgaac

SEQ ID NO: 381 - RNA, 25 nt, synthetic construct
  source 1..25: mol_type = other RNA; note = PICALM NAT siRNA (UPNA-179)
actcaatctgctttctaaaaatctt

SEQ ID NO: 382 - RNA, 27 nt, synthetic construct
  source 1..27: mol_type = other RNA; note = UPNA-179 complementary strand 5 prime to 3 prime
aagatttttagaaagcagattgagttc

SEQ ID NO: 383 - RNA, 25 nt, synthetic construct
  source 1..25: mol_type = other RNA; note = PICALM NAT siRNA (UPNA-180)
ccctgacaattcccaggtatatcta

SEQ ID NO: 384 - RNA, 27 nt, synthetic construct
  source 1..27: mol_type = other RNA; note = UPNA-180 complementary strand 5 prime to 3 prime
tagatatacctgggaattgtcagggac

SEQ ID NO: 385 - RNA, 25 nt, synthetic construct
  source 1..25: mol_type = other RNA; note = PICALM NAT siRNA (UPNA-181)
cgataaatgagaagaaccttgaaat

SEQ ID NO: 386 - RNA, 27 nt, synthetic construct
  source 1..27: mol_type = other RNA; note = UPNA-181 complementary strand 5 prime to 3 prime
atttcaaggttcttctcatttatcgta

SEQ ID NO: 387 - RNA, 25 nt, synthetic construct
  source 1..25: mol_type = other RNA; note = PICALM NAT siRNA (UPNA-182)
ggaagcgtgtttgtgagttacgata

SEQ ID NO: 388 - RNA, 27 nt, synthetic construct
  source 1..27: mol_type = other RNA; note = UPNA-182 complementary strand 5 prime to 3 prime
tatcgtaactcacaaacacgcttccaa

SEQ ID NO: 389 - RNA, 25 nt, synthetic construct
  source 1..25: mol_type = other RNA; note = PICALM NAT siRNA (UPNA-183)
acacatgtcctgaaattcgatagtg

SEQ ID NO: 390 - RNA, 27 nt, synthetic construct
  source 1..27: mol_type = other RNA; note = UPNA-183 complementary strand 5 prime to 3 prime
cactatcgaatttcaggacatgtgtgt

SEQ ID NO: 391 - RNA, 25 nt, synthetic construct
  source 1..25: mol_type = other RNA; note = PICALM NAT siRNA (UPNA-184)
gtgtttgtgagttacgataaatgag

SEQ ID NO: 392 - RNA, 27 nt, synthetic construct
  source 1..27: mol_type = other RNA; note = UPNA-184 complementary strand 5 prime to 3 prime
ctcatttatcgtaactcacaaacacgc

SEQ ID NO: 393 - RNA, 25 nt, synthetic construct
  source 1..25: mol_type = other RNA; note = PICALM NAT siRNA (UPNA-185)
tgagttacgataaatgagaagaacc

SEQ ID NO: 394 - RNA, 27 nt, synthetic construct
  source 1..27: mol_type = other RNA; note = UPNA-185 complementary strand 5 prime to 3 prime
ggttcttctcatttatcgtaactcaca

SEQ ID NO: 395 - RNA, 25 nt, synthetic construct
  source 1..25: mol_type = other RNA; note = PICALM NAT siRNA (UPNA-186)
agcgtgtttgtgagttacgataaat

SEQ ID NO: 396 - RNA, 27 nt, synthetic construct
  source 1..27: mol_type = other RNA; note = UPNA-186 complementary strand 5 prime to 3 prime
atttatcgtaactcacaaacacgcttc

SEQ ID NO: 397 - RNA, 25 nt, synthetic construct
  source 1..25: mol_type = other RNA; note = PICALM NAT siRNA (UPNA-187)
gtgagttacgataaatgagaagaac

SEQ ID NO: 398 - RNA, 27 nt, synthetic construct
  source 1..27: mol_type = other RNA; note = UPNA-187 complementary strand 5 prime to 3 prime
gttcttctcatttatcgtaactcacaa

SEQ ID NO: 399 - RNA, 25 nt, synthetic construct
  source 1..25: mol_type = other RNA; note = PICALM NAT siRNA (UPNA-188)
atgtcctgaaattcgatagtgatga

SEQ ID NO: 400 - RNA, 27 nt, synthetic construct
  source 1..27: mol_type = other RNA; note = UPNA-188 complementary strand 5 prime to 3 prime
tcatcactatcgaatttcaggacatgt

SEQ ID NO: 401 - RNA, 25 nt, synthetic construct
  source 1..25: mol_type = other RNA; note = PICALM NAT siRNA (UPNA-189)
gagttacgataaatgagaagaacct

SEQ ID NO: 402 - RNA, 27 nt, synthetic construct
  source 1..27: mol_type = other RNA; note = UPNA-189 complementary strand 5 prime to 3 prime
aggttcttctcatttatcgtaactcac

SEQ ID NO: 403 - RNA, 25 nt, synthetic construct
  source 1..25: mol_type = other RNA; note = PICALM NAT siRNA (UPNA-190)
gtcctgaaattcgatagtgatgatg

SEQ ID NO: 404 - RNA, 27 nt, synthetic construct
  source 1..27: mol_type = other RNA; note = UPNA-190 complementary strand 5 prime to 3 prime
catcatcactatcgaatttcaggacat

SEQ ID NO: 405 - RNA, 25 nt, synthetic construct
  source 1..25: mol_type = other RNA; note = PICALM NAT siRNA (UPNA-191)
gcgtgtttgtgagttacgataaatg

SEQ ID NO: 406 - RNA, 27 nt, synthetic construct
  source 1..27: mol_type = other RNA; note = UPNA-191 complementary strand 5 prime to 3 prime
catttatcgtaactcacaaacacgctt

SEQ ID NO: 407 - RNA, 25 nt, synthetic construct
  source 1..25: mol_type = other RNA; note = PICALM NAT siRNA (UPNA-192)
aagcgtgtttgtgagttacgataaa

SEQ ID NO: 408 - RNA, 27 nt, synthetic construct
  source 1..27: mol_type = other RNA; note = UPNA-192 complementary strand 5 prime to 3 prime
tttatcgtaactcacaaacacgcttcc

SEQ ID NO: 409 - RNA, 25 nt, synthetic construct
  source 1..25: mol_type = other RNA; note = PICALM NAT siRNA (UPNA-193)
gatgatgcatctactttgcaattat

SEQ ID NO: 410 - RNA, 27 nt, synthetic construct
  source 1..27: mol_type = other RNA; note = UPNA-193 complementary strand 5 prime to 3 prime
ataattgcaaagtagatgcatcatcac

SEQ ID NO: 411 - RNA, 25 nt, synthetic construct
  source 1..25: mol_type = other RNA; note = PICALM NAT siRNA (UPNA-194)
acttacctgactaaaggcatctgat

SEQ ID NO: 412 - RNA, 27 nt, synthetic construct
  source 1..27: mol_type = other RNA; note = UPNA-194 complementary strand 5 prime to 3 prime
atcagatgcctttagtcaggtaagttt

SEQ ID NO: 413 - RNA, 25 nt, synthetic construct
  source 1..25: mol_type = other RNA; note = PICALM NAT siRNA (UPNA-195)
cacttatcaatgcaaagcttttgat

SEQ ID NO: 414 - RNA, 27 nt, synthetic construct
  source 1..27: mol_type = other RNA; note = UPNA-195 complementary strand 5 prime to 3 prime
atcaaaagctttgcattgataagtgtc

SEQ ID NO: 415 - RNA, 25 nt, synthetic construct
  source 1..25: mol_type = other RNA; note = PICALM NAT siRNA (UPNA-196)
aacttacctgactaaaggcatctga

SEQ ID NO: 416 - RNA, 27 nt, synthetic construct
  source 1..27: mol_type = other RNA; note = UPNA-196 complementary strand 5 prime to 3 prime
tcagatgcctttagtcaggtaagttta

SEQ ID NO: 417 - RNA, 25 nt, synthetic construct
  source 1..25: mol_type = other RNA; note = PICALM NAT siRNA (UPNA-197)
ccttccatgttattagatcaacaaa

SEQ ID NO: 418 - RNA, 27 nt, synthetic construct
  source 1..27: mol_type = other RNA; note = UPNA-197 complementary strand 5 prime to 3 prime
tttgttgatctaataacatggaaggtt

SEQ ID NO: 419 - RNA, 25 nt, synthetic construct
  source 1..25: mol_type = other RNA; note = PICALM NAT siRNA (UPNA-198)
cttacctgactaaaggcatctgatc

SEQ ID NO: 420 - RNA, 27 nt, synthetic construct
  source 1..27: mol_type = other RNA; note = UPNA-198 complementary strand 5 prime to 3 prime
gatcagatgcctttagtcaggtaagtt

SEQ ID NO: 421 - RNA, 25 nt, synthetic construct
  source 1..25: mol_type = other RNA; note = PICALM NAT siRNA (UPNA-199)
tacctgactaaaggcatctgatcaa

SEQ ID NO: 422 - RNA, 27 nt, synthetic construct
  source 1..27: mol_type = other RNA; note = UPNA-199 complementary strand 5 prime to 3 prime
ttgatcagatgcctttagtcaggtaag

SEQ ID NO: 423 - RNA, 25 nt, synthetic construct
  source 1..25: mol_type = other RNA; note = PICALM NAT siRNA (UPNA-200)
agaatgcttaaactcatgtacagaa

SEQ ID NO: 424 - RNA, 27 nt, synthetic construct
  source 1..27: mol_type = other RNA; note = UPNA-200 complementary strand 5 prime to 3 prime
ttctgtacatgagtttaagcattcttg

SEQ ID NO: 425 - RNA, 25 nt, synthetic construct
  source 1..25: mol_type = other RNA; note = PICALM NAT siRNA (UPNA-201)
atgcttaaactcatgtacagaattg

SEQ ID NO: 426 - RNA, 27 nt, synthetic construct
  source 1..27: mol_type = other RNA; note = UPNA-201 complementary strand 5 prime to 3 prime
caattctgtacatgagtttaagcattc

SEQ ID NO: 427 - RNA, 25 nt, synthetic construct
  source 1..25: mol_type = other RNA; note = PICALM NAT siRNA (UPNA-202)
actaaaggcatctgatcaaaagaca

SEQ ID NO: 428 - RNA, 27 nt, synthetic construct
  source 1..27: mol_type = other RNA; note = UPNA-202 complementary strand 5 prime to 3 prime
tgtcttttgatcagatgcctttagtca

SEQ ID NO: 429 - RNA, 25 nt, synthetic construct
  source 1..25: mol_type = other RNA; note = PICALM NAT siRNA (UPNA-203)
gcttaaactcatgtacagaattgct

SEQ ID NO: 430 - RNA, 27 nt, synthetic construct
  source 1..27: mol_type = other RNA; note = UPNA-203 complementary strand 5 prime to 3 prime
agcaattctgtacatgagtttaagcat

SEQ ID NO: 431 - RNA, 25 nt, synthetic construct
  source 1..25: mol_type = other RNA; note = PICALM NAT siRNA (UPNA-204)
caaaccttccatgttattagatcaa

SEQ ID NO: 432 - RNA, 27 nt, synthetic construct
  source 1..27: mol_type = other RNA; note = UPNA-204 complementary strand 5 prime to 3 prime
ttgatctaataacatggaaggtttgtt

SEQ ID NO: 433 - RNA, 25 nt, synthetic construct
  source 1..25: mol_type = other RNA; note = PICALM NAT siRNA (UPNA-205)
cgatcaacagtgcagtttaattctt

SEQ ID NO: 434 - RNA, 27 nt, synthetic construct
  source 1..27: mol_type = other RNA; note = UPNA-205 complementary strand 5 prime to 3 prime
aagaattaaactgcactgttgatcggt

SEQ ID NO: 435 - RNA, 25 nt, synthetic construct
  source 1..25: mol_type = other RNA; note = PICALM NAT siRNA (UPNA-206)
ccgatcaacagtgcagtttaattct

SEQ ID NO: 436 - RNA, 27 nt, synthetic construct
  source 1..27: mol_type = other RNA; note = UPNA-206 complementary strand 5 prime to 3 prime
agaattaaactgcactgttgatcggtg

SEQ ID NO: 437 - RNA, 25 nt, synthetic construct
  source 1..25: mol_type = other RNA; note = PICALM NAT siRNA (UPNA-207)
gaattgactataaggattaatgcat

SEQ ID NO: 438 - RNA, 27 nt, synthetic construct
  source 1..27: mol_type = other RNA; note = UPNA-207 complementary strand 5 prime to 3 prime
atgcattaatccttatagtcaattctg

SEQ ID NO: 439 - RNA, 25 nt, synthetic construct
  source 1..25: mol_type = other RNA; note = PICALM NAT siRNA (UPNA-208)
agaatgcttaaactcatgtacagaa

SEQ ID NO: 440 - RNA, 27 nt, synthetic construct
  source 1..27: mol_type = other RNA; note = UPNA-208 complementary strand 5 prime to 3 prime
ttctgtacatgagtttaagcattcttg

SEQ ID NO: 441 - RNA, 25 nt, synthetic construct
  source 1..25: mol_type = other RNA; note = PICALM NAT siRNA (UPNA-209)
atgcttaaactcatgtacagaattg

SEQ ID NO: 442 - RNA, 27 nt, synthetic construct
  source 1..27: mol_type = other RNA; note = UPNA-209 complementary strand 5 prime to 3 prime
caattctgtacatgagtttaagcattc

SEQ ID NO: 443 - RNA, 25 nt, synthetic construct
  source 1..25: mol_type = other RNA; note = PICALM NAT siRNA (UPNA-210)
gcttaaactcatgtacagaattgct

SEQ ID NO: 444 - RNA, 27 nt, synthetic construct
  source 1..27: mol_type = other RNA; note = UPNA-210 complementary strand 5 prime to 3 prime
agcaattctgtacatgagtttaagcat

SEQ ID NO: 445 - RNA, 25 nt, synthetic construct
  source 1..25: mol_type = other RNA; note = PICALM NAT siRNA (UPNA-211)
gttgcgtcattacaggaacacttcc

SEQ ID NO: 446 - RNA, 27 nt, synthetic construct
  source 1..27: mol_type = other RNA; note = UPNA-211 complementary strand 5 prime to 3 prime
ggaagtgttcctgtaatgacgcaacca

SEQ ID NO: 447 - RNA, 25 nt, synthetic construct
  source 1..25: mol_type = other RNA; note = PICALM NAT siRNA (UPNA-212)
gtaaaggacacagttcttctctcca

SEQ ID NO: 448 - RNA, 27 nt, synthetic construct
  source 1..27: mol_type = other RNA; note = UPNA-212 complementary strand 5 prime to 3 prime
tggagagaagaactgtgtcctttactg

SEQ ID NO: 449 - RNA, 25 nt, synthetic construct
  source 1..25: mol_type = other RNA; note = PICALM NAT siRNA (UPNA-213)
ggttgcgtcattacaggaacacttc

SEQ ID NO: 450 - RNA, 27 nt, synthetic construct
  source 1..27: mol_type = other RNA; note = UPNA-213 complementary strand 5 prime to 3 prime
gaagtgttcctgtaatgacgcaaccaa

SEQ ID NO: 451 - RNA, 25 nt, synthetic construct
  source 1..25: mol_type = other RNA; note = PICALM NAT siRNA (UPNA-214)
gaattgaagggttagtcaccaaaaa

SEQ ID NO: 452 - RNA, 27 nt, synthetic construct
  source 1..27: mol_type = other RNA; note = UPNA-214 complementary strand 5 prime to 3 prime
tttttggtgactaacccttcaattctt

SEQ ID NO: 453 - RNA, 25 nt, synthetic construct
  source 1..25: mol_type = other RNA; note = PICALM NAT siRNA (UPNA-215)
aagacagatcttagtcttaaatcat

SEQ ID NO: 454 - RNA, 27 nt, synthetic construct
  source 1..27: mol_type = other RNA; note = UPNA-215 complementary strand 5 prime to 3 prime
atgatttaagactaagatctgtctttt

SEQ ID NO: 455 - RNA, 25 nt, synthetic construct
  source 1..25: mol_type = other RNA; note = PICALM NAT siRNA (UPNA-216)
agaattgaagggttagtcaccaaaa

SEQ ID NO: 456 - RNA, 27 nt, synthetic construct
  source 1..27: mol_type = other RNA; note = UPNA-216 complementary strand 5 prime to 3 prime
ttttggtgactaacccttcaattcttt

SEQ ID NO: 457 - RNA, 25 nt, synthetic construct
  source 1..25: mol_type = other RNA; note = PICALM NAT siRNA (UPNA-217)
aaagacagatcttagtcttaaatca

SEQ ID NO: 458 - RNA, 27 nt, synthetic construct
  source 1..27: mol_type = other RNA; note = UPNA-217 complementary strand 5 prime to 3 prime
tgatttaagactaagatctgtcttttt

SEQ ID NO: 459 - RNA, 25 nt, synthetic construct
  source 1..25: mol_type = other RNA; note = PICALM NAT siRNA (UPNA-218)
gaaatacagtaaaggacacagttct

SEQ ID NO: 460 - RNA, 27 nt, synthetic construct
  source 1..27: mol_type = other RNA; note = UPNA-218 complementary strand 5 prime to 3 prime
agaactgtgtcctttactgtatttcaa

SEQ ID NO: 461 - RNA, 25 nt, synthetic construct
  source 1..25: mol_type = other RNA; note = PICALM NAT siRNA (UPNA-219)
actgagttacttgtagcattctaat

SEQ ID NO: 462 - RNA, 27 nt, synthetic construct
  source 1..27: mol_type = other RNA; note = UPNA-219 complementary strand 5 prime to 3 prime
attagaatgctacaagtaactcagtga

SEQ ID NO: 463 - RNA, 25 nt, synthetic construct
  source 1..25: mol_type = other RNA; note = PICALM NAT siRNA (UPNA-220)
gcgtcattacaggaacacttcccat

SEQ ID NO: 464 - RNA, 27 nt, synthetic construct
  source 1..27: mol_type = other RNA; note = UPNA-220 complementary strand 5 prime to 3 prime
atgggaagtgttcctgtaatgacgcaa

SEQ ID NO: 465 - RNA, 25 nt, synthetic construct
  source 1..25: mol_type = other RNA; note = PICALM NAT siRNA (UPNA-221)
aattgaagggttagtcaccaaaaag

SEQ ID NO: 466 - RNA, 27 nt, synthetic construct
  source 1..27: mol_type = other RNA; note = UPNA-221 complementary strand 5 prime to 3 prime
ctttttggtgactaacccttcaattct

SEQ ID NO: 467 - DNA, 645 bp, Homo sapiens
  source 1..645: mol_type = other DNA; note = LRP1 Natural Antisense Transcript (NAT) (ENST00000555461.1)
gtgcacccctgctgcatgaggctcagccccaggggtccaacagaggtcacagtcacagcacagaatggaaggcttgcagggagggctgccctggagggcagagtgaaagcaggggaaagctgcagcctttcttcttcgtcctccttggggagctaattacaggccagcagccactgtgcaggagcggttatccggctgcaggaggtatccttcaacacagccacatatgaaggagccgtctgtgttggtgcatagctggctgcaggtgccgtacactgagcactcatcaaaatctgccgaggaaagggcaagaatgtggtcatttccagtgtgacaagggcaacgaagtgggtgatcatcagacatgatcaacatgtccatccttacagctaccttgaaggacagttcggcagaatctattacagcatgaaatgtgcaaaccttaacacccagcaagtccagtcttggaatccactctagagaaacactcgtgtacatatcactcatccacgcagggaagatttattgagtccctactatgggtcaggtgccaggggtgcatcagtgaactttaaaatagacccacattgctgtcctcatgaaacttatattcagataataaatgaaatatagttactataataa

SEQ ID NO: 468 - DNA, 536 bp, Homo sapiens
  source 1..536: mol_type = other DNA; note = LRP1 NAT (GenBank - BX477794.1)
tgttttttttttttttttttaaagagatggagtcctgatctgtcgcccaggctggagttcagtggcacaatattggcccactgcaacccttgaaccttcccggttcaagtgattctcctgcctaggtcttctgagtagctgggattacaggtgcccaccaccacgcccagctaatttttgtggttttagtagagacggagttttgccatgttggccaagctagtctcaaactcctgacctcaagtgatcggccggcctcagcctcccaaagtgctgggattacaggcttgagccactgcgcctggcccagttttcccatgtcttgaggcaccactacccatgcacctcagaatcctcccttgcctttatccctttgatacagcacatcccaaagtgaatccccacatggtccctggttgctcagactcagtgaaaaaaaaatgaatggtcaagtgagttttggaaaaccccaaacgcttgaaaaattcttggcacacataaacatattcaaggctctgagaagttctgcagcacaa

SEQ ID NO: 469 - DNA, 565 bp, Homo sapiens
  source 1..565: mol_type = other DNA; note = LRP1 NAT (GenBank - DC310139.1)
ggttctctatcgctcatacccagcactaccaagtcaggggccttcctgcagtaccattcagctgcacccagatgccccaagagcaatgagcccacacgcagagctggaggacctgaaaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtagg

SEQ ID NO: 470 - DNA, 551 bp, Homo sapiens
  source 1..551: mol_type = other DNA; note = LRP1 NAT (GenBank - DC401271.1)
accattcagctgcacccagatgccccaagagcaatgagcccacacgcagagctggaggacctgaaaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaaggggg

SEQ ID NO: 471 - DNA, 452 bp, Homo sapiens
  source 1..452: mol_type = other DNA; note = LRP1 NAT (GenBank - AA704922.1)
ttactgatcaaaatacctttatgagaagtacagaatccggttgagaagtgtggtaccccagacaagtagagagctgagaaaagctgcactgagatggatcctacattcctgggtctgggagtgggcctttcccagagataccatcctccagcagtaagagacctccaatagtcgggtctcagacaggttccagctaagagtaaaatcccagatcaatggtgaaaaggagtttaagaatggatgctgaagccaggcatggtggcatgtgtctgtagtcccagctacttgggaagttgacaggggaaaatagtttaagcccaggaggttgaggctgcagtgagctatgatcataccactgcactccagactgggcgacagagcaaaactctgtctcaaaaagaaaaacgaagcctgtgcctagtcaacattgaagcaagaatgcagctcgtgcc

SEQ ID NO: 472 - DNA, 73 bp, Homo sapiens
  source 1..73: mol_type = other DNA; note = ENST00000408438.1
gtgggcgggggcaggtgtgtggtgggtggtggcctgcggtgagcagggccctcacacctgcctcgccccccag

SEQ ID NO: 473 - DNA, 14923 bp, Homo sapiens
  source 1..14923: mol_type = other DNA; note = LDL receptor related protein 1 (LRP1) - ENST00000243077.8
acttcagtccggggaacagcggtgcgagctccaggcccatgcactgaggaggcggaaacaaggggagcccccagagctccatcaagccccctccaaaggctcccctacccggtccacgccccccaccccccctccccgcctcctcccaattgtgcatttttgcagccggaggcggctccgagatggggctgtgagcttcgcccggggagggggaaagagcagcgaggagtgaagcgggggggtggggtgaagggtttggatttcggggcagggggcgcacccccgtcagcaggccctccccaaggggctcggaactctacctcttcacccacgcccctggtgcgctttgccgaaggaaagaataagaacagagaaggaggagggggaaaggaggaaaagggggaccccccaactggggggggtgaaggagagaagtagcaggaccagaggggaaggggctgctgcttgcatcagcccacaccatgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgccgagcggtgaattcctcttgccgagcacaagatgagtttgagtgtgccaatggcgagtgcatcaacttcagcctgacctgcgacggcgtcccccactgcaaggacaagtccgatgagaagccatcctactgcaactcccgccgctgcaagaagactttccggcagtgcagcaatgggcgctgtgtgtccaacatgctgtggtgcaacggggccgacgactgtggggatggctctgacgagatcccttgcaacaagacagcctgtggtgtgggcgagttccgctgccgggacgggacctgcatcgggaactccagccgctgcaaccagtttgtggattgtgaggacgcctcagatgagatgaactgcagtgccaccgactgcagcagctacttccgcctgggcgtgaagggcgtgctcttccagccctgcgagcggacctcactctgctacgcacccagctgggtgtgtgatggcgccaatgactgtggggactacagtgatgagcgcgactgcccaggtgtgaaacgccccagatgccctctgaattacttcgcctgccctagtgggcgctgcatccccatgagctggacgtgtgacaaagaggatgactgtgaacatggcgaggacgagacccactgcaacaagttctgctcagaggcccagtttgagtgccagaaccatcgctgcatctccaagcagtggctgtgtgacggcagcgatgactgtggggatggctcagacgaggctgctcactgtgaaggcaagacgtgcggcccctcctccttctcctgccctggcacccacgtgtgcgtccccgagcgctggctctgtgacggtgacaaagactgtgctgatggtgcagacgagagcatcgcagctggttgcttgtacaacagcacttgtgacgaccgtgagttcatgtgccagaaccgccagtgcatccccaagcacttcgtgtgtgaccacgaccgtgactgtgcagatggctctgatgagtcccccgagtgtgagtacccgacctgcggccccagtgagttccgctgtgccaatgggcgctgtctgagctcccgccagtgggagtgtgatggcgagaatgactgccacgaccagagtgacgaggctcccaagaacccacactgcaccagccaagagcacaagtgcaatgcctcgtcacagttcctgtgcagcagtgggcgctgtgtggctgaggcactgctctgcaacggccaggatgactgtggcgacagctcggacgagcgtggctgccacatcaatgagtgtctcagccgcaagctcagtggctgcagccaggactgtgaggacctcaagatcggcttcaagtgccgctgtcgccctggcttccggctgaaggacgacggccggacgtgtgctgatgtggacgagtgcagcaccaccttcccctgcagccagcgctgcatcaacactcatggcagctataagtgtctgtgtgtggagggctatgcaccccgcggcggcgacccccacagctgcaaggctgtgactgacgaggaaccgtttctgatcttcgccaaccggtactacctgcgcaagctcaacctggacgggtccaactacacgttacttaagcagggcctgaacaacgccgttgccttggattttgactaccgagagcagatgatctactggacagatgtgaccacccagggcagcatgatccgaaggatgcaccttaacgggagcaatgtgcaggtcctacaccgtacaggcctcagcaaccccgatgggctggctgtggactgggtgggtggcaacctgtactggtgcgacaaaggccgggacaccatcgaggtgtccaagctcaatggggcctatcggacggtgctggtcagctctggcctccgtgagcccagggctctggtggtggatgtgcagaatgggtacctgtactggacagactggggtgaccattcactgatcggccgcatcggcatggatgggtccagccgcagcgtcatcgtggacaccaagatcacatggcccaatggcctgacgctggactatgtcactgagcgcatctactgggccgacgcccgcgaggactacattgaatttgccagcctggatggctccaatcgccacgttgtgctgagccaggacatcccgcacatctttgcactgaccctgtttgaggactacgtctactggaccgactgggaaacaaagtccattaaccgagcccacaagaccacgggcaccaacaaaacgctcctcatcagcacgctgcaccggcccatggacctgcatgtcttccatgccctgcgccagccagacgtgcccaatcacccctgcaaggtcaacaatggtggctgcagcaacctgtgcctgctgtcccccgggggagggcacaaatgtgcctgccccaccaacttctacctgggcagcgatgggcgcacctgtgtgtccaactgcacggctagccagtttgtatgcaagaacgacaagtgcatccccttctggtggaagtgtgacaccgaggacgactgcggggaccactcagacgagcccccggactgccctgagttcaagtgccggcccggacagttccagtgctccacaggtatctgcacaaaccctgccttcatctgcgatggcgacaatgactgccaggacaacagtgacgaggccaactgtgacatccacgtctgcttgcccagtcagttcaaatgcaccaacaccaaccgctgtattcccggcatcttccgctgcaatgggcaggacaactgcggagatggggaggatgagagggactgccccgaggtgacctgcgcccccaaccagttccagtgctccattaccaaacggtgcatcccccgggtctgggtctgcgaccgggacaatgactgtgtggatggcagtgatgagcccgccaactgcacccagatgacctgtggtgtggacgagttccgctgcaaggattcgggccgctgcatcccagcgcgttggaagtgtgacggagaggatgactgtggggatggctcggatgagcccaaggaagagtgtgatgaacgcacctgtgagccataccagttccgctgcaagaacaaccgctgcgtgcccggccgctggcagtgcgactacgacaacgattgcggtgacaactccgatgaagagagctgcacccctcggccctgctccgagagtgagttctcctgtgccaacggccgctgcatcgcggggcgctggaaatgcgatggagaccacgactgcgcggacggctcggacgagaaagactgcaccccccgctgtgacatggaccagttccagtgcaagagcggccactgcatccccctgcgctggcgctgtgacgcagacgccgactgcatggacggcagcgacgaggaggcctgcggcactggcgtgcggacctgccccctggacgagttccagtgcaacaacaccttgtgcaagccgctggcctggaagtgcgatggcgaggatgactgtggggacaactcagatgagaaccccgaggagtgtgcccggttcgtgtgccctcccaaccggcccttccgttgcaagaatgaccgcgtctgtctgtggatcgggcgccaatgcgatggcacggacaactgtggggatgggactgatgaagaggactgtgagccccccacagcccacaccacccactgcaaagacaagaaggagtttctgtgccggaaccagcgctgcctctcctcctccctgcgctgcaacatgttcgatgactgcggggacggctctgacgaggaggactgcagcatcgaccccaagctgaccagctgcgccaccaatgccagcatctgtggggacgaggcacgctgcgtgcgcaccgagaaagcggcctactgtgcctgccgctcgggcttccacaccgtgcccggccagcccggatgccaagacatcaacgagtgcctgcgcttcggcacctgctcccagctctgcaacaacaccaagggcggccacctctgcagctgcgctcggaacttcatgaagacgcacaacacctgcaaggccgaaggctctgagtaccaggtcctgtacatcgctgatgacaatgagatccgcagcctgttccccggccacccccattcggcttacgagcaggcattccagggtgacgagagtgtccgcattgatgctatggatgtccatgtcaaggctggccgtgtctattggaccaactggcacacgggcaccatctcctaccgcagcctgccacctgctgcgcctcctaccacttccaaccgccaccggcgacagattgaccggggtgtcacccacctcaacatttcagggctgaagatgcccagaggcatcgccatcgactgggtggccggaaacgtgtactggaccgactcgggccgagatgtgattgaggtggcgcagatgaagggcgagaaccgcaagacgctcatctcgggcatgattgacgagccccacgccattgtggtggacccactgagggggaccatgtactggtcagactggggcaaccaccccaagattgagacggcagcgatggatgggacgcttcgggagacactggtgcaggacaacattcagtggcccacaggcctggccgtggattatcacaatgagcggctgtactgggcagacgccaagctttcagtcatcggcagcatccggctcaatggcacggaccccattgtggctgctgacagcaaacgaggcctaagtcaccccttcagcatcgacgtctttgaggattacatctatggtgtcacctacatcaataatcgtgtcttcaagatccataagtttggccacagccccttggtcaacctgacagggggcctgagccacgcctctgacgtggtcctttaccatcagcacaagcagcccgaagtgaccaacccatgtgaccgcaagaaatgcgagtggctctgcctgctgagccccagtgggcctgtctgcacctgtcccaatgggaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatagggccctgccccgtcggactgcccccagaaagcctcctgccccctgccagtgaagtccttcagtgagcccctccccagccagcccttccctggccccgccggatgtataaatgtaaaaatgaaggaattacattttatatgtgagcgagcaagccggcaagcgagcacagtattatttctccatcccctccctgcctgctccttggcacccccatgctgccttcagggagacaggcagggagggcttggggctgcacctcctaccctcccaccagaacgcaccccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtggctctgggtgaggtaggcgggaaaggatggagtgttttagttcttgggggaggccaccccaaaccccagccccaactccaggggcacctatgagatggccatgctcaacccccctcccagacaggccctccctgtctccagggcccccaccgaggttcccagggctggagacttcctctggtaaacattcctccagcctcccctcccctggggacgccaaggaggtgggccacacccaggaagggaaagcgggcagccccgttttggggacgtgaacgttttaataatttttgctgaattcctttacaactaaataacacagatattgttataaataaaattgtaaaaaaaaaaaaaaaaaaaaa

SEQ ID NO: 474 - RNA, 25 nt, synthetic construct
  source 1..25: mol_type = other RNA; note = LRP1 NAT siRNA (UPNA-222)
atggaagcatccaggatattctaaa

SEQ ID NO: 475 - RNA, 27 nt, synthetic construct
  source 1..27: mol_type = other RNA; note = UPNA-222 complementary strand 5 prime to 3 prime
tttagaatatcctggatgcttccattc

SEQ ID NO: 476 - RNA, 25 nt, synthetic construct
  source 1..25: mol_type = other RNA; note = LRP1 NAT siRNA (UPNA-223)
gcatccaggatattctaaaatctca

SEQ ID NO: 477 - RNA, 27 nt, synthetic construct
  source 1..27: mol_type = other RNA; note = UPNA-223 complementary strand 5 prime to 3 prime
tgagattttagaatatcctggatgctt

SEQ ID NO: 478 - RNA, 25 nt, synthetic construct
  source 1..25: mol_type = other RNA; note = LRP1 NAT siRNA (UPNA-224)
caacatgtccatccttacagctacc

SEQ ID NO: 479 - RNA, 27 nt, synthetic construct
  source 1..27: mol_type = other RNA; note = UPNA-224 complementary strand 5 prime to 3 prime
ggtagctgtaaggatggacatgttgat

SEQ ID NO: 480 - RNA, 25 nt, synthetic construct
  source 1..25: mol_type = other RNA; note = LRP1 NAT siRNA (UPNA-225)
aagccagtcagaaggtattaaaaaa

SEQ ID NO: 481 - RNA, 27 nt, synthetic construct
  source 1..27: mol_type = other RNA; note = UPNA-225 complementary strand 5 prime to 3 prime
ttttttaataccttctgactggcttgc

SEQ ID NO: 482 - RNA, 25 nt, synthetic construct
  source 1..25: mol_type = other RNA; note = LRP1 NAT siRNA (UPNA-226)
agccagtcagaaggtattaaaaaaa

SEQ ID NO: 483 - RNA, 27 nt, synthetic construct
  source 1..27: mol_type = other RNA; note = UPNA-226 complementary strand 5 prime to 3 prime
tttttttaataccttctgactggcttg

SEQ ID NO: 484 - RNA, 25 nt, synthetic construct
  source 1..25: mol_type = other RNA; note = LRP1 NAT siRNA (UPNA-227)
ggcaagccagtcagaaggtattaaa

SEQ ID NO: 485 - RNA, 27 nt, synthetic construct
  source 1..27: mol_type = other RNA; note = UPNA-227 complementary strand 5 prime to 3 prime
tttaataccttctgactggcttgccat

SEQ ID NO: 486 - RNA, 25 nt, synthetic construct
  source 1..25: mol_type = other RNA; note = LRP1 NAT siRNA (UPNA-228)
ccagtcagaaggtattaaaaaaaaa

SEQ ID NO: 487 - RNA, 27 nt, synthetic construct
  source 1..27: mol_type = other RNA; note = UPNA-228 complementary strand 5 prime to 3 prime
tttttttttaataccttctgactggct

SEQ ID NO: 488 - RNA, 25 nt, synthetic construct
  source 1..25: mol_type = other RNA; note = LRP1 NAT siRNA (UPNA-229)
gccagtcagaaggtattaaaaaaaa

SEQ ID NO: 489 - RNA, 27 nt, synthetic construct
  source 1..27: mol_type = other RNA; note = UPNA-229 complementary strand 5 prime to 3 prime
ttttttttaataccttctgactggctt

SEQ ID NO: 490 - RNA, 25 nt, synthetic construct
  source 1..25: mol_type = other RNA; note = LRP1 NAT siRNA (UPNA-230)
ctaacttgatggcaagccagtcaga

SEQ ID NO: 491 - RNA, 27 nt, synthetic construct
  source 1..27: mol_type = other RNA; note = UPNA-230 complementary strand 5 prime to 3 prime
tctgactggcttgccatcaagttaggg

SEQ ID NO: 492 - RNA, 25 nt, synthetic construct
  source 1..25: mol_type = other RNA; note = LRP1 NAT siRNA (UPNA-231)
taacttgatggcaagccagtcagaa

SEQ ID NO: 493 - RNA, 27 nt, synthetic construct
  source 1..27: mol_type = other RNA; note = UPNA-231 complementary strand 5 prime to 3 prime
ttctgactggcttgccatcaagttagg

SEQ ID NO: 494 - RNA, 25 nt, synthetic construct
  source 1..25: mol_type = other RNA; note = LRP1 NAT siRNA (UPNA-232)
caaatcaaagctcagcctttccctg

SEQ ID NO: 495 - RNA, 27 nt, synthetic construct
  source 1..27: mol_type = other RNA; note = UPNA-232 complementary strand 5 prime to 3 prime
cagggaaaggctgagctttgatttgga

SEQ ID NO: 496 - RNA, 25 nt, synthetic construct
  source 1..25: mol_type = other RNA; note = LRP1 NAT siRNA (UPNA-233)
cctaacttgatggcaagccagtcag

SEQ ID NO: 497 - RNA, 27 nt, synthetic construct
  source 1..27: mol_type = other RNA; note = UPNA-233 complementary strand 5 prime to 3 prime
ctgactggcttgccatcaagttagggt

SEQ ID NO: 498 - RNA, 25 nt, synthetic construct
  source 1..25: mol_type = other RNA; note = LRP1 NAT siRNA (UPNA-234)
acttgatggcaagccagtcagaagg

SEQ ID NO: 499 - RNA, 27 nt, synthetic construct
  source 1..27: mol_type = other RNA; note = UPNA-234 complementary strand 5 prime to 3 prime
ccttctgactggcttgccatcaagtta

SEQ ID NO: 500 - RNA, 25 nt, synthetic construct
  source 1..25: mol_type = other RNA; note = LRP1 NAT siRNA (UPNA-235)
agtccctcatcttattccaaatcct

SEQ ID NO: 501 - RNA, 27 nt, synthetic construct
  source 1..27: mol_type = other RNA; note = UPNA-235 complementary strand 5 prime to 3 prime
aggatttggaataagatgagggactct

SEQ ID NO: 502 - RNA, 25 nt, synthetic construct
  source 1..25: mol_type = other RNA; note = LRP1 NAT siRNA (UPNA-236)
cctgaaatcctagagtatgacactg

SEQ ID NO: 503 - RNA, 27 nt, synthetic construct
  source 1..27: mol_type = other RNA; note = UPNA-236 complementary strand 5 prime to 3 prime
cagtgtcatactctaggatttcaggct

SEQ ID NO: 504 - RNA, 25 nt, synthetic construct
  source 1..25: mol_type = other RNA; note = LRP1 NAT siRNA (UPNA-237)
aaaaccctaacttgatggcaagcca

SEQ ID NO: 505 - RNA, 27 nt, synthetic construct
  source 1..27: mol_type = other RNA; note = UPNA-237 complementary strand 5 prime to 3 prime
tggcttgccatcaagttagggttttag

SEQ ID NO: 506 - RNA, 25 nt, synthetic construct
  source 1..25: mol_type = other RNA; note = LRP1 NAT siRNA (UPNA-238)
ctgaaatcctagagtatgacactga

SEQ ID NO: 507 - RNA, 27 nt, synthetic construct
  source 1..27: mol_type = other RNA; note = UPNA-238 complementary strand 5 prime to 3 prime
tcagtgtcatactctaggatttcaggc

SEQ ID NO: 508 - RNA, 25 nt, synthetic construct
  source 1..25: mol_type = other RNA; note = LRP1 NAT siRNA (UPNA-239)
ctagagtatgacactgagtttcaaa

SEQ ID NO: 509 - RNA, 27 nt, synthetic construct
  source 1..27: mol_type = other RNA; note = UPNA-239 complementary strand 5 prime to 3 prime
tttgaaactcagtgtcatactctagga

SEQ ID NO: 510 - RNA, 25 nt, synthetic construct
  source 1..25: mol_type = other RNA; note = LRP1 NAT siRNA (UPNA-240)
gagtccctcatcttattccaaatcc

SEQ ID NO: 511 - RNA, 27 nt, synthetic construct
  source 1..27: mol_type = other RNA; note = UPNA-240 complementary strand 5 prime to 3 prime
ggatttggaataagatgagggactctg

SEQ ID NO: 512 - RNA, 25 nt, synthetic construct
  source 1..25: mol_type = other RNA; note = LRP1 NAT siRNA (UPNA-241)
ggtcttccaaagtctccaaatcaaa

SEQ ID NO: 513 - RNA, 27 nt, synthetic construct
  source 1..27: mol_type = other RNA; note = UPNA-241 complementary strand 5 prime to 3 prime
tttgatttggagactttggaagaccgg

SEQ ID NO: 514 - RNA, 25 nt, synthetic construct
  source 1..25: mol_type = other RNA; note = LRP1 NAT siRNA (UPNA-242)
ggaatctaaaaccctaacttgatgg

SEQ ID NO: 515 - RNA, 27 nt, synthetic construct
  source 1..27: mol_type = other RNA; note = UPNA-242 complementary strand 5 prime to 3 prime
ccatcaagttagggttttagattccga

SEQ ID NO: 516 - RNA, 25 nt, synthetic construct
  source 1..25: mol_type = other RNA; note = LRP1 NAT siRNA (UPNA-243)
tcagggttcccctagaaaatcgagc

SEQ ID NO: 517 - RNA, 27 nt, synthetic construct
  source 1..27: mol_type = other RNA; note = UPNA-243 complementary strand 5 prime to 3 prime
gctcgattttctaggggaaccctgaag

SEQ ID NO: 518 - DNA, 29321 bp, Homo sapiens
  source 1..29321: mol_type = other DNA; note = Human Gene C9orf72 (ENST00000619707.5) from GENCODE V41
ctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctat

SEQ ID NO: 519 - RNA, 1950 nt, Homo sapiens
  source 1..1950: mol_type = mRNA; note = Homo sapiens C9orf72-SMCR8 complex subunit (C9orf72), transcript variant 1, mRNA (NCBI Reference Sequence - NM_145005.7).
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttata

SEQ ID NO: 520 - RNA, 3231 nt, Homo sapiens
  source 1..3231: mol_type = mRNA; note = Homo sapiens C9orf72-SMCR8 complex subunit (C9orf72), transcript variant 3, mRNA (NCBI Reference Sequence - NM_001256054.3)
ggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 521 - RNA, 3338 nt, Homo sapiens
  source 1..3338: mol_type = mRNA; note = Homo sapiens C9orf72-SMCR8 complex subunit (C9orf72), transcript variant 3, mRNA (NCBI Reference -- NM_001256054.3)
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 522 - DNA, 1123 bp, Homo sapiens
  source 1..1123: mol_type = other DNA; note = C9ORF72 NAT (AS3)
acgtctgtctgttttctatgtgcgatgacgttttctcacgaggctagcgaaatggggcggggcaacttgtcctgttcttttatcttaagacccgctctggaggagcgttggcgcaatagcgtgtgcgaaccttaataggggaggctgctggatctggagaaagtgaagacgatttcgtggttttgaatggttttgtttgtgcttggtaggcagtgggcgctcaacacataattggtggatgaaattttgtttttaccgtaagacactgttaagtgcattcaaaactccactgcaaaccctggtaggggacagctccggcactgcgggcgggaatcccacggtcccctgcaaagtcatcgcaattttgcctttacatgtaagaattctctcaagcatgattttcacactggggaatgtcatttttgctagttgcaatatgtggatgagttgtttttttttaacttttgaaaaacgtaccattctgtttgatgtgtaaaaaacacaaagatttttgaaaccttgcgtcttttggtctgcaggtgtatagattccacttactacagatgagtagcatttacaccactcagatgtgtaaaaaaacaaaggttttttaaactgtgtgccttttgatctgcaagtgtgagatggcacttactacagtgagtagcatttaatctttttcatcactaaaaatcacacagaacgttttaatcattcaccgaggaagaaagggaggaataaatacacaaaatggctctcaacgtctacaccttctgcagaaacagacccttttcctactgttctatgctttgtgaaagttgatcatacaaattgggtcattctttttatacccaactaaaatagtgggggtagggggtagaaaagcacttaggacaaatgacactgctcccacagtgtaattctctccaagtccagctgctgcaactgcccgttgtgacctgagaccagttttatctaatagttgctaaaatgacctgctgcagctctaattttatctaccaccatcactcaccagttgaaactcaccagctcctcagatccttaatagtgccaatgaattttctcaaagagcactatgtaacatttctcttttttaacaaaa

SEQ ID NO: 523 - DNA, 707 bp, Homo sapiens
  source 1..707: mol_type = other DNA; note = C9ORF72 NAT (GenBank - CA439471.1 707)
ttttttttttttttttttgatcctttgtacaaaagtagaagtaatgccagacagattacgtacccttgttgtgaacaactggtgcatggcaactgtttgaatagaaatttaccaactgccacaaccaggcaactactctcccagagcctaacaatctcgattgcatctgaaagggccacccctcctgggaaagtgcaggacctccctcctgtttctgaatacaaagcctggtggtgttcaacgcggccagatagacccaatgagcacacggacatgtaatctgtgcacttctttagacaactgattaccatcagtcaagtgatgcccaagtcacaatagtcacttcctttaagcaagtctgtgtcatctcggagctgtgaagcaaccaggtcatgtcccacagaatggggagcacaccgacttgcattgctgccctcatatgcaagtcatcaccactctctagaagcttgggctgaaattgtgcaggcgtctccacacccccatctcatcccgcatgatctcctcgccggcagggaccgtctcgggttcctagcgaaccccgacttggtccgcagaagccgcgcgccgcccaccctccggncttcccccaggcgaggcctctcagtacccgaggctctcttttctcgagcccgcagcggcagcgctcccagcgggtccccgggaaggagacagctcgggtactgagg

SEQ ID NO: 524 - DNA, 562 bp, Homo sapiens
  source 1..562: mol_type = other DNA; note = C9ORF72 NATs (GenBank - BQ778249.1)
ttttttaaatttcaaatatattttattcaaaattctccatttaggagaaaagatatataacaatgtttacacatgctttaataacttatttcactgtacaacttacattctgtataacagtacaataaaccagccaaagaaaataaccagttagcacttaaataagaatctaccatgtaaaaaacacagtatgggacactacaaggtagtatttatatattttttaaatgactgagctacagtacaacagtcatctagttcagtggttgtctaaaacatcaagctgtccacatctttctgattcatgatgggaaagctattatgacctttcacattcgaacatgtcattttgttgtgtaaatttggtgggtggggggcagaagggctctattacctttatccctttcttataaatatattttcccttttatattacttccagaattttaaataaaatatttatttgtgtttgtgtaactacaatgtcaacattgtgttagaattatattaatcagttattttatagccagcaaaatggtccaaacgcattaagaaaacaaaa

SEQ ID NO: 525 - DNA, 496 bp, Homo sapiens
  source 1..496: mol_type = other DNA; note = C9ORF72 NAT (GenBank - BG190336.1 )
ccccttgggaggtgnatccacatgtgcaacaaancaaaagcctcatgcttctgcagttgacattaatgtactcacacttatgaaagtttgccaatccctgatctctgtttaatacattctcgcaaatctctataaaagtcagtagtttgttcttctagaagttagtgggtctacacatgaaattcgttgtaagaaatatcaaagaatgtccagatcctccaaaaagaagagaattaattattgaatttgatttaaaataagcaggtcattgggtgggatttagaaatctgattctaataatatttgacctacagctctattaggaaaaataaaaaggcatgtaactatcttgaaattcaaaccatatccacagttattatgttaaaggagtggttttcaacctagagtggtggcaacttctttctcaaattaaaacaagataaaaggtaagctattctccctcaagaggatggaggatgggaaaaatgttcaaaac

SEQ ID NO: 526 - DNA, 454 bp, Homo sapiens
  source 1..454: mol_type = other DNA; note = FXN NAT (GenBank - AI951739.1)
caactctgccggccgcgggacccgggtgagggtctgggcctgggctgggctgggtgacgccaggaggccggctactgcgcggcgcccgagagtccacatgctgctccgggtctgccgcccgctccgccctccagcgctgggtgctgcggcgacccctggtggccactggccgcaggcactcttctgtgggggagcagctagaggttagacctcaggaagaacttcccagcttagcactattcgtgcatttaacaaaaatggagagcctgctttgtgcaaagcacggagtgcaaccaggacccctgacccaagggagactgcagcctggtggcccgccgcttctaaaattctaaacaggaggaacttgggagctgctgtcttgctgggaagtgggtgcatgcacaaattgaggctgcttggccgccggtatgggtttacagcagttgggtatgtg

SEQ ID NO: 527 - DNA, 418 bp, Homo sapiens
  source 1..418: mol_type = other DNA; note = FXN NAT (GenBank - W90730.1)
aactagaaaatgtagaattatgtgtgtggctcccattctgtttctgttgaacagctgctttagaagtagatgcaaggngtggagaaaagggtggggaagaggccttcaagtgaactcagtttctgatgaattttggagaccagggggagcttagggtcaatccaggacagtcagggctttaaaataattaaacaggatggtggccccagttgccactgccacccacccaactcctcaaatacccaagatgtgcaagggaactatggaacagacaggttaaacaaatgtgattaattcagtatagaaaaaaagaggcccagctggtgacagaggttgctaaccatctatccagaaacccagtgtccacctcctgggcggccgccaagtttgggtgtgtttgtcagtcaatagcccttaa

SEQ ID NO: 528 - DNA, 709 bp, Homo sapiens
  source 1..709: mol_type = other DNA; note = FXN NAT (GenBank - BM977900.1)
ttttttttttttttttttaaatttaaccgagtttaattgaacagtttgagaattgggcagcctcctcagccagagtaggttcagagactccagcacagccacgtggtggaagaacatttatggacagagacagaaaagtggcgtacagaaaacagaagtgaggtatagaaacagccagattgtttacagctcaatatttgccttatttgaaagcagtgtcaagagttgacctcctttgattggccaaaatccaatgtttgtcacaagagtagttcacagtcttgtttacacatctgggtaggctacaattcactaagtacagagaaacctttaggtcaagcttaaactatataaggaggcagctttaggctaaatttgatttaacaatattaatgacatatttatgataaataactatacttttgaaacaaaacatatttggtgtgaagaatgggatcattgcaggacttttccttagttcagctaaagagaaggttcttgtctatcccacagccacaaaaatttaggcttgcagatggtttaaagggtgagtaaagcagggttttattaagtgaaaaggggaaataaagggatgaggggaacagggatcctctgcaaggccagagtctcctgctacagtgcttcccaccaggctgctagatcccaggttcacataggaagaggagggccaggtcctccctgctgcaac

SEQ ID NO: 529 - DNA, 467 bp, Homo sapiens
  source 1..467: mol_type = other DNA; note = FXN NAT (GenBank - BX111535.1)
ttttttttttttttcaatcagcaaatatttattgagccagctttactataccatgctaagtaaaatggtgaaaaaggtggagaagatttggtcctcatggaacttgtagataacacttatgatataatattcagttgaaattacagaatacaaaattcacccagtatgattaccacgatgcaaaaatgaaatgcacaggcaaaggacaggagcaaaataagccctagtgcaaactgtatttgtattcattcagggtagtgacagtactaatgactattcttccttttaccattttagaacttatggtagatgtttttattaaatattaaagtgatataaaatgtgtagagacatcaagaaataatactgttacattaaaaaacagaatataagtagaatagctgcatagtatacctgcaactctccattcagatgaacagggattgaaatcaatcgtgcaagtatta

SEQ ID NO: 530 - DNA, 515 bp, Homo sapiens
  source 1..515: mol_type = other DNA; note = FXN NAT (GenBank - AL713361.1)
ttcttatgaaacaaaattaaatttaaaaaaagcaaaatgcaacacatgaatgctggtatcaagttggtagcttaacagtgtgaagagaaattatataaagtgattttaaaacacaattattgccaggagcaatggctcaccctgtaatcccagcactttgggaggccgagctggccggatcacctgacatcaggagttggagaccggcctggtcaacatggagaagccccatctctactaaaaaaaatataaaaattagccaagtgtggtggcacatgcctgtaatcccagctactcaggaggctgaggcaggacaatcacttgaaccagggaggcagaggttgcagtgagcccagatcaggccgctgcactccagcctgtgcaatagaggcagactctgtctcaaaagaaaacaaaaaaccacacacagacgcaattaattatttgggctggacaaagggctcatgctcttgatcccagcacttcgggaggctgagttgggcaggtcacttgac

SEQ ID NO: 531 - DNA, 376 bp, Homo sapiens
  source 1..376: mol_type = other DNA; note = FXN NAT (GenBank - AA233957.1)
ttgttggggttatttagagtttatttgaacaactatcttaaacatataattataacacatttatttgacatttatagagctctaattgtgtgcaaggaactgtgcaagatgctagggcaacagcagtcaacaaaggagacacatagtcctagcctcatggaaggtacagccaagcaggggtgacacatggtaggtcagtgctgggaaagacaaacatgatatgctgaggacatggaacaatcaagggcatccaggcagaggctgagtggaggaggagtttgcaaggctgtgcatagaacatttttcagggtagaatctaccccccaggcagagatgtggggaataaagaccaaaatggaaacagcactggccaagt

SEQ ID NO: 532 - DNA, 29321 bp, synthetic construct; identical to SEQ ID NO: 518
  source 1..29321: mol_type = other DNA

SEQ ID NO: 533 - DNA, 1950 bp, synthetic construct
  source 1..1950: mol_type = other DNA
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttata

SEQ ID NO: 534 - DNA, 3231 bp, synthetic construct
  source 1..3231: mol_type = other DNA
ggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 535 - DNA, 3338 bp, synthetic construct
  source 1..3338: mol_type = other DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 536 - DNA, 1123 bp, synthetic construct; identical to SEQ ID NO: 522
  source 1..1123: mol_type = other DNA

SEQ ID NO: 537 - DNA, 707 bp, synthetic construct; identical to SEQ ID NO: 523
  misc_feature 601: note = n is a, c, g, or t
  source 1..707: mol_type = other DNA

SEQ ID NO: 538 - DNA, 562 bp, synthetic construct; identical to SEQ ID NO: 524
  source 1..562: mol_type = other DNA

SEQ ID NO: 539 - DNA, 496 bp, synthetic construct; identical to SEQ ID NO: 525
  misc_feature 15: note = n is a, c, g, or t
  misc_feature 34: note = n is a, c, g, or t
  source 1..496: mol_type = other DNA

SEQ ID NO: 540 - DNA, 20 bp, synthetic construct
  source 1..20: mol_type = other DNA
aggtgtagacgttgagagcc

SEQ ID NO: 541 - DNA, 20 bp, synthetic construct
  source 1..20: mol_type = other DNA
tgcagaccaaaagacgcaag

SEQ ID NO: 542 - DNA, 20 bp, synthetic construct
  source 1..20: mol_type = other DNA
ccctaccagggtttgcagtg

SEQ ID NO: 543 - DNA, 20 bp, synthetic construct
  source 1..20: mol_type = other DNA
tccagcagcctcccctatta

SEQ ID NO: 544 - DNA, 20 bp, synthetic construct
  source 1..20: mol_type = other DNA
ggctctgggagagtagttgc

SEQ ID NO: 545 - DNA, 20 bp, synthetic construct
  source 1..20: mol_type = other DNA
gttcgctaggaacccgagac

SEQ ID NO: 546 - DNA, 20 bp, synthetic construct
  source 1..20: mol_type = other DNA
gggacatgacctggttgctt

SEQ ID NO: 547 - DNA, 20 bp, synthetic construct
  source 1..20: mol_type = other DNA
cagtacccgagctgtctcct

SEQ ID NO: 548 - DNA, 21 bp, synthetic construct
  source 1..21: mol_type = other DNA
atgcgtttggaccattttgct

SEQ ID NO: 549 - DNA, 21 bp, synthetic construct
  source 1..21: mol_type = other DNA
gtaatagagcccttctgcccc

SEQ ID NO: 550 - DNA, 21 bp, synthetic construct
  source 1..21: mol_type = other DNA
tcccatcctccatcctcttga

SEQ ID NO: 551 - DNA, 21 bp, synthetic construct
  source 1..21: mol_type = other DNA
tcccacccaatgacctgctta

SEQ ID NO: 552 - DNA, 7505 bp, synthetic construct
  source 1..7505: mol_type = other DNA
gttgtgactgaaacccgtcaatatggcggcgatcggccgcggccgctctctgaagaacctccgagtacgagggcggaatgacagcggcgaggagaacgtcccgctggatctgacccgagaaccttctgataacttaagagagattctccaaaatgtggccagattgcagggagtatcaaatatgagaaagctaggccatctgaataactttactaagcttctttgtgatattggccacagtgaagaaaaactgggctttcactatgaggatatcataatttgtttgcggttagctttattaaatgaagcaaaagaagtgcgagcagcagggctacgagcgcttcgatatctcatccaagactccagtattctccagaaggtgctaaaattgaaagtggactatttaatagctaggtgcattgacatacaacagagcaacgaggtagagaggacacaagcacttcgattagtcagaaagatgattactgtgaatgcttccttgtttcctagttctgtgaccaactcattaattgcagttggaaatgatggacttcaagaaagagacagaatggtccgagcatgcattgccattatctgtgaactagcacttcagaatccagaggtggtggcccttcgaggtggactaaacaccatcttgaaaaatgtgatcgattgccaattaagtcgaataaatgaggccctaattactacaattttgcaccttcttaatcatccaaagactcgacagtatgtgcgagctgatgtagaattagagagaattttagcaccctatactgattttcactacagacatagtccagatacagctgaaggacagctcaaagaagacagagaagcacgatttctagccagtaaaatgggaatcatagcaacattccgatcatgggcaggtattattaatttatgtaaacctggaaattctgggatccagtctctaataggagtactttgcataccaaatatggaaataaggcgaggtctacttgaagtgctttatgatatatttcgtcttcctctacctgttgtgactgaggagttcatagaagcactactcagtgtagatccagggaggttccaagacagttggaggctttcagatggctttgtggcagctgaggcaaaaactattcttcctcatcgtgccagatccaggccagacctcatggataattatttggcactgatactctctgcatttattcgtaatggacttttagagggtctagttgaagtgataacaaacagtgatgatcatatctcagttagagctaccatccttttaggagagcttttacatatggcaaacacaattcttcctcattcacatagccatcatttacactgcttgccaaccctaatgaatatggctgcatcctttgatatccccaaggaaaagagactgcgagccagtgcagccttgaactgtttaaaacgcttccatgaaatgaagaaacgaggacctaagccttatagtcttcatttagaccacattattcagaaagcaattgcaacacaccagaaacgggatcagtatctccgagttcagaaagatatatttatccttaaggatacagaggaagctcttttaattaaccttagagatagccaagtccttcaacataaagagaatcttgaatggaattggaatcttatagggaccattcttaagtggccaaatgtaaatctaagaaactataaagatgaacagttacacaggtttgtacgaagactactttatttttacaagcccagcagtaaattatatgccaacctggatctggattttgccaaggccaaacagctcacggttgtaggttgccagtttacagaatttcttcttgaatctgaagaggatgggcaaggctacttagaagatctagtaaaggatattgttcagtggctcaatgcttcatctggaatgaaacccgaaagaagtcttcaaaataatggtttattgaccacccttagtcaacactactttttatttattggaacactttcttgccaccctcatggagttaaaatgctggaaaaatgcagtgtatttcagtgtctccttaatctttgctccttgaaaaaccaagatcacttgctaaaacttactgtttctagcttggactatagcagagatggattggctagagtcatcctttccaaaattttaactgcagctactgatgcctgcagactctatgcaacaaaacatttaagggtattattgagagctaatgttgaattctttaataattggggaattgagttgttagtgacccagctacatgataaaaacaaaacgatttcctctgaagctcttgatatcctcgatgaagcatgtgaagacaaggccaatcttcatgctctcattcagatgaaaccagcgttatcccaccttggagacaagggtttgcttctcctgctgagatttctctccattccaaaaggattttcctatctgaatgaaagaggttatgtagcaaaacaattggaaaagtggcacagggaatacaactccaaatatgttgacttgattgaggaacaactcaatgaagcacttactacttaccggaagcctgttgatggtgataactatgttcgtcggagtaaccaaagattacagcgtcctcacgtctacctgcctatacacctttatggacaactagtacaccataaaacaggctgccatttgttggaagtacagaatattattacagaactctgtcgtaatgttcgtacaccagatttggataagtgggaagaaattaaaaaactgaaagcatctctttgggccttgggaaatatcggctcatcaaattggggtctcaatttgctacaggaagaaaacgtgattccagatatactaaaacttgcaaaacagtgtgaagttctttccatcagagggacctgtgtatatgtacttgggctcatagctaaaaccaaacaaggctgtgatattctaaaatgtcacaactgggatgctgtgaggcatagtcgcaaacatctgtggccagtggttccagatgatgtggaacaactctgtaatgaactttcatctatcccaagcactctaagtttgaactcggagtcaaccagctctagacataatagtgaaagtgaatctgtgccatcgagtatgttcatattggaggatgaccggtttggcagcagctctactagcacatttttccttgatatcaatgaagatacagagccaacattttatgaccgatctggacccataaaggataaaaattcattccctttctttgcttctagtaaacttgtgaagaatcgtatcttaaattcgcttactttgcctaacaaaaaacatcgtagtagcagtgatccaaaaggagggaaattatcatctgaaagtaagacaagcaacaggcgaatcagaacacttacggagcccagtgttgattttaatcatagtgatgattttacacccatatccactgtacagaaaacattacaattagagacttcatttatggggaataagcacattgaagacactggtagtacaccaagcattggagaaaatgacttaaaattcaccaagaattttggtacagagaatcacagagaaaatacaagccgagagaggttagtagtagaaagttcaacgagctcacatatgaagatacgtagccaaagtttcaatacagacactacaacaagtggcataagttcaatgagctcaagtccttcacgagagacagtaggtgtagatgctacaactatggacacagactgtggaagcatgagtactgtggtaagtactaaaactattaagacaagccactatttgacgccacagtctaaccatctgtctctctccaaatcaaattcggtgtccctggtgcctccaggttcttctcatacgcttcctagaagagcacagtcccttaaagcaccctctattgctacaattaaaagtctagcagattgtaactttagttacacaagttctagagatgcttttggctatgctacactgaaaagactacagcaacaaagaatgcatccatccttatctcactctgaagctttggcatctccagcaaaagatgtgctatttactgataccatcaccatgaaggccaacagttttgagtccagattaacaccaagcaggatcgattttaaaaagaagcatgtcgggggaatcaggagcttaagacctacaataacaaacaaccttttcaggttcatgaaagccttaagttatgcatcattagataaagaagatttattgagtcctattaatcaaaataccctgcaacgatcttcctcagtgcggtccatggtgtccagtgccacatatgggggttcagatgattacattggtcttgctctcccggtggatataaatgatatattccaggtaaaggatattccctattttcagacaaaaaacataccaccacatgatgatcgaggtgcaagagcatttgcccatgatgcaggaggtcttccatctggaactggaggtcttgtaaaaaattcttttcacttgctacgacagcagatgagtcttacggaaataatgaattcaatccattcagatgcctctctgtttttagaaagtacagaagacactggactacaggaacatacagatgataactgcctttattgtgtctgtattgaaattctgggtttccagcccagcaaccaactgagtgcaatatgtagtcattcagactttcaagatattccatattctgattggtgtgagcagactatccataatcctttagaagtggttccctctaagttttcggggatttctggatgcagtgatggggtgtctcaagaaggctcagctagcagcaccaaaagcacagaattgttactaggtgttaaaacaattccagatgatacaccaatgtgccgtatactccttcgcaaagaagttctaagattagtcattaatttgagtagttcagtttcaactaaatgtcatgagactgggcttttaacaattaaggagaagtatcctcaaacatttgatgacatatgcctttactctgaggtttcccatttgctgtcacactgcacattcagacttccgtgtcggaggttcatacaagaattatttcaagatgtacagtttctacaaatgcatgaagaagcagaggctgtgttggcaacaccaccaaagcaacctatagttgatacatctgctgaatcctgacctcatatttatgatggatatagatacatactatatatattcatatttgtggatttcctaaaagcctcagaaaatacgactgactaggcagcaaagacaggagtatcttctgtacactgttccgcagttactggtacatgaacagttggaactgctgactttcctaaccaaaacaacttccttctctcctttgttgagccttttgaggggttcatgattcattaccacagttttaagagtttcagttaccattgtatgcaagagccaagcactgaatacctacataggttttctattttctttcattttaaaagcataatgacagtggaacaataatgggatatgcagaagcacccttcacaagttatttctgaatgatttttagggtaaataatacagatgccttgtttgttaactaacttgtggaaagcaggaatcagtgtctctaaggctgcatcctattaccacaatggggtgtgctataactgctggtattagagagggaactttggccctttcacgtttttcttaatgtttgtaacactacttcagaggtttataacctcaaagcagaagaagagcctcaacaacccgggacttataagttatttttatgttactagacttgcataaagattcttgttttccaactcttcattttgttgcaatgtgttattacaggatatatgaaccaattaaggtttttcactacagttcttgaataaaatttaaaaatcattttttattttaattaaaaatatttcccatttatagaatgcatatatttgcaatggacttccactttcatcaactttccatctcatcgctttaaacaggaacttgaacaagcactgttagtttagacctaaaggataggaaagcattaaataatactttggatctcctgaggaaaagataagtttgcttgcaatttacacattccatggggaaagaagagccatatttccttaaaaaaaacattaataaagcttgttattgagaaaaattgtagtgaaaagccttaagtaccaaattttaaagcagcagtaacttaatttttatatcagtgtttttgttttgcacaaactaaatgcagtggtaggtgggtttatgagtatattaattgcctttatccatttgtgaagttaagttgatgagggcaaggtttttgtttgtttaatttgtatatgtctaaaggtatttggaactttttacaggaattaaacatatatgcaaatttgtatataaaaatagcatggccatcatttgaatgcttgtaaatgaaaggattatcttttttgagatctatatataaatagaaatagaaaatccagctggactgattaggattcttttttaattcatttgtgtataacatttttattacaattacacatcagttttgacacagtcatagcaacattaatattttcccatgatgcagatcctttttgtaatgggcttgttctttgagatctctgtaaagaaccctgtgaactagaaaacataactcacagagatacttttttaaaaaatttatttactggaactgaaagttccagttgggatgaagcatttcatctcacttcataacacctctttgactgcacttcagtgaattgttcttatgtgcactgtgtagcaacttacattataacaaagcagataagggctgtaagctgctgcttatgttgaaaagtggttcttcagattttctctcataaaatccagttgaagataaataatttttttatactttatcactgaacccaagtgtttatttaaatgtcaacagtacttctaagaacgttgcctgtcatcgtggtctttggtcttggataactaaactgcctttccagagaaccaaatgtcagagttactagaccaaatagtggttaaaacctccaaaggaagtaatgtaatcttattcataatgggattaacatattttagacattcattttaaacactacctcagttaatatagagtataaaaatctgtggtttaatccctcaaaagttaacagtaattttttttttgtcttacacacacacacaccccctcccccaccatcactatccctgtaccctcaccttggtcatctatcctgaaataaggcttagttagtattggcctgaatgttttgtgtttttttttttgttttttttttttactgttactttgaaaaatatgtatgtataccttatcatatctgcctatatcacttactttggggagatactcagagctttgtggttatcagtatactaaaaaaaaaaaaaa

SEQ ID NO: 553 - DNA, 394 bp, synthetic construct; identical to SEQ ID NO: 49
  source 1..394: mol_type = other DNA

SEQ ID NO: 554 - DNA, 474 bp, synthetic construct; identical to SEQ ID NO: 47
  source 1..474: mol_type = other DNA

SEQ ID NO: 555 - DNA, 454 bp, synthetic construct; identical to SEQ ID NO: 41
  source 1..454: mol_type = other DNA

SEQ ID NO: 556 - DNA, 694 bp, synthetic construct
  variation 612: note = n is a, c, g, or t
  variation 652: note = n is a, c, g, or t
  variation 655: note = n is a, c, g, or t
  variation 672: note = n is a, c, g, or t
  source 1..694: mol_type = other DNA
gagaaaagtatgtgtatctactgagtcactgggtcctctgtcatgcacataataaaccactagacataaatagtctaaatactccaataagaagacaaagttggtcaaaccagatataaaaaaagaaaaaaactatatgatatttacaggggagacacttaaatgtaaagacaaaaaaagaaagtaaaaggatggtaaaaggtataccatgcaaccactaggcaaaagaaaggtggtaatgtattattagatgacatgaacactcataaaagaagtattactaggaataaagagggttattttataattataaaaggattaagcccaataagaagatttagcagttctaaacatgaagtaacaacaaagcttcaaaatctgaaacaagaaaaagaaaagtcaggaagcattatatgggtgaaaaataattgagtctataattttaaaacaggctgaaaaagaaaacccagccagagagatccattaatgaattataatcatgtaggacaaaaaaaaatacatatcttacatatattttccaggtaatatgaaaacaaggccatatttccctagttgttttataaaacaaacatacataccccccatactaanacccaccaagactttagattaatgggaaaaataatggtcnacnactcctcatgaacatangtgccaaaaaaaaaaaaaaaaa

SEQ ID NO: 557 - DNA, 568 bp, synthetic construct; identical to SEQ ID NO: 46
  source 1..568: mol_type = other DNA

SEQ ID NO: 558 - DNA, 540 bp, synthetic construct
  variation 524: note = n is a, c, g, or t
  source 1..540: mol_type = other DNA
ggtatcaggatggtactggcctcaaaaaatgattaggaaggagttccactttttctattgtttggaacggtttcagaaggtgtggtaccagctcctctttgtacctctggtagaatttggctgtgaatccatctggtcctgggctttttttggttggtagactattaattactgcctcaatttcagaacttgttattttctattcttcttcttggtttagtcttgggagggtgtatgtgtccaggaatttatccatttattcgagattttctagtttatttgcgtagaggtgtttatagtattctcttataacagtctgtatttctgtgggatcagtggtgatatcccctttatcattttttattgcacctatttgattcttctctcttttcttctttattaagtctggctagcggtctattttgttaatcttttcaaaaaaccagctcctggattcattcgttttttttgaagggtttttcgtgtatctccttcagttctcgtttgatctcagtatttcttgncttctgctagcttttg

SEQ ID NO: 559 - DNA, 700 bp, synthetic construct; identical to SEQ ID NO: 51
  variation 651: note = n is a, c, g, or t
  source 1..700: mol_type = other DNA

SEQ ID NO: 560 - DNA, 551 bp, synthetic construct
  source 1..551: mol_type = other DNA
aatatgttgtagtgaacacagtataggtgctataattctctctaattctacatcagctcgacacatactgtcgagtctttggctgattaagaaggtgcaaaattgtcgtacttagggcctcattgtattcgacttactcggcaatcgatcacatttttcaagatggtgtttagtccacctcgtaagggccaccacctctggatactgaagtgctagttcacagataatggcaatgcatgctcggaccattctgtctctttcttgaagtccatcatttccaactgcaattcaatgagttggtcacagacactaggcaacacaaggaagcattcacagtaatcatctttctgactaatcgaagtgcttgtgtcctctctacctcgttgccctgttgtatgtcaatgcacctcagctcattaaatagtccactttcaattttagcaccttctggagcatactggagtcttggatgagatatcgaagcgctcgtagccctgctgctcgcacttcttttgcttcatttaatcaaagtacacgcaaacaaattatga

SEQ ID NO: 561 - DNA, 355 bp, synthetic construct; identical to SEQ ID NO: 42
  source 1..355: mol_type = other DNA

SEQ ID NO: 562 - DNA, 515 bp, synthetic construct; identical to SEQ ID NO: 44
  source 1..515: mol_type = other DNA

SEQ ID NO: 563 - DNA, 611 bp, synthetic construct; identical to SEQ ID NO: 55
  source 1..611: mol_type = other DNA

SEQ ID NO: 564 - DNA, 472 bp, synthetic construct
  variation 22: note = n is a, c, g, or t
  variation 24..25: note = n is a, c, g, or t
  variation 150: note = n is a, c, g, or t
  variation 155: note = n is a, c, g, or t
  variation 175: note = n is a, c, g, or t
  source 1..472: mol_type = other DNA
atattaacataaaaaccataantnncgccttttactctcacacatagagcatacctttctgactaatcggagtgcttgtgtcctctctacctcgttgctctgctgtatgtcaatgcacctaaacaatacacaaaatattaatcattgatnaaatnctattaaacaactttttgtntcacaatactgatagatgaaacccattttcacaatgtcaatatggtatattattgttttggtatatgatttctcagtaccagcatatatttatttaattaacccatacatctcgagggataaatatctgtcatttaaaagaccaattttttcttttaaaacgttaggaaagaacagtatagtaatttacaagactacatctattcataaagaaagaataataaacataaagtgtaaaaagtctactaaaaaaattcacttatctctgataaaattcagaccattttataaaaattgt

SEQ ID NO: 565 - DNA, 915 bp, synthetic construct; identical to SEQ ID NO: 45
  variation 844: note = n is a, c, g, or t
  variation 880: note = n is a, c, g, or t
  variation 899: note = n is a, c, g, or t
  source 1..915: mol_type = other DNA

SEQ ID NO: 566 - DNA, 452 bp, synthetic construct; identical to SEQ ID NO: 52
  source 1..452: mol_type = other DNA

SEQ ID NO: 567 - DNA, 1052 bp, synthetic construct
  source 1..1052: mol_type = other DNA
gtacaagaaagttgggtataataatattttaaaactagaaaagaatattaaagttcctacctctaattctacatcagctcgcacatactgtcgagtctttggatgattaagaaggtgcaaaattgtagtaattagggcctcatttattcgacttaattggcaatcgatcacatttttcaagatggtgtttagtccacctcgaagggccaccacctctggattctgaagtgctagttcacagataatggcaatgcatgctcggaccattctgtctctttcttgaagtccatcatttccaactgcaattaatgagttggtcacagaactaggaaacaaggaagcattcacagtaatcatctttctgactaatcgaagtgcttgtgtcctctctacctcgttgctctgttgtatgtcaatgcacctagctattaaatagtccactttcaattttagcaccttctggagaatactggagtcttggatgagatatcgaagcgctcgtagccctgctgctcgcacttcttttgcttcatttaataaagctaaccgcaaacaaattatgatatcctcatagtgaaagcccagtttttcttcactgtggccaatatcacaaagaagcttagtaaagttattcagatggcctagctttctcatatttgatactccctgcaatctggccacattttggagaatctctcttaagttatcagaaggttctcgggtcagatccagcgggacgttctcctcgccgctgtcattccgccctcgtactcggaggtcttcagagagcgggcgcggccgatcgccgccatgccaacttttttgtacaaagttggcattataaaaaaagcatgctcatcaatttgttgcaccgaacggtccctatcagtcaaaataaaatcattaatttgggcccaagctaagactgggcgtcgttttacaacgtcgtgactgggaaaaacatcccatgctagcgttaacgccaaaaataagggaactgccaggccttcaaaaaaaaaccaaaaggcttcattccgaaaaactggggcctt

SEQ ID NO: 568 - DNA, 565 bp, synthetic construct; identical to SEQ ID NO: 38
  variation 184: note = n is a, c, g, or t
  source 1..565: mol_type = other DNA

SEQ ID NO: 569 - DNA, 192 bp, synthetic construct; identical to SEQ ID NO: 40
  source 1..192: mol_type = other DNA

SEQ ID NO: 570 - DNA, 489 bp, synthetic construct; identical to SEQ ID NO: 48
  source 1..489: mol_type = other DNA

SEQ ID NO: 571 - DNA, 266 bp, synthetic construct; identical to SEQ ID NO: 54
  variation 157: note = n is a, c, g, or t
  variation 198: note = n is a, c, g, or t
  variation 212: note = n is a, c, g, or t
  variation 242: note = n is a, c, g, or t
  variation 254: note = n is a, c, g, or t
  source 1..266: mol_type = other DNA

SEQ ID NO: 572 - DNA, 426 bp, synthetic construct; identical to SEQ ID NO: 53
  variation 223: note = n is a, c, g, or t
  source 1..426: mol_type = other DNA

SEQ ID NO: 573 - DNA, 473 bp, synthetic construct; identical to SEQ ID NO: 50
  variation 440: note = n is a, c, g, or t
  variation 451: note = n is a, c, g, or t
  variation 457: note = n is a, c, g, or t
  source 1..473: mol_type = other DNA

SEQ ID NO: 574 - DNA, 6838 bp, synthetic construct
  source 1..6838: mol_type = other DNA
gtcctggcaatatggcgtcctccttgatgggctgatgagatgagtttcactgtagctccaaaccagagggcaaagctcccatgacccaataagcccacattgtccctttcctccgtggttccgtgtcgcccgtttctcaggactcgttctcaggcaggagagagcctcggggctgaaggccaggaccagccaggccgcgcggacctgaggttgaggaaccgggtgcaggcgagcacgatgggccggtcgtggctctggttgcagcagctcagacgagtgcgggacccgcagggctgagagtggctggaggagacccagggccctttgaacccgatcccttggccggagacctcagcccagtcggcccagtgggcgaaccggcaccaagagcggcctgcctgtcttcggaactgctgaggcggtggaggccgagagcagggtcatcgtgaggcctgaagtctcttacgcttttggcagctcccctcgcagcccctctggaaacgtacagcctcaggagcagccagtggcttgggacctggggtggtgtgtgtctgcggagcttcttgggctgccccatttcctagcggcccccacctccccacttcccgctcagagttagagataaggatctcagacttttgcctgagtaagggtctccgcactctttatccatttggttttcgatttcccgtttttgtttcttatttcaccaattctggtacacgctagtttttaaggctggaggttctcgagcgcttgctgccaaggactcccccaccccctcccccactgatggagtccgaaatgctgcaatcgcctcttctgggcctgggggaggaagatgaggctgatcttacagactggaacctacctttggcttttatgaaaaagaggcactgtgagaaaattgaaggctccaaatccttagctcagagctggaggatgaaggatcggatgaagacagtcagtgttgccttagttttgtgcctgaatgttggtgtggaccctcccgatgtggtgaagaccacgccctgtgcacgcttggaatgctggatcgatcctctgtcgatgggtcctcagaaagctctggaaaccatcggtgcaaatttacagaagcagtacgagaactggcagccaagggcccggtacaagcagagccttgacccaactgtggatgaagtcaagaagctctgcacgtccttacgtcgcaacgccaaggaggagcgagtcctctttcactacaatggccacggggtgccccggcccacagtcaacggggaggtctgggtcttcaacaagaactacacgcagtacatccctctgtccatatatgacctgcagacgtggatgggcagcccgtcgatcttcgtctacgactgctccaatgctggcttgatcgtcaagtccttcaagcagttcgcactacagcgggagcaggagctggaggtagctgcaatcaacccaaatcaccctcttgctcagatgcctttgcctccgtcgatgaaaaactgcatccagctggcagcctgcgaggccaccgagctgctgcccatgatccccgacctcccggctgacctattcacctcctgcctcaccacccccatcaagatcgccctgcgctggttttgcatgcagaaatgtgtcagtctggtgcctggcgtcacactggatttgatagaaaagatccctggccgcctgaacgacaggaggacgcccctgggtgaactgaactggatcttcacagccatcacagacaccatcgcgtggaacgtgctcccccgggatctcttccaaaagctcttcagacaggacttgctggtggctagtctgtttcgaaattttttattggcggaaaggattatgaggtcgtataactgcactcccgtcagcagcccgcgtctgccgcccacgtacatgcacgccatgtggcaagcctgggacctggctgttgacatctgtctgtctcagctgccgacgatcatcgaggaaggcactgcgtttcggcacagcccgttcttcgccgagcagctgaccgcattccaggtgtggctcaccatgggcgtggagaaccgaaacccacccgaacagctgcccatcgtcctgcaggtgctgttaagccaagtgcaccggctgagagcattggacttgcttggaagatttttggacctgggtccctgggcagtgagcctggccttgtctgtcggcatcttcccctacgtgctgaagctgctccagagctcggcccgagagctgcggccacttctcgttttcatctgggccaagatcctcgcagtggacagctcgtgccaagcggacctcgtgaaggacaacggccacaagtacttcctgtcggtcctggcggacccctacatgccagctgaacaccggaccatgacggctttcattctcgccgtgatcgtcaacagctatcacacggggcaggaagcctgccttcagggaaacctcattgccatctgcctggagcagctcaacgacccgcaccccttgctgcgccagtgggtggccatctgcctcggcaggatctggcagaacttcgactcggcgaggtggtgcggcgtgagggacagcgctcatgagaagctctacagcctcctctccgaccccattcccgaggtccgctgcgcagcggtcttcgcccttggcacgttcgtgggcaactctgcagagaggacggaccactccaccaccatcgaccacaacgtggccatgatgctggcccagctggtcagcgacgggagccccatggtccggaaggagctggtggtggctctgagtcatcttgtggttcagtatgaaagcaatttctgcaccgtggccctgcagttcatagaagaggaaaagaactacgccttgccttctccagcaaccacagagggagggagtttgaccccagtgcgagacagcccgtgcacccccagacttcgttctgtgagctcctatggaaacatccgtgctgtcgccacagccaggagcctcaacaaatctttgcagaacctgagtttgacagaggaatctggtggcgcggtggcgttctcccccggaaacctcagcaccagcagcagcgccagcagcaccctgggcagccccgagaatgaggagcatatcctgtccttcgagaccatcgacaagatgcgccgcgccagctcctactcctccctcaactccctcatcggagtttcctttaacagtgtttacactcagatttggagagtcctgctgcacctggctgctgacccctatccagaggtctcggacgtggccatgaaagtactcaacagcatcgcctacaaggccaccgtgaacgcccggccgcagcgcgtcctggacacctcctccctcacgcagtcggcccccgccagccccaccaacaagggcgtgcacatccaccaggcggggggctcccctccggcgtccagcaccagcagctccagcctgaccaacgatgtggccaagcagccggtcagccgagacttgccttctggccggccgggcaccacaggccccgctggggcgcagtacacccctcactcccaccagttcccccggacacggaagatgttcgacaagggcccagagcagactgcggacgacgcggacgatgctgctggacacaaaagtttcatctccgccacggtgcagacggggttctgcgactggagcgcccgctattttgcccagcccgtcatgaagatcccagaagagcacgacctggagagtcagatccgcaaggagcgggagtggcggttcctgcgaaacagccgtgtcaggaggcaggcccagcaagtcattcagaagggcattacgagattggacgaccaaatatttctgaacaggaaccccggcgtcccctctgtggtgaaattccaccccttcacgccgtgcatcgccgtagccgacaaggacagcatctgcttttgggactgggagaaaggggagaagctggattatttccacaatgggaaccctcggtacacgagggtcactgccatggagtatctgaacggccaggactgctcgcttctgctgacggccacagacgatggtgccatcagggtctggaagaattttgctgatttggaaaagaacccagagatggtgaccgcgtggcaggggctctcggacatgctgccaacgacgcgaggagctgggatggtggtggactgggagcaggagaccggcctcctcatgagctcaggagacgtgcggatcgtccggatctgggacacagaccgtgagatgaaggtgcaggacatccctacgggcgcagacagctgtgtgacgagtctgtcctgtgattcccaccgctcactcatcgtggctggcctcggtgacggctccatccgcgtctacgacagaaggatggcactcagcgaatgccgcgtcatgacgtaccgggagcacacagcctgggtggtgaaggcctccctgcagaagcgtcccgacggccacatcgtgagtgtgagcgtcaatggagatgtgcgcatctttgatccccggatgcctgagtcggtaaatgtgcttcagatcgtgaaggggctgacggccctggacatccacccccaggcggacctgatcgcatgtggctccgtcaatcagttcaccgccatctacaacagcagcggagagctcatcaacaacatcaagtactacgacggcttcatgggccagcgggtcggcgccatcagctgcctggccttccacccgcactggcctcacctggccgtgggaagcaacgactactacatctccgtgtactcggtggagaagcgtgtcagatagcggcgtgacccgggcccaccaggccacggccgcctgctgtacatagtgaagctgtcactcgccggggcacggggcgtcggctgctgcggccccgcagtgtgaacgttggctgctgccttagctgctgatgacggcaggagggccctgctactcgcttttgtctgtcttcgctgtcgtgtctggaatgtcagggaaggggagggctcgggttgacggtggcttcccactgagcaccagcatccaggtgcacccccgcggccacggcgcctctgtccctctcctgttctgtgtttctctgagacgctgaaaggggaaacacctcactttatttccatgtaatcagagcattagctgcagaaaaaccccccgacagagccctggcggagaggcaggcgctggggctcctacgggtccctggggcagctgtccccatcaggccaagagcgagcgagaggcgctgccccagccaggcccaccacctctcacagtcagtgcacgcaagcagggacatttcctagccagctgggggacactggaaattcgggaaaccaagagagaggaagaaggagacgcccctccaactggcgggtgtgaaggaagccgcccaggggtccgggctgtccttggccgctggcagcatcactgagcaggaagcgcacagcccaccctccccgcacctccaggtctctggactccagttttggcccctctcacacagagctgtcagcaggggccgctgtggcggtgcacaggggaggcaggtccttggcgaggtagcccctgccttaatccacggggctcctttccctccgaagggctgctcttccccacaggcgcggggacagcagcccgacctgtggtctccatgcctgtgccctcacacaggtgtagcacacgcatgtgcagatggcaccacggccggcacctgggggcacacacatgcaggcggcgtggtctccctgctctgtccccacacgttcctcacatacaggcaagaggcactgccgggtcccggacggctccgggtgacaccagccccgtctccagccttgagccgcccatgctgatgcgacctcggctgacagctgggcctgtggtgcagacaggagctgtgtggacagtcccgcccaggaggggccgcagggcgtgtatgagcagttttgcaaacagaacacaaccacaatgatggtattttgaaaagtgttctttccgtgttcgtcgggaatcaggattattgagaggtgaaggagccaggtggcttcattctggcggtgagaggcccacgaccacgggagtgagagctggtgtggcgaggcccggctctcctgcggtgtggctggtggcctgccgtggccaagagcatcttctgggtggatggaaccctgcctggtcacatttggccagagacacacctggccctcagggggctgagctggagactgagctggggctggccgggacgtgacaaggcaggacagaggcggcccctccgctgctcctttttggaatgcgagctcccaccagaagaaggttccggcacgaatcccatccccacgtctgggccgagaaagcagcccgggtccggaaggtgtagagagtcccggcctcactcagctcacagggcgtgccaggcggcaacaccagaatcttccagaagcccagctccacccgcacacgcagcttcccatccagtccttcaactcaattcttacccaacacgcgtttctgtttgttttgagacaaaatcaccacctgtcaaaaggcaggtggctccagaggggtcaagacccccccccgcccccgctccaccctggagcccacccccatgggcaccgcgtgccgcctgcacgtgggctgtcttcacaggtctgatgtgaaaattcaatcacgacgttaaccggctcgagagagcgccggcctagaggctcattatctatttattttaccaaacgcgaattgagacggactttgacaaaacacgaaatggtaatgtgaagctaagagcagagagtgaccaacagtaaacaacacgcgcaga

SEQ ID NO: 575 - DNA, 12874 bp, synthetic construct
  source 1..12874: mol_type = other DNA
gtggaaactcatggaactgttcctccagattaacacttcaggggttatggaagctggaggaagctgagcttttactacatcttttgggggttatttatccattcacctactgaaggacatcttggttgcttccaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 576 - DNA, 1123 bp, synthetic construct
  source 1..1123: mol_type = other DNA
agcggggggttcccgcccgcgcctctccctccacacctccccgcaagcagagggagccggctccggccttggccagcccagagacaggctcccacagtgcagcggcgggctgaagggctcctcaagcactgccagagtggacaccgaggccgaggagacgccaagagcgatgaaacaagcttctggttcaagagtttcttcaggttccactacgggctgttcttctacaggtgcgccgatgtccacagtgctaccttctgatgagctactgctttcattcagtttctgctgacttaatatgtgagctgacagaatgcagagaccacgttgtatatgaagcatccactttgccttgtacaccagggcattcaataaccacttaataactacaaccctgatgatccgattcactactaaagtcttccgtgtttaaattttcaaagtcagattctcctacagcaattggtacagtcacagtaagactggggttgtttatgaatgacatgtaatcactttcatcaataatgtatttttcaacactgctgccagttcctataccacttgtagttccatttacatctttaagatagtcaagatctttcccaatttctgctgtatgattggacatacaactgtctttcttgttgtttagatcatcaagtggtttaatttcatctaaaatcttttgtttcctaatgaaggactgttgaataaattcatatatttttcttttcacataagctactcctttgtgcatcctatccacagcaatttggagattattcatttcattatcatcatcagtggctgcaaggttgtctgcactaaatgagctcagaagcaaggccagaaagagattcaggaccttaaaaacaacaaaaacatgattataattttacaccaatgtagggaagagcagattacaatcacttattctttcttttaagtgtggaaaaaactctaagttctaaaacttgatgagaaggaaacaccacagcatagtgattagaagatgggtgatctgaatttgtgactggctcaatagcacatccttggacaaagacatgatttctgttgctctcaagttctcccattcgtaaagtgaaattgaatgagctaatct

SEQ ID NO: 577 - DNA, 26 bp, synthetic construct
  source 1..26: mol_type = other DNA
  modified_base order(1,3): mod_base = gm
  modified_base order(2,20): mod_base = OTHER; note = 2'-O-Methyladenosine
  modified_base order(8,15): mod_base = um
  modified_base order(11,24,25,26): mod_base = cm
gagccagtcacaaattcagatcaccc

SEQ ID NO: 578 - DNA, 21 bp, synthetic construct
  source 1..21: mol_type = other DNA
gtggtataggaactggcagca

SEQ ID NO: 579 - DNA, 21 bp, synthetic construct
  source 1..21: mol_type = other DNA
  modified_base order(1,3,10,19): mod_base = gm
  modified_base order(2,7): mod_base = um
  modified_base order(5,13,17,20): mod_base = cm
  modified_base 21: mod_base = OTHER; note = 2'-O-methyladenosine
gtggcatagggacgggcagca

SEQ ID NO: 580 - DNA, 21 bp, synthetic construct
  source 1..21: mol_type = other DNA
gtggacaggatgcacaaagga

SEQ ID NO: 581 - DNA, 18 bp, synthetic construct
  source 1..18: mol_type = other DNA
aaggattaaaggtagcaa

SEQ ID NO: 582 - DNA, 18 bp, synthetic construct
  source 1..18: mol_type = other DNA
cataagtcacagtgcaag

SEQ ID NO: 583 - DNA, 20 bp, synthetic construct
  source 1..20: mol_type = other DNA; note = Hu-SCN1A forward primer
tgggttactcagaacttgga

SEQ ID NO: 584 - DNA, 18 bp, synthetic construct
  source 1..18: mol_type = other DNA; note = Hu-SCN1A reverse primer
gcattcacaaccaccctc

SEQ ID NO: 585 - DNA, 32 bp, synthetic construct
  source 1..32: mol_type = other DNA; note = /56-FAM/ZEN/3IABkFQ PCR Probe
caaatctctcaggacactaagagctctgagac

SEQ ID NO: 586 - DNA, 23 bp, synthetic construct
  source 1..23: mol_type = other DNA; note = C9orf72 Intron1 forward primer
ggtcagagaaatgagagggaaag

SEQ ID NO: 587 - DNA, 18 bp, synthetic construct
  source 1..18: mol_type = other DNA; note = C9orf72 Intron1 reverse primer
cgagtgggtgagtgagga

SEQ ID NO: 588 - DNA, 24 bp, synthetic construct
  source 1..24: mol_type = other DNA; note = C9orf72 Intron 1 probe (FAM)
aaatgcgtcgagctctgaggagag